(19) United States
(12) Patent Application Publication
(10) Pub. No.: US 2026/0234655 A1
(43) Pub. Date: Aug. 13, 2026

(54) Nuclear Targeted DNA Delivery and Compositions for Use in Practicing the Same

(71) Applicant: Seawolf Therapeutics, Inc., San Diego, CA (US)

(72) Inventors: Rob BURKE, Carlsbad, CA (US); Brian LAMB, San Diego, CA (US); Niv SCOTT, San Diego, CA (US); Marco PROIETTO, San Diego, CA (US); Sataree KHUANSUWAN, La Jolla, CA (US); Samuel Wyman BEPPLER, San Diego, CA (US); Oliver John DANSEREAU, La Jolla, CA (US); Elizabeth ALCAMO, San Mateo, CA (US)

(21) Appl. No.: 19/124,043

(22) PCT Filed: Oct. 25, 2023
(86) PCT No.: PCT/US2023/035932

§ 371 Date: Apr. 24, 2025

Related U.S. Application Data
(60) Provisional application No. 63/419,890, filed on Oct. 27, 2022.
(60) Provisional application No. 63/430,950, filed on Dec. 7, 2022.
(60) Provisional application No. 63/454,505, filed on Mar. 24, 2023.

(51) Int. Cl. C12N 15/85; A61K 9/00; A61K 9/51; A61K 31/7105; A61K 31/711; A61K 48/00; C12N 15/11; C12N 15/88

(52) U.S. Cl. CPC C12N 15/85; A61K 9/0019; A61K 9/5123; A61K 31/7105; A61K 31/711; A61K 48/0008; A61K 48/005; C12N 15/11; C12N 15/88; C12N 2800/107; C12N 2830/34

(57) ABSTRACT

Methods of nuclear targeted DNA delivery are provided. Aspects of the methods include contacting a cell with: (a) a nuclear targeted deoxyribonucleic acid (NTDNA) that includes a DNA nuclear targeting sequence (DTS) and a cargo nucleic acid heterologous to the DTS. Also provided are compositions for use in practicing methods of the invention.

### CROSS REFERENCE TO RELATED APPLICATIONS

This application is a National Stage Entry of International Application No, PCT/US2023/035932, filed Oct. 25, 2023, which application claims the benefit of U.S. Provisional Application No. 63/419,890, filed Oct. 27, 2022; U.S. Provisional Application No. 63/430,950, filed Dec. 7, 2022; and U.S. Provisional Application No. 63/454,505, filed Mar. 24, 2023, the entirety of each of which is hereby incorporated by reference.

### INCORPORATION BY REFERENCE OF SEQUENCE LISTING PROVIDED AS A SEQUENCE LISTING XML FILE

A Sequence Listing is provided herewith as a Sequence Listing XML, “SWTX-001WO_SEQLIST”, created on Apr. 28, 2025, and having a size of 413,018 bytes. The contents of the Sequence Listing XML are incorporated herein by reference in their entirety.

### INTRODUCTION

There are many instances in which nuclear delivery of a nucleic acid is desired, where such instances include research, diagnostic and therapeutic applications. An example of such a therapeutic application is gene therapy. In the field of gene therapy, viral vectors, such as vectors based on the virus AAV, are commonly employed to deliver genes to the nucleus. However, AAV's genome is limited in size, so any gene greater than 4.7 kB will not be suitable for use with AAV vectors, which limits the utility of such vectors for many indications. In addition, viral vectors, such as AAV, induce an antibody response, such that they can only be delivered once, which is not suitable for some indications, such as indications in the liver where the cells are slowly dividing and will lose the transduced genome, thereby requiring redosing. Furthermore, viral vector such as AAV are toxic at the doses that are required for a therapeutic benefit in some indications. Accordingly, what is needed is a new delivery vehicle for delivering nucleic acids, such as DNA, to cells, particularly in patients in need of gene therapy but also in vitro during research.

That next generation delivery vehicle is a nanoparticle. Of particular interest are lipid nanoparticles, given how LNPs have been de-risked by being used in Onpattro and the Covid vaccine. The problem is that, in contrast to AAV's ability to carry its DNA cargo all the way into the nucleus, nanoparticles only deliver their cargo to the cytoplasm. In the case of dividing cells, delivery to the cytosol may be sufficient as the nuclear envelop dissolves in the course of replication and new DNA gets captured as it re-forms. However, it is a problem for nondividing or slowly dividing cells. What is needed to make LNPs be a successful delivery vehicle in such cells is a mechanism to drive the transport of the DNA from the cytoplasm into the nucleus. The inventors have identified methods and compositions to satisfy the above need.

### SUMMARY

Methods of nuclear targeted DNA delivery are provided. Aspects of the methods include contacting a cell with a nuclear targeted deoxyribonucleic acid (NTDNA) that includes a DNA nuclear targeting sequence (DTS) and a cargo nucleic acid heterologous to the DTS. Also provided are compositions for use in practicing methods of the invention.

### BRIEF DESCRIPTION OF THE FIGURES

FIG. 1 demonstrates the premise behind using DTSs to increase nuclear uptake. DNA targeting sequences (DTSs) encoded on a DNA cargo are recognized by cytosolic DNA binding proteins such as transcription factors that are present in the cytoplasm and can bind to and translocate DNA cargo to the nucleus to facilitate gene expression.

FIG. 2 illustrates two library designs for use in screening DTSs. DTS libraries are designed with nuclear targeting factor binding sites (NTFBSs, or more simply, TFBSs) in a tandem array with spacer sequences of varying lengths. In some instances (panel a), a single TFBS is arrayed to understand the contribution of that particular transcription factor to DNA translocation and expression. In other instances (panel b), TFBSs from different transcription factors are arrayed in various combinations to test the abilities of the cognate transcription factors to synergize with one another. The putative DTS is placed on the cargo upstream of the promoter of an expression cassette (shown here), downstream of the poly A tail of the expression cassette, or within an intron of the expression sequence. If desired, for example to screen DTSs in pools, a unique molecular identifier (UMI), i.e., expression barcode, may be included on the cargo, for example within the expression cassette. DNA is then introduced to cells either in vitro, for example in primary human hepatocytes or HepG2 cells, or in vivo, for example in mice or NHPs. Cells are collected post-transfection and the efficiency of DNA delivery is assessed by DNA copy number within cells, RNA expression levels, or protein quantification analysis. TFBSs that are selected for screening include those that are the binding sites for transcription factors that are highly expressed in hepatocytes and transcription factors that are leveraged by viruses.

FIG. 3 illustrates the utility of implementing multiple in vitro experimental designs to identify DTSs for in vivo assessment. (a) Screening DTS libraries in nondividing primary human hepatocytes enables the identification of hits (open circles) by both quantitative live-cell imaging and FACS. (b) Screening DTS libraries in serum-starved, growth arrested cells (HepG2 shown here) reproduces data from HepG2 cells grown in the presence of serum but with a greater dynamic range, allowing one to identify hits (in vitro) with greater sensitivity (open circles).

FIG. 4 shows that DTSs that comprise the binding site for NF-κB or that are derived from the enhancer element of SV40 mediate robust nuclear translocation of NTDNA and expression of genes encoded on that NTDNA in nondividing primary human hepatocytes.

FIG. 5 illustrates the identification of several novel and robust DTSs through one-by-one arrayed screening of a combinatorial DTS library. Plasmid NTDNAs comprising combinatorial DTSs and a GFP reporter expression cassette are delivered to growth-arrested HepG2 cells and GFP expression is assessed over time. Statistics and hierarchical clustering methods for analysis of kinetics experiments (panel A), along with endpoint analysis of expression at the final time point at 24 hours post-transfection (panel B), both reveal large increases in gene expression with novel DTSs that are equivalent to or better than the NFKB DTS. DTS.203: CREB1, PPARA, ONECUT1, HNF4A, PPARA. DTS.233: HNF1A, NR113, PPARA, HNF1A, PPARA. DTS.276: FOXA1, HNF4A, HNF1A, HNF4A, HNF1A.

FIG. 6 illustrates the strong correlation between the DTSs identified through a pooled DTS screening approach, which relies on detecting changes in mRNA abundance (x-axis), and a one-by-one arrayed screening approach, which relies on detecting changes in protein abundance (y-axis). The study was performed by transfecting pDNAs containing DTSs into growth-arrested HepG2 cells. Assessment of the pooled DTS screen was performed at the mRNA level using RNA-seq analysis to quantify each UMI, and assessment of the arrayed DTS screen was performed by measuring GFP fluorescence intensity.

FIGS. 7A-7B provide data on the activity of DTSs in mouse liver. Plasmids containing DTSs and barcodes embedded in their expression cassettes were formulated together as a single formulation and administered by bolus iv injection into mice. mRNA levels were quantified using RNA-seq analysis of each barcode in the pool to identify DTSs that drive increased gene expression. (A) RNA abundance of expression cassettes associated with different DTSs in mouse liver 1 day post-LNP dose. (B) RNA abundance of DTSs in mouse liver 4 days post-LNP dose.

FIG. 8 provides the results of an analysis of all DTS datasets by machine learning to identify the transcription factors of greatest importance in DTS activity. A gradient boosted decision tree machine learning algorithm was independently applied to each individual dataset, and then the most important features were identified by the % improvement they provided to the algorithm's predictions. The same top 3 transcription factors were identified in each dataset, indicating their importance within DTS elements.

FIG. 9 shows how inducible DTSs (iDTSs) can further increase gene expression when a cognate binding protein is induced to translocate to the nucleus following stimulation with an inducing agent. In this study, dexamethasone—a known activator of glucocorticoid receptor (GR) relocalization to the nucleus—is used as an inducing agent. Plasmid NTDNA comprising a GR-responsive DTS and a GFP reporter expression cassette is transfected into growth-arrested HepG2 cells, the cells are contacted with various amounts of dexamethasone, and GFP expression is assessed.

FIG. 10 provides data on several iDTSs that promote significant translocation and expression of their cargos, as identified by screening combinatorial libraries. Plasmid NTDNAs comprising combinatorial DTSs and a GFP reporter expression cassette are delivered to growth-arrested HepG2 cells and GFP expression assessed 20 hours later. Note that while the activator does not induce activity of NTDNAs having binding sequences for constitutively active transcription factors, it does induce translocation of NTDNAs comprising DTSs having binding sequences for inducible transcription factors. iDTS-1-1: NR3C1, NR3C1, NR3C1, NR3C1, NR3C1. iDTS-1-2: HNF4A, NR113, NR3C1, NR3C1, HNF4A. iDTS-1-3: NR3C1, CREB3L3, NR3C1, PPARA, POU2F1. iDTS-1-4: NR3C1, CEBPA, CEBPA, CEBPA, NR3C1.

FIGS. 11A-11F provide the schematics and NLS/NES domains for various TetR proteins that were tested as mRNA/DNA coformulations with NTDNAs comprising a TetO sequence as a DTS. (A) Schematics of TetR engineered proteins. (B) TetR set 1.X. (C) TetR set 2.X. (D) TetR set 3.X. (E) TetR set 4.X (F) TetR set 5.X.

FIG. 12 provides data on the use of mRNA expressing a nuclear targeting factor (NTF) to bind DNA and facilitate translocation into the nucleus. Plasmid NTDNAs comprising DTSs with TetR binding sites, e.g., the tetracycline response element (TRE) that contains 7 copies of the TetO sequence, are delivered along with mRNA expressing TetR-NLS fusion proteins to growth-arrested HepG2 cells. Forty-eight hours after transfection, GFP expression was assessed by measuring GFP fluorescence intensity and toxicity was assessed by counting the rounded (dead) cells. (A) Illustrations of DNA payloads with TetR binding sites and mRNAs designed to produce different TetR fusion proteins, with nuclear localization signals (NLSs) and nuclear export signals (NESs) placed at the N-terminus, C-terminus, or both. (B) GFP intensity and rounded cell counts were determined 48 h post-transfection.

FIGS. 13A-13E provide additional data on the use of mRNA expressing a nuclear targeting factor (NTF) to bind DNA and facilitate translocation into the nucleus. Plasmid NTDNAs comprising DTSs with TetR binding sites (e.g., TetO sequences) are delivered along with mRNA expressing TetR-NLS fusion proteins to growth-arrested HepG2 cells. GFP expression was assessed by measuring GFP fluorescence intensity for 21 h post-transfection. (A) GFP intensity was measured 21 h after co-transfection of doggybone DNA (dbDNA) and TetR mRNA. (B) GFP intensity was measured 21 h after co-transfection of plasmid DNA (pDNA) and TetR mRNA. (C) Fold change of the increase in GFP intensity was calculated relative to the DNA Alone (e.g., the DNA containing TetO sequences but without addition of TetR mRNA) and is presented for both pDNA and dbDNA. (D) Time course of GFP intensity up to 21 h after co-transfection of plasmid DNA (pDNA) and TetR mRNA. (E) Time course of GFP intensity up to 21 h after co-transfection of doggybone DNA (dbDNA) and TetR mRNA.

FIGS. 14A-14K provide additional data on the use of mRNA expressing a nuclear targeting factor (NTF) to bind DNA and facilitate translocation into the nucleus. Plasmid NTDNAs comprising DTSs with TetR binding sites (e.g., TetO sequences) are delivered along with mRNA expressing TetR-NLS fusion proteins to growth-arrested HepG2 cells. GFP expression was assessed by measuring GFP fluorescence intensity for 24 h post-transfection. (A) GFP intensity was measured 18 h after co-transfection of dbDNA containing TetO sequences and TetR mRNA. (B) GFP intensity was measured 18 h after co-transfection of dbDNA lacking TetO sequences and TetR mRNA. (C) Time course of GFP intensity up to 24 h after co-transfection of 17.5 ng of dbDNA containing TetO sequences and 35 ng of TetR mRNA, compared to several negative controls. (D) GFP intensity 24 h after co-transfection of 17.5 ng of dbDNA containing TetO sequences and 35 ng of TetR mRNA, compared to several negative controls. (E) Fold change of the increase in GFP intensity was calculated relative to the dbDNA Alone (e.g., the dbDNA containing TetO sequences but without addition of TetR mRNA) at 9 h post-transfection. (F) Fold change of the increase in GFP intensity was calculated relative to the dbDNA Alone (e.g., the dbDNA containing TetO sequences but without addition of TetR mRNA) at 18 h post-transfection. (G) Fold change of the increase in GFP intensity was calculated relative to the dbDNA Alone (e.g., the dbDNA containing TetO sequences but without addition of TetR mRNA) at 24 h post-transfection. (H) Fold change of the increase in GFP intensity was calculated relative to the dbDNA Alone (e.g., the dbDNA containing TetO sequences but without addition of TetR mRNA) at 24 h post-transfection. (I) Fold change of the increase in GFP intensity was calculated relative to the pDNA Alone (e.g., the pDNA containing TetO sequences but without addition of TetR mRNA) at 24 h post-transfection. (J) Toxicity was assessed by counting the number of round (dead) cells at 18 h after the co-transfection of dbDNA+TetR mRNA. (K) Toxicity was assessed by counting the number of round (dead) cells at 18 h after the co-transfection of pDNA+TetR mRNA.

FIGS. 15A-15B illustrate the binding of and translocation of NTDNA to the nucleus by several different species of nuclear targeting factors (NTFs). Plasmid NTDNAs comprising DTSs with NTF binding sites are co-delivered with mRNA expressing NTF proteins. Each NTF protein comprises a DNA-Binding Domain (DBD) and at least one NLS. The DBD of each NTF mediates binding to the DTS encoded on the NTDNA, and the NLS facilitates nuclear translocation of the NTF-NTDNA complex. (A) GFP expression from the NTDNA was assessed by measuring GFP fluorescence intensity 18 h after co-transfection of plasmid NTDNA and varying amounts of NTF mRNA into growth-arrested HepG2 cells. (B) GFP expression from the NTDNA was assessed by measuring GFP fluorescence intensity 18 h after co-transfection of plasmid NTDNA and varying amounts of NTF mRNA into primary human hepatocytes (PHH).

FIGS. 16A-16B illustrate the changes in gene expression that are observed for two different NTFs, Gal4 and TetR as a result of changing the number of NTF binding sites encoded into the DTS. Several plasmid NTDNAs were constructed with DTSs comprising 1-7 copies of the TetO sequence (the binding site for TetR), or 1, 2, 3, 4, 5, 6, 7 or 10 copies of the upstream activation sequence (UAS) (the binding site for Gal4). (A) GFP expression from the NTDNA comprising TetO binding sites was assessed by measuring GFP fluorescence intensity 24 h after transfection of plasmid NTDNA alone or co-transfection of plasmid NTDNA and varying amounts of TetR mRNA into growth-arrested HepG2 cells. (B) GFP expression from the NTDNA comprising UAS binding sites was assessed by measuring GFP fluorescence intensity 12 h after transfection of plasmid NTDNA alone or co-transfection of plasmid NTDNA and varying amounts of Gal4 mRNA into growth-arrested HepG2 cells.

FIG. 17 illustrates that several strategies can be employed to co-administer mRNA expressing NTFs and NTDNA payloads so as to achieve greater gene expression from the NTDNA than NTDNA administered alone. LNPs or PBS were administered to mice by i.v. bolus and human EPO levels were measured 3 d post-dose. These LNP formulations utilized mRNA expressing TetR and DNA expressing EPO and comprising a DTS with 7xTetO repeats. Circles: NTDNA-LNPs alone; squares: administration of LNPs comprising NTDNA and mRNA encoding the NTF; upward triangles: co-dosing of LNPs comprising NTDNA and LNPs comprising mRNA encoding the NTF; downward triangle: dosing first with LNPs comprising mRNA encoding the NTF, followed by dosing with LNPs comprising the NTDNA.

FIGS. 18A-18F illustrate that nuclear targeting factors (NTFs) are able to facilitate nuclear translocation of NTDNA in vivo. In all instances, mice were dosed by i.v. bolus injection and gene product (FIX) from the NTDNA's expression cassette was measured for 28 days: (A) LNPs formulated with DNA comprising 7xTetO DTS sequence and an expression cassette encoding human FIX+/−mRNA encoding a TetR-NLS fusion protein (v1); (B) LNPs co-formulated with DNA comprising 7xTetO DTS sequence and an expression cassette encoding human FIX, +/−mRNA encoding a TetR-NLS fusion protein (v2); (C) LNPs formulated with DNA comprising a 5xUAS DTS and an expression cassette encoding human FIX+/−mRNA encoding a Gal4-NLS fusion protein; (D) LNPs formulated with DNA comprising a 7xArc DTS and an expression cassette encoding human FIX+/−mRNA encoding an Arc-NLS fusion protein; (E) LNPs formulated with DNA comprising a 7xMnt DTS and an expression cassette encoding human (FIX+/−mRNA encoding a Mnt-NLS fusion protein; (F) LNPs formulated with DNA comprising a 7xBac434 DTS and an expression cassette encoding human FIX+/−mRNA encoding a Bac434-NLS fusion protein. Filled circles: LNPs coformulated with DNA and RNA; open squares: LNPs formulated with DNA alone; closed triangles: PBS.

### INCORPORATION BY REFERENCE

All publications and patent applications mentioned in this specification are herein incorporated by reference to the same extent as if each individual publication or patent application was specifically and individually indicated to be incorporated by reference.

### Definitions

The terms “polypeptide,” “polypeptide sequence,” “peptide,” “peptide sequence,” “protein,” “protein sequence” and “amino acid sequence” are used interchangeably herein to designate a linear series of amino acid residues connected one to the other by peptide bonds, which series may include proteins, polypeptides, oligopeptides, peptides, and fragments thereof. The protein may be made up of naturally occurring amino acids and/or synthetic (e.g., modified or non-naturally occurring) amino acids. Thus “amino acid”, or “peptide residue”, as used herein means both naturally occurring and synthetic amino acids. The terms “polypeptide”, “peptide”, and “protein” includes fusion proteins, including, but not limited to, fusion proteins with a heterologous amino acid sequence, fusions with heterologous and homologous leader sequences, with or without N-terminal methionine residues; immunologically tagged proteins; fusion proteins with detectable fusion partners, e.g., fusion proteins including as a fusion partner a fluorescent protein, beta-galactosidase, luciferase, and the like. Furthermore, it should be noted that a dash at the beginning or end of an amino acid sequence indicates either a peptide bond to a further sequence of one or more amino acid residues or a covalent bond to a carboxyl or hydroxyl end group. However, the absence of a dash should not be taken to mean that such peptide bond or covalent bond to a carboxyl or hydroxyl end group is not present, as it is conventional in representation of amino acid sequences to omit such.

The term “polynucleotide,” “polynucleotide sequence,” “oligonucleotide,” “oligonucleotide sequence,” “oligomer,” “oligo,” “nucleic acid sequence” or “nucleotide sequence” used interchangeably herein, refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer having purine and pyrimidine bases or other natural, chemically or biochemically modified, non-natural, or derivatized nucleotide bases.

The terms “derivative” and “variant” refer without limitation to any compound such as nucleic acid or protein that has a structure or sequence derived from the compounds disclosed herein and whose structure or sequence is sufficiently similar to those disclosed herein such that it has the same or similar activities and utilities or, based upon such similarity, would be expected by one skilled in the art to exhibit the same or similar activities and utilities as the referenced compounds, thereby also interchangeably referred to “functionally equivalent” or as “functional equivalents.” Modifications to obtain “derivatives” or “variants” may include, for example, addition, deletion and/or substitution of one or more of the nucleic acids or amino acid residues.

The functional equivalent or fragment of the functional equivalent, in the context of a protein, may have one or more conservative amino acid substitutions. The term “conservative amino acid substitution” refers to substitution of an amino acid for another amino acid that has similar properties as the original amino acid. The groups of conservative amino acids are as follows:

| Group | Name of the amino acids |
|---|---|
| Aliphatic | Gly, Ala, Val, Leu, Ile |
| Hydroxyl or Sulfhydryl/Selenium-containing | Ser, Cys, Thr, Met |
| Cyclic | Pro |
| Aromatic | Phe, Tyr, Trp |
| Basic | His, Lys, Arg |
| Acidic and their Amide | Asp, Glu, Asn, Gln |

Conservative substitutions may be introduced in any position of a preferred predetermined peptide or fragment thereof. It may however also be desirable to introduce non-conservative substitutions, particularly, but not limited to, a non-conservative substitution in any one or more positions. A non-conservative substitution leading to the formation of a functionally equivalent fragment of the peptide would for example differ substantially in polarity, in electric charge, and/or in steric bulk while maintaining the functionality of the derivative or variant fragment.

“Percentage of sequence identity” is determined by comparing two optimally aligned sequences over a comparison window, wherein the portion of the polynucleotide or polypeptide sequence in the comparison window may have additions or deletions (i.e., gaps) as compared to the reference sequence (which does not have additions or deletions) for optimal alignment of the two sequences. In some cases the percentage can be calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The terms “identical” or percent “identity” in the context of two or more nucleic acid or polypeptide sequences, refer to two or more sequences or subsequences that are the same or have a specified percentage of amino acid residues or nucleotides that are the same (e.g., 60%, 65%, 70%, 75%, 80%, 85%, 90%, 95%, 98%, or 99% identity over a specified region, e.g., the entire polypeptide sequences or individual domains of the polypeptides), when compared and aligned for maximum correspondence over a comparison window or designated region as measured using one of the following sequence comparison algorithms or by manual alignment and visual inspection. Such sequences are then said to be “substantially identical.” This definition also refers to the complement of a test sequence.

The term “complementary” or “substantially complementary,” interchangeably used herein, means that a nucleic acid (e.g. DNA or RNA) has a sequence of nucleotides that enables it to non-covalently bind, i.e. form Watson-Crick base pairs and/or G/U base pairs to another nucleic acid in a sequence-specific, antiparallel, manner (i.e., a nucleic acid specifically binds to a complementary nucleic acid). As is known in the art, standard Watson-Crick base-pairing includes: adenine (A) pairing with thymidine (T), adenine (A) pairing with uracil (U), and guanine (G) pairing with cytosine (C).

A DNA sequence that “encodes” a particular RNA is a DNA nucleic acid sequence that is transcribed into RNA when placed under the control of appropriate regulatory sequences. A DNA polynucleotide may encode an RNA (mRNA) that is translated into protein, or a DNA polynucleotide may encode an RNA that is not translated into protein (e.g. tRNA, rRNA, or a guide RNA; also called “non-coding” RNA or “ncRNA”). A protein coding sequence or a sequence that encodes a particular protein or polypeptide, is a nucleic acid sequence that is transcribed into mRNA (in the case of DNA) and is translated (in the case of mRNA) into a polypeptide in vitro or in vivo when placed under the control of appropriate regulatory sequences.

As used herein, “codon” refers to a sequence of three nucleotides that together form a unit of genetic code in a DNA or RNA molecule. As used herein the term “codon degeneracy” refers to the nature in the genetic code permitting variation of the nucleotide sequence without affecting the amino acid sequence of an encoded polypeptide. Nucleotides are typically referred to as the following: A (adenine), G (guanine), C (cytosine), T (thymine), U (uracil), N (=A or C or G or T/U), B (=C or G or T/U), D (=A or G or T/U), H (=A or C or T/U), K (=G or T/U), M (=A or C), R (=A or G), S (=C or G), V (=A or C or G), W (=A or T/U), (=C or T/U). When describing the RNA herein, it will be appreciated by the ordinarily skilled artisan that in the course of transcription, a DNA sequence will be transcribed into an mRNA sequence having the same nucleotide sequence but for thymine, which will be encoded as uracil in the RNA. Accordingly, the ordinarily skilled artisan will readily be able to convert the DNA sequences disclosed herein or known in the art to mRNA sequences.

The term “codon-optimized” or “codon optimization” refers to genes or coding regions of nucleic acid molecules for transformation of various hosts, refers to the alteration of codons in the gene or coding regions of the nucleic acid molecules to reflect the typical codon usage of the host organism without altering the polypeptide encoded by the DNA. Such optimization includes replacing at least one, or more than one, or a significant number, of codons with one or more codons that are more frequently used in the genes of that organism. Codon usage tables are readily available, for example, at the “Codon Usage Database” available at www.kazusa.or.jp/codon/(visited Mar. 20, 2008). By utilizing the knowledge on codon usage or codon preference in each organism, one of ordinary skill in the art can apply the frequencies to any given polypeptide sequence, and produce a nucleic acid fragment of a codon-optimized coding region which encodes the polypeptide, but which uses codons optimal for a given species. Codon-optimized coding regions can be designed by various methods known to those skilled in the art.

The term “recombinant” or “engineered” when used with reference, for example, to a cell, a nucleic acid, a protein, or a vector, indicates that the cell, nucleic acid, protein or vector has been modified by or is the result of laboratory methods. Thus, for example, recombinant or engineered proteins include proteins produced by laboratory methods. Recombinant or engineered proteins can include amino acid residues not found within the native (non-recombinant or wild-type) form of the protein or can be include amino acid residues that have been modified, e.g., labeled. The term can include any modifications to the peptide, protein, or nucleic acid sequence. Such modifications may include the following: any chemical modifications of the peptide, protein or nucleic acid sequence, including of one or more amino acids, deoxyribonucleotides, or ribonucleotides; addition, deletion, and/or substitution of one or more of amino acids in the peptide or protein; and addition, deletion, and/or substitution of one or more of nucleic acids in the nucleic acid sequence.

The term “genomic DNA” or “genomic sequence” refers to the DNA of a genome of an organism including, but not limited to, the DNA of the genome of a bacterium, fungus, archea, plant or animal.

As used herein, “transgene,” “exogenous gene” or “exogenous sequence,” in the context of nucleic acid, refers to a nucleic acid sequence or gene that was not present in the genome of a cell but artificially introduced into the genome, e.g., via genome-edition.

As used herein, “endogenous gene” or “endogenous sequence,” in the context of nucleic acid, refers to a nucleic acid sequence or gene that is naturally present in the genome of a cell, without being introduced via any artificial means.

The term “expression cassette” refers to a DNA coding sequence operably linked to a promoter. “Operably linked” refers to a juxtaposition wherein the components so described are in a relationship permitting them to function in their intended manner. For instance, a promoter is operably linked to a coding sequence if the promoter affects its transcription or expression. The terms “recombinant expression vector,” or “DNA construct” are used interchangeably herein to refer to a DNA molecule having a vector and at least one insert. Recombinant expression vectors are usually generated for the purpose of expressing and/or propagating the insert(s), or for the construction of other recombinant nucleotide sequences. The nucleic acid(s) may or may not be operably linked to a promoter sequence and may or may not be operably linked to DNA regulatory sequences.

The term “operably linked” means that the nucleotide sequence of interest is linked to regulatory sequence(s) in a manner that allows for expression of the nucleotide sequence. The term “regulatory sequence” is intended to include, for example, promoters, enhancers and other expression control elements (e.g., polyadenylation signals). Such regulatory sequences are well known in the art and are described, for example, in Goeddel; Gene Expression Technology: Methods in Enzymology 185, Academic Press, San Diego, Calif. (1990). Regulatory sequences include those that direct constitutive expression of a nucleotide sequence in many types of host cells, and those that direct expression of the nucleotide sequence only in certain host cells (e.g., tissue-specific regulatory sequences). It will be appreciated by those skilled in the art that the design of the expression vector can depend on such factors as the choice of the target cell, the level of expression desired, and the like.

A cell has been “genetically modified” or “transformed” or “transfected” by exogenous DNA, e.g., a recombinant expression vector, when such DNA has been introduced inside the cell. The presence of the exogenous DNA results in permanent or transient genetic change. The transforming DNA may or may not be integrated (covalently linked) into the genome of the cell. The genetically modified (or transformed or transfected) cells that have therapeutic activity, e.g., treating hemophilia A, can be used and referred to as therapeutic cells.

The term “concentration” used in the context of a molecule such as peptide fragment refers to an amount of molecule, e.g., the number of moles of the molecule, present in a given volume of solution.

The terms “individual,” “subject” and “host” are used interchangeably herein and refer to any subject for whom diagnosis, treatment or therapy is desired. In some aspects, the subject is a mammal. In some aspects, the subject is a human being. In some aspects, the subject is a patient. In some aspects, the subject is a human patient. In some aspects, the subject can have or is suspected of having a disorder or health condition associated with a gene-of-interest (GOI). In some aspects, the subject is a human who is diagnosed with a risk of disorder or health condition associated with a GOI at the time of diagnosis or later. In some cases, the diagnosis with a risk of disorder or health condition associated with a GOI can be determined based on the presence of one or more mutations in the endogenous GOI or genomic sequence near the GOI in the genome that may affect the expression of GOI.

The term “treatment” referring to a disease or condition means that at least an amelioration of the symptoms associated with the condition afflicting an individual is achieved, where amelioration is used in a broad sense to refer to at least a reduction in the magnitude of a parameter, e.g., a symptom, associated with the condition (e.g., hemophilia A) being treated. As such, treatment also includes situations where the pathological condition, or at least symptoms associated therewith, are completely inhibited, e.g., prevented from happening, or eliminated entirely such that the host no longer suffers from the condition, or at least the symptoms that characterize the condition. Thus, treatment includes: (i) prevention, that is, reducing the risk of development of clinical symptoms, including causing the clinical symptoms not to develop, e.g., preventing disease progression; (ii) inhibition, that is, arresting the development or further development of clinical symptoms, e.g., mitigating or completely inhibiting an active disease.

The terms “effective amount,” “pharmaceutically effective amount,” or “therapeutically effective amount” as used herein mean a sufficient amount of the composition to provide the desired utility when administered to a subject having a particular condition. The term “therapeutically effective amount” therefore refers to an amount of therapeutic cells or a composition having therapeutic cells that is sufficient to promote a particular effect when administered to a subject in need of treatment. An effective amount would also include an amount sufficient to prevent or delay the development of a symptom of the disease, alter the course of a symptom of the disease (for example but not limited to, slow the progression of a symptom of the disease), or reverse a symptom of the disease. It is understood that for any given case, an appropriate “effective amount” can be determined by one of ordinary skill in the art using routine experimentation,

The term “pharmaceutically acceptable excipient” as used herein refers to any suitable substance that provides a pharmaceutically acceptable carrier, additive or diluent for administration of a compound(s) of interest to a subject. “Pharmaceutically acceptable excipient” can encompass substances referred to as pharmaceutically acceptable diluents, pharmaceutically acceptable additives, and pharmaceutically acceptable carriers.

### DETAILED DESCRIPTION

Methods of nuclear targeted DNA delivery are provided. Aspects of the methods include contacting a target cell with a nuclear targeted deoxyribonucleic acid (NTDNA) that includes a DNA nuclear targeting sequence (DTS) and a cargo nucleic acid heterologous to the DTS. Also provided are compositions for use in practicing methods of the invention.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

While the apparatus and method has or will be described for the sake of grammatical fluidity with functional explanations, it is to be expressly understood that the claims, unless expressly formulated under 35 U.S.C. § 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 U.S.C. § 112 are to be accorded full statutory equivalents under 35 U.S.C. § 112.

### Methods

As summarized above, methods of delivering a cargo nucleic acid sequence into the nucleus of a cell are provided. Methods of the invention provide for transportation of a cargo nucleic acid from the cytosol of a cell into the nucleus of cell. As such, methods of invention may be characterized as methods of transporting a cargo nucleic acid into the nucleus of a cell. Accordingly, the method may be viewed as nuclear targeted DNA delivery methods.

As reviewed above, aspects of embodiments of the invention include contacting the cell with: a nuclear targeted deoxyribonucleic acid (NTDNA) that includes a DNA nuclear targeting sequence (DTS) and a cargo nucleic acid heterologous to the DTS; so as to deliver the cargo nucleic acid sequence to the nucleus of the cell. Therefore, aspects of the invention include methods of DTS-mediated nuclear delivery of DNA.

Methods of the invention typically provide for an improvement, or an enhancement, in delivery and/or expression of DNA to the nucleus over DNA delivery methods that depend on the delivery of DNA that does not comprise a DTS. In some instances, the improvement is a 2-fold increase or more in the amount of DNA in the nucleus relative to the amount of DNA that would be found in the nucleus if that DNA did not include a DTS, for example, a 2-fold, 3-fold, 4-fold, 5-fold, 6-fold, 7-fold, 8-fold, 10-fold, 15-fold, 20-fold, 30-fold, 40-fold, or 50-fold increase in the amount of DNA in the nucleus. This may be detected as an increase in the expression of the cargo nucleic acid heterologous to the DTS, for example, as a 2-fold, 3-fold, 4-fold, 5-fold, 6-fold, 7-fold, 8-fold, 10-fold, 15-fold, 20-fold, 30-fold, 40-fold, 50-fold, 100-fold, or 200-fold increase in RNA or protein encoded by the cargo nucleic acid over that expressed in a cell that has not been contacted with DNA or a cell that has been contacted with a DNA that does not include a DTS.

Various aspects of the methods are now reviewed in greater detail.

### Nuclear Targeted Deoxyribonucleic Acids (NTDNAs)

Embodiments of the methods include contacting a cell with a NTDNA. NTDNAs are double stranded deoxyribonucleic acids that may vary in length, ranging in some instances from 15nt to 15,000 nt, such as 100 to 10,000 nt and including 100 to 5000nt. NTDNAs employed in methods of the invention comprise both a DNA nuclear targeting sequence (DTS) and a cargo nucleic acid sequence that is heterologous to the DTS. As such, the NTDNAs include a DTS domain and a cargo nucleic acid domain. Each of these domains is now described further in greater detail.

A DTS refers to a nucleotide sequence that mediates the translocation of a polynucleotide that comprises it into the nucleus of a cell. Without wishing to be bound by theory, it is believed that DTSs leverage the movement of nuclear-acting DNA binding proteins as they move from the cytoplasm into the nucleus. These nuclear-acting DNA binding proteins act like nuclear targeting factors for DNA, binding to sequences on the DNA and dragging the DNA into the nucleus as the DNA binding protein translocates into the nucleus. Accordingly, the nuclear-acting DNA binding proteins that are leveraged are referred to herein as nuclear targeting factors (NTFs), and the DNA sequences to which they bind are referred to herein as nuclear targeting factor binding sites (NTFBSs, or more simply, TFBSs).

In some embodiments, the DTS comprises a TFBS that is bound by a nuclear targeting factor that is active in the target cell. For example, the nuclear targeting factor may be constitutively expressed in the target cell. As another example, the nuclear targeting factor may be typically latent in the cytoplasm of the target cell but become active when the cell is contacted by the NTDNA, for example as part of a cellular response to the NTDNA or the formulation comprising the NTDNA, for example as part of an inflammatory response, e.g. transcription factors that are responsive TLR9, cGAS/STING, AIM2, IFI16, or DDX41 activation, e.g. NF-kB, IRF3, IRF7, and others as known in the art. As another example, the nuclear targeting factor may be provided to the cell, e.g., as a protein or as an mRNA encoding a protein. In examples in which the nuclear acting DNA binding protein is endogenous to the target cell, delivery of the DNA into the nucleus of the cell happens without the need for additional exogenous factors. Accordingly, in some embodiments, methods for delivering NTDNAs to the nucleus of the cell that utilize DTSs that leverage such DNA binding proteins consist essentially of contacting the cell with the NTDNA comprising a DTS. Table 1 provides examples of TFBSs that may be utilized in DTSs and the nuclear acting DNA binding proteins that they would leverage in hepatocytes to achieve nuclear transport of associated cargo DNA without needing to provide additional agents.

TABLE 1
NTFBSs and the DNA binding proteins that they leverage for nuclear translocation

| DNA binding protein | TFBS |
|---|---|
| ATF3 | TGACGTCA (SEQ ID NO: 01) |
| CEBPA v1 | ATTGCGCAAT (SEQ ID NO: 02) |
| CEBPA v2 | ATTGTGCAAT (SEQ ID NO: 03) |
| CEBPA v3 | ATTGCACAAT (SEQ ID NO: 04) |
| CEBPA v4 | ATTACAAAAT (SEQ ID NO: 05) |
| CEBPA v5 | TGTTTGTTAAGGC (SEQ ID NO: 06) |
| CEBPA v6 | TGGTATGATTTTGTAATGGGGTAGGA (SEQ ID NO: 07) |
| CREB1 v1 | TGACGTCA (SEQ ID NO: 08) |
| CREB1 v2 | CTGACGTCAG (SEQ ID NO: 09) |
| CREB1 v3 | GCACGTCA (SEQ ID NO: 10) |
| CREB3L3 v1 | CCACGTTG (SEQ ID NO: 11) |
| CREB3L3 v2 | CCACGTAG (SEQ ID NO: 12) |
| CREB3L3 v3 | CCACGCTG (SEQ ID NO: 13) |
| CREB3L3 v4 | CCATGAACTTTG (SEQ ID NO: 14) |
| CREB3L3 v5 | CAAACGTGGTTT (SEQ ID NO: 15) |
| CREB3L3 v6 | TCCACGTGGTATT (SEQ ID NO: 16) |
| CREB3L3 v7 | TACACGTAATC (SEQ ID NO: 17) |
| CREB3L3 v8 | CACACGTGATC (SEQ ID NO: 18) |
| E2F1 v1 | GGCGCCAA (SEQ ID NO: 19) |
| E2F1 v2 | AAAAATGGCGCCAAAATG (SEQ ID NO: 20) |
| EBF1 (COE1) | TCCCTAGGGA (SEQ ID NO: 21) |
| EGR3 (PILOT) v1 | CGCCCACGCA (SEQ ID NO: 22) |
| EGR3 (PILOT) v2 | TACGCCCACGCATT (SEQ ID NO: 23) |
| ELF5 v1 (ESE-2) | CCGGAAGT (SEQ ID NO: 24) |
| ELF5 v2 | AACCCGGAAGTG (SEQ ID NO: 25) |
| ELK1 v1 | CCGGAAG (SEQ ID NO: 26) |
| ELK1 v2 | CACTTCCGCCGGAAGTG (SEQ ID NO: 27) |
| ETS1 v1 | ACAGGAAGT (SEQ ID NO: 28) |
| ETS1 v2 | ACCGGAAGTACTTCCGGT (SEQ ID NO: 29) |
| ETV4 (E1A-F, PEA3) | ACCGGAAGT (SEQ ID NO: 30) |
| FOXA1 (HNF-3α) v1 | TGTTTACTTT (SEQ ID NO: 31) |
| FOXA1 (HNF-3α) v2 | TTGTTTACTT (SEQ ID NO: 32) |
| FOXA2 (HNF-3β) | TGTTTGTTT (SEQ ID NO: 33 |
| FOXP3 | GTAAACA (SEQ ID NO: 34) |
| GATA6 | AGATAAGA (SEQ ID NO: 35) |
| GTF2I (TFII-I) | CCATT (SEQ ID NO: 36) |
| HNF1A (HNF-1α, LF-B1) v1 | GTTAATGATTAAC (SEQ ID NO: 37) |
| HNF1A (HNF-1α, LF-B1) v2 | GTTAATAATCTAC (SEQ ID NO: 38) |
| HNF1A (HNF-1α, LF-B1) v3 | GTTACTTATTCTC (SEQ ID NO: 39) |
| HNF1A (HNF-1α, LF-B1) v4 | GGTTAATAATTAAC (SEQ ID NO: 40) |
| HNF1A (HNF-1α, LF-B1) v5 | AATTATTTATTACCA (SEQ ID NO: 41) |
| HNF1A (HNF-1α, LF-B1) v6 | AGTATGGTTAATGATCTACAG (SEQ ID NO: 42) |
| HNF1A (HNF-1α, LF-B1) v7 | TGGTTAATATTCACCAGC (SEQ ID NO: 43) |
| HNF1A (HNF-1α, LF-B1) v8 | GTTTATCAGTGACTAGTCATTGAT (SEQ ID NO: 44) |
| HNF1A (HNF-1α, LF-B1) v9 | TGATAGCCAACTGCAGCTAATAATAAACCA (SEQ ID NO: 45) |
| HNF1A (HNF-1α, LF-B1) v10 | ATATTTTAGAGAAGAATTAACCTTT (SEQ ID NO: 46) |
| HNF4A (HNF-4α, NR2A1) v1 | AGTCCAAAGTTCA (SEQ ID NO: 47) |
| HNF4A (HNF-4α, NR2A1) v2 | TCGAGCGCTGGGCAAAGGTCACCTGC (SEQ ID NO: 48) |
| HNF4A (HNF-4α, NR2A1) v3 | TCGAAGGGCAGGGGTCAAGGGTTCAGT (SEQ ID NO: 49) |
| HNF4A (HNF-4α, NR2A1) v4 | TCGAGCGCAGGTCAAAGGTCACCTGC (SEQ ID NO: 50) |
| HNF4A (HNF-4α, NR2A1) v5 | TCGAGCGCAGGTCAAAAGGTCACCTGC (SEQ ID NO: 51) |
| HNF4A (HNF-4α, NR2A1) v6 | AGGTTAAAGGTCT (SEQ ID NO: 52) |
| HNF4A (HNF-4α, NR2A1) v7 | AGGTCAAAGTCCA (SEQ ID NO: 53) |
| HNF4A (HNF-4α, NR2A1) v8 | TGGGTCCAGAGGGCAAAA (SEQ ID NO: 54) |
| HNF4A (HNF-4α, NR2A1) v9 | CGCCCCAGCACACATGATCAGA (SEQ ID NO: 55) |
| HNF4A (HNF-4α, NR2A1) v10 | AACACGGGAGGTCAAAGATTGCGCCC (SEQ ID NO: 56) |
| IKZF1 (IKI, IKAROS, Lyf-1, ZNFN1A1) | TTGGGAAT (SEQ ID NO: 57) |
| IRF1 | GAAAGTGAAAGT (SEQ ID NO: 58) |
| JUN | TGAGTCA (SEQ ID NO: 59) |
| LEF1 (TCF-1α) | AGATCAAAG (SEQ ID NO: 60 |
| LRRFIP1 (GCF2, TRIP) | AGCCCCCGGCG (SEQ ID NO: 61) |
| MAZ (Zif87, ZNF801, Pur-1, SAF) | GGGGAGGG (SEQ ID NO: 62) |
| MYB | CAGTTG (SEQ ID NO: 63) |
| NFATC2 (NFATp, NFAT1) | TTTCCATGGAAA (SEQ ID NO: 64) |
| NF-IC | TTGGCTATATGCCAA (SEQ ID NO: 65) |
| NFYA (CP1A, CBF-B) | CCAAT (SEQ ID NO: 66) |
| ONECUT-1 (HNF6, HNF-6α, OC-1) v1 | TATTGATT (SEQ ID NO: 67) |
| ONECUT-1 (HNF6, HNF-6α, OC-1) v2 | TCCATTGATTTAG (SEQ ID NO: 68) |
| ONECUT-1 (HNF6, HNF-6α, OC-1) v3 | AAAAAATCAATAAT (SEQ ID NO: 69) |
| ONECUT-1 (HNF6, HNF-6α, OC-1) v4 | GAAAAAAAAATCAATATCGGGCCT (SEQ ID NO: 70) |
| ONECUT-1 (HNF6, HNF-6α, OC-1) v5 | GTCTGCTAAGTCAATAATCAGAAT (SEQ ID NO: 71) |
| PAX5 | GTCATGCGTGAC (SEQ ID NO: 72) |
| PLAG1 | CCCCCTTGGGCCCC (SEQ ID NO: 73) |
| POU2F1 (Oct-1, OTF-1) v1 | TATGCAAAT (SEQ ID NO: 74) |
| POU2F1 (Oct-1, OTF-1) v2 | AATATGCAAATTAG (SEQ ID NO: 75) |
| POU2F1 (Oct-1, OTF-1) v3 | TTATGCATATGCATAA (SEQ ID NO: 76) |
| POU2F1 (Oct-1, OTF-1) v4 | ATATGATTATGCAAATTTATAGA (SEQ ID NO: 77) |
| PPARα (PPARA, NR1C1) v1 | AGGTCATCAGGTCA (SEQ ID NO: 78) |
| PPARα (PPARA, NR1C1) v2 | AGGTCAAAGGTCA (SEQ ID NO: 79) |
| PPARα (PPARA, NR1C1) v3 | AGGTCAATGACCT (SEQ ID NO: 80) |
| PPARα (PPARA, NR1C1) v4 | GTGTCAAAGGTCA (SEQ ID NO: 81) |
| PPARα (PPARA, NR1C1) v5 | AACTAGGTCAAAGGTCA (SEQ ID NO: 82) |
| PPARα (PPARA, NR1C1) v6 | CAAAACTAGGTCAAAGGTCA (SEQ ID NO: 83) |
| PPARα (PPARA, NR1C1) v7 | AACTAGGTCAAAGGTCAAAG (SEQ ID NO: 84) |
| SMAD1 | GTCTAGAC (SEQ ID NO: 85) |
| SP1v1 | GGGGCGGGG (SEQ ID NO: 86) |
| SP1v2 | GCCACGCCCCC (SEQ ID NO: 87) |
| SPI1 (PU.1) | AAAAGAGGAAGTG (SEQ ID NO: 88) |
| SRY (TDF) | AAACAATAACATTGTTT (SEQ ID NO: 89) |
| STAT1 | TTCCCGGAA (SEQ ID NO: 90) |
| TBP | TATAAAA (SEQ ID NO: 91) |
| TCF7L2 (TCF-4) | ACTTCAAAGG (SEQ ID NO: 92) |
| TFAP2A (AP-2α) | GCCTGAGGC (SEQ ID NO: 93) |
| TP53 (p53) | GGACATGCCCGGGCATGTCC (SEQ ID NO: 94) |
| USF2 | GTCACGTGAC (SEQ ID NO: 95) |
| WT1 | CGCCCCCGC (SEQ ID NO: 96) |
| XBP1 | GATGACGTCATC (SEQ ID NO: 97) |
| YY1 | GCCGCCATTTT (SEQ ID NO: 98) |
| ZNF423 (OAZ, Zfp423) | GCACCCAAGGGTGC (SEQ ID NO: 99) |

In other embodiments, the DTS comprises a TFBS that is bound by a nuclear targeting factor (NTF) that becomes active in the target cell when the target cell is contacted by an inducing agent. Put another way, the DTS comprises a sequence that mediates nuclear translocation of a polynucleotide that comprises it when the cell is additionally contacted by an inducing agent. Such DTSs may be referred to as “inducible DTSs”, or “IDTSs”. By an inducing agent, it is meant an agent that activates a DNA binding protein that resides outside the nucleus (e.g., at the plasma membrane, in the cytosol, etc.) to translocate to the nucleus. In some instances, the inducing agent binds directly to the DNA binding protein that is being induced to translocate. In other instances, the inducing agent binds to an upstream protein that then activates a DNA binding protein to be induced to translocate. As one nonlimiting example, the DTS may comprise a nucleic acid sequence which is recognized and bound by a nuclear targeting factor, e.g., a cytosolic protein that, in response to inducer activity, moves from the cytosol into the nucleus. As the DTS of the NTDNA is bound to the nuclear target factor, translocation of the nuclear targeting factor into the nucleus brings the NTDNA into the nucleus, thereby mediating delivery of the NTDNA (and cargo nucleic acid thereof) into the nucleus. DTS sequences finding use in NTDNAs may vary as desired. In some embodiments, iDTS sequences employed in NTDNAs comprise DNA binding domains of nuclear targeting factors.

One non-limiting example of iDTSs are nucleotide sequences that comprise the binding domains of Nuclear Receptors. Nuclear Receptors (NRs) are ligand-activated transcription factors that bind to small molecules to regulate gene expression and other cellular processes. This family includes receptors for steroid hormones and derivatives (such as estrogen, progesterone, glucocorticoids, Vitamin D, oxysterols and bile acids, among others) as well as receptors for retinoic acids, thyroid hormones and fatty acids and their derivatives. These ligands are able to diffuse directly through cellular membranes as a result of their lipophilic nature (reviewed in Beato et al, Steroids 1996 April; 61 (4): 240-51; Holzer et al, Curr Top Dev Biol. 2017; 125:1-38. 2017). The 48 human nuclear receptors share a conserved modular structure that consists of a sequence specific DNA-binding domain and a ligand-binding domain, in addition to various other protein-protein interaction domains. Upon interaction with ligand, NRs bind to the regulatory regions of target genes as homo- or heterodimers, or more rarely, as monomers. At the promoter, NRs interact with other activators and repressors to regulate gene expression (reviewed Beato et al, supra; Simons et al, Mol Endocrinol. 2014 February; 28 (2): 173-82; Hah and Kraus, Mol Cell Endocrinol. 2014 Jan. 25; 382 (1): 652-664). Of interest in the present invention are the class of nuclear receptors that reside in the cytoplasm in the absence of ligand. Ligand-binding to these receptors promotes nuclear translocation, and the translocation of cytoplasmic DNA to which they bind. (https://reactome.org/content/detail/R-HSA-9006931). iDTS that may be present in NTDNAs of the invention include, but are not limited to, those bound by nuclear receptors. Another non-limiting example of iDTSs are nucleotide sequences that comprise the binding domains of transcription factors that reside quiescent in the cytoplasm and become indirectly activated when the cell comes in contact with an inducing reagent. By “indirectly activated” it is meant that they become activated by a protein that binds the inducing reagent. Examples of such transcription factors include NF-kB, CREB, and the like.

Table 2 provides examples of TFBSs that could be utilized in iDTSs, the nuclear targeting factor that would be leveraged to achieve nuclear transport of associated cargo DNA, and examples of inducing agents that could be used to drive that translocation event.

TABLE 2
iDTSs, the DNA binding proteins that they leverage for nuclear translocation,
and examples of inducing agents that activate them.

| DNA binding protein | TFBS | Inducing agent examples |
|---|---|---|
| HNF-4α (NR2A1) | AGTCCAAAGTTCA (SEQ ID NO: 100) | linoleic acid |
| COUP-TFI (NR2F1) | TGACCTTTGACCT (SEQ ID NO: 101) | orphan receptor (unknown) |
| Estrogen receptor α | AGGTCACAGTGACCT (SEQ ID NO: 102) | 17ß-estradiol; estriol; estrone |
| (ERα) (ESR1) | | |
| (NR3A1) | | |
| ESRRB (ERR2, | TCAAGGTCA (SEQ ID NO: 103) | |
| ERRß, NR3B2) | | |
| Glucocorticoid | GGAACATTATGTACC (SEQ ID NO: 104) | aldosterone; corticosterone; |
| receptor (GR) | | cortisol; deoxycorticosterone; |
| (NR3C1) v1 | | deoxycortisone |
| Glucocorticoid | AGAACAAAATGTTCT (SEQ ID NO: 105) | |
| receptor (GR) | | |
| (NR3C1) v2 | | |
| Glucocorticoid | AAGAACAAAATGTTCTT (SEQ ID NO: 106) | |
| receptor (GR) | | |
| (NR3C1) v3 | | |
| Glucocorticoid | AGAACATTTTGTACG (SEQ ID NO: 107) | |
| receptor (GR) | | |
| (NR3C1) v4 | | |
| Glucocorticoid | AAGAACATTTTGTACGT (SEQ ID NO: 108) | |
| receptor (GR) | | |
| (NR3C1) v5 | | |
| Glucocorticoid | AGAACATCCCTGTACA (SEQ ID NO: 109) | |
| receptor (GR) | | |
| (NR3C1) v6 | | |
| NR3C4 (Androgen | AGAACATGATGTTCT (SEQ ID NO: 110) | dihydrotestosterone; |
| receptor (AR)) | | testosterone |
| NR1H3 (LXRA) v1 | AATAGAGGTCACTAAAGGTCAAGC (SEQ ID | 24(S), 25-epoxycholesterol |
| | NO: 111) | 24(S)-hydroxycholesterol |
| NR1H3 (LXRA) v2 | AAACTAGGTCACGAAAGGTCAAAGTC (SEQ | 27-hydroxycholesterol |
| | ID NO: 112) | 22R-hydroxycholesterol |
| NR1H2 (LXRB) | TGACCTCTACTGACCT (SEQ ID NO: 113) | 24(S),25-epoxycholesterol; |
| | | 24(S)-hydroxycholesterol; 27- |
| | | hydroxycholesterol; 22R- |
| | | hydroxycholesterol |
| MLXIPL.v1 | ATCACGTGATTATCACGTGAT (SEQ ID | Glucose |
| | NO: 114) | |
| NFKB1 v1 | GGGAATTTCC (SEQ ID NO: 115) | INFa, PMA, CpG |
| NFKB1 v2 | GGGACTTTCC (SEQ ID NO: 116) | TNFa, PMA, CpG |
| NR1I2.(PXR) v1 | AGGCAGAGGGCAGAAAGGTCAAGGG (SEQ | 17β-estradiol |
| | ID NO: 117) | 3-keto-lithocholic acid |
| NR112 (PXR) v2 | CAGAGGTCACAGAGTTCAAGC (SEQ ID | lithocholic acid |
| | NO: 118) | |
| NR1I3 (CAR) v1 | CAGAGTTCATGAGAGTTCAAGC (SEQ ID | Xenobiotics and androstanes |
| | NO: 119) | |
| NR1I3 (CAR) v2 | GCCCCCAGGGCTGAGTGACAGAAAAACAG | |
| | (SEQ ID NO: 120) | |
| NR1I3 (CAR) v3 | GCATTACAGACTGGGTGACAGAGTGAGAC | |
| | (SEQ ID NO: 121) | |
| NR1I3 (CAR) v4 | AATTATGGTTCTGGGTGATTCAAGTAACA | |
| | (SEQ ID NO: 122) | |
| NR1I3 (CAR) v5 | GCAATAAAATCTGGGTCACAGGAGTTGGA | |
| | (SEQ ID NO: 123) | |
| NR1I3 (CAR) v6 | CCTCTTCTCTGTGGGTGACCAGCGTCCTA | |
| | (SEQ ID NO: 124) | |
| PPARα (PPARA, | AGGTCATCAGGTCA (SEQ ID NO: 125) | LTB4, |
| NR1C1) v1 | | pristanic acid, |
| PPARα (PPARA, | AGGTCAAAGGTCA (SEQ ID NO: 126) | 8S-HETE |
| NR1C1) v2 | | |
| PPARα (PPARA, | AGGTCAATGACCT (SEQ ID NO: 127) | |
| NR1C1) v3 | | |
| PPARα (PPARA, | GTGTCAAAGGTCA (SEQ ID NO: 128) | |
| NR1C1) v4 | | |
| PPARα (PPARA, | AACTAGGTCAAAGGTCA (SEQ ID NO: 129) | |
| NR1C1) v5 | | |
| PPARα (PPARA, | CAAAACTAGGTCAAAGGTCA (SEQ ID | |
| NR1C1) v6 | NO: 130) | |
| PPARα (PPARA, | AACTAGGTCAAAGGTCAAAG (SEQ ID | |
| NR1C1) v7 | NO: 131) | |
| RAR-α (RARA, NR1B1) | TGACCTTTTGACCTTT (SEQ ID NO: 132) | tretinoin |
| RXRα (RXRA, NR2B1) | TGACCTTTGACCCC (SEQ ID NO: 133) | alitretinoin; docosahexaenoic |
| SREBF1.v1 | ATCACCCCAC (SEQ ID NO: 134) | sterols |
| SREBF1.v2 | ATCACGTGAT (SEQ ID NO: 135) | |
| VDR (NR111) v1 | GAGTTCATTGAGTTCA (SEQ ID NO: 136) | calcitriol-26,23-lactone; 1,25- |
| | | dihydroxyvitamin D3; 3-keto- |
| VDR (NR111) v2 | CAGGGGTCATCGGGTTCAAAC (SEQ ID | lithocholic acid; lithocholic |
| | NO: 137) | acid |

In other embodiments, the DTS comprises a TFBS that is bound by a nuclear targeting factor that is provided to the cell as an mRNA encoding a protein. As will be appreciated by one of ordinary skill in the art, any protein that binds to DNA and that traffics to the nucleus when delivered to the cytoplasm can be provided to the cell to mediate nuclear translocation. Thus, for example, any of the naturally occurring proteins described in Tables 1 and 2 may be exogenously provided. As another example, a protein that is not native to the cell, i.e., that is heterologous to the cell, may be provided. Such a protein may be naturally occurring or engineered. Examples include any of the proteins listed in Table 3. Exemplary proteins of each class and the DNA sequences to which they bind are well known in the art and include those described in greater detail below.

TABLE 3
Classes of nuclear targeting factors that may be co-delivered with NTDNA to achieve DNA
nuclear transport

| Protein/DNA binding system | Example NTF protein |
|---|---|
| Bacterial regulatory proteins | Tetracycline repressor (TetR), Purine |
| | synthesis repressor (PurR); Lac repressor (LacR) |
| Yeast regulatory proteins | Gal4; GCN4 |
| Bacteriophage regulatory proteins | Arc; Mnt; Bac434 |
| Mammalian regulatory proteins | YY1; HEY1; MyoD; GR; ER; PR |
| Zing finger proteins (ZFP) | ZFP-CCR5 |
| Transcription Activator-Like Effectors (TALE) | TALE-CCR5 |
| Inactivated nucleases | Inactivated I-SceI |
| Inactivated Site-Specific Recombinases | Cre; Piggybac |
| Transcription factor DNA-binding domain | STAT1; ESR1; Gal4 |
| Inactivated RNA-guided endonuclease | dCas9; HypaCas9; miniCas9; Cas12a |
| Single strand DNA binding protein | hSSB1; POT1 |

In some embodiments, the TFBS is a binding sequence for a protein selected from the group consisting of HNF1A, PPARA, HNF4A, CEBPA, NR3C1, ONECUT1, TBP, NFKB, NR1I3, FOXA1, and ELF5. In some embodiments, the HNF1A binding sequence is v4 or v6. In some embodiments, the PPARA binding sequence is v5, v6 or v7. In some embodiments, the HNF4A binding sequence is v2, v4 or v7. In some embodiments, the CEBPA binding sequence is v6. In some embodiments, the NR3C1 binding sequence is v2, v3, v4 or v5. In some embodiments, the ONECUT binding sequence is v5. In some embodiments, the TBP binding sequence is v1. In some embodiments, the NFkB binding sequence is v2. In some embodiments, the NR113 binding sequence is v2, v4, v6. In some embodiments, the FOXA1 binding sequence is v1. In some embodiments, the ELF5 binding sequence is v1.

In some embodiments, the DTS is a combination of binding sequences for HNF1A, NR.113, PPARA, HNF1A, and PPARA. In certain embodiments, the DTS is a combination of binding sequences HNF1A.v4, NR113.v6, PPARA.v5, HNF1A.v6, and PPARA.v6. In certain embodiments, the DTS is

(SEQ ID NO: 138)
GGTTAATAATTAACAGATTACTACTGATACCTCTTCTCTGTGGGTGACC
AGCGTCCTAAGATTACTACTGATAAACTAGGTCAAAGGTCAAGATTACT
ACTGATAAGTATGGTTAATGATCTACAGAGATTACTACTGATACAAAAC
TAGGTCAAAGGTCA.

In some embodiments, the DTS is a combination of binding sequences for FOXA1, HNF4A.v4, HNF1A, HNF4A, and HNF1A. In certain embodiments, the DTS is a combination of binding sequences FOXA1.v1, HNF4A.v4, HNF1A.v4, HNF4A.v2, and HNF1A.v6. In certain embodiments, the DTS is

(SEQ ID NO: 139)
TGTTTACTTTAGATTACTACTGATATCGAGCGCAGGTCAAAGGTCACCT
GCAGATTACTACTGATAGGTTAATAATTAACAGATTACTACTGATATCG
AGCGCTGGGCAAAGGTCACCTGCAGATTACTACTGATAAGTATGGTTAA
TGATCTACAG.

In some embodiments, the DTS comprises the binding sequence for NFkB. In certain embodiments, the binding sequence is NFkB.v2. In certain embodiments, the DTS is:

(SEQ ID NO: 140)
GGGACTTTCCAGATTACTACTGATAGGGACTTTCCAGATTACTACTGAT
AGGGACTTTCCAGATTACTACTGATAGGGACTTTCCAGATTACTACTGA
TAGGGACTTTCC.

In some embodiments, the DTS is a combination of binding sequences for CREB1, PPARA, ONECUT1, HNF4A, and PPARA. In certain embodiments, the DTS is a combination of binding sequences CREB1.v2, PPARA.v6, ONECUT1.v5, HNF4A.v9, and PPARA.v2. In certain embodiments, the DTS is:

(SEQ ID NO: 141)
CTGACGTCAGAGATTACTACTGATACAAAACTAGGTCAAAGGTCAAGAT
TACTACTGATAGTCTGCTAAGTCAATAATCAGAATAGATTACTACTGAT
ACGCCCCAGCACACATGATCAGAAGATTACTACTGATAAGGTCAAAGGT
CA.

In some embodiments, the TFBS is a binding sequence for a Tet Repressor (TetR) protein, for example, a TetO sequence such as YCTATCANTGATAGA (SEQ ID NO: 142), for example, TCCCTATCAGTGATAGAGA (SEQ ID NO:143) or TCGAGTTTACTCCCTATCAGTGATAGAGAACG (SEQ ID NO:144). In some embodiments, the DTS comprises 2, 3, 4, 5, 6, 7, 8, 9, or 10 copies of the TetR TFBS. In certain embodiments, the DTS comprises 7 copies of the TetR TFBS. In certain embodiments, the DTS comprises 10 or more copies of the TetR TFBS. In some embodiments, the DTS comprises a sequence having 80% identity or more to a sequence listed in Table 4, for example, 85% or 90% identity or more, in some cases 95% identity or more, e.g., 96%, 97%, 98%, or 99% identity to a sequence in Table 4. In some instances, the DTS binding sequence is identical to a sequence in Table 4. In some instances, the DTS consists essential of a sequence in Table 4. In certain embodiments, the DTS comprises a tetracycline response element (TRE), as known in the art. In certain embodiments, the DTS consists essentially of a TRE.

TABLE 4
Examples of DTSs that comprise a TetR binding sequence (e.g., TetO)
in varying numbers and sequences,

| DTS | Sequence (5′ to 3′) |
|---|---|
| TetO Motif | YCTATCANTGATAGA (SEQ ID NO: 145) |
| TetO-1 (Core) | TCCCTATCAGTGATAGAGA (SEQ ID NO: 146) |
| TetO-2 | CTCGAGTTTACTCCCTATCAGTGATAGAGAACGTATGTCGAGTTTACTCCCTAT |
| | CAGTGATAGAGAACGATGTCGAGTTTAC (SEQ ID NO: 147) |
| TetO-3 | CTCGAGTTTACTCCCTATCAGTGATAGAGAACGTATGTCGAGTTTACTCCCTAT |
| | CAGTGATAGAGAACGATGTCGAGTTTACTCCCTATCAGTGATAGAGAACGTAT |
| | GTCGAGTTTAC (SEQ ID NO: 148) |
| TetO-4 | CTCGAGTTTACTCCCTATCAGTGATAGAGAACGTATGTCGAGTTTACTCCCTAT |
| | CAGTGATAGAGAACGATGTCGAGTTTACTCCCTATCAGTGATAGAGAACGTAT |
| | GTCGAGTTTACTCCCTATCAGTGATAGAGAACGTATGTCGAGTTTAC (SEQ ID |
| | NO: 149) |
| TetO-5 | CTCGAGTTTACTCCCTATCAGTGATAGAGAACGTATGTCGAGTTTACTCCCTAT |
| | CAGTGATAGAGAACGATGTCGAGTTTACTCCCTATCAGTGATAGAGAACGTAT |
| | GTCGAGTTTACTCCCTATCAGTGATAGAGAACGTATGTCGAGTTTACTCCCTAT |
| | CAGTGATAGAGAACGTATGTCGAGTTTA (SEQ ID NO: 150) |
| TetO-6 | CTCGAGTTTACTCCCTATCAGTGATAGAGAACGTATGTCGAGTTTACTCCCTAT |
| | CAGTGATAGAGAACGATGTCGAGTTTACTCCCTATCAGTGATAGAGAACGTAT |
| | GTCGAGTTTACTCCCTATCAGTGATAGAGAACGTATGTCGAGTTTACTCCCTAT |
| | CAGTGATAGAGAACGTATGTCGAGTTTATCCCTATCAGTGATAGAGAACGTAT |
| | GTCGAGTTTAC (SEQ ID NO: 151) |
| TetO-7 | CTCGAGTTTACTCCCTATCAGTGATAGAGAACGTATGTCGAGTTTACTCCCTAT |
| | CAGTGATAGAGAACGATGTCGAGTTTACTCCCTATCAGTGATAGAGAACGTAT |
| | GTCGAGTTTACTCCCTATCAGTGATAGAGAACGTATGTCGAGTTTACTCCCTAT |
| | CAGTGATAGAGAACGTATGTCGAGTTTATCCCTATCAGTGATAGAGAACGTAT |
| | GTCGAGTTTACTCCCTATCAGTGATAGAGAACGTATGTCGAGGTAGGCGTGTA |
| | CGGTGGGAGGCCTATATAAGCAGAGCTCGTTTAGTGAACCGTCAGATCGCCTG |
| | GAGAATTCA (SEQ ID NO: 152) |
| TetO-7-m | CTCGAGTTTACTCCCTATCAGTGATAGAGAACGTATGTCGAGTTTACTCCCTAT |
| | CAGTGATAGAGAACGATGTCGAGTTTACTCCCTATCAGTGATAGAGAACGTAT |
| | GTCGAGTTTACTCCCTATCAGTGATAGAGAACGTATGTCGAGTTTACTCCCTAT |
| | CAGTGATAGAGAACGTATGTCGAGTTTATCCCTATCAGTGATAGAGAACGTAT |
| | GTCGAGTTTACTCCCTATCAGTGATAGAGAACGTATGTCGAGGTA (SEQ ID |
| | NO: 153) |

In some embodiments, the TFBS is a binding sequence for the DNA binding domain of a gene editing system, e.g., as described herein or as known in the art, e.g., the guide RNA of a Cas nuclease, the zinc finger domain of a zinc finger nuclease, the TALE DNA binding domain of a TALEN.

In some embodiments, the TFBS is a binding sequence for a zinc-finger containing protein (“ZF protein”). As will be appreciated by the ordinarily skilled artisan, any ZF protein and its cognate ZF binding sequence may be used as a nuclear targeting factor (NTF) and cognate TFBS in the compositions and methods of the present disclosure. In some embodiments the DTS comprises a ZF-responsive TFBS having a sequence identity of 90% or more to AAACTGCAAAAG (SEQ ID NO:455).

In some embodiments, the TFBS is a binding sequence for a TAL effector DNA-binding domain-containing protein (“TALE protein”). A TALE is a protein of 32 fixed amino acids and 2 variable residues, the 2 residues being engineered to recognize specific nucleotides (e.g., NN for G, NI for A, HD for C, etc.). As will be appreciated by the ordinarily skilled artisan, any TALE protein and its cognate TALE binding sequence may be used in the compositions and methods of the present disclosure, such examples being found in, e.g., Li et al. 2011 (Modularly assembled designer TAL effector nucleases for targeted gene knockout and gene replacement in eukaryotes. Nucleic Acids Research, Volume 39, Issue 14, pp 6315-6325) and Kim et. al. 2013 (A library of TAL effector nucleases spanning the human genome. Nature Biotechnology volume 31, pages 251-258 (2013). In some embodiments, the TALE TFBS has 90% identity or more to a sequence is selected from the group consisting of TTCATTACACCTGCAGCT (SEQ ID NO:286), ATAAACCCCCTCCAA (SEQ ID NO:287), and TCGAGTTTACTCCCTATCAGTGATAGAGAACG (SEQ ID NO:154).

In some embodiments, the TFBS is a binding sequence for a GAL4 protein. In some such embodiments, the GAL4 TFBS comprises the sequence CGG-N₁₁-CCG, for example, CGGAGGACTGTCCTCCG (SEQ ID NO:155). In some embodiments, the DTS comprises 2, 3, 4, 5, 6, 7, 8, 9, or 10 copies of the GALA TFBS. In certain embodiments, the DTS comprises 5 copies of the GAL4 TFBS. In certain embodiments, the DTS comprises 7 copies of the GAL4 TFBS. In certain embodiments, the DTS comprises 10 or more copies of the GAL4 TFBS. In certain embodiments, the DTS comprises an upstream activation sequence (UAS) for the native GAL4 protein, as known in the art. In certain embodiments, the DTS consists essentially of a UAS. In some embodiments, the DTS comprises a sequence having 80% identity or more to a sequence listed in Table 5, for example, 85% or 90% identity or more, in some cases 95% identity or more, e.g., 96%, 97%, 98%, or 99% identity to a sequence in Table 5. In some instances, the DTS is identical to a sequence in Table 5.

TABLE 5
Examples of DTSs comprising a GAL4 TFBS (c.g., UAS)

| DTS | Sequence (5′ to 3′) |
|---|---|
| UAS Motif | CGG-Nh-CCG (SEQ ID NO: 156) |
| UAS Core A | CGGTGGCTTCTAATCCG (SEQ ID NO: 157) |
| UAS Core B | CGGGTGACAGCCCTCCG (SEQ ID NO: 158) |
| UAS Core C | CGGAGGAGAGTCTTCCG (SEQ ID NO: 159) |
| UAS Core D | CGGAGTACTGTCCTCCG (SEQ ID NO: 160) |
| UAS Core E | CGGAGGACTGTCCTCCG (SEQ ID NO: 161) |
| UAS-1 | ACACTAGAGTCGGAGGACTGTCCTCCGTGAGTCCTAG (SEQ ID NO: 162) |
| UAS-2 | CGGAGGACTGTCCTCCGTGAGTCCTAGCGGAGGACTGTCCTCCG (SEQ ID |
| | NO: 163) |
| UAS-3 | CGGAGGACTGTCCTCCGTGAGTCCTAGCGGAGGACTGTCCTCCGTCTTCACAG |
| | GCGGAGGACTGTCCTCCG (SEQ ID NO: 164) |
| UAS-4 | CGGAGGACTGTCCTCCGTGAGTCCTAGCGGAGGACTGTCCTCCGTCTTCACAG |
| | GCGGAGGACTGTCCTCCGTAGGGTTCCTCGGAGGACTGTCCTCCG (SEQ ID |
| | NO: 165) |
| UAS-5 | CGGAGGACTGTCCTCCGTGAGTCCTAGCGGAGGACTGTCCTCCGTCTTCACAG |
| | GCGGAGGACTGTCCTCCGTAGGGTTCCTCGGAGGACTGTCCTCCGACACTAGA |
| | GTCGGAGGACTGTCCTCCG (SEQ ID NO: 166) |
| UAS-5-NR | CGGTGGCTTCTAATCCGTGAGTCCTAGCGGGTGACAGCCCTCCGTCTTCACAG |
| | GCGGAGGAGAGTCTTCCGTAGGGTTCCTCGGAGTACTGTCCTCCGACACTAGA |
| | GTCGGAGGACTGTCCTCCG (SEQ ID NO: 167) |
| UAS-6 | CGGAGGACTGTCCTCCGTGAGTCCTAGCGGAGGACTGTCCTCCGTCTTCACAG |
| | GCGGAGGACTGTCCTCCGTAGGGTTCCTCGGAGGACTGTCCTCCGACACTAGA |
| | GTCGGAGGACTGTCCTCCGGATAGTATCACGGAGGACTGTCCTCCG (SEQ ID |
| | NO: 168) |
| UAS-7 | CGGAGGACTGTCCTCCGTGAGTCCTAGCGGAGGACTGTCCTCCGTCTTCACAG |
| | GCGGAGGACTGTCCTCCGTAGGGTTCCTCGGAGGACTGTCCTCCGACACTAGA |
| | GTCGGAGGACTGTCCTCCGGATAGTATCACGGAGGACTGTCCTCCGAGCAAA |
| | CGAACGGAGGACTGTCCTCCG (SEQ ID NO: 169) |
| UAS-10 | CGGAGGACTGTCCTCCGTGAGTCCTAGCGGAGGACTGTCCTCCGTCTTCACAG |
| | GCGGAGGACTGTCCTCCGTAGGGTTCCTCGGAGGACTGTCCTCCGACACTAGA |
| | GTCGGAGGACTGTCCTCCGGATAGTATCACGGAGGACTGTCCTCCGAATGGAC |
| | TAACGGAGGACTGTCCTCCGAGCAAACGAACGGAGGACTGTCCTCCGTACAC |
| | CGATCCGGAGGACTGTCCTCCGGCGCTCGGCACGGAGGACTGTCCTCCG (SEQ |
| | ID NO: 170) |
| UAS-20 | CGGAGGACTGTCCTCCGTGAGTCCTAGCGGAGGACTGTCCTCCGTCTTCACAG |
| | GCGGAGGACTGTCCTCCGTAGGGTTCCTCGGAGGACTGTCCTCCGACACTAGA |
| | GTCGGAGGACTGTCCTCCGGATAGTATCACGGAGGACTGTCCTCCGAATGGAC |
| | TAACGGAGGACTGTCCTCCGACTTTAGCGCCGGAGGACTGTCCTCCGTACACC |
| | GATCCGGAGGACTGTCCTCCGGCGCTCGGCACGGAGGACTGTCCTCCGCGAA |
| | AACGGGCGGAGGACTGTCCTCCGTGCGTGGATTCGGAGGACTGTCCTCCGAG |
| | CCAGGAGCCGGAGGACTGTCCTCCGAAGACCACGGCGGAGGACTGTCCTCCG |
| | ACCAGTACCCCGGAGGACTGTCCTCCGATGTCTTGTACGGAGGACTGTCCTCC |
| | GCTGACTATCGCGGAGGACTGTCCTCCGCCTACGTCATCGGAGGACTGTCCTC |
| | CGATAGAAACCTCGGAGGACTGTCCTCCGCATGTCTAACCGGAGGACTGTCCT |
| | CCG (SEQ ID NO: 171) |

In some embodiments, the TFBS is a binding sequence for an Arc protein, where Arc is a bacteriophage regulatory protein. In some such embodiments, the Arc TFBS comprises the sequence RYRVTAGANNNNNTCTABYRY (SEQ ID NO: 172), for example, ATGATAGAAGCACTCTACTAT (SEQ ID NO:173). In some embodiments, the Arc TFBS comprises a sequence having 80%, 85%, 90% identity or more to ATGATAGAAGCACTCTACTAT (SEQ ID NO:173). In some embodiments, the DTS comprises 2, 3, 4, 5, 6, 7, 8, 9, or 10 copies of the Arc TFBS. In certain embodiments, the DTS comprises 5 copies of the Arc TFBS. In certain embodiments, the DTS comprises 7 copies of the Arc TFBS. In certain embodiments, the DTS comprises 10 or more copies of the Arc TFBS. In some embodiments, the DTS comprises a sequence having 80%, 85%, or 90% identity or more to the sequence ATGATAGAAGCACTCTACTATTGAGTCCTAGATGATAGAAGCACTCTACTATTCTTCACAGG ATGATAGAAGCACTCTACTATTAGGGTTCCTATGATAGAAGCACTCTACTATACACTAGAGT ATGATAGAAGCACTCTACTATGATAGTATCAATGATAGAAGCACTCTACTATAGCAAACGAA ATGATAGAAGCACTCTACTAT (SEQ ID NO:174), in some cases 95% identity or more to this sequence, in certain cases sharing 100% identity with this sequence.

In some embodiments, the TFBS is a binding sequence for a Mnt protein, where Mnt is a bacteriophage regulatory protein. In some such embodiments, the Mnt TFBS comprises the sequence GGNCCACNGTGGNCC (SEQ ID NO:303), for example, ATAGGTCCACGGTGGACCATA (SEQ ID NO: 175). In some embodiments, the Mnt TFBS comprises a sequence having 80%, 85%, 90% identity or more to ATAGGTCCACGGTGGACCATA (SEQ ID NO: 175). In some embodiments, the DTS comprises 2, 3, 4, 5, 6, 7, 8, 9, or 10 copies of the Mnt TFBS. In certain embodiments, the DTS comprises 5 copies of the Mnt TFBS. In certain embodiments, the DTS comprises 7 copies of the Mnt TFBS. In certain embodiments, the DTS comprises 10 or more copies of the Mnt TFBS. In some embodiments, the DTS comprises a sequence having 80%, 85%, or 90% identity or more to the sequenceATAGGTCCACGGTGGACCATATGAGTCCTAGATAGGTCCACGGTGGACCATATCTTC ACAGGATAGGTCCACGGTGGACCATATAGGGTTCCTATAGGTCCACGGTGGACCATAACACT AGAGTATAGGTCCACGGTGGACCATAGATAGTATCAATAGGTCCACGGTGGACCATAAGCA AACGAAATAGGTCCACGGTGGACCATA (SEQ ID NO:176), in some cases 95% identity or more to this sequence, in certain cases sharing 100% identity with this sequence.

In some embodiments, the TFBS is a binding sequence for a purine synthesis repressor (PurR) protein. In some such embodiments, the PurR TFBS comprises a sequence having 80%, 85%, 90% identity or more to ACGCAAACGTTTTCGT (SEQ ID NO:307), in some cases 95% identity or more to this sequence, in certain cases sharing 100% identity with this sequence. In some embodiments, the DTS comprises 2, 3, 4, 5, 6, 7, 8, 9, or 10 copies of the PurR TFBS. In certain embodiments, the DTS comprises 5 copies of the PurR TFBS. In certain embodiments, the DTS comprises 7 copies of the PurR TFBS. In certain embodiments, the DTS comprises 10 or more copies of the PurR TFBS. I In some embodiments, the DTS comprises a sequence having 80%, 85%, or 90% identity or more to the sequence ACGCAAACGTTTTCGTTGAGTCCTAGACGCAAACGTTTTCGTTCTTCACAGGACGCAAACGT TTTCGTTAGGGTTCCTACGCAAACGTTTTCGTACACTAGAGTACGCAAACGTTTTCGTGATAG TATCAACGCAAACGTTTTCGTAGCAAACGAAACGCAAACGTTTTCGT (SEQ ID NO: 177), in some cases 95% identity or more to this sequence, in certain cases sharing 100% identity with this sequence.

In some embodiments, the TFBS is a binding sequence for a Bac434 protein, where Bac434 is a bacteriophage regulatory protein. In some such embodiments, the Bac434 TFBS comprises a sequence that has 80%, 85%, 90% identity or more to ACAAGAAAGTTTGT (SEQ ID NO:178), ACAAGATACATTGT (SEQ ID NO:179), or ACAAGAAAAACTGT (SEQ ID NO: 180), in some cases 95% identity or more to ACAAGAAAGTTTGT (SEQ ID NO:181), ACAAGATACATTGT (SEQ ID NO: 182), or ACAAGAAAAACTGT (SEQ ID NO:183), in certain cases sharing 100% identity with ACAAGAAAGTTTGT (SEQ ID NO:184), ACAAGATACATTGT (SEQ ID NO:185), or ACAAGAAAAACTGT (SEQ ID NO:186). In some embodiments, the DTS comprises 2, 3, 4, 5, 6, 7, 8, 9, or 10 copies of the Bac434 TFBS. In certain embodiments, the DTS comprises 5 copies of the Bac434 TFBS. In certain embodiments, the DTS comprises 7 copies of the Bac434 TFBS. In certain embodiments, the DTS comprises 10 or more copies of the Bac434 TFBS. In certain embodiments, the DTS comprises a sequence having 80%, 85%, or 90% identity or more to a sequence listed in Table 6, for example, 85% or 90% identity or more, in some cases 95% identity or more, e.g., 96%, 97%, 98%, or 99% identity to a sequence in Table 6. In some instances, the DTS is identical to a sequence in Table 6.

TABLE 6
Examples of DTSs comprising a Bac434 TFBS

| DTS | Sequence (5′ to 3′) |
|---|---|
| Bac434-OR1-Core | ACAAGAAAGTTTGT |
| | (SEQ ID NO: 187) |
| Bac434-OR2-Core | ACAAGATACATTGT |
| | (SEQ ID NO: 188) |
| Bac434-OR3-Core | ACAAGAAAAACTGT |
| | (SEQ ID NO: 189) |
| Bac434-OR321-Core | ACAAGAAAAACTGTATTTGACAAACAAGAT |
| | ACATTGTATGAAAATACAAGAAAGTTTGT |
| | (SEQ ID NO: 190) |
| Bac434-OR1-7 | ACAAGAAAGTTTGTTGAGTCCTAGACAAGA |
| | AAGTTTGTTCTTCACAGGACAAGAAAGTTT |
| | GTTAGGGTTCCTACAAGAAAGTTTGTACAC |
| | TAGAGTACAAGAAAGTTTGTGATAGTATCA |
| | ACAAGAAAGTTTGTAGCAAACGAAACAAGA |
| | AAGTTTGT |
| | (SEQ ID NO: 191) |
| Bac434-OR3-7 | ACAAGAAAAACTGTTGAGTCCTAGACAAGA |
| | AAAACTGTTCTTCACAGGACAAGAAAAACT |
| | GTTAGGGTTCCTACAAGAAAAACTGTACAC |
| | TAGAGTACAAGAAAAACTGTGATAGTATCA |
| | ACAAGAAAAACTGTAGCAAACGAAACAAGA |
| | AAAACTGT |
| | (SEQ ID NO: 192) |
| Bac434-OR321-3 | CCCAATCTTCACAAGAAAAACTGTATTTGA |
| | CAAACAAGATACATTGTATGAAAATACAAG |
| | AAAGTTTGTTGATGGAGGCACAAGAAAAAC |
| | TGTATTTGACAAACAAGATACATTGTATGA |
| | AAATACAAGAAAGTTTGTTGATGGAGGCAC |
| | AAGAAAAACTGTATTTGACAAACAAGATAC |
| | ATTGTATGAAAATACAAGAAAGTTTGTTGA |
| | TGGAGGC |
| | (SEQ ID NO: 193) |

In some embodiments, the TFBS is a binding sequence for a GCN4 protein. In some such embodiments, the GCN4 TFBS comprises a sequence having 80%, 85%, 90% identity or more to TGACTC, in some cases 95% identity or more to TGACTC, in certain cases 100% identity with TGACTC, for example, AGTGACTCATT (SEQ ID NO:450). In some embodiments, the DTS comprises 2, 3, 4, 5, 6, 7, 8, 9, or 10 copies of the GCN4 TFBS. In certain embodiments, the DTS comprises 5 copies of the GCN4 TFBS. In certain embodiments, the DTS comprises 7 copies of the GCN4 TFBS. In certain embodiments, the DTS comprises 10 or more copies of the GCN4 TFBS. In some embodiments, the DTS comprises a sequence having 80%, 85%, or 90% identity or more to the sequence AGTGACTCATTTGAGTCCTAGAGTGACTCATTTCTTCACAGGAGTGACTCATTTAGGGTTCCT AGTGACTCATTACACTAGAGTAGTGACTCATTGATAGTATCAAGTGACTCATTAGCAAACGA AAGTGACTCATT (SEQ ID NO:194), in some cases 95% identity or more to this sequence, in certain cases sharing 100% identity with this sequence.

In some embodiments, the TFBS is a binding sequence for a Lactose Inhibitor (LacI) protein, also referred to herein as a Lactose Repressor (LacR) protein, for example, a LacO sequence, e.g., TTGTTATCCGCTCACAA (SEQ ID NO:195). In some such embodiments, the LacR TFBS comprises a sequence having 80%, 85%, 90% identity or more to TTGTTATCCGCTCACAA (SEQ ID NO:196). In certain embodiments, the DTS comprises a lactose operon (LacO), as is known in the art. In certain embodiments, the DTS consists essentially of a LacO. In some embodiments, the DTS comprises 2, 3, 4, 5, 6, 7, 8, 9, or 10 copies of the LacR TFBS. In certain embodiments, the DTS comprises 5 copies of the LacR TFBS. In certain embodiments, the DTS comprises 7 copies of the LacR TFBS. In certain embodiments, the DTS comprises 10 or more copies of the LacR TFBS. In some embodiments, the DTS comprises a sequence that has 80%, 85%, or 90% identity or more to the sequence TTGTTATCCGCTCACAATTCCACATGTGGCCACAAATTGTTATCCGCTCACAATTCCACATGT GGCCACAAATTGTTATCCGCTCACAATTCCACATGTGGCCACAAATTGTTATCCGCTCACAA TTCCACATGTGGCCACAAATTGTTATCCGCTCACAATTCCACATGTGGCCACAAATTGTTATC CGCTCACAATTCCACATGTGGCCACAAATTGTTATCCGCTCACAA (SEQ ID NO:197), in some cases 95% identity or more to this sequence, in certain cases sharing 100% identity with this sequence.

In some embodiments, the TFBS is a binding sequence for an endonuclease, e.g., a I-SceI D44A protein. In some embodiments the DTS comprises a TFBS having 90% identity or more to the sequence

(SEQ ID NO: 198)
TAGGGATAACAGGGTAAT.

It will be appreciated by one of ordinary skill in the art that DNA binding proteins can tolerate some degree of nucleotide substitution in their binding sequences and that the TFBSs provided herein are but examples of sequences that may be used. In some embodiments, the TFBS shares 80% identity or more with a sequence disclosed herein, for example, 85%, 90%, 95% identity or more, e.g., 96%, 97%, 98%, or 99% identity or more, in certain instances 100% identity to the sequence. Publicly available databases such as Uniprot, Cis-BP, Transfac, and GrassiusX can be consulted to identify which nucleotides can be varied and which should be conserved in designing DTSs for use in the inventions of the present disclosure.

A given DTS may include 2 or more different TFBSs (i.e., TFBSs that differ from each other by nucleotide sequence and are therefore distinct), such as 2, 3, 4, 5, 6, 7, 8, 9 or 10 different TFBSs. A given DTS may include 1 or more copies of the same TFBS, such as 2 or more, 3 or more, 4 or more, 5 or more, 6 or more, 7 or more, 8 or more, 9 or more, copies of the same TFBS, where in some instances the number of identical TFBS copies does not exceed 10.

The length of a given DTS may vary depending on the number of TFBSs comprised by it, the lengths of the TFBS sequence to which each nuclear targeting factor binds, and the number nucleotides between TFBSs (the spacer sequence). Where two or more TFBSs (either the same or different) are present in a given DTS, the distance between any two TFBSs may vary as desired, ranging in some instances from 5 to 100 bp, such as 10 to 75 bp, including 15 to 50 bp. For example, the TFBSs may be separated from one another by 0, 1, 2, 3, 4, 5, 6, 7, 8, 9, 10, 11, 12, 13, 14, 15, 16, 17, 18, 19, or 20 or more nts, for example, 0-5 nts, 6-10 nts, 11-15 nts, 16-20 nts, 21-25 nts, 26-30 nts, 31-35 nts, 36-40 nts, in some instances 40-50 nts. In some instances, the length of a given DTS ranges from 10 to 500 nt, such as 100 to 300 nt and including 150 to 200 nt.

A given NTDNA may include a single DTS or a plurality of DTSs, as desired. Where a given NTDNA includes a plurality of DTSs, the disparate DTSs may be the same or different. As such, a given NTDNA may include 2 or more different DTSs (i.e., DTSs that differ from each other by nucleotide sequence and are therefore distinct).

The DTS may be incorporated in the NTDNA in any of a variety of positions. For example, the DTS may be placed 5′ of the promoter, 3′ of the promoter and 5′ of the expression cassette, within an intron of the expression cassette, or 3′ of the expression cassette. In some instances, the DTSs is placed 5′ of the promoter. In some embodiments, the DTS is placed 3′ of the expression cassette.

As reviewed above, in addition to a DTS component (e.g., made up of one or more TFBS sequences), NTDNAs employed in methods of the invention also include a cargo nucleic acid that is heterologous to the DTS. By “heterologous” to the DTS it is meant that the cargo nucleic acid is not naturally associated with the DTS, e.g., is not part of the same gene as the DTS in nature. The cargo nucleic acid may vary as desired. In some instances, the cargo nucleic acid is 500 nt or more, for example 1 kb, 2 kb, 3 kb, 4 kb, or 5 kb or more, e.g., 6 kb, 7 kb, 8 kb, 9 kb, 10 kb or more, in some cases, 15 kb or more. The cargo nucleic has may have any desired sequence. In some instances, the cargo nucleic acid may include one or more of coding sequences, promoters, sequences homologous to the genomic DNA of the targeted nucleus (e.g., to provide for genomic integration of the cargo nucleic acid), untranslated sequences (5′ UTR, 3′ UTR), polyadenylation sequences, and the like.

A cargo nucleic acid to be delivered to the nucleus may be configured to be maintained episomally or integrated into the genome, as desired. As such, in some instances a cargo nucleic acid is configured to be maintained episomally in the nucleus of a target cell, such that it is not genomically integrated. In other instances, the cargo nucleic acid may be configured to be integrated into the genome of a target cell. In such instances, integration may be accomplished using any convenient protocol, such as by using a gene editing system, e.g., as described in greater detail below.

In some instances, the cargo nucleic acid includes a coding sequence. By a coding sequence it is meant a nucleic acid sequence that encodes for any gene product, e.g., micro RNA (miRNA), small hairpin RNA (shRNA), circular RNA (circRNA), long noncoding RNA (lncRNA), mRNA, peptide, polypeptide, protein. Where desired, a given coding sequence can encode multiple gene products, e.g., separated by IRES sequence, 2A sequence, or the like. For example, a coding sequence to be delivered to the nucleus by a NTDNA can be configured so that it can be integrated into the genome in operable linkage with its native promoter, for example to replace a mutant coding sequence or to be in operable linkage with an active promoter at a safe harbor (in which instances, the cargo nucleic acid need not include, and in some instances does not include, a promoter).

In some instances, a cargo nucleic acid may include a promoter. As used herein, the term “promoter” refers to any nucleic acid sequence that regulates the expression of another nucleic acid sequence by driving transcription of the nucleic acid sequence, which can be a heterologous target gene encoding a protein or an RNA. Promoters can be constitutive, inducible, repressible, tissue-specific, or any combination thereof. A promoter is a control region of a nucleic acid sequence at which initiation and rate of transcription of the remainder of a nucleic acid sequence are controlled. A promoter can also contain genetic elements at which regulatory proteins and molecules can bind, such as RNA polymerase and other transcription factors. Within the promoter sequence will be found a transcription initiation site, as well as protein binding domains responsible for the binding of RNA polymerase. Eukaryotic promoters will often, but not always, contain “TATA” boxes and “CAT” boxes. Various promoters, including inducible promoters, may be used to drive the expression of transgenes. A promoter sequence may be bounded at its 3′ terminus by the transcription initiation site and extends upstream (5′ direction) to include the minimum number of bases or elements necessary to initiate transcription at levels detectable above background. Promoters useful in NTDNAs of embodiments of the invention include, for example constitutively active promoters, such as the CMV promoter, CAG promoter (which combines the CMV enhancer with the chicken-actin (CBA) promoter), β-actin promoter, SV-40 promoter, 4xGRM6-SV40, hTTR, hAAT, 3x-Serpina, ubiquitin B/C, EFI-Alpha, EFS, and HBV promoter, etc. Promoters useful in NTDNAs of embodiments of the invention also include promoters having more cell-type specific expression patterns, for example for hepatocytes, may include, without limitation the TTR, hAAT (and derivatives), 3xSerpina-TTR, HBV, UbiC, and P3-hybrid promoter. A cargo nucleic acid may include a promoter sequence to be delivered to the nucleus so that it can be integrated into the genome, for example to replace a mutant promoter.

In some instances, the cargo nucleic acid includes an expression cassette. By an expression cassette it is meant a nucleic acid sequence comprising a promoter, e.g., as described above, operably linked to a coding sequence, e.g., as described above (also referred to herein as a transgene). In some instances, the expression cassette may also comprise one or more nucleic acid sequences including a 5′ untranslated region (5′ UTR), 3′ untranslated region (3′ UTR), polyA tail, miRNA regulatory element. In embodiments, the expression cassette may include a transgene and one or more regulatory sequences that allows and/or controls the expression of the transgene, e.g., where the expression cassette can include one or more of, e.g., in this order: an enhancer/promoter, an ORF reporter (transgene), a post-transcription regulatory element (e.g., WPRE), and a polyadenylation and termination signal (e.g., BGH polyA). The expression cassette can also comprise an internal ribosome entry site (IRES) and/or a 2A element. The cis-regulatory elements include, but are not limited to, a promoter, a riboswitch, an insulator, a mir-regulatable element, a post-transcriptional regulatory element, a tissue- and cell type-specific promoter and an enhancer. As desired, the expression cassette can comprise 4000 or more nucleotides, 5000 or more nucleotides, 10,000 or more nucleotides or 20,000 or more nucleotides, or 30,000 or more nucleotides, or 40,000 or more nucleotides or 50,000 or more nucleotides, and in some instances may range between 4000-10,000 nucleotides or 10,000-50,000 nucleotides, or more than 50,000 nucleotides. The expression cassette to be delivered to the nucleus may be configured so that it can be maintained episomally. Alternatively, an expression cassette to be delivered to the nucleus may be configured so that it can be integrated into the genome.

The coding sequence e.g., transgene, of the expression cassette may vary. In some embodiments, the expression cassette can include a transgene in the range of 500 to 50,000 nucleotides in length. In some embodiments, the expression cassette can include a transgene in the range of 500 to 75,000 nucleotides in length. In some embodiments, the expression cassette can include a transgene which is in the range of 500 to 10,000 nucleotides in length. In some embodiments, the expression cassette can include a transgene which is in the range of 1000 to 10,000 nucleotides in length. In some embodiments, the expression cassette can include a transgene which is in the range of 500 to 5,000 nucleotides in length. The NTDNA constructs of embodiments of the invention do not have the size limitations of encapsidated AAV vectors, and thus enable nuclear delivery of large-size expression cassettes to provide efficient transgene.

A given expression cassette can include, for example, an expressible exogenous sequence (e.g., open reading frame) or transgene that encodes a protein that is either absent, inactive, or insufficient activity in the recipient subject or a gene that encodes a protein having a desired biological or a therapeutic effect. The transgene can encode a gene product that can function to correct the expression of a defective gene or transcript. In principle, the expression cassette can include any gene that encodes a protein, polypeptide or RNA that is either reduced or absent due to a mutation or which conveys a therapeutic benefit when overexpressed is considered to be within the scope of the disclosure. The expression cassette can include any transgene useful for treating a disease or disorder in a subject. NTDNAs, e.g., as described herein, can be used to deliver and express any gene of interest in a subject, where such genes include, but are not not limited to, nucleic acids encoding polypeptides, or non-coding nucleic acids (e.g., RNAi, miRs etc.), as well as exogenous genes and nucleotide sequences, including virus sequences in a subjects' genome, e.g., HIV virus sequences and the like. In some instances, a NTDNA (e.g., as disclosed herein) is used for therapeutic purposes (e.g., for medical, diagnostic, or veterinary uses) or immunogenic polypeptides. In certain embodiments, a NTDNA is useful to express any gene of interest in the subject, which includes one or more polypeptides, peptides, ribozymes, peptide nucleic acids, siRNAs, RNAis, antisense oligonucleotides, antisense polynucleotides, or RNAs (coding or non-coding; e.g., siRNAs, shRNAs, micro-RNAs, and their antisense counterparts (e.g., antagoMiR)), antibodies, antigen binding fragments, or any combination thereof. As such, expression cassettes can encode polypeptides, sense or antisense oligonucleotides, or RNAs (coding or non-coding; e.g., siRNAs, shRNAs, micro-RNAs, and their antisense counterparts (e.g., antagoMiR)). Expression cassettes can include an exogenous sequence that encodes a reporter protein to be used for experimental or diagnostic purposes, such as β-lactamase, β-galactosidase (LacZ), alkaline phosphatase, thymidine kinase, green fluorescent protein (GFP), chloramphenicol acetyltransferase (CAT), luciferase, EPO, and others well known in the art.

Sequences provided in a given expression cassette, expression construct of a NTDNA, e.g., as described herein, can be codon optimized for the target host cell. As used herein, the term “codon optimized” or “codon optimization” refers to the process of modifying a nucleic acid sequence for enhanced expression in the cells of the vertebrate of interest, e.g., mouse or human, by replacing at least one, more than one, or a significant number of codons of the native sequence (e.g., a prokaryotic sequence) with codons that are more frequently or most frequently used in the genes of that vertebrate. Various species exhibit particular bias for certain codons of a particular amino acid. Typically, codon optimization does not alter the amino acid sequence of the original translated protein. In some embodiments, a transgene expressed by the NTDNA is a therapeutic gene. In some embodiments, a therapeutic gene is an antibody, or antibody fragment, or antigen-binding fragment thereof, e.g., a neutralizing antibody or antibody fragment and the like. In some instances, a therapeutic gene is one or more therapeutic agent(s), including, but not limited to, for example, protein(s), polypeptide(s), peptide(s), enzyme(s), antibodies, antigen binding fragments, as well as variants, and/or active fragments thereof, for use in the treatment, prophylaxis, and/or amelioration of one or more symptoms of a disease, dysfunction, injury, and/or disorder. Of interest in certain embodiments are transgenes that are heterologous relative to the DTS of the NTDNA. For example, a coding sequence to be delivered to the nucleus so that it can be integrated into the genome in operable linkage with its native promoter, for example to replace a mutant coding sequence or to be in operable linkage with an active promoter at a safe harbor (in which instances, the polynucleotide does not comprise a promoter); or the coding sequence of an expression cassette to be delivered to the nucleus to be either maintained episomally or integrated into the genome. By a coding sequence it is meant a nucleic acid sequence that encodes for any gene product, e.g., micro RNA (miRNA), small hairpin RNA (shRNA), circular RNA (circRNA), long noncoding RNA (lncRNA), mRNA, peptide, polypeptide, protein. Can encode multiple gene products, e.g., separated by IRES sequence, 2A sequence, or the like.

Where desired, a given cargo nucleic acid may include flanking sequences that are homologous to genomic regions of the cell, e.g., to promote genomic integration of the cargo nucleic acid. When present, such sequences may vary in length, ranging in some instances from 30 to 5,000 nt, such as 50 to 1000 nt. While the sequences of such regions may vary depending on the genomic integration location of interest, examples of such sequences include, but are not limited to: actin, ADA, albumin, α-globin, β-globin, CD2, CD3, CD5, CD7, CCR5, Ela, IL2RG, Ins1, Ins2, NCF1, p50, p65, PF4, PGC-γ, PTEN, TERT, TRAC, UBC, and VWF, and the like.

In embodiments where the NTDNA is to be integrated into the genome of a target cell in process mediated by a gene editing system, e.g., as described below, the NTDNA may be serve as a Donor DNA or Donor Template in such a system. Site-directed polypeptides, such as a DNA endonuclease, can introduce double-strand breaks or single-strand breaks in nucleic acids, e.g., genomic DNA. The double-strand break can stimulate a cell's endogenous DNA-repair pathways (e.g., homology-dependent repair (HDR) or non-homologous end joining or alternative non-homologous end joining (A-NHEJ) or microhomology-mediated end joining (MMEJ). NHEJ can repair cleaved target nucleic acid without the need for a homologous template. This can sometimes result in small deletions or insertions (indels) in the target nucleic acid at the site of cleavage, and can lead to disruption or alteration of gene expression. HDR, which is also known as homologous recombination (HR) can occur when a homologous repair template, or donor, is available.

The homologous donor template has sequences that are homologous to sequences flanking the target nucleic acid cleavage site. The sister chromatid is generally used by the cell as the repair template. However, for the purposes of genome editing, the repair template is often supplied as an exogenous nucleic acid, such as a plasmid, duplex oligonucleotide, single-strand oligonucleotide, double-stranded oligonucleotide, or viral nucleic acid. With exogenous donor templates, it is common to introduce an additional nucleic acid sequence (such as a transgene) or modification (such as a single or multiple base change or a deletion) between the flanking regions of homology so that the additional or altered nucleic acid sequence also becomes incorporated into the target locus. MMEJ results in a genetic outcome that is similar to NHEJ in that small deletions and insertions can occur at the cleavage site. MMEJ makes use of homologous sequences of a few base pairs flanking the cleavage site to drive a favored end-joining DNA repair outcome. In some instances, it can be possible to predict likely repair outcomes based on analysis of potential microhomologies in the nuclease target regions.

Thus, in some cases, homologous recombination is used to insert an exogenous polynucleotide sequence into the target nucleic acid cleavage site. An exogenous polynucleotide sequence is termed a donor polynucleotide (or donor or donor sequence or polynucleotide donor template) herein, and may in embodiments of the present invention be an NTDNA or component thereof, e.g., cargo nucleic acid component of an NTDNA. In some embodiments, the donor polynucleotide, a portion of the donor polynucleotide, a copy of the donor polynucleotide, or a portion of a copy of the donor polynucleotide is inserted into the target nucleic acid cleavage site. In some embodiments, the donor polynucleotide is an exogenous polynucleotide sequence, i.e., a sequence that does not naturally occur at the target nucleic acid cleavage site.

When an exogenous DNA molecule is supplied in sufficient concentration inside the nucleus of a cell in which the double strand break occurs, the exogenous DNA can be inserted at the double strand break during the NHEJ repair process and thus become a permanent addition to the genome. These exogenous DNA molecules are referred to as donor templates in some embodiments. If the donor template contains a coding sequence for a gene-of-interest optionally together with relevant regulatory sequences such as promoters, enhancers, polyA sequences and/or splice acceptor sequences (also referred to herein as a “donor cassette”), the gene of interest can be expressed from the integrated copy in the genome resulting in permanent expression for the life of the cell. Moreover, the integrated copy of the donor DNA template can be transmitted to the daughter cells when the cell divides.

In the presence of sufficient concentrations of a donor DNA template that contains flanking DNA sequences with homology to the DNA sequence either side of the double strand break (referred to as homology arms), the donor DNA template can be integrated via the HDR pathway. The homology arms act as substrates for homologous recombination between the donor template and the sequences either side of the double strand break. This can result in an error free insertion of the donor template in which the sequences either side of the double strand break are not altered from that in the un-modified genome.

Supplied donors for editing by HDR vary markedly but generally contain the intended sequence with small or large flanking homology arms to allow annealing to the genomic DNA. The homology regions flanking the introduced genetic changes can be 30 bp or smaller, or as large as a multi-kilobase cassette that can contain promoters, cDNAs, etc. Both single-stranded and double-stranded oligonucleotide donors can be used. These oligonucleotides range in size from less than 100 nt to over many kb, though longer ssDNA can also be generated and used. Double-stranded donors are often used, including PCR amplicons, plasmids, and mini-circles.

In some embodiments, an exogenous sequence, e.g., cargo nucleic acid, that is intended to be inserted into a genome is a gene-of-interest (GOI) or functional derivative thereof. The exogenous gene can include a nucleotide sequence encoding a GOI product, e.g., GOI protein, or functional derivative thereof. The functional derivative of a GOI can include a nucleic acid sequence encoding a functional derivative of a GOI protein that has a substantial activity of a wildtype GOI protein such as. the wildtype human GOI protein, e.g., at least about 30%, about 40%, about 50%, about 60%, about 70%, about 80%, about 90%, about 95% or about 100% of the activity that the wildtype GOI protein exhibits. In some embodiments, the functional derivative of a GOI protein can have at least about 30%, about 40%, about 50%, about 60%, about 70%, about 80%, about 85%, about 90%, about 95%, about 96%, about 97%, about 98% or about 99% amino acid sequence identity to the GOI protein, e.g., the wildtype GOI protein. In some embodiments, one having ordinary skill in the art can use a number of methods known in the field to test the functionality or activity of a compound, e.g., peptide or protein. The functional derivative of the GOI protein can also include any fragment of the wildtype GOI protein or fragment of a modified GOI protein that has conservative modification on one or more of amino acid residues in the full length, wildtype GOI protein. Thus, in some embodiments, the functional derivative of a nucleic acid sequence of a GOI can have at least about 30%, about 40%, about 50%, about 60%, about 70%, about 80%, about 85%, about 90%, about 95%, about 96%, about 97%, about 98% or about 99% nucleic acid sequence identity to the GOI, e g. the wildtype GOI.

In some embodiments where the insertion of a GOI or functional derivative thereof is concerned, a cDNA of GOI or functional derivative thereof can be inserted into a genome of a patient having defective GOI or its regulatory sequences. In such a case, a donor DNA or donor template can be an expression cassette or vector construct having the sequence encoding GOI or functional derivative thereof, e.g., cDNA sequence. In some embodiments, the expression vector contains a sequence encoding a modified GOI protein, which is described elsewhere in the disclosures, can be used.

In some embodiments, according to any of the donor templates described herein comprising a donor cassette, the donor cassette is flanked on one or both sides by a gRNA target site. For example, such a donor template may comprise a donor cassette with a gRNA target site 5′ of the donor cassette and/or a gRNA target site 3′ of the donor cassette. In some embodiments, the donor template comprises a donor cassette with a gRNA target site 5′ of the donor cassette. In some embodiments, the donor template comprises a donor cassette with a gRNA target site 3′ of the donor cassette. In some embodiments, the donor template comprises a donor cassette with a gRNA target site 5′ of the donor cassette and a gRNA target site 3′ of the donor cassette. In some embodiments, the donor template comprises a donor cassette with a gRNA target site 5′ of the donor cassette and a gRNA target site 3′ of the donor cassette, and the two gRNA target sites comprise the same sequence. In some embodiments, the donor template comprises at least one gRNA target site, and the at least one gRNA target site in the donor template comprises the same sequence as a gRNA target site in a target locus into which the donor cassette of the donor template is to be integrated. In some embodiments, the donor template comprises at least one gRNA target site, and the at least one gRNA target site in the donor template comprises the reverse complement of a gRNA target site in a target locus into which the donor cassette of the donor template is to be integrated. In some embodiments, the donor template comprises at least one gRNA target site, and the at least one gRNA target site in the donor template does not comprise the same sequence as a gRNA target site in a target locus into which the donor cassette of the donor template is to be integrated. In some embodiments, the donor template comprises a donor cassette with a gRNA target site 5′ of the donor cassette and a gRNA target site 3′ of the donor cassette, and the two gRNA target sites in the donor template comprises the same sequence as a gRNA target site in a target locus into which the donor cassette of the donor template is to be integrated. In some embodiments, the donor template comprises a donor cassette with a gRNA target site 5′ of the donor cassette and a gRNA target site 3′ of the donor cassette, and the two gRNA target sites in the donor template comprises the reverse complement of a gRNA target site in a target locus into which the donor cassette of the donor template is to be integrated. In some embodiments, the donor template comprises a donor cassette with a gRNA target site 5′ of the donor cassette and a gRNA target site 3′ of the donor cassette, and the two gRNA target sites in the donor template do not comprise the same sequence as a gRNA target site in a target locus into which the donor cassette of the donor template is to be integrated.

A given NTDNA may or may not be configured to be maintained in a bacterium, as desired. For example, in some instances a NTDNA is configured to be maintained in a bacterium. In such instances, the NTDNA may include a bacterial origin of replication and a selection element, e.g., a plasmid or a nanoplasmid. In other instances, a NTDNA may be configured to not be maintained in a bacterium. In such instances, the structure of the NTDNA may vary, wherein examples of such structures include minicircle DNA (mcDNA), a linear DNA, a covalently closed DNA (doggybone DNA, or “dbDNA”, ministring DNA, etc.), double stranded linear DNA comprising terminal repeat sequences at both ends, a 3DNA structure, etc., and the like.

### Inducing Agent

As summarized above, in some embodiments, in addition to the NTDNA, a cell may also be contacted with an inducing agent that activates the nuclear targeting factor to which the DTS binds to mediate nuclear entry of the NTDNA. In other words, the inducing agent modulates the nuclear targeting factor so that it translocates from the cytosol to the nucleus, and in doing so brings the NTDNA associated with it (via the binding interaction between the DTS and the nuclear targeting factor) into the nucleus. As such, activity of the inducing agent on the nuclear targeting factor causes the nuclear targeting factor to mediate entry of the NTDNA (and therefore cargo nucleic acid thereof) into the nucleus. Inducing agents may vary depending on the nuclear targeting factor of a given system. Examples of inducing agents include those that act directly on nuclear targeting factors to cause translocation thereof from the cytosol to the nucleus. Examples of such inducing agents include, but are not limited to, steroid hormones and derivatives (such as estrogen, progesterone, glucocorticoids, Vitamin D, oxysterols and bile acids, among others) as well as receptors for retinoic acids, thyroid hormones and fatty acids and their derivatives. These ligands are able to diffuse directly through cellular membranes as a result of their lipophilic nature (reviewed in Beato et al, Steroids 1996 April; 61 (4): 240-51; Holzer et al, Curr Top Dev Biol. 2017; 125:1-38. 2017). Also of interest as inducing agents are agents that indirectly activate the nuclear targeting factor. For example, an inducing agent may indirectly activate a transcription factor that resides quiescent in the cytoplasm and becomes indirectly activated when the cell comes in contact with the inducing reagent. By “indirectly activated” it is meant that nuclear targeting factor, e.g., transcription factor, becomes activated by a protein that binds the inducing reagent. Examples of such transcription factors include NF-kB, CREB, and the like. Examples of inducible nuclear targeting factors, the TFBSs to which they bind, and the inducible ligand that activates them may be found in Table 2 above.

Within embodiments of the invention, specific combinations of nuclear targeting factors, DTSs and inducing agents may vary, as desired. Examples of combinations of interest that may be employed in a given embodiment include, but are not limited to, those embodiments provided in Table 2, above.

### Exogenous Nuclear Targeting Factors

As summarized above, in some embodiments, in addition to the NTDNA, a cell may also be contacted with an mRNA that encodes the nuclear targeting factor to which the DTS binds to mediate nuclear entry of the NTDNA. Without wishing to be bound by theory, it is believed that the exogenously provided nuclear targeting factor is translated from the mRNA and translocates from the cytosol to the nucleus, and in doing so brings the NTDNA associated with it (via the binding interaction between the DTS and the nuclear targeting factor) into the nucleus. Typically, the nuclear targeting factor will comprise a nuclear localization sequence (NLS) or a fragment thereof. In some instances, the NLS will be native, or endogenous, to the nuclear targeting factor. In other instances, a DNA binding protein will be engineered to comprise an NLS so as to create the nuclear targeting factor. If engineered into the NTF, the NLS may be engineered to be anywhere within the NTF. In some instances, the NLS is engineered to be at the N terminus. In some instances, the NLS is engineered to be at the C terminus. In some instances, the NLS is engineered to be at both the N terminus and the C terminus. When engineered to reside at a terminus, the NLS is typically engineered to be within 0-20 amino acids of the terminus, in some instances, within 0-10 amino acids of the terminus, in certain instances within 0-5 amino acids of the terminus, in some such cases, at the terminus. Typically, the NLS is engineered to be at a site that is distinct from the protein domain that mediates binding to the DNA. In some instances, the NLS may be flanked by one or more amino acids, e.g., 2, 3, 4, 5, 6, 7, 8, 9, 10, 11, 12, 13, 14, 15, 16, 17, 18, 19, or 20 amino acids. In some instances, the nuclear targeting factor will comprise one NLS, in other instances multiple NLSs. In some instances, in which multiple NLSs are employed, the same NLS is employed multiple times. In other instances, in which multiple NLSs are employed, different NLSs are used. Often, when multiple NLSs are employed, they will be separated by a linker, e.g., a 6xK linker. Exemplary NLSs and their origins are provided in Table 7.

TABLE 7
Exemplary NLS sequences

| NLS origin | Sequence |
|---|---|
| SV40 Large T antigen SMALL | PKKKRKVED (SEQ ID NO: 199) |
| SV40 Large T antigen | KKKSSSDDEATADSQHSTPPKKKRKVEDPKDFPSE |
| | LLS (SEQ ID NO: 200) |
| Optimized SV40 Large T antigen | PSSDDEATADSQHAAPPKKKRKVEDPKDFPSELLS |
| | (SEQ ID NO: 201) |
| Heterogeneous nuclear RNP A1.1 M9 | GNQSSNFGPMKGGNFGGRSSGPYGGGGQYFAKPR |
| domain | NQGGYGG (SEQ ID NO: 202) |
| Heterogeneous nuclear RNP A1.2 M9 | FGNYNNQSSNFGPMKGGNFGGRSSGPYGGGGQYF |
| domain | AKPRNQGGYGG (SEQ ID NO: 203) |
| Heterogeneous nuclear RNP A1.3 M9 | FGNYNNQSSNFGPMKGGNFGGRSSGPYGGGGQYF |
| domain | AKPRNQGGYGGSSSSSSYGSGRRF (SEQ ID NO: 204) |
| HIV viral protein 1 (VP1) | DTWTGVEALIRILQQLLFIHFRIGCRHSRIGIIQQRR |
| | TRNGA (SEQ ID NO: 205 |
| HIV viral protein 2 (VP2) | GDTWAGVEAIIRILQQLLFIHFRIGCRHSRIGVTRQR |
| | RARNGASRS (SEQ ID NO: 206) |
| HIV viral protein 3 (VP3) | EQAPEDQGPQREPHNEWTLELLEELKNEAVRHFPR |
| | IWLHGLGQHIYETYGDTWAGVEAIIRILQQLLFIHF |
| | RIGCRHSRIGVTRQRRARNGASRS (SEQ ID NO: 207) |
| Influenza virus A nuclear protein (INF-A) | ASQGTKRSYEQMETDGERQ (SEQ ID NO: 208) |
| SV40 short fused to Influenza A NLS | PKKKRKVASQGTKRSYEQMETDGERQ (SEQ ID |
| | NO: 209) |
| Influenza A NLS fused to SV40 short | MASQGTKRSYEQMETDGERQPKKKRKV (SEQ ID |
| | NO: 210) |
| Cellular myc transcription factor (c-myc) | PAAKRVKLD (SEQ ID NO: 211) |
| c-myc fused to INF-A | PAAKRVKLDASQGTKRSYEQMETDGERQ (SEQ ID |
| | NO: 212) |
| c-myc NLS fused to Influenza A NLS, | PAAKRVKLDASQGTKRSYEQMETDGERQKKKSSS |
| fused to SV40 long version | DDEATADSQHSTPPKKKRKVEDPKDFPSELLS |
| | (SEQ ID NO: 213) |
| c-myc NLS fused to Influenza A NLS, | PAAKRVKLDASQGTKRSYEQMETDGERQPAAKR |
| fused to a second c-myc NLS | VKLD (SEQ ID NO: 214) |
| Influenza A NLS fused to c-myc NLS | MASQGTKRSYEQMETDGERQPAAKRVKLD (SEQ |
| | ID NO: 215) |
| Biparticle nucleus localization signal | KRTADGSEFESPKKKRKV (SEQ ID NO: 216) |

In some embodiments, the nuclear targeting factor comprises a nuclear export signal. In some embodiments, the NES is NMD3 ribosome export adaptor. In some such embodiments, the NES comprises a sequence having 85% identity or more to NELALKLAGLDINKT (SEQ ID NO:217). In some such embodiments, the NES comprises a sequence having 85% identity or more to EHVNKMNSDRVPDVVLIKKSYDRTKRQRRRNWKLKELA (SEQ ID NO:218).

In some embodiments, the nuclear targeting factor comprises a tag, a linker or other sequence that may be used, e.g., to preserve the structure of an appended NLS or NES, to purify protein in recombinant protein production, to visualize the protein in cells, etc. Examples of tags or linkers include but are not limited to CS3, HIS, Flag, GGS, RIGID, SpyTag, Strep tags, etc. Exemplary linkers are provided in Table 8.

TABLE 8
Exemplary linkers.

| NAME | AMINO ACID |
|---|---|
| S1 (StrepTag 1X) | WSHPQFEK |
| | (SEQ ID NO: 219) |
| S2 (StrepTag 2X) | WSHPQFEKWSHPQFEK |
| | (SEQ ID NO: 220) |
| S3 (StrepTag 3X) | WSHPQFEKWSHPQFEKWSHPQFEK |
| | (SEQ ID NO: 221) |
| GGS 1X | GGS |
| | (SEQ ID NO: 222) |
| GGS 2X | GGSGGS |
| | (SEQ ID NO: 223) |
| GGS 3X | GGSGGSGGS |
| | (SEQ ID NO: 224) |
| GGGGS 1X | GGGGS |
| | (SEQ ID NO: 225) |
| GGGGS 2X | GGGGSGGGGS |
| | (SEQ ID NO: 226) |
| GGGGS 3X | GGGGSGGGGSGGGGS |
| | (SEQ ID NO: 227) |
| GGGGS 4X | GGGGSGGGGSGGGGSGGGGS |
| | (SEQ ID NO: 228) |
| QSGSGSTATGSG linker | QSGSGSTATGSG |
| | (SEQ ID NO: 229) |
| Rigid | GTPTPTPTPTG |
| | (SEQ ID NO: 230) |
| M_Flex | SGGGSGGSGSS |
| | (SEQ ID NO: 231) |
| W_Flex | GSAGSAAGSGEF |
| | (SEQ ID NO: 232) |

Any method for making mRNA may be employed to generate the subject mRNA encoding the NTF. As one example, mRNA may be chemically synthesized using solid-phase methods. As another example, mRNA may be synthesized from a DNA template by an in vitro transcription (IVT) reaction, in which a DNA template comprising a promoter sequence for an RNA polymerase operably linked to a sequence encoding the target mRNA (in this instance, the nuclear targeting factor) is contacted with the RNA polymerase, which binds the template at the promoter region and starts the RNA synthesis. As yet another example, the mRNA may be synthesized in vivo and purified from the biological source. The mRNA may comprise naturally occurring ribonucleotides and/or chemically modified ribonucleotides. Typically, some chemically modified nucleotides may be included to reduce the immunogenicity of the mRNA, for example, N¹-methylpseudouridine, 2-thiouridine (s²U), 5-methylcytidine (m⁵C), N⁶-methyladenosine (m⁶A), 2′-O-methyluridine (Um), 2′-O-methylcytidine (Cm), 2′-O-methyladenosine (Am), and 2′-O-methylguanosine (Gm), N⁴-Acetyl-cytidine 5-triphosphate (AC4C). The mRNA may comprise a 5′ cap or analog thereof, e.g., an m⁷GpppG cap, anti-reverse cap analog (ARCA), a two-headed cap, an S cap, a 2S cap, and the like. The mRNA will typically comprise a 5′ untranslated region (UTR). The mRNA will typically comprise a 3′ UTR, e.g., as described in Table 9. The mRNA may comprise a tail modification, e.g., a ribose modified adenosine, 8-azaadenosine, cordycepin, and the like.

TABLE 9
Exemplary 3′ UTRs

| UTR | Sequence |
|---|---|
| T7 promoter | TAATACGACTCACTATAGG (SEQ ID NO: 233) |
| T7 promoter v2 | TAATACGACTCACTATAAG (SEQ ID NO: 234) |
| T7 promoter m6Am | TAATACGACTCACTATAAGTCA (SEQ ID NO: 235) |
| T7 enhancer | GGGATAAT (SEQ ID NO: 236) |
| T7 modified enhancer | GGGATAATAG (SEQ ID NO: 237) |
| Kozak | GCCACCATG (SEQ ID NO: 238) |
| modified Kozak | GCCGCCACCATG (SEQ ID NO: 239) |
| m6a modification site | GGACA (SEQ ID NO: 240) |
| m6a modification site 2 | TGACT (SEQ ID NO: 241) |
| m6a modification site 3 | AGACCAGACA (SEQ ID NO: 242) |
| m6a modification site 4 | GGACTGGACTGGACT (SEQ ID NO: 243) |
| CAP binding site | TAATGTGAGTTAGCTCACTCAT (SEQ ID NO: 244) |
| 2′-O-methyl site | AGATCGAGGA (SEQ ID NO: 245) |
| 5-methylcytidine sute | CGGGGAACTCTCCAATTCCCC (SEQ ID NO: 246) |
| b-globin | ACTCTTCTGGTCCCCACAGACTCAGAGAGAA (SEQ ID NO: 247) |
| b-globin v2 | AAGAGAGACTCAGACACCCCTGGTCTTCTCAACTCTTCTGG |
| | TCCCCACAGACTCAGAGAGAA (SEQ ID NO: 248) |
| Translation Initiator of Short | 5′ AAGATCG (SEQ ID NO: 249) |
| UTR (TISU) sequence | |
| Synthetic UTR | CCGTGACGCAGGGATAAAGCAATCAAGGTTGTGTCAGTGA |
| | CGTGCTATAA (SEQ ID NO: 250) |
| Complement Factor 3 (C3) | GAGCCAGATAAAAAGCCAGCTCCAGCAGGCGCTGCTCAC |
| | TCCTCCCCATCCTCTCCCTCTGTCCCTCTGTCCCTCTGACCC |
| | TGCACTGTCCC (SEQ ID NO: 251) |
| CYP2e1 | ACAGGATTGTCTCCCGGGCTGGCAGCAGGGCCCCAGC |
| | (SEQ ID NO: 252) |
| Sv40 PolyA | AACTTGTTTATTGCAGCTTATAATGGTTACAAATAAAGCAATA |
| | GCATCACAAATTTCACAAATAAAGCATTTTTTTCACTGCATTC |
| | TAGTTGTGGTTTGTCCAAACTCATCAATGTATCTTA (SEQ ID |
| | NO: 253) |
| Amino terminal enhancer of | CTGGTACTGCATGCACGCAATGCTAGCTGCCCCTTTCCCGTCC |
| Split | TGGTACCCCGAGTCTCCCCCGACCTCGGGTCCCAGGTATGCTC |
| | CCACCTCCACCTGCCCACTCACCACCTCTGCTAGTTCCAGACA |
| | CCTCC (SEQ ID NO: 254) |
| mtRNR1 | CAAGCACGCAGCAATGCAGCTCAAAACCTTAGCCTAGCACAC |
| | CCCCACGGGAAACAGCAGTGATTAACCTTTAGCAATAAACGA |
| | AAGTTTAACTAAGCTATACTAACCCCAGGGTTGGTCAATTTCG |
| | TCCAGCCACACC (SEQ ID NO: 255) |
| a-globin | GCTGGAGCCTCGGTGGCCATGCTTCTTGCCCCTTGGGCCTCCC |
| | CCCAGCCCCTCCTCCCCTTCCTGCACCCGTACCCCCGTGGTCTT |
| | TGAATAAAGTCTGAGTGGGCGGCA (SEQ ID NO: 256) |
| UTR30 (Sample PJ et al. | CCGTAAATCGGATAGGGCGGAAACGATGGACACGAAGTCTAT |
| Nature Biotechnol 2019 | AGTCGGGT (SEQ ID NO: 257) |
| UTR31 (Sample PJ et al., | CTCACGGGATGACCCACATTGCGATGATGTAGTTCCGGGCCGC |
| supra) | CAAATAG (SEQ ID NO: 258) |
| UTR32 (Sample PJ et al., | AAGTACTGCAGGCCCGAATCACTCGGCCAAATGGCCTGCATA |
| supra) | ATGCTGAT (SEQ ID NO: 259) |
| UTR33 (Sample PJ et al., | TAAACTTCCAAGTCGCCTCGCAGGCTCCCACCAATGCCGCCGC |
| supra) | CTAAGGG (SEQ ID NO: 260) |
| UTR34 (Sample PJ et al., | CTCAGGAGGGTGGTAAGCCACCGCCCACGCGTGGGGCACTCG |
| supra) | TGTATTCT (SEQ ID NO: 261) |
| UTR35 (Sample PJ et al., | AGCGGACATCAGCCATTGAATACATGAAACAAATCGCAAAAG |
| supra) | CAATAGCT (SEQ ID NO: 262) |
| UTR36 (Sample PJ et al., | CGTCGCTCCGGGGAGCTGTGTGTACGAGTAAAAAATTTAGATC |
| supra) | ACCAAAA (SEQ ID NO: 263) |
| UTR37 (Sample PJ et al., | ACAATCGCCAGGGACAGATAGGAAGTTATAGACAGCAGGCCG |
| supra) | ACACAAGT (SEQ ID NO: 264) |
| UTR38 (Sample PJ et al., | GAAAAATCTATAGCAGAAGTCAGCGGTAGACGCACGGCATAG |
| supra) | CATCCAAC (SEQ ID NO: 265) |
| UTR39 (Sample PJ et al., | GGGCGCTCGAGCAGGTTCAGAAGGAGATCAAAAACCCCCAAG |
| supra) | GATCAAAC (SEQ ID NO: 266) |

For example, when the DTS comprises a binding sequence for a Tet Repressor (TetR) protein, e.g., a TCCCTATCAGTGATAGAGA (SEQ ID NO:267) or a variant thereof, the cell may also be contacted with an mRNA encoding a TetR protein. In some embodiments, the TetR protein has been engineered to comprise an NLS. In some embodiments, the NLS is an NLS listed in Table 7. In some certain embodiments, the NLS is the NLS of SV40 Large T antigen or an optimized variant thereof, the NLS of the influenza A nuclear protein INF-A, the NLS of c-myc, or a fusion of the c-myc NLS to the influenza A nuclear protein INF-A NLS. In some embodiments, the NLS is located proximal to the N terminus. In other embodiments, the NLS is located proximal to the C-terminus. In some embodiments, the NLS is fused to the TetR protein with a linker.

In some embodiments, the TetR protein comprises a sequence having a sequence identity of 90% or more, in some instances 92%, 93%, 94%, 95% or more, in certain instances 96%, 97%, 98%, 99% identity or more, e.g., is 100% identical to the wild type Escherichia coli transcriptional regulator TetR protein sequence:

(SEQ ID NO: 268)
MSRLDKSKVINSALELLNEVGIEGLTTRKLAQKLGVEQPTLYWHVKNKR
ALLDALAIEMLDRHHTHFCPLEGESWQDFLRNNAKSFRCALLSHRDGAK
VHLGTRPTEKQYETLENQLAFLCQQGFSLENALYALSAVGHFTLGCVLE
DQEHQVAKEERETPTTDSMPPLLRQAIELFDHQGAEPAFLFGLELIICG
LEKQLKCESGS.

In some embodiments, the TetR protein is encoded by a polynucleotide comprising a sequence having a sequence identity of 80% or more to a sequence listed in Table 10, for example 85%, 90%, or 95% sequence identity or more, in some instances 96%, 97%, 98%, or 99% sequence identity, in some cases 100% sequence identity across the length of a sequence in Table 10. In some embodiments, the polynucleotide sequence has been codon optimized for expression in a particular host cell, e.g., codon optimized for expression in human cells or mouse cells.

TABLE 10
RNA polynucleotide sequences for expression of TetR protein

| TetR variant | TetR RNA Sequence (5′ to 3′) |
|---|---|
| Wild Type | AUGAGCAGACUGGACAAGAGCAAAGUGAUCAACAGCGCCCUGGAACUGCU |
| | GAACGAAGUGGGCAUCGAGGGCCUGACCACAAGAAAGCUGGCCCAGAAGC |
| | UGGGCGUCGAGCAGCCUACACUGUACUGGCACGUGAAGAACAAGCGGGCC |
| | CUGCUGGAUGCCCUGGCCAUUGAGAUGCUGGACCGGCACCACACACACUU |
| | UUGCCCUCUGGAAGGCGAGAGCUGGCAGGACUUCCUGAGAAACAACGCCA |
| | AGAGCUUCAGAUGCGCCCUGCUGAGCCAUAGAGAUGGCGCCAAAGUGCAC |
| | CUGGGCACCAGACCUACAGAGAAGCAGUACGAGACACUGGAAAACCAGCU |
| | GGCCUUCCUGUGCCAGCAGGGAUUCAGCCUGGAAAACGCCCUGUAUGCCC |
| | UGUCUGCCGUGGGCCACUUUACACUGGGAUGCGUGCUGGAAGAUCAAGAG |
| | CACCAGGUGGCCAAAGAGGAAAGAGAGACACCCACCACCGACAGCAUGCC |
| | UCCACUGCUGAGACAGGCCAUCGAGCUGUUCGAUCACCAAGGCGCCGAACC |
| | UGCCUUUCUGUUCGGACUGGAACUGAUCAUCUGCGGCCUCGAAAAGCAGC |
| | UGAAGUGCGAGAGCGGCUCC (SEQ ID NO: 269) |
| Human | AUGAGCAGGCUGGACAAGAGCAAGGUGAUCAACAGCGCCCUGGAGCUGCU |
| (Jcat) | GAACGAGGUGGGCAUCGAGGGCCUGACCACCAGGAAGCUGGCCCAGAAGC |
| | UGGGCGUGGAGCAGCCCACCCUGUACUGGCACGUGAAGAACAAGAGGGCC |
| | CUGCUGGACGCCCUGGCCAUCGAGAUGCUGGACAGGCACCACACCCACUUC |
| | UGCCCCCUGGAGGGCGAGAGCUGGCAGGACUUCCUGAGGAACAACGCCAA |
| | GAGCUUCAGGUGCGCCCUGCUGAGCCACAGGGACGGCGCCAAGGUGCACC |
| | UGGGCACCAGGCCCACCGAGAAGCAGUACGAGACgCUGGAGAACCAGCUGG |
| | CCUUCCUGUGCCAGCAGGGCUUCAGCCUGGAGAACGCCCUGUACGCCCUGA |
| | GCGCCGUGGGCCACUUCACCCUGGGCUGCGUGCUGGAGGACCAGGAGCACC |
| | AGGUGGCCAAGGAGGAGAGGGAGACgCCCACCACCGACAGCAUGCCCCCCC |
| | UGCUGAGGCAGGCCAUCGAGCUGUUCGACCACCAGGGCGCCGAGCCCGCCU |
| | UCCUGUUCGGCCUGGAGCUGAUCAUCUGCGGCCUGGAGAAGCAGCUGAAG |
| | UGCGAGAGCGGCAGC (SEQ ID NO: 270) |
| Human | AUGAGCAGGCUGGAUAAGAGCAAGGUGAUUAACUCCGCCCUGGAGCUGCU |
| (Vector | GAACGAAGUGGGAAUCGAGGGCCUGACUACAAGGAAGUUGGCCCAGAAGC |
| Builder) | UGGGCGUGGAGCAGCCUACCCUGUACUGGCACGUGAAGAACAAGAGAGCC |
| | CUGCUGGACGCCCUGGCCAUCGAGAUGCUGGACAGGCAUCACACUCACUU |
| | UUGCCCUCUCGAGGGCGAGAGCUGGCAGGACUUCCUGAGGAACAACGCCA |
| | AGUCAUUUCGGUGUGCCCUGCUGAGCCAUCGAGAUGGGGCCAAGGUGCAC |
| | CUGGGAACCAGACCCACAGAGAAGCAGUACGAGACACUGGAAAACCAGCU |
| | GGCUUUCCUGUGCCAGCAGGGCUUCUCCCUGGAGAACGCCCUGUACGCCCU |
| | GAGCGCCGUGGGCCAUUUCACCCUGGGUUGUGUGCUGGAGGAUCAGGAGC |
| | ACCAGGUGGCCAAGGAGGAAAGGGAAACCCCUACCACCGACUCAAUGCCA |
| | CCUCUGCUGCGCCAGGCCAUCGAGCUGUUUGACCACCAGGGAGCCGAGCCC |
| | GCCUUCCUGUUCGGGCUGGAGCUGAUUAUUUGCGGCCUGGAGAAGCAGCU |
| | GAAGUGUGAGAGUGGCAGC (SEQ ID NO: 271) |
| Human | AUGUCCCGACUGGACAAAUCCAAGGUUAUCAAUAGCGCCCUGGAGCUGCU |
| (Novopro) | CAAUGAAGUGGGUAUCGAGGGCCUGACGACACGCAAGCUUGCACAGAAGU |
| | UGGGCGUCGAGCAGCCAACUCUGUAUUGGCAUGUGAAGAACAAGAGGGCC |
| | CUGCUCGACGCCCUCGCGAUUGAAAUGCUGGACAGACACCACACACAUUU |
| | UUGUCCUUUGGAGGGCGAGUCCUGGCAGGACUUUCUUAGAAAUAACGCCA |
| | AGUCCUUCCGCUGCGCACUGCUCAGCCACCGAGAUGGGGCUAAAGUGCAU |
| | CUUGGAACACGCCCCACCGAGAAACAGUACGAAACUCUCGAGAAUCAGCU |
| | UGCAUUUCUGUGUCAGCAAGGGUUCAGUCUGGAAAACGCCCUGUACGCUC |
| | UGUCCGCCGUGGGGCAUUUUACACUGGGAUGUGUGCUGGAAGACCAAGAG |
| | CAUCAGGUCGCCAAGGAAGAAAGAGAGACUCCGACAACCGACUCCAUGCC |
| | UCCCUUGCUCCGGCAGGCCAUUGAGCUGUUCGACCACCAGGGCGCCGAACC |
| | UGCCUUCUUGUUCGGUCUUGAGCUGAUCAUCUGCGGACUGGAAAAGCAAC |
| | UGAAAUGUGAGAGCGGGUCC (SEQ ID NO: 272) |
| Human | AUGAGUCGACUUGAUAAGAGCAAGGUUAUAAAUUCCGCUUUGGAACUGUU |
| (IDT) | GAAUGAGGUGGGCAUCGAGGGUCUUACGACCCGCAAAUUGGCACAAAAGU |
| | UGGGAGUUGAACAGCCAACGCUUUAUUGGCACGUUAAAAAUAAGCGGGCU |
| | CUGCUGGACGCUCUGGCUAUAGAAAUGCUUGACCGGCAUCAUACCCAUUU |
| | UUGUCCUCUGGAAGGCGAAUCUUGGCAGGACUUUCUCAGGAAUAACGCCA |
| | AAUCUUUUAGAUGCGCUCUUUUGUCACACCGCGAUGGAGCCAAGGUCCAU |
| | CUUGGCACGCGGCCAACGGAGAAACAAUAUGAGACCCUCGAAAACCAGCU |
| | CGCCUUCCUCUGUCAACAGGGAUUCAGUCUUGAGAAUGCCCUGUACGCUC |
| | UUUCUGCGGUCGGGCAUUUUACACUCGGAUGUGUUUUGGAGGACCAAGAA |
| | CAUCAAGUAGCGAAAGAGGAACGGGAAACUCCCACGACCGAUUCAAUGCC |
| | GCCACUCCUCCGACAAGCUAUUGAAUUGUUCGAUCAUCAAGGGGCUGAAC |
| | CUGCCUUCCUCUUCGGUCUGGAACUUAUAAUCUGCGGGCUCGAGAAACAG |
| | UUGAAAUGCGAGUCAGGAUCA (SEQ ID NO: 273) |
| Human | AUGAGCAGACUGGACAAGAGCAAGGUGAUCAACAGCGCCCUGGAGCUGCU |
| (Genewiz) | GAACGAGGUGGGCAUCGAGGGCCUGACCACAAGAAAGCUGGCUCAGAAGC |
| | UGGGCGUGGAGCAGCCCACCCUGUACUGGCACGUGAAGAACAAGAGAGCC |
| | CUGCUGGACGCCCUGGCCAUCGAGAUGCUGGACAGACACCACACCCACUUC |
| | UGCCCCCUGGAGGGCGAGAGCUGGCAAGACUUCCUGAGAAACAACGCCAA |
| | GAGCUUCAGAUGCGCCCUGCUGAGCCACAGAGACGGCGCCAAGGUGCACC |
| | UGGGCACAAGACCCACCGAGAAGCAGUACGAAACCCUGGAGAAUCAGCUG |
| | GCCUUCCUGUGUCAGCAAGGCUUCAGCCUGGAGAACGCCCUGUACGCCCUG |
| | AGCGCCGUGGGCCACUUCACCCUGGGCUGCGUGCUGGAGGACCAAGAGCA |
| | CCAAGUGGCCAAGGAGGAGAGAGAAACCCCCACCACCGACAGCAUGCCCCC |
| | CCUGCUGAGACAAGCCAUCGAGCUGUUCGACCACCAAGGCGCCGAGCCCGC |
| | CUUCCUGUUCGGCCUGGAGCUGAUCAUCUGCGGCCUGGAGAAGCAGCUGA |
| | AGUGCGAAAGCGGCAGC (SEQ ID NO: 274) |
| Human | AUGAGCAGACUGGAUAAGAGCAAGGUCAUCAACAGCGCCCUCGAACUGCU |
| (Genesmart) | GAACGAGGUGGGCAUCGAAGGCCUUACAACAAGAAAGCUGGCUCAGAAGC |
| | UGGGCGUGGAACAACCUACCCUGUAUUGGCAUGUGAAGAACAAGCGGGCU |
| | CUGCUGGACGCCCUGGCCAUCGAGAUGCUGGACAGACACCACACCCACUUU |
| | UGCCCACUGGAGGGCGAGAGCUGGCAGGACUUCCUGAGAAAUAACGCCAA |
| | GUCCUUCCGGUGCGCCCUCCUGAGCCACCGGGACGGCGCCAAGGUGCACCU |
| | GGGAACCAGACCUACCGAGAAGCAGUACGAGACACUGGAAAACCAGCUGG |
| | CCUUCCUGUGCCAGCAGGGCUUCAGCCUGGAGAAUGCCCUGUACGCCCUGU |
| | CUGCCGUGGGCCACUUCACCCUGGGUUGUGUGCUGGAAGAUCAGGAGCAC |
| | CAAGUGGCCAAAGAGGAAAGAGAGACACCUACAACCGACAGCAUGCCCCC |
| | CCUGCUGCGGCAGGCCAUUGAGCUGUUCGACCACCAGGGAGCCGAGCCUGC |
| | UUUUCUGUUCGGCCUGGAGCUGAUCAUCUGOGGCCUGGAAAAACAGCUGA |
| | AGUGUGAAUCUGGCUCC (SEQ ID NO: 275) |
| Human | AUGAGCAGACUGGACAAGAGCAAAGUGAUCAACAGCGCCCUGGAACUGCU |
| (Geneart) | GAACGAAGUGGGCAUCGAGGGCCUGACCACAAGAAAGCUGGCCCAGAAGC |
| | UGGGCGUCGAGCAGCCUACACUGUACUGGCACGUGAAGAACAAGCGGGCC |
| | CUGCUGGAUGCCCUGGCCAUUGAGAUGCUGGACCGGCACCACACACACUU |
| | UUGCCCUCUGGAAGGCGAGAGCUGGCAGGACUUCCUGAGAAACAACGCCA |
| | AGAGCUUCAGAUGCGCCCUGCUGAGCCAUAGAGAUGGCGCCAAAGUGCAC |
| | CUGGGCACCAGACCUACAGAGAAGCAGUACGAGACACUGGAAAACCAGCU |
| | GGCCUUCCUGUGCCAGCAGGGAUUCAGCCUGGAAAACGCCCUGUAUGCCC |
| | UGUCUGCCGUGGGCCACUUUACACUGGGAUGCGUGCUGGAAGAUCAAGAG |
| | CACCAGGUGGCCAAAGAGGAAAGAGAGACACCCACCACCGACAGCAUGCC |
| | UCCACUGCUGAGACAGGCCAUCGAGCUGUUCGAUCACCAAGGCGCCGAACC |
| | UGCCUUUCUGUUCGGACUGGAACUGAUCAUCUGCGGCCUCGAGAAGCAGC |
| | UGAAGUGCGAGUCUGGAUCU (SEQ ID NO: 276) |
| Mouse | AUGAGCAGGCUGGACAAGAGCAAGGUGAUCAACAGCGCCCUGGAGCUGCU |
| (Jcat) | GAACGAGGUGGGCAUCGAGGGCCUGACCACCAGGAAGCUGGCCCAGAAGC |
| | UGGGCGUGGAGCAGCCCACCCUGUACUGGCACGUGAAGAACAAGAGGGCC |
| | CUGCUGGACGCCCUGGCCAUCGAGAUGCUGGACAGGCACCACACCCACUUC |
| | UGCCCCCUGGAGGGCGAGAGCUGGCAGGACUUCCUGAGGAACAACGCCAA |
| | GAGCUUCAGGUGCGCCCUGCUGAGCCACAGGGACGGCGCCAAGGUGCACC |
| | UGGGCACCAGGCCCACCGAGAAGCAGUACGAGACCCUGGAGAACCAGCUG |
| | GCCUUCCUGUGCCAGCAGGGCUUCAGCCUGGAGAACGCCCUGUACGCCCUG |
| | AGCGCCGUGGGCCACUUCACCCUGGGCUGCGUGCUGGAGGACCAGGAGCA |
| | CCAGGUGGCCAAGGAGGAGAGGGAGACCCCCACCACCGACAGCAUGCCCCC |
| | CCUGCUGAGGCAGGCCAUCGAGCUGUUCGACCACCAGGGCGCCGAGCCCGC |
| | CUUCCUGUUCGGCCUGGAGCUGAUCAUCUGCGGCCUGGAGAAGCAGCUGA |
| | AGUGCGAGAGCGGCAGC (SEQ ID NO: 277) |
| Mouse | AUGUCCCGCCUGGACAAGUCUAAGGUGAUCAAUUCCGCCCUGGAACUGCU |
| (Vector | GAAUGAGGUGGGAAUCGAGGGCCUGACCACAAGGAAGCUGGCCCAGAAGC |
| Builder) | UGGGCGUGGAGCAGCCUACACUGUACUGGCAUGUGAAGAACAAGCGGGCC |
| | CUCCUGGACGCCCUGGCCAUCGAAAUGCUGGACCGCCACCACACCCACUUC |
| | UGUCCCCUGGAGGGCGAGAGCUGGCAGGAUUUCCUGCGGAAUAAUGCCAA |
| | GAGCUUCAGAUGUGCUCUGCUGAGCCACAGAGAUGGCGCAAAGGUGCACC |
| | UGGGCACCAGACCCACCGAGAAGCAGUAUGAGACUCUGGAGAACCAGCUG |
| | GCAUUCCUGUGCCAACAGGGGUUUUCCCUGGAGAACGCCCUGUACGCCCU |
| | GAGCGCCGUGGGACACUUCACCCUGGGCUGUGUGCUGGAGGACCAGGAGC |
| | ACCAGGUGGCCAAGGAGGAAAGAGAGACACCAACUACCGACUCCAUGCCU |
| | CCUCUCCUCCGGCAGGCCAUUGAACUGUUUGAUCAUCAGGGCGCCGAGCCA |
| | GCCUUUCUGUUUGGCCUGGAACUGAUCAUCUGUGGCCUGGAGAAGCAGCU |
| | GAAGUGCGAAUCUGGCAGC (SEQ ID NO: 278) |
| Mouse | AUGUCUAGGUUGGAUAAAUCAAAGGUCAUCAAUUCUGCUCUGGAGCUCCU |
| (Novopro) | CAAUGAAGUGGGAAUCGAAGGUCUCACUACUCGGAAGCUGGCACAAAAAC |
| | UCGGGGUGGAACAACCUACCCUUUAUUGGCACGUGAAAAACAAAAGGGCC |
| | UUGCUGGACGCUCUGGCCAUAGAGAUGUUGGACCGACAUCACACGCAUUU |
| | CUGCCCUCUGGAGGGGGAAUCCUGGCAAGAUUUCCUGCGCAAUAACGCAA |
| | AGUCUUUCCGAUGCGCUCUGCUCUCUCAUAGGGACGGCGCUAAGGUCCAC |
| | CUGGGAACCAGACCUACGGAGAAGCAGUAUGAAACCCUGGAGAACCAGCU |
| | GGCAUUCCUCUGCCAGCAGGGCUUCAGUCUCGAGAACGCACUGUACGCAC |
| | UGUCUGCUGUGGGUCACUUCACCCUGGGCUGUGUGUUGGAGGACCAGGAG |
| | CACCAGGUGGCCAAGGAAGAGCGCGAAACCCCUACAACGGAUUCUAUGCC |
| | CCCCUUGCUUAGGCAGGCCAUCGAGCUCUUCGAUCACCAGGGGGCCGAACC |
| | UGCCUUUUUGUUUGGCCUGGAACUCAUUAUAUGUGGGCUGGAGAAGCAGC |
| | UGAAAUGUGAGAGUGGAAGC (SEQ ID NO: 279) |
| Mouse | AUGUCACGAUUGGACAAAAGUAAGGUUAUAAACUCUGCUCUCGAACUUCU |
| (IDT) | GAACGAAGUUGGGAUCGAAGGUUUGACCACCCGCAAGUUGGCUCAAAAAC |
| | UUGGUGUGGAGCAGCCUACCCUGUAUUGGCACGUAAAGAACAAGAGAGCC |
| | UUGCUGGACGCCCUUGCAAUCGAGAUGCUGGACAGACACCACACCCAUUU |
| | UUGUCCCUUGGAAGGGGAGAGUUGGCAAGAUUUUCUGCGGAACAAUGCUA |
| | AGAGCUUCCGCUGUGCCCUGCUUUCCCAUCGAGAUGGCGCAAAAGUGCAC |
| | UUGGGAACUCGGCCCACUGAGAAGCAAUACGAGACUCUUGAGAACCAACU |
| | CGCAUUUCUUUGUCAACAAGGAUUUUCCCUGGAGAAUGCUCUGUAUGCUU |
| | UGAGUGCAGUAGGCCAUUUCACCCUCGGCUGCGUACUGGAGGACCAAGAG |
| | CAUCAAGUUGCCAAAGAAGAACGGGAAACCCCAACUACAGAUAGCAUGCC |
| | UCCUUUGCUCCGACAGGCCAUAGAACUCUUUGAUCAUCAAGGUGCAGAGC |
| | CAGCAUUCCUCUUCGGCCUUGAGCUCAUUAUUUGCGGACUUGAGAAGCAA |
| | CUGAAGUGUGAAAGCGGAAGU (SEQ ID NO: 280) |
| Mouse | AUGUCUAGGCUGGACAAGAGCAAGGUGAUCAACAGCGCCCUGGAGCUGCU |
| (Genewiz) | GAACGAGGUGGGCAUCGAGGGCCUGACCACAAGGAAGCUGGCUCAGAAGC |
| | UGGGCGUGGAGCAGCCUACCCUGUACUGGCACGUGAAGAACAAGAGGGCC |
| | CUGCUGGACGCCCUGGCCAUCGAGAUGCUGGAUAGGCACCACACCCACUUC |
| | UGCCCUCUGGAGGGCGAGAGCUGGCAAGACUUCCUGAGGAACAACGCCAA |
| | GAGCUUUAGGUGCGCCCUGCUGAGCCAUAGGGACGGCGCCAAGGUGCACC |
| | UGGGCACAAGGCCUACCGAGAAGCAGUACGAAACCCUGGAGAAUCAGCUG |
| | GCCUUCCUGUGUCAGCAAGGCUUCAGCCUGGAGAACGCCCUGUACGCCCUG |
| | AGCGCCGUGGGCCACUUCACCCUGGGCUGCGUGCUGGAGGACCAAGAGCA |
| | CCAAGUGGCCAAGGAGGAGAGGGAAACCCCUACCACCGACAGCAUGCCUCC |
| | UCUGCUGAGGCAAGCCAUCGAGCUGUUCGACCACCAAGGCGCCGAGCCUGC |
| | CUUCCUGUUCGGCCUGGAGCUGAUCAUCUGCGGCCUGGAGAAGCAGCUGA |
| | AGUGCGAAAGCGGCAGC (SEQ ID NO: 281) |
| Mouse | AUGUCUCGUCUCGACAAGUCAAAAGUCAUCAAUAGUGCUCUGGAGCUGCU |
| (Genesmart) | UAAUGAAGUAGGGAUAGAAGGUCUGACCACCAGGAAGCUGGCCCAGAAGC |
| | UGGGAGUUGAACAACCUACCCUCUACUGGCACGUGAAAAACAAGCGCGCA |
| | CUUUUAGACGCCCUGGCGAUUGAGAUGCUGGAUCGGCACCAUACACAUUU |
| | CUGCCCCCUAGAAGGAGAAUCCUGGCAGGACUUUCUCCGAAACAACGCCA |
| | AAUCCUUCCGCUGUGCACUGCUGAGCCAUCGAGAUGGAGCGAAAGUGCAC |
| | CUGGGGACGCGGCCUACUGAGAAACAGUACGAAACUCUAGAGAACCAGUU |
| | GGCCUUCCUCUGCCAGCAGGGAUUCAGUUUAGAGAAUGCACUCUAUGCUC |
| | UCUCUGCAGUGGGCCACUUCACAUUGGGCUGCGUUUUGGAAGACCAGGAG |
| | CAUCAGGUGGCCAAGGAGGAAAGAGAGACACCUACUACAGAUAGCAUGCC |
| | UCCACUGCUGAGACAAGCCAUUGAACUGUUUGACCACCAGGGUGCUGAGC |
| | CAGCCUUUUUGUUUGGUUUAGAACUUAUCAUCUGUGGCCUAGAGAAGCAG |
| | CUGAAAUGUGAGUCUGGCAGC (SEQ ID NO: 282) |
| Mouse | AUGAGCAGACUGGACAAGAGCAAAGUGAUCAACAGCGCCCUGGAACUGCU |
| (Gencart) | GAACGAAGUGGGCAUCGAGGGCCUGACCACAAGAAAGCUGGCUCAGAAGC |
| | UGGGCGUCGAGCAGCCUACACUGUACUGGCACGUGAAGAACAAGAGAGCC |
| | CUGCUGGACGCCCUGGCCAUCGAGAUGCUGGAUAGACACCACACACACUU |
| | UUGCCCUCUGGAAGGCGAGAGCUGGCAGGACUUCCUGAGAAACAACGCCA |
| | AGAGCUUCAGAUGCGCCCUGCUGAGCCAUAGAGAUGGCGCCAAAGUGCAC |
| | CUGGGCACCAGACCUACAGAGAAGCAGUACGAGACACUGGAAAACCAGCU |
| | GGCCUUCCUGUGCCAGCAGGGAUUCUCUCUGGAAAACGCCCUGUACGCCCU |
| | GAGCGCUGUGGGCCACUUUACACUGGGAUGCGUGCUGGAAGAUCAAGAGC |
| | ACCAGGUGGCCAAAGAGGAAAGAGAGACACCCACCACCGACAGCAUGCCU |
| | CCACUGCUGAGACAGGCUAUCGAGCUGUUCGACCACCAAGGCGCCGAGCCU |
| | GCUUUUCUGUUCGGACUGGAACUGAUCAUCUGCGGCCUCGAGAAGCAGCU |
| | GAAGUGCGAGUCUGGAUCU (SEQ ID NO: 283) |

As another example, when the DTS comprises a binding sequence for a ZF protein, the cell may also be contacted with an mRNA encoding a ZF protein. For example, when the DTS comprises the sequence AAACTGCAAAAG (SEQ ID NO:455) or a variant thereof, the cell may also be contacted with an mRNA encoding the ZF protein ZF-CCR5. In some embodiments, the ZF protein has been engineered to comprise an NLS. In some embodiments, the NLS is proximal to the N terminus. In other embodiments, the NLS is proximal to the C-terminus. In some embodiments, the ZF protein comprises an NLS at both the N terminus and the C terminus. In some embodiments, the NLS is fused to the ZF protein with a linker.

In some embodiments, the ZF protein comprises a sequence having a sequence identity of 90% or more, in some instances 92%, 93%, 94%, 95% or more, in certain instances 96%, 97%, 98%, 99% identity or more, e.g. is 100% identical to the ZF-CCR5 fusion protein: MRPFQCRICMRNFSRSDNLSVHIRTHTGEKPFACDICGRKFAQKINLQVHTKIHTGEKPFQCRICM RNFSRSDVLSEHIRTHTGEKPFACDICGRKFAQRNHRTTHTKIHLR (SEQ ID NO:284), for example, the variant

(SEQ ID NO: 285)
MAPKKKRKVGIHGVPAAMAERPFQCRICMRNFSRSDNLSVHIRTH
TGEKPFACDICGRKFAQKINLQVHTKIHTGEKPFQCRICMRNFSR
SDVLSEHIRTHTGEKPFACDICGRKFAQRNHRTTHTKIHLR.

As another example, when the DTS comprises a binding sequence for a TALE protein, e.g., TTCATTACACCTGCAGCT (SEQ ID NO:286), ATAAACCCCCTCCAA (SEQ ID NO:287), or TCGAGTTTACTCCCTATCAGTGATAGAGAACG (SEQ ID NO:288), or variants thereof, the cell may also be contacted with an mRNA encoding a TALE protein. In some embodiments, the TALE protein has been engineered to comprise an NLS. In some embodiments, the NLS is proximal to the N terminus. In other embodiments, the NLS is proximal to the C-terminus. In some embodiments, the TALE protein comprises an NLS at both the N terminus and the C terminus. In some embodiments, the NLS is fused to the TALE protein with a linker.

In some embodiments, the TALE protein comprises a sequence having a sequence identity of 90% or more, in some instances 92%, 93%, 94%, 95% or more, in certain instances 96%, 97%, 98%, 99% identity or more, e.g., is 100% identical to TMVLAQNRKKSLHCFEGLFTAVVTSNSDHLVRSQ (SEQ ID NO: 289), for example MLTPDQVVAIASNIGGKQALETVQRLLPVLCQDHGLTPDQVVAIASNGGGKQALETVQRLLPVL CQDHGLTPDQVVAIASNIGGKQALETVQRLLPVLCQDHGLTPDQVVAIASNIGGKQALETVQRL LPVLCQDHGLTPDQVVAIASNIGGKQALETVQRLLPVLCQDHGLTPDQVVAIASHDGGKQALET VQRLLPVLCQDHGLTPDQVVAIASHDGGKQALETVQRLLPVLCQDHGLTPDQVVAIASHDGGK QALETVQRLLPVLCQDHGLTPDQVVAIASHDGGKQALETVQRLLPVLCQDHGLTPDQVVAIASH DGGKQALETVQRLLPVLCQDHGLTPDQVVAIASNGGGKQALETVQRLLPVLCQDHGLTPDQVV AIASHDGGKQALETVQRLLPVLCQDHGLTPDQVVAIASHDGGKQALETVQRLLPVLCQDHGLTP DQVVAIASNIGGKQALETVQRLLPVLCQDHGLTPDQVVAIASNIGGKQALETVQRLLPVLCQDH G (SEQ ID NO:290). Other exemplary TALEs and methods for engineering them may be found in Li et a. 2011 (supra) and Kim et al. 2011 (supra).

As another example, when the DTS comprises the binding sequence for a GAL4 protein, for example CGG-N₁₁-CCG or a variant thereof, the cell may also be contacted with an mRNA encoding a GAL4 protein. In some embodiments, the GAL4 protein has been engineered to comprise an NLS. In some embodiments, the NLS is proximal to the N terminus. In other embodiments, the NLS is proximal to the C-terminus. In some embodiments, the GAL4 protein is engineered to comprise an NLS at both the N-terminus and the C-terminus. In some embodiments, the NLS is fused to the GAL4 protein with a linker. In some embodiments, the NLS is selected from those listed in Table 7. In some certain embodiments, the NLS is the NLS of SV40 Large T antigen or an optimized variant thereof, the NLS of the influenza A nuclear protein INF-A, the NLS of c-myc, or a fusion of the c-myc NLS to the influenza A nuclear protein INF-A NLS.

In some embodiments, the GAL4 protein comprises a sequence having a sequence identity of 90% or more, in some instances 92%, 93%, 94%, 95% or more, in certain instances 96%, 97%, 98%, 99% identity or more, e.g., is 100% identical to wild type Saccharomyces cerevisiae GAL4 protein MKLLSSIEQACDICRLKKLKCSKEKPKCAKCLKNNWECRYSPKTKRSPLTRAHLTEVESRLERLE (SEQ ID NO:291). In some cases, the GAL4 protein comprises a sequence having a sequence identity of 90% or more, in some instances 92%, 93%, 94%, 95% or more, in certain instances 96%, 97%, 98%, 99% identity or more, e.g., is 100% identical to:

(SEQ ID NO: 292)
MKLLSSIEQACDICRLKKLKCSKEKPKCAKCLKNNWECRYSPKTK
RSPLTRAHLTEVESRLERLEQLFLLIFPREDLDMILKMDSLQDIK
ALLTGLFVQDNVNKDAVTDRLASVETDMPLTLRQHRISATSSSEE
SSNKGQRQLTVS.

In some embodiments, the GAL4 protein is encoded by a polynucleotide comprises a sequence having a sequence identity of 80% or more to a sequence listed in Table 11, for example 85%, 90%, or 95% sequence identity or more, in some instances 96%, 97%, 98%, or 99% sequence identity, in some cases 100% sequence identity across the length of a sequence in Table 11. In some embodiments, the GAL4 polynucleotide has been codon optimized for expression in a particular host cell, e.g., codon optimized for expression in human or mouse cells.

TABLE 11
RNA polynucleotide sequences encoding GAL4 protein.

| GAL4 variant | GAL4 mRNA Sequence (5' to 3') |
|---|---|
| Wild Type | AUGAAACUGCUGUCAUCCAUCGAGCAAGCUUGUGACAUAUGCCGGCUGAAG |
| | AAAUUGAAAUGUAGCAAGGAGAAGCCAAAAUGUGCGAAAUGCCUCAAGAA |
| | UAAUUGGGAAUGCAGAUAUUCCCCUAAGACUAAGCGUAGCCCCUUGACCCG |
| | GGCCCACCUUACUGAGGUCGAGAGCAGAUUAGAGCGCCUCGAGCAAUUGUU |
| | CCUGCUCAUCUUCCCCAGAGAGGAUCUGGAUAUGAUCUUAAAGAUGGACAG |
| | CCUUCAAGACAUUAAGGCUCUGCUCACUGGGCUGUUCGUGCAGGACAACGU |
| | CAACAAGGACGCUGUGACGGACCGCCUGGCUUCCGUAGAAACGGACAUGCC |
| | CCUGACUCUGAGGCAACACCGCAUUUCUGCUACGAGUUCUUCCGAGGAAUC |
| | CUCAAAUAAGGGACAGCGUCAACUGACCGUCUCU (SEQ ID NO: 293) |
| Human | AUGAAGCUGCUGAGCAGCAUCGAGCAGGCCUGCGACAUCUGCAGGCUGAAG |
| (Jcat) | AAGCUGAAGUGCAGCAAGGAGAAGCCCAAGUGCGCCAAGUGCCUGAAGAAC |
| | AACUGGGAGUGCAGGUACAGCCCCAAGACCAAGAGGAGCCCCCUGACCAGG |
| | GCCCACCUGACCGAGGUGGAGAGCAGGCUGGAGAGGCUGGAGCAGCUGUUC |
| | CUGCUGAUCUUCCCCAGGGAGGACCUGGACAUGAUCCUGAAGAUGGACAGC |
| | CUGCAGGACAUCAAGGCCCUGCUGACCGGCCUGUUCGUGCAGGACAACGUG |
| | AACAAGGACGCCGUGACCGACAGGCUGGCCAGCGUGGAGACgGACAUGCCC |
| | CUGACCCUGAGGCAGCACAGGAUCAGCGCCACCAGCAGCAGCGAGGAGAGC |
| | AGCAACAAGGGCCAGAGGCAGCUGACCGUGAGC (SEQ ID NO: 294) |
| Human | AUGAAGCUGCUGUCUAGCAUCGAGCAGGCGUGUGACAUCUGCAGACUGAAG |
| (Vector | AAACUGAAAUGCAGUAAAGAGAAGCCAAAGUGUGCUAAGUGUCUGAAAAA |
| Builder) | CAAUUGGGAGUGUAGAUACUCCCCCAAGACAAAGAGAAGCCCACUGACAAG |
| | GGCCCACCUGACCGAGGUGGAGAGUAGACUGGAGAGACUGGAGCAGCUGUU |
| | UCUGCUGAUCUUCCCAAGGGAGGACCUGGACAUGAUUCUGAAGAUGGACUC |
| | CCUGCAGGAUAUCAAAGCCCUGCUGACCGGACUGUUCGUGCAGGACAACGU |
| | GAACAAGGACGCCGUGACCGACAGGCUGGCCAGCGUGGAGACgGACAUGCC |
| | CCUGACACUGAGACAGCACCGGAUCAGCGCCACCAGCUCUUCCGAGGAGAG |
| | UUCCAACAAGGGACAGCGGCAGUUGACAGUGUCC (SEQ ID NO: 295) |
| Human | AUGAAGCUGCUGAGCAGCAUCGAGCAAGCCUGCGACAUCUGUAGGCUGAAG |
| (Genewiz) | AAGCUGAAGUGCAGCAAGGAGAAGCCUAAGUGCGCCAAGUGCCUGAAGAAC |
| | AACUGGGAGUGUAGGUACAGCCCUAAGACGAAGAGGAGCCCUCUGACAAGG |
| | GCCCACCUGACCGAGGUGGAGUCUAGGCUGGAGAGGCUGGAGCAGCUGUUC |
| | CUGCUGAUCUUCCCUAGGGAGGACCUGGACAUGAUCCUGAAGAUGGACAGC |
| | CUGCAAGACAUCAAGGCCCUGCUGACCGGCCUGUUCGUGCAAGACAACGUG |
| | AACAAGGACGCCGUGACCGAUAGGCUGGCUAGCGUGGAAACCGACAUGCCU |
| | CUGACCCUGAGGCAGCAUAGGAUCAGCGCCACAAGCAGCAGCGAGGAGAGC |
| | AGCAACAAGGGACAGAGGCAGCUGACCGUGAGC (SEQ ID NO: 296) |
| Human | AUGAAGUUACUAUCUUCGAUUGAGCAAGCCUGUGACAUCUGCAGACUUAAG |
| (Genesmart) | AAACUGAAAUGCAGCAAAGAGAAGCCCAAAUGUGCUAAGUGCCUGAAGAAC |
| | AACUGGGAGUGUCGUUACAGCCCAAAGACCAAGCGCUCACCUCUUACUCGA |
| | GCUCACCUGACAGAGGUGGAAAGUCGCCUGGAGCGGUUAGAGCAGCUGUUC |
| | CUGCUCAUCUUCCCCAGAGAAGACCUGGACAUGAUCCUCAAGAUGGACAGC |
| | CUGCAGGAUAUUAAAGCCCUGCUCACAGGCCUGUUCGUGCAGGACAAUGUC |
| | AAUAAAGAUGCUGUGACUGAUAGACUUGCAUCUGUAGAAACGGACAUGCCU |
| | CUGACUCUGCGGCAGCAUCGGAUAUCUGCCACCUCCUCCUCAGAAGAAAGU |
| | AGCAACAAGGGCCAGAGGCAGUUGACCGUUUCU (SEQ ID NO: 297) |

As another example, when the DTS comprises a binding sequence for an Arc protein, e.g., RYRVTAGANNNNNTCTABYRY (SEQ ID NO:298) or a variant thereof, the cell may also be contacted with an mRNA encoding an Arc protein. In some embodiments, the Arc protein has been engineered to comprise an NLS. In some embodiments, the NLS is proximal to the N terminus. In other embodiments, the NLS is proximal to the C-terminus. In some embodiments, the Arc protein is engineered to comprise an NLS at both the N-terminus and the C-terminus. In some embodiments, the NLS is fused to the Arc protein with a linker. In some embodiments, the NLS is selected from those listed in Table 7. In some certain embodiments, the NLS is the NLS of SV40 Large T antigen or an optimized variant thereof, the NLS of the influenza A nuclear protein INF-A, the NLS of c-myc, or a fusion of the c-myc NLS to the influenza A nuclear protein INF-A NLS.

In some embodiments, the Arc protein comprises a sequence having a sequence identity of 90% or more, in some instances 92%, 93%, 94%, 95% or more, in certain instances 96%, 97%, 98%, 99% identity or more, e.g., is 100% identical to the sequence encoding the Salmonella phage Arc-like repressor:
MKGMSKMPQFNLRWPREVLDLVRKVAEENGRSVNSEIYQRVMESFKKEGRIGA (SEQ ID NO: 299). For example, the Arc protein may be the st11 variant MKGMSKMPQFNLRWPREVLDLVRKVAEENGRSVNSEIYQRVMASFAKEGRIAAKNQH E (SEQ ID NO: 300), or a protein having a sequence identity of 90% or more, in some instances 92%, 93%, 94%, 95% or more, in certain instances 96%, 97%, 98%, 99% identity or more to the st11 variant.

In some embodiments, the Arc protein is encoded by a polynucleotide comprising a sequence having a sequence identity of 80% or more, for example 85%, 90%, or 95% sequence identity or more, in some instances 96%, 97%, 98%, or 99% sequence identity, in some cases 100% sequence identity to the Salmonella phage Arc-like repressor wild type mRNA sequence:
AUGAAGGGCAUGAGCAAGAUGCCCCAGUUCAACCUGCGCUGGCCCCGCGAGGUGCUGGAC CUGGUGCGCAAGGUGGCCGAGGAGAACGGCCGCAGCGUGAACAGCGAGAUCUACCAGCGC GUGAUGGAGAGCUUCAAGAAGGAGGGCCGCAUCGGCGCC (SEQ ID NO:301). In some embodiments, the Arc protein is encoded by a polynucleotide comprises a sequence having a sequence identity of 80% or more, for example 85%, 90%, or 95% sequence identity or more, in some instances 96%, 97%, 98%, or 99% sequence identity, in some cases 100% sequence identity to the sequence encoding the st11 Arc variant:
AUGAAGGGCAUGAGCAAGAUGCCCCAGUUCAACCUGCGCUGGCCCCGCGAGGUGCUGGAC CUGGUGCGCAAGGUGGCCGAGGAGAACGGCCGCAGCGUGAACAGCGAGAUCUACCAGCGC GUGAUGGCCAGCUUCGCCAAGGAGGGCCGCAUCGCOGCCAAGAACCAGCACGAG (SEQ ID NO: 302). In some instances, the polynucleotide has been codon optimized for expression in a particular host cell, e.g., codon optimized for expression in human cells.

As another example, when the DTS comprises the binding sequence for a Mnt protein, e.g., GGNCCACNGTGGNCC (SEQ ID NO:303) or a variant thereof, for example ATAGGTCCACGGTGGACCATA (SEQ ID NO:304), the cell may also be contacted with an mRNA encoding a Mnt protein. In some embodiments, the Mnt protein has been engineered to comprise an NLS. In some embodiments, the NLS is proximal to the N terminus. In other embodiments, the NLS is proximal to the C-terminus. In some embodiments, the Mnt protein is engineered to comprise an NLS at both the N-terminus and the C-terminus. In some embodiments, the NLS is fused to the Mnt protein with a linker. In some embodiments, the NLS is selected from those listed in Table 7. In some certain embodiments, the NLS is the NLS of SV40 Large T antigen or an optimized variant thereof, the NLS of the influenza A nuclear protein INF-A, the NLS of c-myc, or a fusion of the c-myc NLS to the influenza A nuclear protein INF-A NLS.

In some embodiments, the Mnt protein comprises a sequence having a sequence identity of 90% or more, in some instances 92%, 93%, 94%, 95% or more, in certain instances 96%, 97%, 98%, 99% identity or more, e.g., is 100% identical to native wild-type Mnt:

(SEQ ID NO: 305)
MARDDPHFNFRMPMEVREKLKFRAEANGRSMNSELLQIVQDALSK
PSPVTGYRNDAERLADEQSELVKKMVFDTLKDLYKKTT.

In some embodiments, the Mnt protein is encoded by a polynucleotide comprising a sequence having a sequence identity of 80% or more, for example 85%, 90%, or 95% sequence identity or more, in some instances 96%, 97%, 98%, or 99% sequence identity, in some cases 100% sequence identity across the sequence:
AUGGCCCGCGACGACCCCCACUUCAACUUCCGCAUGCCCAUGGAGGUGCGCGAGAAGCUG AAGUUCCGCGCCGAGGCCAACGGCCGCAGCAUGAACAGCGAGCUGCUGCAGAUCGUGCAG GACGCCCUGAGCAAGCCCAGCCCCGUGACCGGCUACCGCAACGACGCCGAGCGCCUGGCC GACGAGCAGAGCGAGCUGGUGAAGAAGAUGGUGUUCGACACCCUGAAGGACCUGUACAA GAAGACCACC (SEQ ID NO:306). In some embodiments, the Mnt polynucleotide has been codon optimized for expression in a particular host cell, e.g., codon optimized for expression in human or mouse cells.

As another example, when the DTS comprises the binding sequence for a PurR protein, e.g., ACGCAAACGTTTTCGT (SEQ ID NO:307) or a variant thereof, the cell may also be contacted with an mRNA encoding a PurR protein. In some embodiments, the PurR protein has been engineered to comprise an NLS. In some embodiments, the NLS is proximal to the N terminus. In other embodiments, the NLS is proximal to the C-terminus. In some embodiments, the PurR protein is engineered to comprise an NLS at both the N-terminus and the C-terminus. In some embodiments, the NLS is fused to the PurR protein with a linker. In some embodiments, the NLS is selected from those listed in Table 7.

In some embodiments, the PurR protein comprises a sequence having a sequence identity of 90% or more, in some instances 92%, 93%, 94%, 95% or more, in certain instances 96%, 97%, 98%, 99% identity or more, e.g. is 100% identical to wild-type Escherichia coli HTH-type transcriptional repressor PurR:

(SEQ ID NO: 308)
MATIKDVAKRANVSTTTVSHVINKTRFVAEETRNAVWAAIKELHY
SPSAVARSLKVNHTKSIGLLATSSEAAYFAENIEAVEKNCFQKGY
TLILGNAWNNLEKQRAYLSMMAQKRVDGLLVMCSEYPEPLLAMLE
EYRHIPMVVMDWGEAKADFTDAVIDNAFEGGYMAGRYLIERGHRE
IGVIPGPLERNTGAGRLAGFMKAMEEAMIKVPESWIVQGDFEPES
GYRAMQQILSQPHRPTAVFCGGDIMAMGALCAADEMGLRVPQDVS
LIGYDNVRNARYFTPALTTIHQPKDSLGETAFNMLLDRIVNKREE
PQSIEVHPRLIERRSVADGPFRDYRR.

In some embodiments, the PurR protein is encoded by a polynucleotide comprising a sequence having a sequence identity of 80% or more, for example 85%, 90%, or 95% or more, in some instances 96%, 97%, 98%, or 99% identity, in some cases 100% sequence identity across the length of the sequence:
AUGGCCACCAUCAAGGACGUGGCCAAGCGCGCCAACGUGAGCACCACCACCGUGAGCCAC GUGAUCAACAAGACCCGCUUCGUGGCCGAGGAGACGCGCAACGCCGUGUGGGCCGCCAUC AAGGAGCUGCACUACAGCCCCAGCGCCGUGGCCCGCAGCCUGAAGGUGAACCACACCAAG AGCAUCGGCCUGCUGGCCACCAGCAGCGAGGCCGCCUACUUCGCCGAGAUCAUCGAGGCC GUGGAGAAGAACUGCUUCCAGAAGGGCUACACCCUGAUCCUGGGCAACGCCUGGAACAAC CUGGAGAAGCAGCGCGCCUACCUGAGCAUGAUGGCCCAGAAGCGCGUGGACGGCCUGCUG GUGAUGUGCAGCGAGUACCCCGAGCCCCUGCUGGCCAUGCUGGAGGAGUACCGCCACAUC CCCAUGGUGGUGAUGGACUGGGGCGAGGCCAAGGCCGACUUCACCGACGCCGUGAUCGAC AACGCCUUCGAGGGCGGCUACAUGGCCGGCCGCUACCUGAUCGAGCGCGGCCACCGCGAG AUCGGCGUGAUCCCCGGCCCCCUGGAGCGCAACACCGGCGCCGGCCGCCUGGCCGGCUUC AUGAAGGCCAUGGAGGAGGCCAUGAUCAAGGUGCCCGAGAGCUGGAUCGUGCAGGGCGA CUUCGAGCCCGAGAGCGGCUACCGCGCCAUGCAGCAGAUCCUGAGCCAGCCCCACCGCCCC ACCGCCGUGUUCUGCGGCGGCGACAUCAUGGCCAUGGGCGCCCUGUGCGCCGCCGACGAG AUGGGCCUGCGCGUGCCCCAGGACGUGAGCCUGAUCGGCUACGACAACGUGCGCAACGCC CGCUACUUCACCCCCGCCCUGACCACCAUCCACCAGCCCAAGGACAGCCUGGGCGAGACGG CCUUCAACAUGCUGCUGGACCGCAUCGUGAACAAGCGCGAGGAGCCCCAGAGCAUCGAGG UGCACCCCCGCCUGAUCGAGCGCCGCAGCGUGGCCGACGGCCCCUUCCGCGACUACCGCCG C (SEQ ID NO:309). In some embodiments, the polynucleotide encoding the PurR protein has been codon optimized for expression in a particular host cell, e.g., codon optimized for expression in human or mouse cells.

As another example, when the DTS comprises the binding sequence for a Bac434 protein, e.g., ACAAGAAAGTTTGT (SEQ ID NO:445), ACAAGATACATTGT (SEQ ID NO:446), or ACAAGAAAAACTGT (SEQ ID NO:447) or a variant thereof, the cell may also be contacted with an mRNA encoding a Bac434 protein. In some embodiments, the Bac434 protein has been engineered to comprise an NLS. In some embodiments, the NLS is proximal to the N terminus. In other embodiments, the NLS is proximal to the C-terminus. In some embodiments, the Bac434 protein is engineered to comprise an NLS at both the N-terminus and the C-terminus. In some embodiments, the NLS is fused to the Bac434 protein with a linker. In some embodiments, the NLS is selected from those listed in Table 7.

In some embodiments, the Bac434 protein comprises a sequence having a sequence identity of 90% or more, in some instances 92%, 93%, 94%, 95% or more, in certain instances 96%, 97%, 98%, 99% identity or more, e.g., is 100% identical to the wild type Escherichia coli Phage 434 Repressor protein CI:

(SEQ ID NO: 448)
MSISSRVKSKRIQLGLNQAELAQKVGTTQQSIEQLENGKTKRPRF
LPELASALGVSVDWLLNGTSDSNVRFVGHVEPKGKYPLISMVRAG
SWCEA.

In some embodiments, the Bac434 protein is encoded by a polynucleotide comprising a sequence having a sequences identity of 80% or more, for example 85%, 90%, or 95% sequence identity or more, in some instances 96%, 97%, 98%, or 99% sequence identity, in some cases 100% sequence identity to: AUGAGCAUCAGCAGCCGCGUGAAGAGCAAGCGCAUCCAGCUGGGCCUGAACCAGGCCGAG CUGGCCCAGAAGGUGGGCACCACCCAGCAGAGCAUCGAGCAGCUGGAGAACGGCAAGACC AAGCGCCCCCGCUUCCUGCCCGAGCUGGCCAGCGCCCUGGGCGUGAGCGUGGACUGGCUG CUGAACGGCACCAGCGACAGCAACGUGCGCUUCGUGGGCCACGUGGAGCCCAAGGGCAAG UACCCCCUGAUCAGCAUGGUGCGCGCCGGCAGCUGGUGCGAGGCC (SEQ ID NO:449). In some embodiments, the polynucleotide sequence encoding the Bac434 protein has been codon optimized for expression in a particular host cell, e.g., codon optimized for expression in human or mouse cells.

As another example, when the DTS comprises the binding sequence for a GCN4 protein, e.g., AGTGACTCATT (SEQ ID NO:450) or a variant thereof, the cell may also be contacted with an mRNA encoding a GCN4 protein. In some embodiments, the GCN4 protein has been engineered to comprise an NLS. In some embodiments, the NLS is proximal to the N terminus. In other embodiments, the NLS is proximal to the C-terminus. In some embodiments, the GCN4 protein is engineered to comprise an NLS at both the N-terminus and the C-terminus. In some embodiments, the NLS is fused to the GCN4 protein with a linker. In some embodiments, the NLS is selected from those listed in Table 7.

In some embodiments, the GCN4 protein comprises a sequence having a sequence identity of 90% or more, in some instances 92%, 93%, 94%, 95% or more, in certain instances 96%, 97%, 98%, 99% identity or more, e.g. is 100% identical to the wild type sequence for Saccharomyces cerevisiae GCN4:

(SEQ ID NO: 451)
MSEYQPSLFALNPMGFSPLDGSKSTNENVSASTSTAKPMVGQLIF
DKFIKTEEDPIIKQDTPSNLDFDFALPQTATAPDAKTVLPIPELD
DAVVESFFSSSTDSTPMFEYENLEDNSKEWTSLFDNDIPVTTDDV
SLADKAIESTEEVSLVPSNLEVSTTSFLPTPVLEDAKLTQTRKVK
KPNSVVKKSHHVGKDDESRLDHLGVVAYNRKQRSIPLSPIVPESS
DPAALKRARNTEAARRSRARKLQRMKQLEDKVEELLSKNYHLENE
VARLKKLVGER.

In some embodiments, the GCN4 protein is encoded by a polynucleotide comprising a sequence having a sequences identity of 80% or more, for example 85%, 90%, or 95% sequence identity or more, in some instances 96%, 97%, 98%, or 99% sequence identity, in some cases 100% sequence identity to AUGUCCGAAUAUCAACCAUCUUUAUUUGCAUUGAAUCCAAUGGGGUUUUCCCCACUUGA UGGAUCCAAAUCCACAAAUGAAAAUGUUUCUGCAUCAACAAGUACUGCAAAGCCAAUGG UCGGGCAAUUGAUCUUUGAUAAAUUUAUUAAGACAGAAGAAGAUCCGAUUAUAAAACAA GAUACUCCCAGUAAUUUGGAUUUUGAUUUUGCGUUGCCUCAAACAGCUACAGCUCCAGA UGCUAAAACAGUCUUACCAAUUCCAGAACUCGAUGAUGCUGUCGUCGAAAGUUUCUUUU CAAGUUCCACGGAUAGUACACCAAUGUUUGAGUAUGAAAAUUUGGAAGAUAAUUCAAAA GAAUGGACUUCUCUCUUUGACAAUGACAUUCCAGUCACUACUGAUGAUGUUUCCCUUGCA GAUAAAGCUAUUGAAUCUACAGAAGAAGUAUCUUUAGUCCCAUCCAAUCUUGAAGUUUC CACUACGAGUUUUCUCCCUACGCCUGUACUUGAAGAUGCAAAAUUGACGCAAACGCGGAA AGUCAAGAAACCAAAUUCUGUUGUCAAGAAAUCUCAUCAUGUUGGAAAAGAUGAUGAAU CUAGGCUCGAUCAUCUUGGAGUUGUCGCUUAUAAUAGGAAACAAAGAUCUAUACCACUC UCCCCUAUAGUCCCAGAAAGUAGUGAUCCUGCUGCUUUAAAACGGGCUAGGAAUACAGA AGCGGCUCGAAGGUCCAGGGCCCGGAAAUUACAACGGAUGAAACAACUCGAAGAUAAAG UAGAAGAAUUGCUUUCUAAGAAUUAUCAUCUUGAGAAUGAAGUUGCACGGUUGAAGAAA CUCGUUGGGGAACGG (SEQ ID NO:452). In some embodiments, the GCN polynucleotide has been codon optimized for expression in a particular host cell, e.g., codon optimized for expression in human or mouse cells.

As another example, when the DTS comprises the binding sequence for a Lac Repressor protein (“LacR”), e.g., TTGTTATCCGCTCACAA (SEQ ID NO:453) or a variant thereof, the cell may also be contacted with an mRNA encoding a LacR protein. In some embodiments, the LacR protein has been engineered to comprise an NLS. In some embodiments, the NLS is proximal to the N terminus. In other embodiments, the NLS is proximal to the C-terminus. In some embodiments, the LacR protein is engineered to comprise an NLS at both the N-terminus and the C-terminus. In some embodiments, the NLS is fused to the LacR protein with a linker. In some embodiments, the NLS is selected from those listed in Table 7.

In some embodiments, the LacR protein comprises a sequence having a sequence identity of 90% or more, in some instances 92%, 93%, 94%, 95% or more, in certain instances 96%, 97%, 98%, 99% identity or more, e.g. is 100% identical to the wild type sequence for the Escherichia coli DNA-binding transcriptional repressor LacI:

(SEQ ID NO: 454)
MKPVTLYDVAEYAGVSYQTVSRVVNQASHVSAKTREKVEAAMAEL
NYIPNRVAQQLAGKQSLLIGVATSSLALHAPSQIVAAIKSRADQL
GASVVVSMVERSGVEACKAAVHNLLAQRVSGLIINYPLDDQDAIA
VEAACTNVPALFLDVSDQTPINSIIFSHEDGTRLGVEHLVALGHQ
QIALLAGPLSSVSARLRLAGWHKYLTRNQIQPIAEREGDWSAMSG
FQQTMQMLNEGIVPTAMLVANDQMALGAMRAITESGLRVGADISV
VGYDDTEDSSCYIPPLTTIKQDFRLLGQTSVDRLLQLSQGQAVKG
NQLLPVSLVKRKTTLAPNTQTASPRALADSLMQLARQVSRLESGQ.

In some embodiments, the LacR protein is encoded by a polynucleotide comprising a sequence having a sequence identity of 80% or more, for example 85%, 90%, or 95% or more, in some instances 96%, 97%, 98%, or 99% identity, in some cases 100% sequence identity across the length of the sequence:
AUGAAACCAGUCACACUCUAUGAUGUAGCGGAAUAUGCUGGGGUAUCCUAUCAAACAGU UAGUAGAGUUGUUAAUCAAGCUUCACAUGUUUCCGCCAAAACGAGGGAAAAGGUUGAAG CAGCUAUGGCUGAACUUAAUUAUAUACCAAAUCGAGUAGCGCAACAACUCGCAGGGAAA CAAUCUUUACUCAUUGGAGUCGCAACAUCCUCACUCGCUCUCCAUGCUCCUUCUCAAAUA GUCGCUGCUAUUAAAAGUAGAGCUGAUCAAUUAGGGGCAUCUGUCGUAGUCUCUAUGGU CGAAAGAUCUGGUGUAGAAGCUUGUAAAGCUGCAGUCCAUAAUCUCCUCGCUCAACGGG UCAGCGGGCUUAUAAUUAAUUAUCCUCUUGAUGAUCAAGAUGCAAUAGCUGUCGAAGCA GCUUGUACUAAUGUCCCUGCGUUGUUUCUUGAUGUCUCAGAUCAAACACCAAUAAAUUC AAUUAUAUUUAGUCAUGAAGAUGGUACAAGGCUCGGGGUUGAACAUCUCGUAGCACUCG GUCAUCAACAAAUAGCACUCCUUGCUGGGCCACUCUCUUCCGUUUCUGCACGGCUCAGGC UCGCAGGAUGGCAUAAAUAUUUGACACGUAAUCAAAUUCAACCUAUAGCUGAAAGGGAA GGUGAUUGGUCCGCUAUGAGUGGGUUUCAACAAACAAUGCAAAUGUUGAAUGAAGGGAU AGUUCCAACUGCAAUGCUCGUUGCUAAUGAUCAAAUGGCGCUUGGUGCGAUGCGAGCUA UUACAGAAUCCGGACUUCGGGUUGGUGCUGAUAUAUCCGUAGUCGGAUAUGAUGAUACA GAAGAUUCCUCAUGUUAUAUUCCACCUCUUACAACUAUUAAACAAGAUUUUAGGCUUCU CGGGCAAACAUCCGUCGAUAGACUUUUACAACUCUCCCAAGGACAAGCAGUAAAAGGAAA UCAAUUAUUGCCGGUUAGUCUUGUCAAACGUAAAACAACGCUUGCGCCAAAUACACAAAC GGCUUCUCCUAGAGCACUUGCUGAUAGUCUUAUGCAACUCGCACGACAAGUCUCUCGAUU GGAAUCUGGACAA (SEQ ID NO:310). In some embodiments, the polynucleotide encoding the LacR protein has been codon optimized for expression in a particular host cell, e.g., codon optimized for expression in human or mouse cells.

As another example, when the DTS comprises the binding sequence for a I-SceI D44A protein, e.g., TAGGGATAACAGGGTAAT (SEQ ID NO:311) or a variant thereof, the cell may also be contacted with an mRNA encoding a I-SceI D44A protein. In some embodiments, the I-SceI protein has been engineered to comprise an NLS. In some embodiments, the NLS is proximal to the N terminus. In other embodiments, the NLS is proximal to the C-terminus. In some embodiments, the I-SceI protein is engineered to comprise an NLS at both the N-terminus and the C-terminus. In some embodiments, the NLS is fused to the I-SceI protein with a linker.

In some embodiments, the I-SceI protein comprises a sequence having a sequence identity of 90% or more, in some instances 92%, 93%, 94%, 95% or more, in certain instances 96%, 97%, 98%, 99% identity or more, e.g. is 100% identical to MKNIKKNQVMNLGPNSKLLKEYKSQLIELNIEQFEAGIGLILGAAYIRSRDEGKTYCMQFEWKN KAYMDHVCLLYDQWVLSPPHKKERVNHLGNLVITWGAQTFKHQAFNKLANLFIVNNKKTIPNN LVENYLTPMSLAYWFMDDGGKWDYNKNSTNKSIVLNTQSFTFEEVEYLVKGLRNKFQLNCYV KINKNKPIIYIDSMSYLIFYNLIKPYLIPQMMYKLPNTISSETFLK (SEQ ID NO:312), e.g. a sequence having 85% identity more to:

(SEQ ID NO: 313)
MPKKKRKVPKKHAAPPKKKRKVEDPRFMYPYDVPDYAGMKNIKKN
QVMNLGPNSKLLKEYKSQLIELNIEQFEAGIGLILGAAYIRSRDE
GKTYCMQFEWKNKAYMDHVCLLYDQWVLSPPHKKERVNHLGNLVI
TWGAQTFKHQAFNKLANLFIVNNKKTIPNNLVENYLTPMSLAYWF
MDDGGKWDYNKNSTNKSIVLNTQSFTFEEVEYLVKGLRNKFQLNC
YVKINKNKPIIYIDSMSYLIFYNLIKPYLIPQMMYKLPNTISSET
FLK.

As another example, when the TFBS is the binding sequence for a gene editing system, e.g. the guide RNA for a Cas nuclease, the zinc finger of a zinc finger nuclease, the TALE of a TALEN, e.g. as described below and as known in the art, the cell may also be contacted with an mRNA encoding the Cas nuclease, the ZF nuclease, or the TALEN. In some certain embodiments, the nuclease of the gene editing system is catalytically active. Put another way, the nuclease is able to modify DNA, for example to nick it, to create a double-stranded break, to replace one or more nucleotides within it. In other certain embodiments, the gene editing system is catalytically attenuated, e.g., its activity is reduced 50% or more, in some instances 60%, 70%, 80%, 90% or more, in some cases 95% or more, e.g., the catalytic activity has been abrogated.

### Target Cells

Cells employed in embodiments of the invention, referred to herein as “target cells” may vary. It should be appreciated that the target cell may be of any origin, for example from an organism. In some embodiments, the target cell is a mammalian cell. Some non-limiting examples of a mammalian cell include, without limitation, a mouse cell, a rat cell, hamster cell, a rodent cell, and a nonhuman primate cell. In some embodiments, the target cell is a human cell. It should also be appreciated that the target cell may be of any cell type. For example, the target cell may be a stem cell, which may include embryonic stem cells, induced pluripotent stem cells (iPS cells), fetal stem cells, cord blood stem cells, or adult stem cells (i.e., tissue specific stem cells). In other cases, the target cell may be any differentiated cell type found in a subject. Cells of interest include both dividing cells and non-dividing cells. Examples of specific target cells of interest include, but are not limited to: hepatocytes, stellate cells, T lymphocytes, B lymphocytes, NK cells, skeletal muscle cells, cardiomyocytes, neurons, astrocytes, oligodendrocytes, dendritic cells, skin cells, photoreceptors, RPE cells, radial glia, etc.

In some embodiments, the target cell is a cell in vitro, and the method includes contacting the cell in vitro. In some embodiments, the target cell is a cell in a subject, and the methods include administering the NTDNA and if necessary an inducing agent (where either or both may be present in the same or different suitable delivery vehicle, as desired) to the subject. In some embodiments, the subject is a mammalian subject, for example, a rodent, a mouse, a rat, a hamster, or a non-human primate. In some embodiments, the subject is a human subject.

In embodiments of the invention in which an inducing agent is employed, a target cell may be contacting simultaneously or sequentially with the inducing agent, as desired. In some instances, the target cell is contacted simultaneously with the NTDNA and the inducing agent. As such, the NTDNA an inducing agent are contacted at the same time with the target cells. In other instances, the NTDNA and the inducing agent are sequentially contacted with the cell. For example, the target cell may be contacted with the NTDNA before being contacted with the inducing agent. Alternatively, the target cell may be contacted with the NTDNA after being contacted with the inducing agent.

In embodiments of the invention in which the nuclear targeting factor is provided to the cell, the target cell may be contacted simultaneously or sequentially with an mRNA encoding the NTF. In some instances, the target cell is contacted simultaneously with the NTDNA and the NTF. In certain such embodiments, the NTDNA and NTF are provided as a single formulation in a delivery vehicle (as described in greater detail below) that is administered to the target cell. In other embodiments, the NTDNA and NTF are provided as separate formulations that are administered simultaneously to the cell, i.e., as an admixture. Preferably, the NTDNA and NTF are prepared as a single formulation in the delivery vehicle for contacting with the cell.

### Genomic Integration

In some instances, the NTDNA is contacted with a target cell in conjunction with a gene editing system, e.g., that is configured to provide genomic integration of the cargo nucleic acid component of the NTDNA. In such embodiments, as the NTDNA is contacted with the target cell in conjunction with a gene editing system, it may be contacted with the target cell simultaneously or sequentially with the gene editing system. In some instances, one or more components of a gene editing system may present with the NTDNA in a delivery composition, such as a cytosolic delivery composition (e.g., LNP), such as described in greater detail below. Gene editing systems that may be employed in such embodiments may vary, as desired. In some embodiments, the employed gene editing system is configured to genomically integrate the cargo nucleic acid into a specific safe harbor location in the genome, for example to a genomic location within or near an endogenous albumin locus. Generally, one skilled in the art will understand that a safe harbor locus is a location within a genome that can be used for integrating exogenous nucleic acids, where the addition of exogenous nucleic acids into the safe harbor locus does not cause significant effect on the growth of the host cell by the addition of the nucleic acids alone. In some embodiments, the cargo nucleic acid may be inserted into the specific safe harbor location in the genome that may either utilize the promoter found at that safe harbor locus, or allow the expressional regulation of a coding sequence of the cargo nucleic acid by an exogenous promoter that is fused to the cargo nucleic acid coding sequence prior to insertion.

Gene editing can be conducted using nucleases engineered to target specific sequences. To date there are four major types of nucleases: meganucleases and their derivatives, zinc finger nucleases (ZFNs), transcription activator like effector nucleases (TALENs), and CRISPR-Cas9 nuclease systems. The nuclease platforms vary in difficulty of design, targeting density and mode of action, particularly as the specificity of ZFNs and TALENs is through protein-DNA interactions, while RNA-DNA interactions primarily guide Cas9. Cas9 cleavage also requires an adjacent motif, the PAM, which differs between different CRISPR systems. Cas9 from Streptococcus pyogenes cleaves using a NRG PAM, CRISPR from Neisseria meningitidis can cleave at sites with PAMs including NNNNGATT, NNNNNGTTT and NNNNGCTT. A number of other Cas9 orthologs target protospacer adjacent to alternative PAMs. CRISPR endonucleases, such as Cas9, can be used in various embodiments of the methods of the disclosure. However, the teachings described herein, such as therapeutic target sites, could be applied to other forms of endonucleases, such as ZFNs, TALENs, HEs, or MegaTALs, or using combinations of nucleases. These different systems are now described in further detail.

A given NTDNA delivery cytosolic delivery composition may include a one or more elements of a given gene editing system. Gene editing system employed in embodiments of the invention may include a number of different elements, such as nucleic acid elements, e.g., genome-targeting nucleic acids or Guide RNAs, nucleic acids, e.g., mRNAs encoding endonucleases; polypeptide components, e.g., endonucleases, etc. These various components are now reviewed in greater detail in conjunction with the description of representative endonuclease based genomic integration systems.

In some embodiments, the methods of genome edition and compositions therefore use a nucleic acid sequence (or oligonucleotide) encoding a site-directed polypeptide or DNA endonuclease. The nucleic acid sequence encoding the site-directed polypeptide can be DNA or RNA. If the nucleic acid sequence encoding the site-directed polypeptide is RNA, it can be covalently linked to a gRNA sequence or exist as a separate sequence. In some embodiments, a peptide sequence of the site-directed polypeptide or DNA endonuclease can be used instead of the nucleic acid sequence thereof.

The modifications of the target DNA due to NHEJ and/or HDR can lead to, for example, mutations, deletions, alterations, integrations, gene correction, gene replacement, gene tagging, transgene insertion, nucleotide deletion, gene disruption, translocations and/or gene mutation. The process of integrating non-native nucleic acid, e.g., a cargo nucleic acid of a NTDNA, into genomic DNA is an example of genome editing. A site-directed polypeptide is a nuclease used in genome editing to cleave DNA. The site-directed can be administered to a cell or a patient as either: one or more polypeptides, or one or more mRNAs encoding the polypeptide. In some embodiments, a site-directed polypeptide has a plurality of nucleic acid-cleaving (i.e., nuclease) domains. Two or more nucleic acid-cleaving domains can be linked together via a linker. In some embodiments, the linker has a flexible linker. Linkers can have 1, 2, 3, 4, 5, 6, 7, 8, 9, 10, 11, 12, 13, 14, 15, 16, 17, 18, 19, 20, 21, 22, 23, 24, 25, 30, 35, 40 or more amino acids in length.

CRISPR. In the context of a CRISPR/Cas or CRISPR/Cpf1 system, the site-directed polypeptide can bind to a guide RNA that, in turn, specifies the site in the target DNA to which the polypeptide is directed. In some embodiments of CRISPR/Cas or CRISPR/Cpf1 systems herein, the site-directed polypeptide is an endonuclease, such as a DNA endonuclease.

Naturally-occurring wild-type Cas9 enzymes have two nuclease domains, a HNH nuclease domain and a RuvC domain. Herein, the “Cas9” refers to both naturally-occurring and recombinant Cas9s. Cas9 enzymes contemplated herein have a HINH or HNH-like nuclease domain, and/or a RuvC or RuvC-like nuclease domain.

HINH or HNH-like domains have a McrA-like fold. HNH or HNH-like domains has two antiparallel β-strands and an α-helix. HNH or HNH-like domains has a metal binding site (e.g., a divalent cation binding site). HNH or HINH-like domains can cleave one strand of a target nucleic acid (e.g., the complementary strand of the crRNA targeted strand).

RuvC or RuvC-like domains have an RNaseH or RNaseH-like fold. RuvC/RNaseH domains are involved in a diverse set of nucleic acid-based functions including acting on both RNA and DNA. The RNaseH domain has 5 β-strands surrounded by a plurality of a-helices. RuvC/RNaseH or RuvC/RNaseH-like domains have a metal binding site (e.g., a divalent cation binding site). RuvC/RNaseH or RuvC/RNaseH-like domains can cleave one strand of a target nucleic acid (e.g., the non-complementary strand of a double-stranded target DNA).

In some embodiments, the site-directed polypeptide has an amino acid sequence having at least 10%, at least 15%, at least 20%, at least 30%, at least 40%, at least 50%, at least 60%, at least 70%, at least 75%, at least 80%, at least 85%, at least 90%, at least 95%, at least 99%, or 100% amino acid sequence identity to a wild-type exemplary site-directed polypeptide [e.g., Cas9 from S. pyogenes, US2014/0068797 Sequence ID No. 8 or Sapranauskas et al., Nucleic Acids Res, 39 (21): 9275-9282 (2011)], and various other site-directed polypeptides). In some embodiments, the site-directed polypeptide has an amino acid sequence having at least 10%, at least 15%, at least 20%, at least 30%, at least 40%, at least 50%, at least 60%, at least 70%, at least 75%, at least 80%, at least 85%, at least 90%, at least 95%, at least 99%, or 100% amino acid sequence identity to the nuclease domain of a wild-type exemplary site-directed polypeptide (e.g., Cas9 from S. pyogenes, supra). In some embodiments, a site-directed polypeptide has at least 70, 75, 80, 85, 90, 95, 97, 99, or 100% identity to a wild-type site-directed polypeptide (e.g., Cas9 from S. pyogenes, supra) over 10 contiguous amino acids. In some embodiments, a site-directed polypeptide has at most: 70, 75, 80, 85, 90, 95, 97, 99, or 100% identity to a wild-type site-directed polypeptide (e.g., Cas9 from S. pyogenes, supra) over 10 contiguous amino acids. In some embodiments, a site-directed polypeptide has at least: 70, 75, 80, 85, 90, 95, 97, 99, or 100% identity to a wild-type site-directed polypeptide (e.g., Cas9 from S. pyogenes, supra) over 10 contiguous amino acids in a HNH nuclease domain of the site-directed polypeptide. In some embodiments, a site-directed polypeptide has at most: 70, 75, 80, 85, 90, 95, 97, 99, or 100% identity to a wild-type site-directed polypeptide (e.g., Cas9 from S. pyogenes, supra) over 10 contiguous amino acids in a HNH nuclease domain of the site-directed polypeptide. In some embodiments, a site-directed polypeptide has at least: 70, 75, 80, 85, 90, 95, 97, 99, or 100% identity to a wild-type site-directed polypeptide (e.g., Cas9 from S. pyogenes, supra) over 10 contiguous amino acids in a RuvC nuclease domain of the site-directed polypeptide. In some embodiments, a site-directed polypeptide has at most: 70, 75, 80, 85, 90, 95, 97, 99, or 100% identity to a wild-type site-directed polypeptide (e.g., Cas9 from S. pyogenes, supra) over 10 contiguous amino acids in a RuvC nuclease domain of the site-directed polypeptide.

In some embodiments, the site-directed polypeptide has a modified form of a wild-type exemplary site-directed polypeptide. The modified form of the wild-type exemplary site-directed polypeptide has a mutation that reduces the nucleic acid-cleaving activity of the site-directed polypeptide. In some embodiments, the modified form of the wild-type exemplary site-directed polypeptide has less than 90%, less than 80%, less than 70%, less than 60%, less than 50%, less than 40%, less than 30%, less than 20%, less than 10%, less than 5%, or less than 1% of the nucleic acid-cleaving activity of the wild-type exemplary site-directed polypeptide (e.g., Cas9 from S. pyogenes, supra). The modified form of the site-directed polypeptide can have no substantial nucleic acid-cleaving activity. When a site-directed polypeptide is a modified form that has no substantial nucleic acid-cleaving activity, it is referred to herein as “enzymatically inactive.”

In some embodiments, the modified form of the site-directed polypeptide has a mutation such that it can induce a single-strand break (SSB) on a target nucleic acid (e.g., by cutting only one of the sugar-phosphate backbones of a double-strand target nucleic acid). In some embodiments, the mutation results in less than 90%, less than 80%, less than 70%, less than 60%, less than 50%, less than 40%, less than 30%, less than 20%, less than 10%, less than 5%, or less than 1% of the nucleic acid-cleaving activity in one or more of the plurality of nucleic acid-cleaving domains of the wild-type site directed polypeptide (e.g., Cas9 from S. pyogenes, supra). In some embodiments, the mutation results in one or more of the plurality of nucleic acid-cleaving domains retaining the ability to cleave the complementary strand of the target nucleic acid, but reducing its ability to cleave the non-complementary strand of the target nucleic acid. In some embodiments, the mutation results in one or more of the plurality of nucleic acid-cleaving domains retaining the ability to cleave the non-complementary strand of the target nucleic acid, but reducing its ability to cleave the complementary strand of the target nucleic acid. For example, residues in the wild-type exemplary S. pyogenes Cas9 polypeptide, such as Asp 10, His840, Asn854 and Asn856, are mutated to inactivate one or more of the plurality of nucleic acid-cleaving domains (e.g., nuclease domains). In some embodiments, the residues to be mutated correspond to residues Asp10, His840, Asn854 and Asn856 in the wild-type exemplary S. pyogenes Cas9 polypeptide (e.g., as determined by sequence and/or structural alignment). Non-limiting examples of mutations include D10A, H840A, N854A or N856A. One skilled in the art will recognize that mutations other than alanine substitutions are suitable.

In some embodiments, a D10A mutation is combined with one or more of H840A, N854A, or N856A mutations to produce a site-directed polypeptide substantially lacking DNA cleavage activity. In some embodiments, a H840A mutation is combined with one or more of D10A, N854A, or N856A mutations to produce a site-directed polypeptide substantially lacking DNA cleavage activity. In some embodiments, a N854A mutation is combined with one or more of H840A, D10A, or N856A mutations to produce a site-directed polypeptide substantially lacking DNA cleavage activity. In some embodiments, a N856A mutation is combined with one or more of H840A, N854A, or D10A mutations to produce a site-directed polypeptide substantially lacking DNA cleavage activity. Site-directed polypeptides that have one substantially inactive nuclease domain are referred to as “nickases”.

In some embodiments, variants of RNA-guided endonucleases, for example Cas9, can be used to increase the specificity of CRISPR-mediated genome editing. Wild type Cas9 is generally guided by a single guide RNA designed to hybridize with a specified about.20 nucleotide sequence in the target sequence (such as an endogenous genomic locus). However, several mismatches can be tolerated between the guide RNA and the target locus, effectively reducing the length of required homology in the target site to, for example, as little as 13 nt of homology, and thereby resulting in elevated potential for binding and double-strand nucleic acid cleavage by the CRISPR/Cas9 complex elsewhere in the target genome—also known as off-target cleavage. Because nickase variants of Cas9 each only cut one strand, in order to create a double-strand break it is necessary for a pair of nickases to bind in close proximity and on opposite strands of the target nucleic acid, thereby creating a pair of nicks, which is the equivalent of a double-strand break. This requires that two separate guide RNAs—one for each nickase—must bind in close proximity and on opposite strands of the target nucleic acid. This requirement essentially doubles the minimum length of homology needed for the double-strand break to occur, thereby reducing the likelihood that a double-strand cleavage event will occur elsewhere in the genome, where the two guide RNA sites—if they exist—are unlikely to be sufficiently close to each other to enable the double-strand break to form. As described in the art, nickases can also be used to promote HDR versus NHEJ. HDR can be used to introduce selected changes into target sites in the genome through the use of specific donor sequences that effectively mediate the desired changes. Descriptions of various CRISPR/Cas systems for use in gene editing can be found, e.g., in international patent application publication number WO2013/176772, and in Nature Biotechnology 32, 347-355 (2014), and references cited therein.

In some embodiments, the site-directed polypeptide (e.g., variant, mutated, enzymatically inactive and/or conditionally enzymatically inactive site-directed polypeptide) targets nucleic acid. In some embodiments, the site-directed polypeptide (e.g., variant, mutated, enzymatically inactive and/or conditionally enzymatically inactive endoribonuclease) targets DNA. In some embodiments, the site-directed polypeptide (e.g., variant, mutated, enzymatically inactive and/or conditionally enzymatically inactive endoribonuclease) targets RNA.

In some embodiments, the site-directed polypeptide has one or more non-native sequences (e.g., the site-directed polypeptide is a fusion protein). In some embodiments, the site-directed polypeptide has an amino acid sequence having at least 15% amino acid identity to a Cas9 from a bacterium (e.g., S. pyogenes), a nucleic acid binding domain, and two nucleic acid cleaving domains (i.e., a HINH domain and a RuvC domain). In some embodiments, the site-directed polypeptide has an amino acid sequence having at least 15% amino acid identity to a Cas9 from a bacterium (e.g., S. pyogenes), and two nucleic acid cleaving domains (i.e., a HNH domain and a RuvC domain). In some embodiments, the site-directed polypeptide has an amino acid sequence having at least 15% amino acid identity to a Cas9 from a bacterium (e.g., S. pyogenes), and two nucleic acid cleaving domains, wherein one or both of the nucleic acid cleaving domains have at least 50% amino acid identity to a nuclease domain from Cas9 from a bacterium (e.g., S. pyogenes). In some embodiments, the site-directed polypeptide has an amino acid sequence having at least 15% amino acid identity to a Cas9 from a bacterium (e.g., S. pyogenes), two nucleic acid cleaving domains (i.e., a HNH domain and a RuvC domain), and non-native sequence (for example, a nuclear localization signal) or a linker linking the site-directed polypeptide to a non-native sequence. In some embodiments, the site-directed polypeptide has an amino acid sequence having at least 15% amino acid identity to a Cas9 from a bacterium (e.g., S. pyogenes), two nucleic acid cleaving domains (i.e., a HNH domain and a RuvC domain), wherein the site-directed polypeptide has a mutation in one or both of the nucleic acid cleaving domains that reduces the cleaving activity of the nuclease domains by at least 50%. In some embodiments, the site-directed polypeptide has an amino acid sequence having at least 15% amino acid identity to a Cas9 from a bacterium (e.g., S. pyogenes), and two nucleic acid cleaving domains (i.e., a HNH domain and a RuvC domain), wherein one of the nuclease domains has mutation of aspartic acid 10, and/or wherein one of the nuclease domains has mutation of histidine 840, and wherein the mutation reduces the cleaving activity of the nuclease domain(s) by at least 50%.

In some embodiments, the one or more site-directed polypeptides, e.g., DNA endonucleases, include two nickases that together effect one double-strand break at a specific locus in the genome, or four nickases that together effect two double-strand breaks at specific loci in the genome. Alternatively, one site-directed polypeptide, e.g., DNA endonuclease, affects one double-strand break at a specific locus in the genome.

In some embodiments, a polynucleotide encoding a site-directed polypeptide can be used to edit genome. In some of such embodiments, the polynucleotide encoding a site-directed polypeptide is codon-optimized according to methods standard in the art for expression in the cell containing the target DNA of interest. For example, if the intended target nucleic acid is in a human cell, a human codon-optimized polynucleotide encoding Cas9 is contemplated for use for producing the Cas9 polypeptide.

A CRISPR (Clustered Regularly Interspaced Short Palindromic Repeats) genomic locus can be found in the genomes of many prokaryotes (e.g., bacteria and archaea). In prokaryotes, the CRISPR locus encodes products that function as a type of immune system to help defend the prokaryotes against foreign invaders, such as virus and phage. There are three stages of CRISPR locus function: integration of new sequences into the CRISPR locus, expression of CRISPR RNA (crRNA), and silencing of foreign invader nucleic acid. Five types of CRISPR systems (e.g., Type I, Type II, Type III, Type U, and Type V) have been identified.

A CRISPR locus includes a number of short repeating sequences referred to as “repeats.” When expressed, the repeats can form secondary hairpin structures (e.g., hairpins) and/or have unstructured single-stranded sequences. The repeats usually occur in clusters and frequently diverge between species. The repeats are regularly interspaced with unique intervening sequences referred to as “spacers,” resulting in a repeat-spacer-repeat locus architecture. The spacers are identical to or have high homology with known foreign invader sequences. A spacer-repeat unit encodes a crisprRNA (crRNA), which is processed into a mature form of the spacer-repeat unit. A crRNA has a “seed” or spacer sequence that is involved in targeting a target nucleic acid (in the naturally occurring form in prokaryotes, the spacer sequence targets the foreign invader nucleic acid). A spacer sequence is located at the 5′ or 3′ end of the crRNA.

A CRISPR locus also has polynucleotide sequences encoding CRISPR Associated (Cas) genes. Cas genes encode endonucleases involved in the biogenesis and the interference stages of crRNA function in prokaryotes. Some Cas genes have homologous secondary and/or tertiary structures.

crRNA biogenesis in a Type II CRISPR system in nature requires a trans-activating CRISPR RNA (tracrRNA). The tracrRNA is modified by endogenous RNaseIII, and then hybridizes to a crRNA repeat in the pre-crRNA array. Endogenous RNaseIII is recruited to cleave the pre-crRNA. Cleaved crRNAs are subjected to exoribonuclease trimming to produce the mature crRNA form (e.g., 5′ trimming). The tracrRNA remains hybridized to the crRNA, and the tracrRNA and the crRNA associate with a site-directed polypeptide (e.g., Cas9). The crRNA of the crRNA-tracrRNA-Cas9 complex guides the complex to a target nucleic acid to which the crRNA can hybridize. Hybridization of the crRNA to the target nucleic acid activates Cas9 for targeted nucleic acid cleavage. The target nucleic acid in a Type II CRISPR system is referred to as a protospacer adjacent motif (PAM). In nature, the PAM is essential to facilitate binding of a site-directed polypeptide (e.g., Cas9) to the target nucleic acid. Type II systems (also referred to as Nmeni or CASS4) are further subdivided into Type II-A (CASS4) and II-B (CASS4a). Jinek et al., Science, 337 (6096): 816-821 (2012) showed that the CRISPR/Cas9 system is useful for RNA-programmable genome editing, and international patent application publication number WO 2013/176772 provides numerous examples and applications of the CRISPR/Cas endonuclease system for site-specific gene editing.

Type V CRISPR systems have several important differences from Type II systems. For example, Cpf1 is a single RNA-guided endonuclease that, in contrast to Type II systems, lacks tracrRNA. In fact, Cpf1-associated CRISPR arrays are processed into mature crRNAS without the requirement of an additional trans-activating tracrRNA. The Type V CRISPR array is processed into short mature crRNAs of 42-44 nucleotides in length, with each mature crRNA beginning with 19 nucleotides of direct repeat followed by 23-25 nucleotides of spacer sequence. In contrast, mature crRNAs in Type II systems start with 20-24 nucleotides of spacer sequence followed by about 22 nucleotides of direct repeat. Also, Cpf1 utilizes a T-rich protospacer-adjacent motif such that Cpf1-crRNA complexes efficiently cleave target DNA preceded by a short T-rich PAM, which is in contrast to the G-rich PAM following the target DNA for Type II systems. Thus, Type V systems cleave at a point that is distant from the PAM, while Type II systems cleave at a point that is adjacent to the PAM. In addition, in contrast to Type II systems, Cpf1 cleaves DNA via a staggered DNA double-stranded break with a 4 or 5 nucleotide 5′ overhang. Type II systems cleave via a blunt double-stranded break. Similar to Type II systems, Cpf1 contains a predicted RuvC-like endonuclease domain, but lacks a second HNH endonuclease domain, which is in contrast to Type II systems.

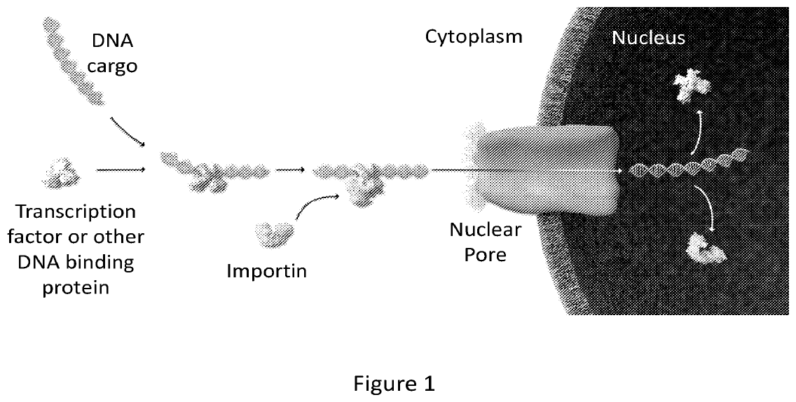

Exemplary CRISPR/Cas polypeptides include the Cas9 polypeptides in FIG. 1 of Fonfara et al., Nucleic Acids Research, 42:2577-2590 (2014). The CRISPR/Cas gene naming system has undergone extensive rewriting since the Cas genes were discovered.

A genome-targeting nucleic acid interacts with a site-directed polypeptide (e.g., a nucleic acid-guided nuclease such as Cas9), thereby forming a complex. The genome-targeting nucleic acid (e.g., gRNA, such as described in greater detail below) guides the site-directed polypeptide to a target nucleic acid.

In some embodiments the site-directed polypeptide and genome-targeting nucleic acid can each be administered separately to a cell or a patient. On the other hand, in some other embodiments the site-directed polypeptide can be pre-complexed with one or more guide RNAs, or one or more crRNA together with a tracrRNA. The pre-complexed material can then be administered to a cell or a patient. Such pre-complexed material is known as a ribonucleoprotein particle (RNP).

Genome-Targeting Nucleic Acid or Guide RNA. Genomic editing components may include a genome-targeting nucleic acid that can direct the activities of an associated polypeptide (e.g., a site-directed polypeptide or DNA endonuclease) to a specific target sequence within a target nucleic acid. In some embodiments, the genome-targeting nucleic acid is an RNA. A genome-targeting RNA is referred to as a “guide RNA” or “gRNA” herein. A guide RNA has at least a spacer sequence that hybridizes to a target nucleic acid sequence of interest and a CRISPR repeat sequence. In Type II systems, the gRNA also has a second RNA called the tracrRNA sequence. In the Type II guide RNA (gRNA), the CRISPR repeat sequence and tracrRNA sequence hybridize to each other to form a duplex. In the Type V guide RNA (gRNA), the crRNA forms a duplex. In both systems, the duplex binds a site-directed polypeptide such that the guide RNA and site-direct polypeptide form a complex. The genome-targeting nucleic acid provides target specificity to the complex by virtue of its association with the site-directed polypeptide. The genome-targeting nucleic acid thus directs the activity of the site-directed polypeptide.

In some embodiments, the genome-targeting nucleic acid is a double-molecule guide RNA. In some embodiments, the genome-targeting nucleic acid is a single-molecule guide RNA. A double-molecule guide RNA has two strands of RNA. The first strand has in the 5′ to 3′ direction, an optional spacer extension sequence, a spacer sequence and a minimum CRISPR repeat sequence. The second strand has a minimum tracrRNA sequence (complementary to the minimum CRISPR repeat sequence), a 3′ tracrRNA sequence and an optional tracrRNA extension sequence. A single-molecule guide RNA (sgRNA) in a Type II system has, in the 5′ to 3′ direction, an optional spacer extension sequence, a spacer sequence, a minimum CRISPR repeat sequence, a single-molecule guide linker, a minimum tracrRNA sequence, a 3′ tracrRNA sequence and an optional tracrRNA extension sequence. The optional tracrRNA extension may have elements that contribute additional functionality (e.g., stability) to the guide RNA. The single-molecule guide linker links the minimum CRISPR repeat and the minimum tracrRNA sequence to form a hairpin structure. The optional tracrRNA extension has one or more hairpins. A single-molecule guide RNA (sgRNA) in a Type V system has, in the 5′ to 3′ direction, a minimum CRISPR repeat sequence and a spacer sequence.

By way of illustration, guide RNAs used in the CRISPR/Cas/Cpf1 system, or other smaller RNAs can be readily synthesized by chemical means as illustrated below and described in the art. While chemical synthetic procedures are continually expanding, purifications of such RNAs by procedures such as high performance liquid chromatography (HPLC), which avoids the use of gels such as PAGE) tends to become more challenging as polynucleotide lengths increase significantly beyond a hundred or so nucleotides. One approach used for generating RNAs of greater length is to produce two or more molecules that are ligated together. Much longer RNAs, such as those encoding a Cas9 or Cpf1 endonuclease, are more readily generated enzymatically. Various types of RNA modifications can be introduced during or after chemical synthesis and/or enzymatic generation of RNAs, e.g., modifications that enhance stability, reduce the likelihood or degree of innate immune response, and/or enhance other attributes, as described in the art.

In some embodiments of genome-targeting nucleic acids, a spacer extension sequence can modify activity, provide stability and/or provide a location for modifications of a genome-targeting nucleic acid. A spacer extension sequence can modify on-or off-target activity or specificity. In some embodiments, a spacer extension sequence is provided. A spacer extension sequence can have a length of more than 1, 5, 10, 15, 20, 25, 30, 35, 40, 45, 50, 60, 70, 80, 90, 100, 120, 140, 160, 180, 200, 220, 240, 260, 280, 300, 320, 340, 360, 380, 400, 1000, 2000, 3000, 4000, 5000, 6000, or 7000 or more nucleotides. A spacer extension sequence can have a length of about 1, 5, 10, 15, 20, 25, 30, 35, 40, 45, 50, 60, 70, 80, 90, 100, 120, 140, 160, 180, 200, 220, 240, 260, 280, 300, 320, 340, 360, 380, 400, 1000, 2000, 3000, 4000, 5000, 6000, or 7000 or more nucleotides. A spacer extension sequence can have a length of less than 1, 5, 10, 15, 20, 25, 30, 35, 40, 45, 50, 60, 70, 80, 90, 100, 120, 140, 160, 180, 200, 220, 240, 260, 280, 300, 320, 340, 360, 380, 400, 1000, 2000, 3000, 4000, 5000, 6000, 7000 or more nucleotides. In some embodiments, a spacer extension sequence is less than 10 nucleotides in length. In some embodiments, a spacer extension sequence is between 10-30 nucleotides in length. In some embodiments, a spacer extension sequence is between 30-70 nucleotides in length.

In some embodiments, the spacer extension sequence has another moiety (e.g., a stability control sequence, an endoribonuclease binding sequence, a ribozyme). In some embodiments, the moiety decreases or increases the stability of a nucleic acid targeting nucleic acid. In some embodiments, the moiety is a transcriptional terminator segment (i.e., a transcription termination sequence). In some embodiments, the moiety functions in a eukaryotic cell. In some embodiments, the moiety functions in a prokaryotic cell. In some embodiments, the moiety functions in both eukaryotic and prokaryotic cells. Non-limiting examples of suitable moieties include: a 5′ cap (e.g., a 7-methylguanylate cap (m7 G)), a riboswitch sequence (e.g., to allow for regulated stability and/or regulated accessibility by proteins and protein complexes), a sequence that forms a dsRNA duplex (i.e., a hairpin), a sequence that targets the RNA to a subcellular location (e.g., nucleus, mitochondria, chloroplasts, and the like), a modification or sequence that provides for tracking (e.g., direct conjugation to a fluorescent molecule, conjugation to a moiety that facilitates fluorescent detection, a sequence that allows for fluorescent detection, etc.), and/or a modification or sequence that provides a binding site for proteins (e.g., proteins that act on DNA, including transcriptional activators, transcriptional repressors, DNA methyltransferases, DNA demethylases, histone acetyltransferases, histone deacetylases, and the like).

The spacer sequence hybridizes to a sequence in a target nucleic acid of interest. The spacer of a genome-targeting nucleic acid interacts with a target nucleic acid in a sequence-specific manner via hybridization (i.e., base pairing). The nucleotide sequence of the spacer thus varies depending on the sequence of the target nucleic acid of interest.

In a CRISPR/Cas system herein, the spacer sequence is designed to hybridize to a target nucleic acid that is located 5′ of a PAM of the Cas9 enzyme used in the system. The spacer can perfectly match the target sequence or can have mismatches. Each Cas9 enzyme has a particular PAM sequence that it recognizes in a target DNA. For example, S. pyogenes recognizes in a target nucleic acid a PAM that has the sequence 5′-NRG-3′, where R has either A or G, where N is any nucleotide and N is immediately 3′ of the target nucleic acid sequence targeted by the spacer sequence.

In some embodiments, the target nucleic acid sequence has 20 nucleotides. In some embodiments, the target nucleic acid has less than 20 nucleotides. In some embodiments, the target nucleic acid has more than 20 nucleotides. In some embodiments, the target nucleic acid has at least: 5, 10, 15, 16, 17, 18, 19, 20, 21, 22, 23, 24, 25, 30 or more nucleotides. In some embodiments, the target nucleic acid has at most: 5, 10, 15, 16, 17, 18, 19, 20, 21, 22, 23, 24, 25, 30 or more nucleotides. In some embodiments, the target nucleic acid sequence has 20 bases immediately 5′ of the first nucleotide of the PAM. For example, in a sequence having 5′-NNNNNNNNNNNNNNNNNNNNNRG-3′, the target nucleic acid has the sequence that corresponds to the Ns, wherein N is any nucleotide, and the underlined NRG sequence (R is G or A) is the Streptococcus pyogenes Cas9 PAM. In some embodiments, the PAM sequence used in the compositions and methods of the present disclosure as a sequence recognized by S.p. Cas9 is NGG.

In some embodiments, the spacer sequence that hybridizes to the target nucleic acid has a length of at least about 6 nucleotides (nt). The spacer sequence can be at least about 6 nt, about 10 nt, about 15 nt, about 18 nt, about 19 nt, about 20 nt, about 25 nt, about 30 nt, about 35 nt or about 40 nt, from about 6 nt to about 80 nt, from about 6 nt to about 50 nt, from about 6 nt to about 45 nt, from about 6 nt to about 40 nt, from about 6 nt to about 35 nt, from about 6 nt to about 30 nt, from about 6 nt to about 25 nt, from about 6 nt to about 20 nt, from about 6 nt to about 19 nt, from about 10 nt to about 50 nt, from about 10 nt to about 45 nt, from about 10 nt to about 40 nt, from about 10 nt to about 35 nt, from about 10 nt to about 30 nt, from about 10 nt to about 25 nt, from about 10 nt to about 20 nt, from about 10 nt to about 19 nt, from about 19 nt to about 25 nt, from about 19 nt to about 30 nt, from about 19 nt to about 35 nt, from about 19 nt to about 40 nt, from about 19 nt to about 45 nt, from about 19 nt to about 50 nt, from about 19 nt to about 60 nt, from about 20 nt to about 25 nt, from about 20 nt to about 30 nt, from about 20 nt to about 35 nt, from about 20 nt to about 40 nt, from about 20 nt to about 45 nt, from about 20 nt to about 50 nt, or from about 20 nt to about 60 nt. In some embodiments, the spacer sequence has 20 nucleotides. In some embodiments, the spacer has 19 nucleotides. In some embodiments, the spacer has 18 nucleotides. In some embodiments, the spacer has 17 nucleotides. In some embodiments, the spacer has 16 nucleotides. In some embodiments, the spacer has 15 nucleotides.

In some embodiments, the percent complementarity between the spacer sequence and the target nucleic acid is at least about 30%, at least about 40%, at least about 50%, at least about 60%, at least about 65%, at least about 70%, at least about 75%, at least about 80%, at least about 85%, at least about 90%, at least about 95%, at least about 97%, at least about 98%, at least about 99%, or 100%. In some embodiments, the percent complementarity between the spacer sequence and the target nucleic acid is at most about 30%, at most about 40%, at most about 50%, at most about 60%, at most about 65%, at most about 70%, at most about 75%, at most about 80%, at most about 85%, at most about 90%, at most about 95%, at most about 97%, at most about 98%, at most about 99%, or 100%. In some embodiments, the percent complementarity between the spacer sequence and the target nucleic acid is 100% over the six contiguous 5′-most nucleotides of the target sequence of the complementary strand of the target nucleic acid. In some embodiments, the percent complementarity between the spacer sequence and the target nucleic acid is at least 60% over about 20 contiguous nucleotides. In some embodiments, the length of the spacer sequence and the target nucleic acid can differ by 1 to 6 nucleotides, which can be thought of as a bulge or bulges.

In some embodiments, the spacer sequence is designed or chosen using a computer program. The computer program can use variables, such as predicted melting temperature, secondary structure formation, predicted annealing temperature, sequence identity, genomic context, chromatin accessibility, % GC, frequency of genomic occurrence (e.g., of sequences that are identical or are similar but vary in one or more spots as a result of mismatch, insertion or deletion), methylation status, presence of SNPs, and the like.

Zinc Finger Nucleases. Zinc finger nucleases (ZFNs) are modular proteins having an engineered zinc finger DNA binding domain linked to the catalytic domain of the type II endonuclease FokI. Because FokI functions only as a dimer, a pair of ZFNs must be engineered to bind to cognate target “half-site” sequences on opposite DNA strands and with precise spacing between them to enable the catalytically active FokI dimer to form. Upon dimerization of the FokI domain, which itself has no sequence specificity per se, a DNA double-strand break is generated between the ZFN half-sites as the initiating step in genome editing.

The DNA binding domain of each ZFN generally has 3-6 zinc fingers of the abundant Cys2-His2 architecture, with each finger primarily recognizing a triplet of nucleotides on one strand of the target DNA sequence, although cross-strand interaction with a fourth nucleotide also can be important. Alteration of the amino acids of a finger in positions that make key contacts with the DNA alters the sequence specificity of a given finger. Thus, a four-finger zinc finger protein will selectively recognize a 12 bp target sequence, where the target sequence is a composite of the triplet preferences contributed by each finger, although triplet preference can be influenced to varying degrees by neighboring fingers. An important aspect of ZFNs is that they can be readily re-targeted to almost any genomic address simply by modifying individual fingers, although considerable expertise is required to do this well. In most applications of ZFNs, proteins of 4-6 fingers are used, recognizing 12-18 bp respectively. Hence, a pair of ZFNs will generally recognize a combined target sequence of 24-36 bp, not including the 5-7 bp spacer between half-sites. The binding sites can be separated further with larger spacers, including 15-17 bp. A target sequence of this length is likely to be unique in the human genome, assuming repetitive sequences or gene homologs are excluded during the design process. Nevertheless, the ZFN protein-DNA interactions are not absolute in their specificity so off-target binding and cleavage events do occur, either as a heterodimer between the two ZFNs, or as a homodimer of one or the other of the ZFNs. The latter possibility has been effectively eliminated by engineering the dimerization interface of the FokI domain to create “plus” and “minus” variants, also known as obligate heterodimer variants, which can only dimerize with each other, and not with themselves. Forcing the obligate heterodimer prevents formation of the homodimer. This has greatly enhanced specificity of ZFNs, as well as any other nuclease that adopts these FokI variants.

A variety of ZFN-based systems have been described in the art, modifications thereof are regularly reported, and numerous references describe rules and parameters that are used to guide the design of ZFNs; see, e.g., Segal et al., Proc Natl Acad Sci USA 96 (6): 2758-63 (1999); Dreier B et al., J Mol Biol. 303 (4): 489-502 (2000); Liu Q et al., J Biol Chem. 277 (6): 3850-6 (2002); Dreier et al., J Biol Chem 280 (42): 35588-97 (2005); and Dreier et al., J Biol Chem. 276 (31): 29466-78 (2001).

Transcription Activator-Like Effector Nucleases (TALENs). TALENs represent another format of modular nucleases whereby, as with ZFNs, an engineered DNA binding domain is linked to the FokI nuclease domain, and a pair of TALENs operate in tandem to achieve targeted DNA cleavage. The major difference from ZFNs is the nature of the DNA binding domain and the associated target DNA sequence recognition properties. The TALEN DNA binding domain derives from TALE proteins, which were originally described in the plant bacterial pathogen Xanthomonas sp. TALEs have tandem arrays of 33-35 amino acid repeats, with each repeat recognizing a single base pair in the target DNA sequence that is generally up to 20 bp in length, giving a total target sequence length of up to 40 bp. Nucleotide specificity of each repeat is determined by the repeat variable diresidue (RVD), which includes just two amino acids at positions 12 and 13. The bases guanine, adenine, cytosine and thymine are predominantly recognized by the four RVDs: Asn-Asn, Asn-Ile, His-Asp and Asn-Gly, respectively. This constitutes a much simpler recognition code than for zinc fingers, and thus represents an advantage over the latter for nuclease design. Nevertheless, as with ZFNs, the protein-DNA interactions of TALENs are not absolute in their specificity, and TALENs have also benefitted from the use of obligate heterodimer variants of the FokI domain to reduce off-target activity.

Additional variants of the FokI domain have been created that are deactivated in their catalytic function. If one half of either a TALEN or a ZFN pair contains an inactive FokI domain, then only single-strand DNA cleavage (nicking) will occur at the target site, rather than a DSB. The outcome is comparable to the use of CRISPR/Cas9/Cpf1 “nickase” mutants in which one of the Cas9 cleavage domains has been deactivated. DNA nicks can be used to drive genome editing by HDR, but at lower efficiency than with a DSB. The main benefit is that off-target nicks are quickly and accurately repaired, unlike the DSB, which is prone to NHEJ-mediated mis-repair.

A variety of TALEN-based systems have been described in the art, and modifications thereof are regularly reported; see, e.g., Boch, Science 326 (5959): 1509-12 (2009); Mak et al., Science 335 (6069): 716-9 (2012); and Moscou et al., Science 326 (5959): 1501 (2009). The use of TALENs based on the “Golden Gate” platform, or cloning scheme, has been described by multiple groups; see, e.g., Cermak et al., Nucleic Acids Res. 39 (12): e82 (2011); Li et al., Nucleic Acids Res. 39 (14): 6315-25 (2011); Weber et al., PLOS One. 6 (2): e16765 (2011); Wang et al., J Genet Genomics 41 (6): 339-47, Epub 2014 Can 17 (2014); and Cermak T et al., Methods Mol Biol. 1239:133-59 (2015).

Homing Endonucleases. Homing endonucleases (HEs) are sequence-specific endonucleases that have long recognition sequences (14-44 base pairs) and cleave DNA with high specificity—often at sites unique in the genome. There are at least six known families of HEs as classified by their structure, including LAGLIDADG (SEQ ID NO:6), GIY-YIG, His-Cis box, H-N-H, PD-(D/E)xK, and Vsr-like that are derived from a broad range of hosts, including eukarya, protists, bacteria, archaea, cyanobacteria and phage. As with ZFNs and TALENs, HEs can be used to create a DSB at a target locus as the initial step in genome editing. In addition, some natural and engineered HEs cut only a single strand of DNA, thereby functioning as site-specific nickases. The large target sequence of HEs and the specificity that they offer have made them attractive candidates to create site-specific DSBs.

A variety of HE-based systems have been described in the art, and modifications thereof are regularly reported; see, e.g., the reviews by Steentoft et al., Glycobiology 24 (8): 663-80 (2014); Belfort and Bonocora, Methods Mol Biol. 1123:1-26 (2014); Hafez and Hausner, Genome 55 (8): 553-69 (2012); and references cited therein.

MegaTAL/Tev-mTALEN/MegaTev. As further examples of hybrid nucleases, the MegaTAL platform and Tev-mTALEN platform use a fusion of TALE DNA binding domains and catalytically active HEs, taking advantage of both the tunable DNA binding and specificity of the TALE, as well as the cleavage sequence specificity of the HE; see, e.g., Boissel et al., NAR 42:2591-2601 (2014); Kleinstiver et al., G3 4:1155-65 (2014); and Boissel and Scharenberg, Methods Mol. Biol. 1239:171-96 (2015).

In a further variation, the MegaTev architecture is the fusion of a meganuclease (Mega) with the nuclease domain derived from the GIY-YIG homing endonuclease I-TevI (Tev). The two active sites are positioned about 30 bp apart on a DNA substrate and generate two DSBs with non-compatible cohesive ends; see, e.g., Wolfs et al., NAR 42, 8816-29 (2014). It is anticipated that other combinations of existing nuclease-based approaches will evolve and be useful in achieving the targeted genome modifications described herein.

dCas9-FokI or dCpf1-FokI and Other Nucleases. Combining the structural and functional properties of the nuclease platforms described above offers a further approach to genome editing that can potentially overcome some of the inherent deficiencies. As an example, the CRISPR genome editing system generally uses a single Cas9 endonuclease to create a DSB. The specificity of targeting is driven by a 20 or 22 nucleotide sequence in the guide RNA that undergoes Watson-Crick base-pairing with the target DNA (plus an additional 2 bases in the adjacent NAG or NGG PAM sequence in the case of Cas9 from S. pyogenes). Such a sequence is long enough to be unique in the human genome, however, the specificity of the RNA/DNA interaction is not absolute, with significant promiscuity sometimes tolerated, particularly in the 5′ half of the target sequence, effectively reducing the number of bases that drive specificity. One solution to this has been to completely deactivate the Cas9 or Cpf1 catalytic function—retaining only the RNA-guided DNA binding function—and instead fusing a FokI domain to the deactivated Cas9; see, e.g., Tsai et al., Nature Biotech 32:569-76 (2014); and Guilinger et al., Nature Biotech. 32:577-82 (2014). Because FokI must dimerize to become catalytically active, two guide RNAs are required to tether two FokI fusions in close proximity to form the dimer and cleave DNA. This essentially doubles the number of bases in the combined target sites, thereby increasing the stringency of targeting by CRISPR-based systems.

As further example, fusion of the TALE DNA binding domain to a catalytically active HE, such as I-TevI, takes advantage of both the tunable DNA binding and specificity of the TALE, as well as the cleavage sequence specificity of I-TevI, with the expectation that off-target cleavage can be further reduced.

Additional details regarding gene editing systems that find use in embodiments of the invention may be found in United States Published Patent Application Publication No. 20210348159.

### Delivery Compositions and Methods of Delivery

Where desired, the NTDNA may be present in a delivery composition that includes the NTDNA and a delivery vehicle component, e.g., a delivery vehicle component that mediates entry of the NTDNA into the cytosol from an extracellular location. In some aspects, the methods provided herein comprise delivering a NTDNA to a target cell. Also provided herein are cells produced by such methods, and organisms (such as animals, plants, or fungi) comprising or produced from such cells. Methods of delivery of nucleic acids can include lipofection, nucleofection, microinjection, biolistics, liposomes, immunoliposomes, polycation or lipid: nucleic acid conjugates, naked DNA, and agent-enhanced uptake of DNA. Lipofection is described in e.g., U.S. Pat. Nos. 5,049,386, 4,946,787; and 4,897,355 and lipofection reagents are sold commercially (e.g., Transfectam™ and Lipofectin™). Delivery can be to cells (e.g., in vitro or ex vivo administration) or target tissues (e.g., in vivo administration), as indicated above.

Various techniques and methods are known in the art for delivering nucleic acids to cells. For example, an NTNDA can be delivered to a cell by conjugating the nucleic acid with a ligand that is internalized by the cell. For example, the ligand can bind a receptor on the cell surface and internalized via endocytosis. The ligand can be covalently linked to a nucleotide in the nucleic acid. Exemplary conjugates for delivering nucleic acids into a cell are described, example, in WO2015/006740, WO2014/025805, WO2012/037254, WO2009/082606, WO2009/073809, WO2009/018332, WO2006/112872, WO2004/090108, WO2004/091515 and WO2017/177326.

NTDNAs, e.g., as described herein, can also be delivered to a cell by transfection. Useful transfection methods include, but are not limited to, lipid-mediated transfection, cationic polymer-mediated transfection, or calcium phosphate precipitation. Transfection reagents are well known in the art and include, but are not limited to, TurboFect Transfection Reagent (Thermo Fisher Scientific), Pro-Ject Reagent (Thermo Fisher Scientific), TRANSPASS™ P Protein Transfection Reagent (New England Biolabs), CHARIOT™ Protein Delivery Reagent (Active Motif), PROTEOJUICE™ Protein Transfection Reagent (EMD Millipore), 293fectin, LIPOFECTAMINE™ 2000, LIPOFECTAMINE™ 3000 (Thermo Fisher Scientific), LIPOFECTAMINE™ (Thermo Fisher Scientific), LIPOFECTIN™ (Thermo Fisher Scientific), DMRIE-C, CELLFECTIN™ (Thermo Fisher Scientific), OLIGOFECTAMINE™ (Thermo Fisher Scientific), LIPOFECTACE™, FUGENE™ (Roche, Basel, Switzerland), FUGENE™ HD (Roche), TRANSFECTAM™ (Transfectam, Promega, Madison, Wis.), TFX-10TM (Promega), TFX-20™ (Promega), TFX-50™ (Promega), TRANSFECTIN™ (BioRad, Hercules, Calif.), SILENTFECT™ (Bio-Rad), Effectene™ (Qiagen, Valencia, Calif.), DC-chol (Avanti Polar Lipids), GENEPORTER™ (Gene Therapy Systems, San Diego, Calif.), DHARMAFECT 1™ (Dharmacon, Lafayette, Colo.), DHARMAFECT 2™ (Dharmacon), DHARMAFECT 3™ (Dharmacon), DHARMAFECT 4™ (Dharmacon), ESCORT™ III (Sigma, St. Louis, Mo.), and ESCORT™ IV (Sigma Chemical Co.). Nucleic acids, such as NTDNA, can also be delivered to a cell via microfluidics methods, e.g., such as those known to those of skill in the art.

Methods of non-viral delivery of nucleic acids in vivo or ex vivo include electroporation, lipofection (see, U.S. Pat. Nos. 5,049,386; 4,946,787 and commercially available reagents such as Transfectam™ and Lipofectin™), microinjection, biolistics, LNPs, virosomes, liposomes (see, e.g., Crystal, Science 270:404-410 (1995); Blaese et al., Cancer Gene Ther. 2:291-297 (1995); Behr et al., Bioconjugate Chem. 5:382-389 (1994); Remy et al., Bioconjugate Chem. 5:647-654 (1994); Gao et al., Gene Therapy 2:710-722 (1995); Ahmad et al., Cancer Res. 52:4817-4820 (1992); U.S. Pat. Nos. 4,186,183, 4,217,344, 4,235,871, 4,261,975, 4,485,054, 4,501,728, 4,774,085, 4,837,028, and 4,946,787), immunoliposomes, polycation or lipid: nucleic acid conjugates, naked DNA, and agent-enhanced uptake of DNA. Sonoporation using, e.g., the Sonitron 2000 system (Rich-Mar) can also be used for delivery of nucleic acids.

For example, NTNDAs can be formulated into lipid nanoparticles (LNPs), lipidoids, liposomes, lipoplexes, or core-shell nanoparticles. Delivery reagents such as liposomes, nanocapsules, microparticles, microspheres, lipid nanoparticles, vesicles, and the like, can be used for the introduction of the compositions of the present disclosure into suitable host cells. In particular, the nucleic acids can be formulated for delivery either encapsulated in a lipid particle, a liposome, a vesicle, a nanosphere, a nanoparticle, a gold particle, or the like. Such formulations can be preferred for the introduction of pharmaceutically acceptable formulations of the nucleic acids disclosed herein.

Various delivery methods known in the art or modifications thereof can be used to deliver NTDNAs as described herein in vitro or in vivo. For example, in some embodiments, NTDNAs are delivered by making transient penetration in cell membrane by mechanical, electrical, ultrasonic, hydrodynamic, or laser-based energy so that DNA entrance into the targeted cells is facilitated. For example, a NTDNA can be delivered by transiently disrupting cell membrane by squeezing the cell through a size-restricted channel or by other means known in the art. In some cases, a NTDNA alone is directly injected as naked DNA into skin, thymus, cardiac muscle, skeletal muscle, or liver cells.

In some cases, a NTDNA is delivered by gene gun. Gold or tungsten spherical particles (1-3 μm diameter) coated with NTDNA can be accelerated to high speed by pressurized gas to penetrate into target tissue cells.

In some embodiments, electroporation is used to deliver NTDNA to a target cell. Electroporation causes temporary destabilization of the cell membrane target cell tissue by insertion of a pair of electrodes into the tissue so that DNA molecules in the surrounding media of the destabilized membrane would be able to penetrate into cytoplasm and nucleoplasm of the cell. Electroporation has been used in vivo for many types of tissues, such as skin, lung, and muscle.

In some cases, NTDNA is delivered by hydrodynamic injection, which is a simple and highly efficient method for direct intracellular delivery of any water-soluble compounds and particles into internal organs and skeletal muscle in an entire limb.

In some cases, NTDNAs are delivered by ultrasound by making nanoscopic pores in membrane to facilitate intracellular delivery of DNA particles into cells of internal organs or tumors, so the size and concentration of plasmid DNA have great role in efficiency of the system. In some cases, NTDNAs are delivered by magnetofection by using magnetic fields to concentrate particles containing nucleic acid into the target cells.

In some cases, chemical delivery systems can be used, for example, by using nanomeric complexes, which include compaction of negatively charged nucleic acid by polycationic nanomeric particles, belonging to cationic liposome/micelle or cationic polymers. Cationic lipids used for the delivery method includes, but not limited to monovalent cationic lipids, polyvalent cationic lipids, guanidine containing compounds, cholesterol derivative compounds, cationic polymers, (e.g., poly(ethylenimine), poly-L-lysine, protamine, other cationic polymers), and lipid-polymer hybrid.

NTDNA, e.g., as described herein, can also be administered directly to an organism for transduction of cells in vivo. Administration is by any of the routes normally used for introducing a molecule into ultimate contact with blood or tissue cells including, but not limited to, injection, infusion, topical application and electroporation. Suitable methods of administering such nucleic acids are available and well known to those of skill in the art, and, although more than one route can be used to administer a particular composition, a particular route can often provide a more immediate and more effective reaction than another route.

Compositions comprising a NTDNA, e.g., as described herein, and a cytosolic delivery vehicle are specifically contemplated herein. In some embodiments, NTDNA is formulated with a lipid delivery system, for example, LNPs as described herein. In some embodiments, such compositions are administered by any route desired by a skilled practitioner. The compositions may be administered to a subject by different routes including orally, parenterally, sublingually, transdermally, rectally, transmucosally, topically, via inhalation, via buccal administration, intrapleurally, intravenous, intra-arterial, intraperitoneal, subcutaneous, intramuscular, intranasal intrathecal, and intraarticular or combinations thereof. For veterinary use, the composition may be administered as a suitably acceptable formulation in accordance with normal veterinary practice. The veterinarian may readily determine the dosing regimen and route of administration that is most appropriate for a particular animal. The compositions may be administered by traditional syringes, needleless injection devices, “microprojectile bombardment gene guns”, or other physical methods such as electroporation (“EP”), hydrodynamic methods or ultrasound.

In some cases, a NTDNA is delivered by hydrodynamic injection, which is a simple and highly efficient method for direct intracellular delivery of any water-soluble compounds and particles into internal organs and skeletal muscle in an entire limb.

In some cases, a NTDNA is delivered by ultrasound by making nanoscopic pores in membrane to facilitate intracellular delivery of DNA particles into cells of internal organs or tumors, so the size and concentration of the NTDNA have a great role in efficiency of the system. In some cases, NTDNAs as described herein are delivered by magnetofection by using magnetic fields to concentrate particles containing nucleic acid into the target cells.

In some cases, chemical delivery systems can be used, for example, by using nanomeric complexes, which include compaction of negatively charged nucleic acid by polycationic nanomeric particles, belonging to cationic liposome/micelle or cationic polymers. Cationic lipids used for the delivery method includes, but not limited to monovalent cationic lipids, polyvalent cationic lipids, guanidine containing compounds, cholesterol derivative compounds, cationic polymers, (e.g., poly(ethylenimine), poly-L-lysine, protamine, other cationic polymers), and lipid-polymer hybrid.

Microparticle/Nanoparticles. In some embodiments, a NTDNA as described herein is delivered by a nanoparticle. One example of a nanoparticle that finds use in the delivery of the subject NTDNAs is a lipid nanoparticle (LNP). Generally, LNPs of the present disclosure may be composed of nucleic acid (NTDNA) molecules, one or more ionizable or cationic lipids (or salts thereof), one or more non-ionic or neutral lipids (e.g., a phospholipid), a molecule that prevents aggregation (e.g., PEG or a PEG-lipid conjugate), and optionally a sterol (e.g., cholesterol). For example, lipid nanoparticles may comprise an ionizable amino lipid (e.g., heptatriaconta-6,9,28,31-tetraen-19-yl 4-(dimethylamino)butanoate, DLin-MC3-DMA, a phosphatidylcholine (1,2-distearoyl-sn-glycero-3-phosphocholine, DSPC), cholesterol and a coat lipid (polyethylene glycol-dimyristolglycerol, PEG-DMG), for example as disclosed by Tam et al. (2013). Advances in Lipid Nanoparticles for siRNA delivery. Pharmaceuticals 5 (3): 498-507.

In some embodiments, a lipid nanoparticle has a mean diameter between about 10 and about 1000 nm. In some embodiments, a lipid nanoparticle has a diameter that is less than 300 nm. In some embodiments, a lipid nanoparticle has a diameter between about 10 and about 300 nm. In some embodiments, a lipid nanoparticle has a diameter that is less than 200 nm. In some embodiments, a lipid nanoparticle has a diameter between about 25 and about 200 nm. In some embodiments, a lipid nanoparticle preparation (e.g., composition comprising a plurality of lipid nanoparticles) has a size distribution in which the mean size (e.g., diameter) is about 70 nm to about 200 nm, and more typically the mean size is about 100 nm or less.

In some aspects, the disclosure provides for a lipid nanoparticle comprising a NTDNA as described herein and an ionizable lipid. The ionizable lipid is typically employed to condense the nucleic acid cargo, e.g., NTDNA at low pH and to drive membrane association and fusogenicity. Generally, ionizable lipids are lipids comprising at least one amino group that is positively charged or becomes protonated under acidic conditions, for example at pH of 6.5 or lower. Ionizable lipids are also referred to as cationic lipids herein. Exemplary ionizable lipids are described in International PCT patent publications WO2015/095340, WO2015/199952, WO2018/011633, WO2017/049245, WO2015/061467, WO2012/040184, WO2012/000104, WO2015/074085, WO2016/081029, WO2017/004143, WO2017/075531, WO2017/117528, WO2011/022460, WO2013/148541, WO2013/116126, WO2011/153120, WO2012/044638, WO2012/054365, WO2011/090965, WO2013/016058, WO2012/162210, WO2008/042973, WO2010/129709, WO2010/144740, WO2012/099755, WO2013/049328, WO2013/086322, WO2013/086373, WO2011/071860, WO2009/132131, WO2010/048536, WO2010/088537, WO2010/054401, WO2010/054406, WO2010/054405, WO2010/054384, WO2012/016184, WO2009/086558, WO2010/042877, WO2011/000106, WO2011/000107, WO2005/120152, WO2011/141705, WO2013/126803, WO2006/007712, WO2011/038160, WO2005/121348, WO2011/066651, WO2009/127060, WO2011/141704, WO2006/069782, WO2012/031043, WO2013/006825, WO2013/033563, WO2013/089151, WO2017/099823, WO2015/095346, and WO2013/086354, and US patent publications US2016/0311759, US2015/0376115, US2016/0151284, US2017/0210697, US2015/0140070, US2013/0178541, US2013/0303587, US2015/0141678, US2015/0239926, US2016/0376224, US2017/0119904, US2012/0149894, US2015/0057373, US2013/0090372, US2013/0274523, US2013/0274504, US2013/0274504, US2009/0023673, US2012/0128760, US2010/0324120, US2014/0200257, US2015/0203446, US2018/0005363, US2014/0308304, US2013/0338210, US2012/0101148, US2012/0027796, US2012/0058144, US2013/0323269, US2011/0117125, US2011/0256175, US2012/0202871, US2011/0076335, US2006/0083780, US2013/0123338, US2015/0064242, US2006/0051405, US2013/0065939, US2006/0008910, US2003/0022649, US2010/0130588, U52013/0116307, US2010/0062967, US2013/0202684, US2014/0141070, US2014/0255472, US2014/0039032, US2018/0028664, U52016/0317458, and US2013/0195920.

Various LNP formulations known in the art can be used to deliver NTDNA as described herein. For example, various LNP formulations and delivery methods using lipid nanoparticles are described in U.S. Pat. Nos. 9,404,127, 9,006,417 9,518,272, and U.S. Patent Application No. 63/415,229. Such particles can be prepared by high energy mixing of ethanolic lipids with aqueous NTDNA at low pH which protonates the ionizable lipid and provides favorable energetics for NTDNA/lipid association and nucleation of particles. The particles can be further stabilized through aqueous dilution and removal of the organic solvent. The particles can be concentrated to the desired level.

Another example of a nanoparticle that finds use in the delivery of the subject NTDNAs is a metal nanoparticle. In some embodiments, a NTDNA as described herein is delivered by a gold nanoparticle. Generally, a nucleic acid can be covalently bound to a gold nanoparticle or non-covalently bound to a gold nanoparticle (e.g., bound by a charge-charge interaction), for example as described by Ding et al. (2014). Gold Nanoparticles for Nucleic Acid Delivery. Mol. Ther. 22 (6); 1075-1083. In some embodiments, gold nanoparticle-nucleic acid conjugates are produced using methods described, for example, in U.S. Pat. No. 6,812,334.

In some embodiments, NTDNAs described herein can be readily formulated in high concentrations of chitosan-nucleic acid polyplex compositions and administered orally in DNA enteric coated pills described in U.S. Pat. Nos. 8,846,102; 9,404,088; and 9,850,323, each of which is incorporated herein by its entirety.

Exosomes. In some embodiments, a NTDNA as described herein is delivered by being packaged in an exosome. Exosomes are small membrane vesicles of endocytic origin that are released into the extracellular environment following fusion of multivesicular bodies with the plasma membrane. Their surface consists of a lipid bilayer from the donor cell's cell membrane, they contain cytosol from the cell that produced the exosome, and exhibit membrane proteins from the parental cell on the surface. Exosomes are produced by various cell types including epithelial cells, B and T lymphocytes, mast cells (MC) as well as dendritic cells (DC). Some embodiments, exosomes with a diameter between 10 nm and 1 μm, between 20 nm and 500 nm, between 30 nm and 250 nm, between 50 nm and 100 nm are envisioned for use. Exosomes can be isolated for a delivery to target cells using either their donor cells or by introducing specific nucleic acids into them. Various approaches known in the art can be used to produce exosomes containing capsid-free AAV vectors of the present invention.

Conjugates. In some embodiments, a NTDNA as described herein as disclosed herein is conjugated (e.g., covalently bound to an agent that increases cellular uptake. An “agent that increases cellular uptake” is a molecule that facilitates transport of a nucleic acid across a lipid membrane. For example, a nucleic acid can be conjugated to a lipophilic compound (e.g., cholesterol, tocopherol, etc.), a cell penetrating peptide (CPP) (e.g., penetratin, TAT, Syn1B, etc.), and polyamines (e.g., spermine). Further examples of agents that increase cellular uptake are disclosed, for example, in Winkler (2013). Oligonucleotide conjugates for therapeutic applications. Ther. Deliv. 4 (7); 791-809.

In some embodiments, a NTDNA as described herein as disclosed herein is conjugated to a polymer (e.g., a polymeric molecule) or a folate molecule (e.g., folic acid molecule). Generally, delivery of nucleic acids conjugated to polymers is known in the art, for example as described in WO2000/34343 and WO2008/022309. In some embodiments, a NTDNA as disclosed herein is conjugated to a poly(amide) polymer, for example as described by U.S. Pat. No. 8,987,377. In some embodiments, a nucleic acid described by the disclosure is conjugated to a folic acid molecule as described in U.S. Pat. No. 8,507,455. In some embodiments, a NTDNA as described herein as disclosed herein is conjugated to a carbohydrate, for example as described in U.S. Pat. No. 8,450,467.

Nanocapsules. Alternatively, nanocapsule formulations of a NTDNA can be used. Nanocapsules can generally entrap substances in a stable and reproducible way. To avoid side effects due to intracellular polymeric overloading, such ultrafine particles (sized around 0.1 μm) should be designed using polymers able to be degraded in vivo. Biodegradable polyalkyl-cyanoacrylate nanoparticles that meet these requirements are contemplated for use.

Liposomes. A NTDNA as described herein can be added to liposomes for delivery to a cell or target organ in a subject. Liposomes are vesicles that possess at least one lipid bilayer and an aqueous core. Liposomes are typical used as carriers for drug/therapeutic delivery in the context of pharmaceutical development. They work by fusing with a cellular membrane and repositioning its lipid structure to deliver a drug or active pharmaceutical ingredient (API). Liposome compositions for such delivery are composed of phospholipids, especially compounds having a phosphatidylcholine group, however these compositions may also include other lipids.

The formation and use of liposomes is generally known to those of skill in the art. Liposomes have been developed with improved serum stability and circulation half-times (U.S. Pat. No. 5,741,516). Further, various methods of liposome and liposome like preparations as potential drug carriers have been described (U.S. Pat. Nos. 5,567,434; 5,552,157; 5,565,213; 5,738,868 and 5,795,587).

### Additional Components

In some embodiments, a delivery composition may include a NTDNA and one or more additional components. In some instances, the one or additional components may be one or more additional components of a gene editing system, e.g., as described above, which one or more additional components mediate genomic integration of the cargo nucleic acid of the NTDNA. As such, a NTDNA lipid nanoparticle may further include on o more of: a guide RNA, an endonuclease or nucleic acid coding sequence therefore, e.g., RNA (such as mRNA) or DNA, etc.

The one or more additional compounds can be a therapeutic agent. The therapeutic agent can be selected from any class suitable for the therapeutic objective. In other words, the therapeutic agent can be selected from any class suitable for the therapeutic objective. In other words, the therapeutic agent can be selected according to the treatment objective and biological action desired. For example, if the NTDNA within the LNP is useful for treating cancer, the additional compound can be an anti-cancer agent (e.g., a chemotherapeutic agent, a targeted cancer therapy (including, but not limited to, a small molecule, an antibody, or an antibody-drug conjugate). In another example, if the LNP containing the NTDNA is useful for treating an infection, the additional compound can be an antimicrobial agent (e.g., an antibiotic or antiviral compound). In yet another example, if the LNP containing the NTDNA is useful for treating an immune disease or disorder, the additional compound can be a compound that modulates an immune response (e.g., an immunosuppressant, immunostimulatory compound, or compound modulating one or more specific immune pathways). In some embodiments, different cocktails of different lipid nanoparticles containing different compounds, such as a NTDNA encoding a different protein or a different compound, such as a therapeutic may be used in the compositions and methods of the invention. In some embodiments, the additional compound is an immune modulating agent. For example, the additional compound is an immunosuppressant. In some embodiments, the additional compound is immune stimulatory agent.

### Compositions

Also provided are compositions that find use in practicing embodiments of the invention. Compositions of the invention include those having a NTDNA, e.g., as described, where the NDTNA may be present in combination with one or more additional components, such as but not limited to, components of a gene editing system, e.g., as described above, such as a gRNA, endonuclease or nucleic acid encoding the same, etc.

In some embodiments, a composition can have a NTDNA delivery vehicle, e.g., as described above, such as liposome or a lipid nanoparticle. Therefore, in some embodiments, any compounds (e.g., a DNA endonuclease or a nucleic acid encoding thereof, gRNA and NTDNA) of the composition can be formulated in a liposome or lipid nanoparticle. In some embodiments, one or more such compounds are associated with a liposome or lipid nanoparticle via a covalent bond or non-covalent bond. In some embodiments, any of the compounds can be separately or together contained in a liposome or lipid nanoparticle. Therefore, in some embodiments, each of a DNA endonuclease or a nucleic acid encoding thereof, gRNA and NTDNA (donor template) is separately formulated in a liposome or lipid nanoparticle. In some embodiments, a DNA endonuclease is formulated in a liposome or lipid nanoparticle with gRNA. In some embodiments, a DNA endonuclease or a nucleic acid encoding thereof, gRNA and donor template are formulated in a liposome or lipid nanoparticle together.

In some embodiments, a composition described above further has one or more additional reagents, where such additional reagents are selected from a buffer, a buffer for introducing a polypeptide or polynucleotide into a cell, a wash buffer, a control reagent, a control vector, a control RNA polynucleotide, a reagent for in vitro production of the polypeptide from DNA, adaptors for sequencing and the like. A buffer can be a stabilization buffer, a reconstituting buffer, a diluting buffer, or the like. In some embodiments, a composition can also include one or more components that can be used to facilitate or enhance the on-target binding or the cleavage of DNA by the endonuclease, or improve the specificity of targeting.

Also provided herein is a pharmaceutical composition comprising the delivery vehicle-encapsulated NTDNA and a pharmaceutically acceptable carrier or excipient. In some aspects, the disclosure provides for a lipid nanoparticle formulation further comprising one or more pharmaceutical excipients. In some embodiments, the lipid nanoparticle formulation further comprises sucrose, tris, trehalose and/or glycine.

In some embodiments, any components of a composition are formulated with pharmaceutically acceptable excipients such as carriers, solvents, stabilizers, adjuvants, diluents, etc., depending upon the particular mode of administration and dosage form. In some embodiments, guide RNA compositions are generally formulated to achieve a physiologically compatible pH, and range from a pH of about 3 to a pH of about 11, about pH 3 to about pH 7, depending on the formulation and route of administration. In some embodiments, the pH is adjusted to a range from about pH 5.0 to about pH 8. In some embodiments, the composition has a therapeutically effective amount of at least one compound as described herein, together with one or more pharmaceutically acceptable excipients. Optionally, the composition can have a combination of the compounds described herein, or can include a second active ingredient useful in the treatment or prevention of bacterial growth (for example and without limitation, anti-bacterial or anti-microbial agents), or can include a combination of reagents of the disclosure. In some embodiments, gRNAs are formulated with other one or more oligonucleotides, e.g. a nucleic acid encoding DNA endonuclease and/or a donor template. Alternatively, a nucleic acid encoding DNA endonuclease and a donor template, separately or in combination with other oligonucleotides, are formulated with the method described above for gRNA formulation.

Suitable excipients can include, for example, carrier molecules that include large, slowly metabolized macromolecules such as proteins, polysaccharides, polylactic acids, polyglycolic acids, polymeric amino acids, amino acid copolymers, and inactive virus particles. Other exemplary excipients include antioxidants (for example and without limitation, ascorbic acid), chelating agents (for example and without limitation, EDTA), carbohydrates (for example and without limitation, dextrin, hydroxyalkylcellulose, and hydroxyalkylmethylcellulose), stearic acid, liquids (for example and without limitation, oils, water, saline, glycerol and ethanol), wetting or emulsifying agents, pH buffering substances, and the like.

In some embodiments, a composition refers to a therapeutic composition having therapeutic cells, e.g., modified via methods of the invention such as described herein, that are used in an ex vivo treatment method. In some embodiments, therapeutic compositions contain a physiologically tolerable carrier together with the cell composition, and optionally at least one additional bioactive agent as described herein, dissolved or dispersed therein as an active ingredient. In some embodiments, the therapeutic composition is not substantially immunogenic when administered to a mammal or human patient for therapeutic purposes, unless so desired. In general, the genetically-modified, therapeutic cells described herein are administered as a suspension with a pharmaceutically acceptable carrier. One of skill in the art will recognize that a pharmaceutically acceptable carrier to be used in a cell composition will not include buffers, compounds, cryopreservation agents, preservatives, or other agents in amounts that substantially interfere with the viability of the cells to be delivered to the subject. A formulation having cells can include, e.g., osmotic buffers that permit cell membrane integrity to be maintained, and optionally, nutrients to maintain cell viability or enhance engraftment upon administration. Such formulations and suspensions are known to those of skill in the art and/or can be adapted for use with the progenitor cells, as described herein, using routine experimentation. In some embodiments, a cell composition can also be emulsified or presented as a liposome composition, provided that the emulsification procedure does not adversely affect cell viability. The cells and any other active ingredient can be mixed with excipients that are pharmaceutically acceptable and compatible with the active ingredient, and in amounts suitable for use in the therapeutic methods described herein. Additional agents included in a cell composition can include pharmaceutically acceptable salts of the components therein. Pharmaceutically acceptable salts include the acid addition salts (formed with the free amino groups of the polypeptide) that are formed with inorganic acids, such as, for example, hydrochloric or phosphoric acids, or such organic acids as acetic, tartaric, mandelic and the like. Salts formed with the free carboxyl groups can also be derived from inorganic bases, such as, for example, sodium, potassium, ammonium, calcium or ferric hydroxides, and such organic bases as isopropylamine, trimethylamine, 2-ethylamino ethanol, histidine, procaine and the like. Physiologically tolerable carriers are well known in the art. Exemplary liquid carriers are sterile aqueous solutions that contain no materials in addition to the active ingredients and water, or contain a buffer such as sodium phosphate at physiological pH value, physiological saline or both, such as phosphate-buffered saline. Still further, aqueous carriers can contain more than one buffer salt, as well as salts such as sodium and potassium chlorides, dextrose, polyethylene glycol and other solutes. Liquid compositions can also contain liquid phases in addition to and to the exclusion of water. Exemplary of such additional liquid phases are glycerin, vegetable oils such as cottonseed oil, and water-oil emulsions. The amount of an active compound used in the cell compositions that is effective in the treatment of a particular disorder or condition will depend on the nature of the disorder or condition, and can be determined by standard clinical techniques.

### Kits

Aspects of the present disclosure also include kits. Some aspects of this disclosure provide kits that include an NTDNA, e.g., as described above. Kits of the invention may further included one or more additional components, e.g., an inducing agent (e.g., as described above), a gene editing system or components thereof, e.g., as described above, etc. In some instances, the various components of a given kit may be combined into a single composition, e.g., with a cytosolic delivery vehicle, as a pharmaceutical composition, etc., such as described above.

In some instances, kits may include an article of manufacture containing materials useful for the treatment of the diseases described above is included. In some embodiments, the article of manufacture comprises a container and a label. Suitable containers include, for example, bottles, vials, syringes, and test tubes. The containers may be formed from a variety of materials such as glass or plastic. In some embodiments, the container holds a composition that is effective for treating a disease described herein and may have a sterile access port. For example, the container may be an intravenous solution bag or a vial having a stopper pierceable by a hypodermic injection needle. The active agent in the composition is a compound of the invention. In some embodiments, the label on or associated with the container indicates that the composition is used for treating the disease of choice. The article of manufacture may further comprise a second container comprising a pharmaceutically-acceptable buffer, such as phosphate-buffered saline, Ringer's solution, or dextrose solution. It may further include other materials desirable from a commercial and user standpoint, including other buffers, diluents, filters, needles, syringes, and package inserts with instructions for use.

Components of the kits may be present in separate containers, or multiple components may be present in a single container. In addition to the above-mentioned components, a subject kit may further include instructions for using the components of the kit, e.g., to practice the subject methods. The instructions are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or sub-packaging) etc. In other embodiments, the instructions are present as an electronic storage data file present on a suitable computer readable storage medium, e.g., CD-ROM, diskette, Hard Disk Drive (HDD), portable flash drive, etc. In yet other embodiments, the actual instructions are not present in the kit, but means for obtaining the instructions from a remote source, e.g., via the internet, are provided. An example of this embodiment is a kit that includes a web address where the instructions can be viewed and/or from which the instructions can be downloaded. As with the instructions, this means for obtaining the instructions is recorded on a suitable substrate.

### Utility

The subject methods and compositions, e.g., as described above, can be used in any application where nuclear delivery of a cargo nucleic acid is desired. Applications of interest include both research and therapeutic applications. Applications of interest include, but are not limited to: research applications, diagnostic applications and therapeutic applications. In some instances, cargo nucleic acids that may be introduced into a nucleus via methods of the invention include those encoding research proteins, diagnostic proteins and therapeutic proteins.

Research proteins are proteins whose activity finds use in a research protocol. As such, research proteins are proteins that are employed in an experimental procedure. The research protein may be any protein that has such utility, where in some instances the research protein is a protein domain that is also provided in research protocols by expressing it in a cell from an encoding vector. Examples of specific types of research proteins include, but are not limited to: transcription modulators of inducible expression systems, members of signal production systems, e.g., enzymes and substrates thereof, hormones, prohormones, proteases, enzyme activity modulators, perturbimers and peptide aptamers, antibodies, modulators of protein-protein interactions, genomic modification proteins, such as CRE recombinase, meganucleases, Zinc-finger nucleases, CRISPR/Cas-9 nuclease, TAL effector nucleases, etc., cellular reprogramming proteins, such as Oct 3/4, Sox2, Klf4, c-Myc, Nanog, Lin-28, etc., and the like.

Diagnostic proteins are proteins whose activity finds use in a diagnostic protocol. As such, diagnostic proteins are proteins that are employed in a diagnostic procedure. The diagnostic protein may be any protein that has such utility. Examples of specific types of diagnostic proteins include, but are not limited to: members of signal production systems, e.g., enzymes and substrates thereof, labeled binding members, e.g., labeled antibodies and binding fragments thereof, peptide aptamers and the like.

Proteins of interest further include therapeutic proteins. Therapeutic proteins are proteins that provide a therapeutic benefit to a patient, and include secreted proteins, transmembrane proteins, and intracellularly acting proteins. As will be appreciated by one of ordinary skill in the art, cargo nucleic acids that encode any protein that is associated with a liver disease or that, upon secretion from the liver, find use in treating another organ in the body, may be delivered using the subject compositions and methods.

Target cells to which nucleic acids may be delivered in accordance with the invention may vary widely. Target cells of interest include, but are not limited to: cell lines, HeLa, HEK, CHO, 293 and the like, Mouse embryonic stem cells, human stem cells, mesenchymal stem cells, primary cells, tissue samples and the like. Some non-limiting examples of a mammalian cell include, without limitation, a mouse cell, a rat cell, hamster cell, a rodent cell, and a nonhuman primate cell. In some embodiments, the target cell is a human cell. It should also be appreciated that the target cell may be of any cell type. For example, the target cell may be a stem cell, which may include embryonic stem cells, induced pluripotent stem cells (iPS cells), fetal stem cells, cord blood stem cells, or adult stem cells (i.e., tissue specific stem cells). In other cases, the target cell may be any differentiated cell type found in a subject. Cells of interest include both dividing cells and non-dividing cells. Examples of specific target cells of interest include, but are not limited to: hepatocytes, stellate cells, T lymphocytes, B lymphocytes, NK cells, skeletal muscle cells, cardiomyocytes, neurons, astrocytes, oligodendrocytes, dendritic cells, skin cells, etc. In some instances, the application of interest is a therapeutic application, for example, in the treatment of a disease. For example, the compositions and methods of the present application may be used to deliver a nucleic acid sequence to the nucleus of a cell to complement a genetic deficiency. As one nonlimiting example, compositions of the present application may be used in the treatment of a genetic deficiency that impacts the function of hepatocytes, or in the treatment of a genetic deficiency elsewhere in the body that can be remedied by leveraging hepatocytes as a biofactory to secrete the deficient protein.

The following example(s) is/are offered by way of illustration and not by way of limitation.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Materials and Methods

General methods in molecular and cellular biochemistry can be found in such standard textbooks as Molecular Cloning: A Laboratory Manual, 3rd Ed. (Sambrook et al., HaRBor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference. Reagents, cloning vectors, cells, and kits for methods referred to in, or related to, this disclosure are available from commercial vendors such as BioRad, Agilent Technologies, Thermo Fisher Scientific, Sigma-Aldrich, New England Biolabs (NEB), Takara Bio USA, Inc., and the like, as well as repositories such as e.g., Addgene, Inc., American Type Culture Collection (ATCC), and the like.

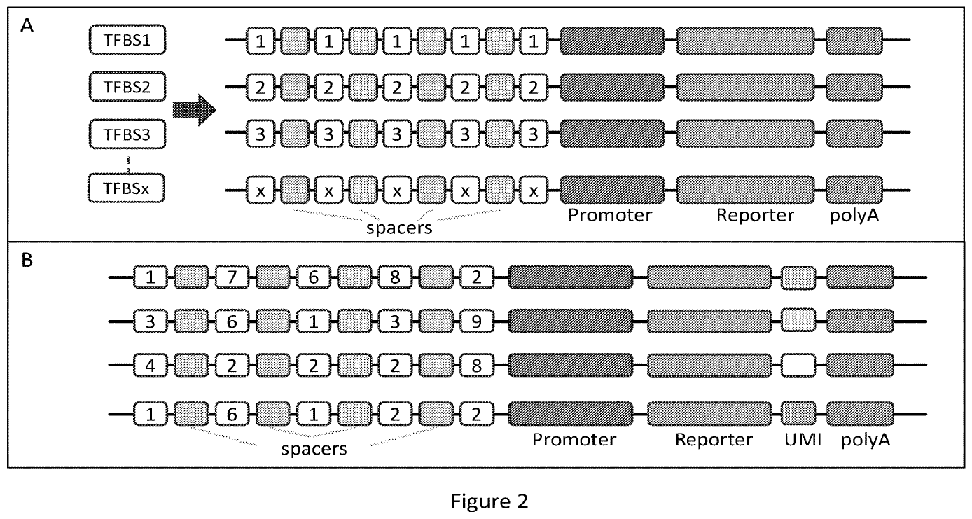

DNA construct design. Plasmids for testing the impact of DTS elements on nuclear translocation and expression were designed as shown in FIG. 2. Each DTS was constructed in pentameric repeat using a 15 bp spacer between each TFBS. The DTS was appended just 5′ to the promoter, effectively in a putative enhancer region. Various promoters were employed in these constructs, including CBh (a variant of the CAG promoter) and TTR. Several reporter genes were employed in these constructs, including enhanced Green Fluorescent Protein (eGFP). The polyadenylation (polyA) signal is a BGH-PolyA sequence. For pooled screening, unique molecular identifiers (UMIs; also known as barcodes) were used to distinguish between DNA constructs in a pool, and the UMIs were appended to the 3′UTR immediately downstream of the stop codon of eGFP, just preceding the BGH-PolyA sequence.

Library cloning. Pooled DTS libraries were designed as described above. To ensure the highest quality library production of oligos for cloning, oligo lengths were standardized to 300 bp. To accommodate this standardized length the core sequence of each DTS was embedded center-justified in a neutral fragment of random DNA sequence. The flanks of each oligo contained unique golden gate restriction enzyme digestion sites (Bsal) for cloning of double stranded DNA into screening vectors. To transform the manufactured oligo pool into double stranded DNA for cloning, oligos were PCR amplified with primers specific to the flanking region for 16 cycles of PCR with Superfi polymerase using the following program: 95° C., 16x (95° C. 0:15, 58° C. 0:15, 68° C. 0:15), 68° C. 2:00. A golden gate vector assembly was then performed by mixing the following components (each with unique golden gate restriction enzyme overhangs) together with Bsal restriction enzyme and T4 ligase in T4 ligase buffer: 1) double-stranded pooled DTS library DNA, 2) the hTTR promoter, 3) the eGFP gene, and 4) a BGH-PolyA fragment containing a machine-mixed N12 UMI. After 10 cycles 37° C./16° C. corresponding to vector digestion and ligation, the ligated DNA was transformed into NEB stable chemically competent E. Coli and plated onto LB-agarose with chloramphenicol antibiotic (34 ug/mL). After overnight incubation at 37° C., and counting of a minimum of 40,000 colonies, bacterial colonies were scraped, aggregated, and DNA extracted using endotoxin-free maxiprep. The proper UMI/DTS barcode correlation (termed here the “library registry”) was determined via excision of the hTTR/eGFP fragments, re-ligation of the vector, PCR amplification of the corresponding proximal DTS and UMI regions, and sequencing on an Illumina Miseq (2×150 bp reads). The DTS library plasmid pool was then transfected in vitro or formulated into LNPs for in vivo administration.

Cell culture. HepG2 cells were plated into a 384-well plate and grown for 48 hours to achieve confluency. Cell media was removed and replaced with culture media supplemented with Aphidicolin (1 uM) using EL406 automated plate washer and dispenser, then cells were transfected with DTS containing 17.5 ng plasmids using Lipofectamine 3000 without supplement. Expression of GFP was monitored using a Biotek Cytation 5 cell imaging reader over 24 hours to evaluate DTS activity. Total GFP per well was calculated and averaged over 4 well replicates.

LNP formulation. LNPs encapsulating nucleic acid payloads were prepared by mixing an organic solution of lipids with an aqueous solution of nucleic acid (e.g., DNA only, mRNA only, or DNA/mRNA mixtures) as described in Prud′homme et al. (J Pharm Sci 2018). Briefly, the lipidic excipients mixture (ionizable lipid, helper lipid, cholesterol, PEG-lipid and potentially other targeting moieties) is dissolved in an organic solvent. An aqueous solution of the nucleic acid is prepared in a low pH buffer of range pH 3.0-4.0. The lipid mixture is then mixed with the aqueous nucleic acid solution at a flow ratio of 1:3 (V/V) using a commercially available mixer device. The resulting solution is immediately diluted with a buffer pH range of 5.0-6.5. The diluted LNP is subjected to dialysis purification against a secondary buffer with the pH range of 7.0-8.0. The LNP solution is concentrated by using 100,000 MWCO Amicon Ultra centrifuge tubes (Millipore Sigma) followed by filtration through 0.2 μm PES sterilizing-grade filter. Particle size is determined by dynamic light scattering (Horiba nanoPartica SZ-100). Encapsulation efficiency is calculated by using Quant-it RiboGreen assay kit.

Quantification of mRNA abundance from pooled DTS screens. For in vitro studies, RNA was isolated by standard Trizol extraction methods and prepped with a Zymo RNA exraction column. For in vivo studies, liver tissue was homogenized in Trizol solution then prepped with a Zymo RNA extraction column to obtain purified RNA. Purified RNA was transformed into cDNA using Maximus H-minus Reverse Transcriptase (Thermofisher) with a custom RT primer targeted to the BGH-polyA sequence. cDNA was PCR amplified using primers specific to GFP and the UMI, then used to estimate transcript copy number and generate amplicons for library prep and Illumina sequencing. Total transcript copy number was estimated at 200-30,000 transcripts per sample in each PCR reaction. The PCR amplified cDNA was then library prepped (NEBNext library prep for Illumina) and sequenced on an Illumina Miseq (2×150 bp reads). Counts for each UMI were translated into the corresponding DTS using the UMI/Barcode registry and changes in abundance of each DTS were statistically analyzed using a custom R script. The log 2 fold change is calculated by comparing the RNA level in the pool vs. its expected abundance, which is derived from the abundance in the corresponding input DNA pool.

In vitro transcription of mRNA. mRNA was generated by in vitro transcription (IVT), using the commercially available mMESSAGE mMACHINE™ T7 Transcription Kit from ThermoFisher or the Hiscribe T7 mRNA kit from New England Biolabs. Incubation times, reagent concentrations, and a titration of reagents have been optimized to increase the yield and the purity of the mRNA. The IVT reaction uses a DNA template that can be a linearized plasmid, a PCR product, or a gene fragment. The mRNA sequences were codon optimized for expression in human and mouse cells. Engineered UTRs have been added to the template in order to facilitate mRNA expression and stability (Table 8). A combination of chemically modified nucleotides (e.g., m6A, m6Am, 2OmeA, Ac4C, m5C, pseudoU, mlpseudo1, 5moU) and a cap (e.g., Arca, CleanCap) have been used to stabilize the RNA and reduce its immunogenicity. A polyA tail has been encoded in the plasmid template or added through the commercially available PolyA reaction kit from ThermoFisher. mRNA was purified by LiCl precipitation or using the commercially available GeneJet RNA cleanup and concentration kit from ThermoFisher.

Co-transfection of DNA and mRNA into HepG2 cells. HepG2 cells were purchased from ATCC and grown in complete media (EMEM+10% fetal bovine serum, ATCC). For co-transfection experiments, cells were seeded at confluence onto collagen coated 384-well plates and cultured for 48 hours in complete media followed by 48 hours in complete media supplemented with 0.75 AM aphidicolin to prevent cell division. After the outgrowth period, cells were transfected using Lipofectamine3000 (Invitrogen) transfection reagent according to the manufacturer's instructions. RNA and DNA were independently complexed with Lipofectamine3000 in Opti-MEM I (Gibco) for 15 minutes. RNA and DNA complexes were then mixed, followed by 1:10 dilution into complete media supplemented with 0.75 μM aphidicolin. Media was aspirated from the plated HepG2 cells and replaced with the transfection mix. DNA was transfected from 17.5 ng to 4.38 ng per well. mRNA was transfected from 70 ng to 0.07 ng per well. Cells were live-imaged using a 4× objective with a GFP filter set and brightfield at 3-hour intervals for 72 hours using a Cytation5 automated microscope (Agilent) attached to a BioSpa8 automated incubator (Agilent). Gen5IPrime (Agilent) image analysis software was used to quantify the number of GFP cells and GFP Intensity per well.

Co-administration of DNA and mRNA to hepatocytes in vivo. DNA and mRNA are independently formulated as described elsewhere into different LNPs formulations, the two formulations are mixed to create a composite LNP admixture, and the composite LNP preparation dosed into miceAdditionally, DNA and mRNA are co-formulated into a single LNP formulation, and the co-formulation is dosed into mice.

Quantification of EPO levels in serum. Blood is collected via a retro-orbital bleed into serum separator tubes and processed to serum. The serum samples may be stored at −80C from collection until analysis. The serum levels of human EPO protein driven by expression from the DNA payload are quantified using the U-PLEX Human EPO Assay from MSD according to the manufacturer's instructions.

Quantification of FIX levels in plasma. Blood is collected via a retro-orbital bleed into K2EDTA tubes and processed to plasma. The plasma samples may be stored at −80C from collection until analysis. The plasma levels of human FIX following administration of LNPs were quantified using a U-Plex assay on the MSD platform. Briefly, a monoclonal mouse anti-human FIX antibody (Prolytix, clone AHIX-5041) was conjugated to biotin and used as the capture reagent on streptavidin-coated plates. A polyclonal goat anti-human FIX antibody (Cedarlane, clone CL20040AP) was conjugated to Sulfo-TAG and used as the detection reagent with the standard setup for quantification of electrochemiluminescence (ECL) signal using the QuickPlex SQ 120 MM instrument from MSD. Pooled normal human plasma (Affinity Biologicals, FRNCP0125), which is a pool of normal citrated human plasma collected from a minimum of 20 donors, was used to generate a standard curve and calculate % of normal human FIX levels. The assay was confirmed to be specific for human FIX and not to cross-react with mouse FIX, demonstrating very low levels of background in untreated mouse plasma samples.

### Example 1: Analysis of DTS Activity Using Multiple In Vitro Experimental Designs

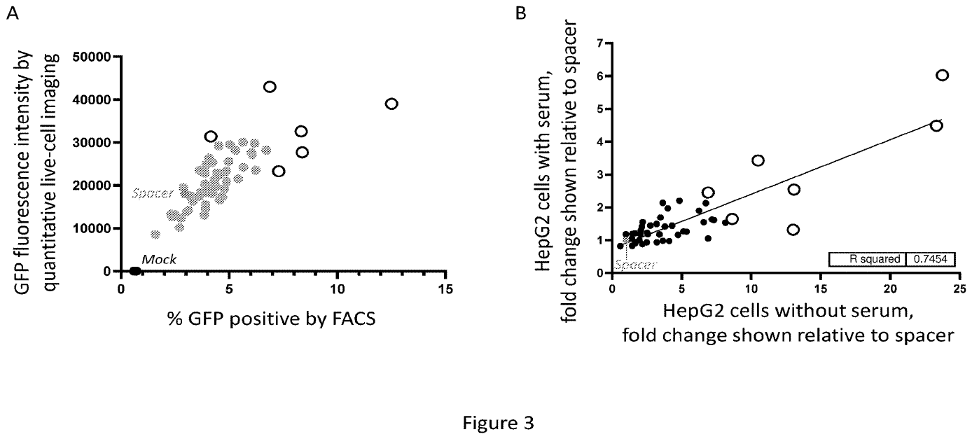

Plasmid DNAs (pDNAs) containing various DTSs were transfected into primary human hepatocytes. GFP expression was quantified 24 h post-transfection by measuring GFP intensity using quantitative live-cell imaging, then cells were harvested and the % of GFP-positive cells was measured by FACS. A strong correlation between total GFP intensity and % GFP-positive cells was observed (FIG. 3A). The DTSs identified as hits (open circles) increased both the GFP intensity and the % GFP-positive cells compared to the Spacer negative control (FIG. 3A). The increase in % of GFP-positive cells is important because it implies a nuclear translocation mechanism for DTS activity rather than simple enhancer activity that would have only increased GFP fluorescence intensity.

pDNAs containing various DTSs were transfected into HepG2 cells grown with or without serum. GFP expression was quantified 24 h post-transfection by measuring GFP intensity using quantitative live-cell imaging. HepG2 cells grown in the presence of serum were actively dividing, leading to DTS activities that produced less than 6-fold changes relative to Spacer negative control (FIG. 3B). However, HepG2 cells that were serum starved were not actively dividing, leading to DTS activities that produced up to 25-fold increases in GFP intensity relative to Spacer negative control (FIG. 3B). The DTSs identified as hits (open circles) produced increased GFP intensity in both experimental setups, with a good correlation between the conditions (FIG. 3B). Importantly, it was observed that non-dividing cells produced a greater dynamic range for assessing DTS function, due to the nuclear membrane remaining intact because the cells do not undergo mitosis.

### Example 2: Establishing the Function of Benchmark DTSs in Primary Human Hepatocytes

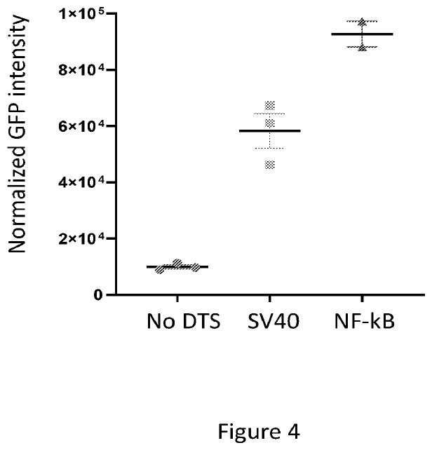

pDNAs containing a DTS with NF-κB binding sites, a DTS derived from the SV40 enhancer, or no DTS were transfected into primary human hepatocytes. GFP expression was quantified 24 h post-transfection by measuring GFP intensity using quantitative live-cell imaging. pDNA containing the NF-kB or SV40-derived DTSs produced robust increases in gene expression compared to the pDNA with no DTS (FIG. 4). The pDNA with a DTS containing NF-κB binding sites drove a nearly 10-fold increase in GFP intensity compared to the pDNA with no DTS (FIG. 4).

### Example 3: Arrayed Screen of Novel DTSs that Drive Improved Gene Expression In Vitro

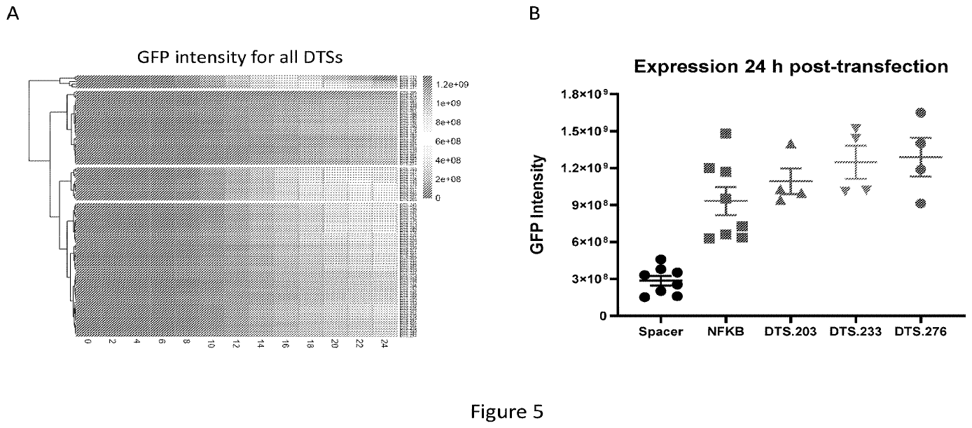

pDNAs containing various DTSs (Table 12) were transfected individually into growth-arrested HepG2 cells. Cell growth was inhibited to prevent dissolution of the nuclear envelope. GFP expression was quantified over a period of 24 h post-transfection by measuring GFP intensity using quantitative live-cell imaging. Statistical analysis and hierarchical clustering methods were used to easily differentiate strong DTSs from weak DTSs in a heatmap (FIG. 5A). The top DTSs at the 24 h time point performed equivalently or better than NFKB, driving robust increases in gene expression compared to the Spacer negative control (FIG. 5B). Unlike NFKB, which demonstrates increased activity in response to inflammation, the top 3 novel DTSs (DTS.203, DTS.233, and DTS.276) comprise TFBSs for constitutively expressed, hepatocyte-specific transcription factors that are not responsive to inflammation. DTS.203: CREB1, PPARA, ONECUT1, HNF4A, PPARA. DTS.233: HNF1A, NR113, PPARA, HNF1A, PPARA. DTS.276: FOXA1, HNF4A, HNF1A, HNF4A, HNF1A.

### Example 4: Establishing the Correlation Between Arrayed Screening and Pooled Screening

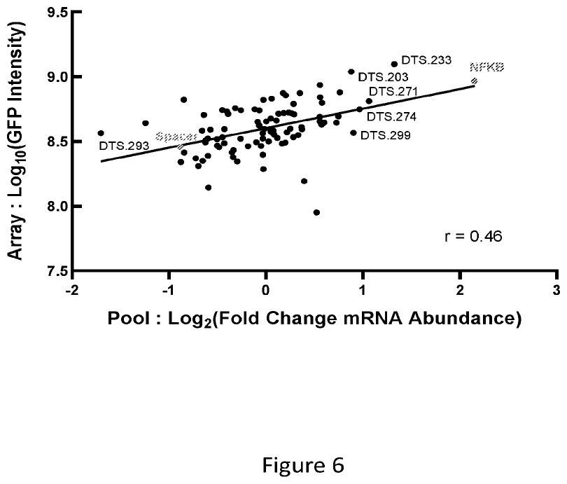

For the arrayed screen, pDNAs containing various DTSs were transfected individually into growth-arrested HepG2 cells. For the pooled screen, pDNAs containing various DTSs were pooled together and transfected as a pool into growth-arrested HepG2 cells. Cell growth was inhibited to prevent dissolution of the nuclear envelope, which improves the accuracy of measuring DTS activity related to nuclear translocation. For the arrayed screen, GFP protein expression was quantified 24 h post-transfection by measuring GFP intensity using quantitative live-cell imaging. For the pooled screen, mRNA abundance for each DTS variant was determined using RNA-seq by deconvoluting the read counts for each UMI. A strong correlation between protein expression in the arrayed screen and mRNA expression in the pooled screen was observed (FIG. 6). Several DTSs, including NFKB and the novel DTS.233, showed robust increases in mRNA abundance and GFP intensity relative to the Spacer negative control (FIG. 6). Pooled screening can thus be used to quickly and reliably identify strong DTSs.

### Example 5: In Vivo Confirmation of DTS Activity in Mouse Liver

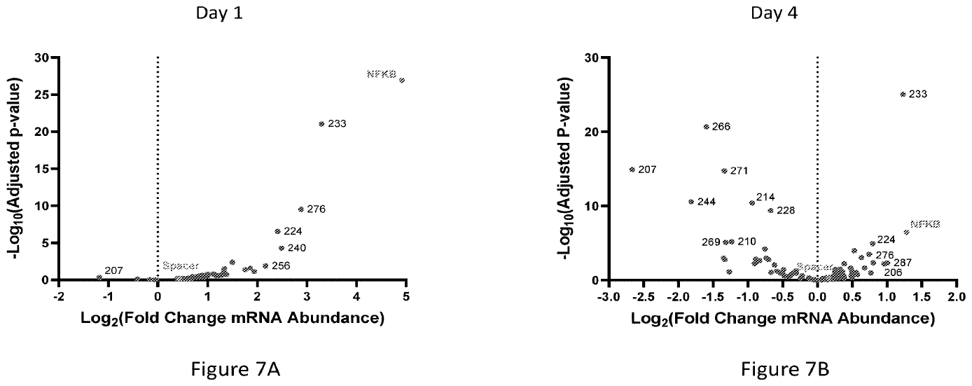

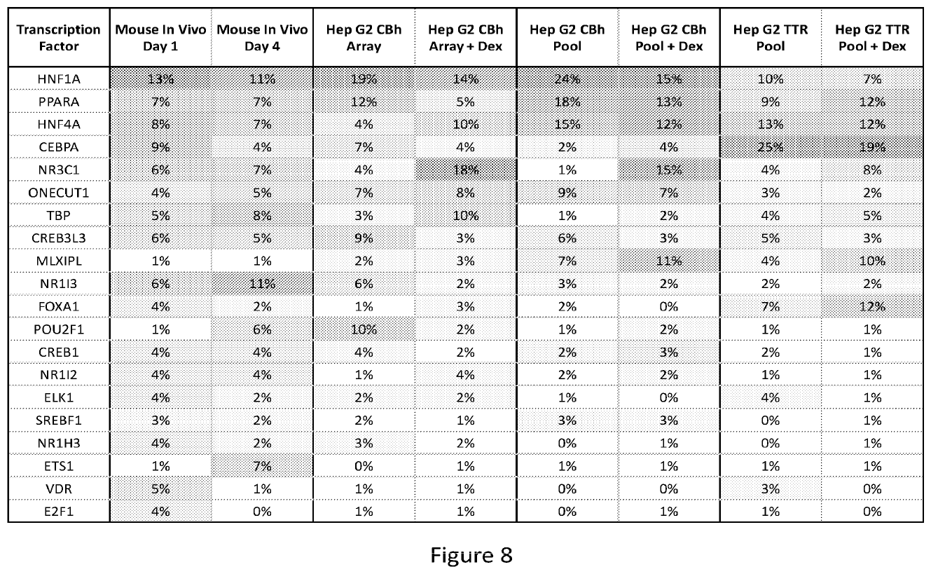

pDNAs containing various DTSs were pooled together and formulated into a lipid nanoparticle (LNP) comprising the ALC-0315 ionizable lipid, as described in the methods above. Wild type female BALB/c mice (approximately 8-12 weeks old) were dosed once by a single i.v. bolus injection into the tail vein at 5 mL/kg body weight. The pDNA-LNP was administered at 0.66 mg/kg based on the weight of the DNA payload to a group of n=5 mice. Both I d and 4 d after dosing, the mice were euthanized, and their livers were harvested and homogenized. Following RNA extraction from the liver homogenate, mRNA abundance for each DTS variant was determined using RNA-seq by deconvoluting the read counts for each UMI. At 1 d post-dose, NFKB produced the largest and most statistically significant increase in mRNA abundance compared to Spacer negative control (FIG. 7A). At 1 d post-dose, the best novel DTSs (DTS.233 and DTS.276) produced 8- to 10-fold increases in mRNA abundance, dramatically improving gene expression compared to the Spacer negative control (FIG. 7A). At 4 d post-dose, the best DTSs continue to show increased mRNA abundance, although the magnitude of the increase is lower likely due to gene silencing driven by the immune-stimulatory plasmid backbone (FIG. 7B). At 4 d post-dose, it can also be observed that many DTSs produced reductions in mRNA abundance, probably due to repressor-like activity (FIG. 7B). Since the combinatorial design of the novel DTSs employed several TFBSs arranged together, it was important to determine the combinations that led to both the increased and decreased gene expression. Positive and negative data can be used to train machine learning algorithms to enable the extraction of key trends. A gradient-boosted decision tree machine learning algorithm was developed and independently applied to each dataset that was generated with the DTS pool, which resulted in the same 3 transcription factors (HNF1A, PPARA, and HNF4A) being identified as overrepresented in strong DTSs and underrepresented in weak DTSs (FIG. 8).

### Example 6: Utilization of Inducing Agents to Improve DTS Activity

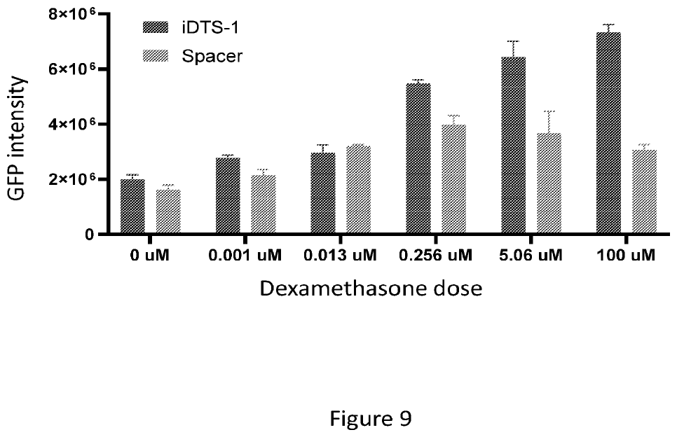

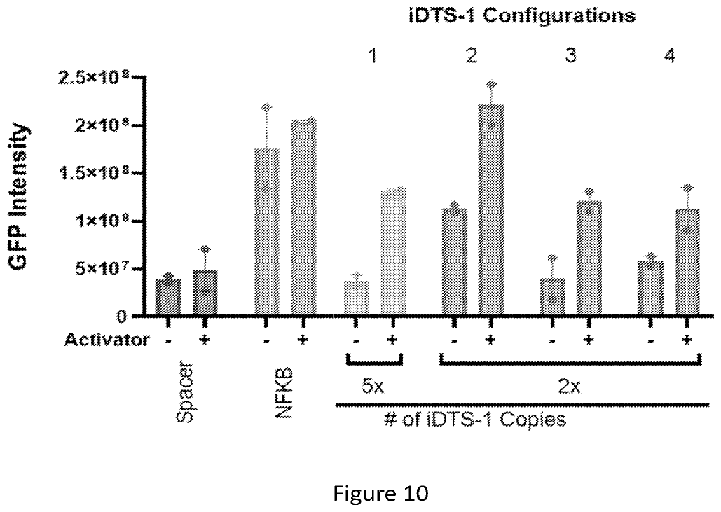

pDNAs containing various DTSs were transfected individually into growth-arrested HepG2 cells. Simultaneously, varying concentrations of an activating agent were added to the cells. Dexamethasone, which is a known activator of the glucocorticoid receptor (GR, also known as NR3C1), was used as the activating agent. GFP expression was quantified 24 h post-transfection by measuring GFP intensity using quantitative live-cell imaging. The inducible DTS (iDTS-1) containing 5 copies of the GR response element (GRE) in a pentameric repeat produced an increase in gene expression relative to Spacer negative control as a function of increased dexamethasone concentration (FIG. 9). Other configurations of iDTS-1 containing 2 copies of the GRE as part of a combinatorial pentameric repeat also showed increased gene expression in response to dexamethasone as the activating agent (FIG. 10). The iDTS-1-2 configuration produced an increase in gene expression that was equivalent to the robust NFKB DTS and significantly higher than the Spacer negative control (FIG. 10).

TABLE 12
DTSs comprising combinations of TFBSs.

| DTS Name | TFBS combinations | Sequence |
|---|---|---|
| DTS.201 | SREBF1.v2/MLXIPL.v1/ | ATCACGTGATAGATTACTACTGATAATCACG |
| | HNFIA.v9/CREB3L3.v4/ | TGATTATCACGTGATAGATTACTACTGATAT |
| | ONECUTI.v5 | GATAGCCAACTGCAGCTAATAATAAACCAAG |
| | | ATTACTACTGATACCATGAACTTTGAGATTA |
| | | CTACTGATAGTCTGCTAAGTCAATAATCAGA |
| | | AT (SEQ ID NO: 314) |
| DTS.202 | ONECUTI.v2/NR113.v5/ | TCCATTGATTTAGAGATTACTACTGATAGCA |
| | CREB1.v2/ONECUT1.v5/ | ATAAAATCTGGGTCACAGGAGTTGGAAGATT |
| | CREB3L3.v7 | ACTACTGATACTGACGTCAGAGATTACTACT |
| | | GATAGTCTGCTAAGTCAATAATCAGAATAGA |
| | | TTACTACTGATATACACGTAATC (SEQ ID |
| | | NO: 315) |
| DTS.203 | CREB1.v2/PPARA.v6/ | CTGACGTCAGAGATTACTACTGATACAAAAC |
| | ONECUTI.v5/HNF4A.v9/ | TAGGTCAAAGGTCAAGATTACTACTGATAGT |
| | PPARA.v2 | CTGCTAAGTCAATAATCAGAATAGATTACTA |
| | | CTGATACGCCCCAGCACACATGATCAGAAGA |
| | | TTACTACTGATAAGGTCAAAGGTCA (SEQ ID |
| | | NO: 316) |
| DTS.204 | EGR3.v2/ONECUTI.v1/ | TACGCCCACGCATTAGATTACTACTGATATA |
| | HNFIA.v6/EGR3.v2/ | TTGATTAGATTACTACTGATAAGTATGGTTA |
| | NR3C1.v6 | ATGATCTACAGAGATTACTACTGATATACGC |
| | | CCACGCATTAGATTACTACTGATAAGAACAT |
| | | CCCTGTACA (SEQ ID NO: 317) |
| DTS.205 | CREB3L3.v5/HNF4A.v10/ | CAAACGTGGTTTAGATTACTACTGATAAACA |
| | HNF1A.v7/SREBF1.v2/ | CGGGAGGTCAAAGATTGCGCCCAGATTACTA |
| | ETS1.v2 | CTGATATGGTTAATATTCACCAGCAGATTAC |
| | | TACTGATAATCACGTGATAGATTACTACTGA |
| | | TAACCGGAAGTACTTCCGGT (SEQ ID NO: 318) |
| DTS.206 | ETS1.v1/NR3C1.v4/ | ACAGGAAGTAGATTACTACTGATAAGAACAT |
| | ELF5.v2/CEBPA.v6/ | TTTGTACGAGATTACTACTGATAAACCCGGA |
| | NR113.v4 | AGTGAGATTACTACTGATATGGTATGATTTT |
| | | GTAATGGGGTAGGAAGATTACTACTGATAAA |
| | | TTATGGTTCTGGGTGATTCAAGTAACA (SEQ |
| | | ID NO: 319) |
| DTS.207 | CEBPA.v2/NR1I2.v2/ | ATTGTGCAATAGATTACTACTGATACAGAGG |
| | ONECUTI.v1/HNF4A.v5/ | TCACAGAGTTCAAGCAGATTACTACTGATAT |
| | POU2F1.v4 | ATTGATTAGATTACTACTGATATCGAGCGCA |
| | | GGTCAAAAGGTCACCTGCAGATTACTACTGA |
| | | TAATATGATTATGCAAATTTATAGA (SEQ ID |
| | | NO: 320) |
| DTS.208 | CREB1.vl/CREB3L3.v4/ | TGACGTCAAGATTACTACTGATACCATGAAC |
| | FOXAl.v1/CREB3L3.v6/ | TTTGAGATTACTACTGATATGTTTACTTTAGA |
| | CEBPA.v4 | |
| | | TTACTACTGATATCCACGTGGTATTAGATTAC |
| | | TACTGATAATTACAAAAT (SEQ ID NO: 321) |
| DTS.209 | NR1H3.v2/POU2F1.v3/ | AAACTAGGTCACGAAAGGTCAAAGTCAGATT |
| | MLXIPL.v1/HNF4A.v6/ | ACTACTGATATTATGCATATGCATAAAGATT |
| | HNFIA.v8 | ACTACTGATAATCACGTGATTATCACGTGAT |
| | | AGATTACTACTGATAAGGTTAAAGGTCTAGA |
| | | TTACTACTGATAGTTTATCAGTGACTAGTCAT |
| | | TGAT (SEQ ID NO: 322) |
| DTS.210 | HNF4A.v4/HNF4A.v2/ | TCGAGCGCAGGTCAAAGGTCACCTGCAGATT |
| | ELK1.v1/ONECUTI.v3/ | ACTACTGATATCGAGCGCTGGGCAAAGGTCA |
| | CREB3L3.v3 | CCTGCAGATTACTACTGATACACTTCCGCCG |
| | | GAAGTGAGATTACTACTGATAAAAAAATCAA |
| | | TAATAGATTACTACTGATACCACGCTG (SEQ |
| | | ID NO: 323) |
| DTS.211 | POU2F1.v4/CREB3L3, v8/ | ATATGATTATGCAAATTTATAGAAGATTACT |
| | CREB3L3.v2/CREB3L3.v1 | ACTGATACACACGTGATCAGATTACTACTGA |
| | /NR3C1.v2 | TACCACGTAGAGATTACTACTGATACCACGT |
| | | TGAGATTACTACTGATAAGAACAAAATGTTC |
| | | T (SEQ ID NO: 324) |
| DTS.212 | PPARA.v6/PPARA.v5/ | CAAAACTAGGTCAAAGGTCAAGATTACTACT |
| | HNF4A.v3/HNF1A.v10/ | GATAAACTAGGTCAAAGGTCAAGATTACTAC |
| | CREB3L3.v2 | TGATATCGAAGGGCAGGGGTCAAGGGTTCA |
| | | GTAGATTACTACTGATAATATTTTAGAGAAG |
| | | AATTAACCTTTAGATTACTACTGATACCACG |
| | | TAG (SEQ ID NO: 325) |
| DTS.213 | HNF4A.v9/HNF1A.v8/ | CGCCCCAGCACACATGATCAGAAGATTACTA |
| | HNF4A.v7/CREB3L3.v3/ | CTGATAGTTTATCAGTGACTAGTCATTGATA |
| | HNF1A.v3 | GATTACTACTGATAAGGTCAAAGTCCAAGAT |
| | | TACTACTGATACCACGCTGAGATTACTACTG |
| | | ATAGTTACTTATTCTC (SEQ ID NO: 326) |
| DTS.214 | CREB3L3.v2/SREBF1.v2/ | CCACGTAGAGATTACTACTGATAATCACGTG |
| | E2F1.v2/PPARA.v4/ | ATAGATTACTACTGATAAAAAATGGCGCCAA |
| | NRIH3.v2 | AATGAGATTACTACTGATAGTGTCAAAGGTC |
| | | AAGATTACTACTGATAAAACTAGGTCACGAA |
| | | AGGTCAAAGTC (SEQ ID NO: 327) |
| DTS.215 | NR1I3.v2/ONECUT1.v5/ | GCCCCCAGGGCTGAGTGACAGAAAAACAGA |
| | POU2F1.v3/NR1H3.v2/ | GATTACTACTGATAGTCTGCTAAGTCAATAA |
| | NR1I3.v6 | TCAGAATAGATTACTACTGATATTATGCATA |
| | | TGCATAAAGATTACTACTGATAAAACTAGGT |
| | | CACGAAAGGTCAAAGTCAGATTACTACTGAT |
| | | ACCTCTTCTCTGTGGGTGACCAGCGTCCTA |
| | | (SEQ ID NO: 328) |
| DTS.216 | POU2F1.v3/SP1.v2/ | TTATGCATATGCATAAAGATTACTACTGATA |
| | PPARA.v3/NR1I3.v6/ | GCCACGCCCCCAGATTACTACTGATAAGGTC |
| | POU2F1.v3 | AATGACCTAGATTACTACTGATACCTCTTCTC |
| | | TGTGGGTGACCAGCGTCCTAAGATTACTACT |
| | | GATATTATGCATATGCATAA (SEQ ID NO: 329) |
| DTS.217 | HNFIA.v5/NR3C1.v5/ | AATTATTTATTACCAAGATTACTACTGATAA |
| | HNFIA.v8/NR1I3.v1/ | AGAACATTTTGTACGTAGATTACTACTGATA |
| | FOXA1.v1 | GTTTATCAGTGACTAGTCATTGATAGATTACT |
| | | ACTGATACAGAGTTCATGAGAGTTCAAGCAG |
| | | ATTACTACTGATATGTTTACTTT (SEQ ID |
| | | NO: 330) |
| DTS.218 | NR113.v4/ONECUTI.v3/ | AATTATGGTTCTGGGTGATTCAAGTAACAAG |
| | NR3C1.v6/PPARA.v1/ | ATTACTACTGATAAAAAAATCAATAATAGAT |
| | PPARA.v4 | TACTACTGATAAGAACATCCCTGTACAAGAT |
| | | TACTACTGATAAGGTCATCAGGTCAAGATTA |
| | | CTACTGATAGTGTCAAAGGTCA (SEQ ID |
| | | NO: 331) |
| DTS.219 | HNF1A.v7/ETS1.v1/ | TGGTTAATATTCACCAGCAGATTACTACTGA |
| | NR1I3.v4/VDR.v2/ | TAACAGGAAGTAGATTACTACTGATAAATTA |
| | CEBPA.v3 | TGGTTCTGGGTGATTCAAGTAACAAGATTAC |
| | | TACTGATACAGGGGTCATCGGGTTCAAACAG |
| | | ATTACTACTGATAATTGCACAAT (SEQ ID |
| | | NO: 332) |
| DTS.220 | CEBPA.v5/NR1H3.v2/ | TGTTTGTTAAGGCAGATTACTACTGATAAAA |
| | NR112.v1/CREB1.v2/ | CTAGGTCACGAAAGGTCAAAGTCAGATTACT |
| | HNF4A.v3 | ACTGATAAGGCAGAGGGCAGAAAGGTCAAG |
| | | GGAGATTACTACTGATACTGACGTCAGAGAT |
| | | TACTACTGATATCGAAGGGCAGGGGTCAAGG |
| | | GTTCAGT (SEQ ID NO: 333) |
| DTS.221 | SP1.v2/PPARA.v3/ | GCCACGCCCCCAGATTACTACTGATAAGGTC |
| | HNFIA.v3/PPARA.v5/ | AATGACCTAGATTACTACTGATAGTTACTTA |
| | VDR.v2 | TTCTCAGATTACTACTGATAAACTAGGTCAA |
| | | AGGTCAAGATTACTACTGATACAGGGGTCAT |
| | | CGGGTTCAAAC (SEQ ID NO: 334) |
| DTS.222 | ONECUT1.v1/NR113.v4/ | TATTGATTAGATTACTACTGATAAATTATGGT |
| | ONECUT1.v2/SREBF1.v1/ | TCTGGGTGATTCAAGTAACAAGATTACTACT |
| | E2F1.v2 | GATATCCATTGATTTAGAGATTACTACTGAT |
| | | AATCACCCCACAGATTACTACTGATAAAAAA |
| | | TGGCGCCAAAATG (SEQ ID NO: 335) |
| DTS.223 | PPARA.v2/CREB3L3.v6/ | AGGTCAAAGGTCAAGATTACTACTGATATCC |
| | ONECUT1.v3/E2F1.v2/ | ACGTGGTATTAGATTACTACTGATAAAAAAA |
| | CREB1.v2 | TCAATAATAGATTACTACTGATAAAAAATGG |
| | | CGCCAAAATGAGATTACTACTGATACTGACG |
| | | TCAG (SEQ ID NO: 336) |
| DTS.224 | HNF4A.v2/NR113.v2/ | TCGAGCGCTGGGCAAAGGTCACCTGCAGATT |
| | NR3C1.v2/NR3C1.v5/ | ACTACTGATAGCCCCCAGGGCTGAGTGACAG |
| | HNF4A.v7 | AAAAACAGAGATTACTACTGATAAGAACAA |
| | | AATGTTCTAGATTACTACTGATAAAGAACAT |
| | | TTTGTACGTAGATTACTACTGATAAGGTCAA |
| | | AGTCCA (SEQ ID NO: 337) |
| DTS.225 | E2F1.v2/POU2F1.v2/ | AAAAATGGCGCCAAAATGAGATTACTACTGA |
| | PPARA.v1/NRIH3.v1/ | TAAATATGCAAATTAGAGATTACTACTGATA |
| | EGR3.v2 | AGGTCATCAGGTCAAGATTACTACTGATAAA |
| | | TAGAGGTCACTAAAGGTCAAGCAGATTACTA |
| | | CTGATATACGCCCACGCATT (SEQ ID NO: 338) |
| DTS.226 | HNF4A.v6/EGR3.v2/ | AGGTTAAAGGTCTAGATTACTACTGATATAC |
| | CREB3L3.v1/ONECUT1.v4 | GCCCACGCATTAGATTACTACTGATACCACG |
| | /CREB3L3.v5 | TTGAGATTACTACTGATAGAAAAAAAAATCA |
| | | ATATCGGGCCTAGATTACTACTGATACAAAC |
| | | GTGGTTT (SEQ ID NO: 339) |
| DTS.227 | CREB3L3.v1/NR1I2.v1/ | CCACGTTGAGATTACTACTGATAAGGCAGAG |
| | CREB3L3.v4/HNF1A.v2/ | GGCAGAAAGGTCAAGGGAGATTACTACTGA |
| | HNFIA.v4 | TACCATGAACTTTGAGATTACTACTGATAGT |
| | | TAATAATCTACAGATTACTACTGATAGGTTA |
| | | ATAATTAAC (SEQ ID NO: 340) |
| DTS.228 | POU2F1.v2/ONECUT1.v4/ | AATATGCAAATTAGAGATTACTACTGATAGA |
| | NR3C1.v5/NR113.v3/ | AAAAAAAATCAATATCGGGCCTAGATTACTA |
| | HNF4A.v5 | CTGATAAAGAACATTTTGTACGTAGATTACT |
| | | ACTGATAGCATTACAGACTGGGTGACAGAGT |
| | | GAGACAGATTACTACTGATATCGAGCGCAGG |
| | | TCAAAAGGTCACCTGC (SEQ ID NO: 341) |
| DTS.229 | CREB3L3.v4/NR1I3.v1/ | CCATGAACTTTGAGATTACTACTGATACAGA |
| | PPARA.v7/CREB1.v3/ | GTTCATGAGAGTTCAAGCAGATTACTACTGA |
| | NR1I3.v2 | TAAACTAGGTCAAAGGTCAAAGAGATTACTA |
| | | CTGATAGCACGTCAAGATTACTACTGATAGC |
| | | CCCCAGGGCTGAGTGACAGAAAAACAG (SEQ |
| | | ID NO: 342) |
| DTS.230 | SREBF1.v1/HNF4A.v3/ | ATCACCCCACAGATTACTACTGATATCGAAG |
| | CREB3L3.v8/PPARA.v3/ | GGCAGGGGTCAAGGGTTCAGTAGATTACTAC |
| | HNF4A.v4 | TGATACACACGTGATCAGATTACTACTGATA |
| | | AGGTCAATGACCTAGATTACTACTGATATCG |
| | | AGCGCAGGTCAAAGGTCACCTGC (SEQ ID |
| | | NO: 343) |
| DTS.231 | PPARA.v4/HNF4A.v8/ | GTGTCAAAGGTCAAGATTACTACTGATATGG |
| | HNF4A.v8/NR1I3.v5/ | GTCCAGAGGGCAAAAAGATTACTACTGATAT |
| | SREBF1.v1 | GGGTCCAGAGGGCAAAAAGATTACTACTGAT |
| | | AGCAATAAAATCTGGGTCACAGGAGTTGGA |
| | | AGATTACTACTGATAATCACCCCAC (SEQ ID |
| | | NO: 344) |
| DTS.232 | ONECUT1.v3/PPARA.v1/ | AAAAAATCAATAATAGATTACTACTGATAAG |
| | CEBPA.v5/NR3C1.v3/ | GTCATCAGGTCAAGATTACTACTGATATGTT |
| | NR1I2.v1 | TGTTAAGGCAGATTACTACTGATAAAGAACA |
| | | AAATGTTCTTAGATTACTACTGATAAGGCAG |
| | | AGGGCAGAAAGGTCAAGGG (SEQ ID NO: 345) |
| DTS.233 | HNFIA.v4/NR113.v6/ | GGTTAATAATTAACAGATTACTACTGATACC |
| | PPARA.v5/HNF1A.v6/ | TCTTCTCTGTGGGTGACCAGCGTCCTAAGATT |
| | PPARA.v6 | ACTACTGATAAACTAGGTCAAAGGTCAAGAT |
| | | TACTACTGATAAGTATGGTTAATGATCTACA |
| | | GAGATTACTACTGATACAAAACTAGGTCAAA |
| | | GGTCA (SEQ ID NO: 346) |
| DTS.234 | HNF1A.v9/HNF1A.v5/ | TGATAGCCAACTGCAGCTAATAATAAACCAA |
| | NR1I3.v3/POU2F1.v3/ | GATTACTACTGATAAATTATTTATTACCAAG |
| | ONECUT1.v3 | ATTACTACTGATAGCATTACAGACTGGGTGA |
| | | CAGAGTGAGACAGATTACTACTGATATTATG |
| | | CATATGCATAAAGATTACTACTGATAAAAAA |
| | | ATCAATAAT (SEQ ID NO: 347) |
| DTS.235 | HNF4A.v10/CREB3L3.v7/ | AACACGGGAGGTCAAAGATTGCGCCCAGATT |
| | VDR.v2/NR112.v1/ | ACTACTGATATACACGTAATCAGATTACTAC |
| | HNFIA.v2 | TGATACAGGGGTCATCGGGTTCAAACAGATT |
| | | ACTACTGATAAGGCAGAGGGCAGAAAGGTC |
| | | AAGGGAGATTACTACTGATAGTTAATAATCT |
| | | AC (SEQ ID NO: 348) |
| DTS.236 | ONECUT1.v4/NR113.v3/ | GAAAAAAAAATCAATATCGGGCCTAGATTAC |
| | HNFIA.v5/HNF4A.v8/ | TACTGATAGCATTACAGACTGGGTGACAGAG |
| | CEBPA.v6 | TGAGACAGATTACTACTGATAAATTATTTAT |
| | | TACCAAGATTACTACTGATATGGGTCCAGAG |
| | | GGCAAAAAGATTACTACTGATATGGTATGAT |
| | | TTTGTAATGGGGTAGGA (SEQ ID NO: 349) |
| DTS.237 | CREBI.v3/E2F1.v2/ | GCACGTCAAGATTACTACTGATAAAAAATGG |
| | HNF4A.v5/ETS1.v1/ | CGCCAAAATGAGATTACTACTGATATCGAGC |
| | HNF4A.v8 | GCAGGTCAAAAGGTCACCTGCAGATTACTAC |
| | | TGATAACAGGAAGTAGATTACTACTGATATG |
| | | GGTCCAGAGGGCAAAA (SEQ ID NO: 350) |
| DTS.238 | HNF1A.v6/POU2F1.v4/ | AGTATGGTTAATGATCTACAGAGATTACTAC |
| | CREB3L3.v3/CEBPA.v4/ | TGATAATATGATTATGCAAATTTATAGAAGA |
| | HNFIA.v9 | TTACTACTGATACCACGCTGAGATTACTACT |
| | | GATAATTACAAAATAGATTACTACTGATATG |
| | | ATAGCCAACTGCAGCTAATAATAAACCA |
| | | (SEQ ID NO: 351) |
| DTS.239 | PPARA.v5/PPARA.v2/ | AACTAGGTCAAAGGTCAAGATTACTACTGAT |
| | CREB3L3.v7/NR3C1.v2/ | AAGGTCAAAGGTCAAGATTACTACTGATATA |
| | MLXIPL.v1 | CACGTAATCAGATTACTACTGATAAGAACAA |
| | | AATGTTCTAGATTACTACTGATAATCACGTG |
| | | ATTATCACGTGAT (SEQ ID NO: 352) |
| DTS.240 | NR1I3.v6/HNF4A.v7/ | CCTCTTCTCTGTGGGTGACCAGCGTCCTAAG |
| | CREBI.v1/NR113.v2/ | ATTACTACTGATAAGGTCAAAGTCCAAGATT |
| | ETS1.v1 | ACTACTGATATGACGTCAAGATTACTACTGA |
| | | TAGCCCCCAGGGCTGAGTGACAGAAAAACA |
| | | GAGATTACTACTGATAACAGGAAGT (SEQ ID |
| | | NO: 353) |
| DTS.241 | NR3C1.v2/HNF1A.v9/ | AGAACAAAATGTTCTAGATTACTACTGATAT |
| | NR113.v2/MLXIPL.v1/ | GATAGCCAACTGCAGCTAATAATAAACCAAG |
| | HNF4A.v9 | ATTACTACTGATAGCCCCCAGGGCTGAGTGA |
| | | CAGAAAAACAGAGATTACTACTGATAATCAC |
| | | GTGATTATCACGTGATAGATTACTACTGATA |
| | | CGCCCCAGCACACATGATCAGA (SEQ ID |
| | | NO: 354) |
| DTS.242 | CEBPA.v6/CEBPA.v3/ | TGGTATGATTTTGTAATGGGGTAGGAAGATT |
| | NR1I3.v6/CREB3L3.v2/ | ACTACTGATAATTGCACAATAGATTACTACT |
| | PPARA.v1 | GATACCTCTTCTCTGTGGGTGACCAGCGTCCT |
| | | AAGATTACTACTGATACCACGTAGAGATTAC |
| | | TACTGATAAGGTCATCAGGTCA (SEQ ID |
| | | NO: 355) |
| DTS.243 | PPARA.v1/ONECUT1.v2/ | AGGTCATCAGGTCAAGATTACTACTGATATC |
| | NR112.v2/POU2F1.v4/ | CATTGATTTAGAGATTACTACTGATACAGAG |
| | HNFIA.v10 | GTCACAGAGTTCAAGCAGATTACTACTGATA |
| | | ATATGATTATGCAAATTTATAGAAGATTACT |
| | | ACTGATAATATTTTAGAGAAGAATTAACCTT |
| | | T (SEQ ID NO: 356) |
| DTS.244 | PPARA.v3/ETS1.v2/ | AGGTCAATGACCTAGATTACTACTGATAACC |
| | HNF4A.v4/NR3C1.v6/ | GGAAGTACTTCCGGTAGATTACTACTGATAT |
| | CREBI.v1 | CGAGCGCAGGTCAAAGGTCACCTGCAGATTA |
| | | CTACTGATAAGAACATCCCTGTACAAGATTA |
| | | CTACTGATATGACGTCA (SEQ ID NO: 357) |
| DTS.245 | NR3C1.v3/CREB3L3.v2/ | AAGAACAAAATGTTCTTAGATTACTACTGAT |
| | NR3C1.v4/PPARA.v2/ | ACCACGTAGAGATTACTACTGATAAGAACAT |
| | POU2F1.v2 | TTTGTACGAGATTACTACTGATAAGGTCAAA |
| | | GGTCAAGATTACTACTGATAAATATGCAAAT |
| | | TAG (SEQ ID NO: 358) |
| DTS.246 | CREB3L3.v7/CREB1.v2/ | TACACGTAATCAGATTACTACTGATACTGAC |
| | PPARA.v4/ELF5,v2/ | GTCAGAGATTACTACTGATAGTGTCAAAGGT |
| | PPARA.v7 | CAAGATTACTACTGATAAACCCGGAAGTGAG |
| | | ATTACTACTGATAAACTAGGTCAAAGGTCAA |
| | | AG (SEQ ID NO: 359) |
| DTS.247 | VDR.v2/HNF4A.v5/ | CAGGGGTCATCGGGTTCAAACAGATTACTAC |
| | HNF4A.v2/CREB3L3.v5/ | TGATATCGAGCGCAGGTCAAAAGGTCACCTG |
| | NR3C1.v3 | CAGATTACTACTGATATCGAGCGCTGGGCAA |
| | | AGGTCACCTGCAGATTACTACTGATACAAAC |
| | | GTGGTTTAGATTACTACTGATAAAGAACAAA |
| | | ATGTTCTT (SEQ ID NO: 360) |
| DTS.248 | HNF1A.v10/VDR.v2/ | ATATTTTAGAGAAGAATTAACCTTTAGATTA |
| | SREBFI.v2/ONECUT1.v2/ | CTACTGATACAGGGGTCATCGGGTTCAAACA |
| | ONECUT1.v2 | GATTACTACTGATAATCACGTGATAGATTAC |
| | | TACTGATATCCATTGATTTAGAGATTACTACT |
| | | GATATCCATTGATTTAG (SEQ ID NO: 361) |
| DTS.249 | NR3C1.v6/NR3C1.v3/ | AGAACATCCCTGTACAAGATTACTACTGATA |
| | NR113.v1/SP1.v2/ | AAGAACAAAATGTTCTTAGATTACTACTGAT |
| | FOXA1.v2 | ACAGAGTTCATGAGAGTTCAAGCAGATTACT |
| | | ACTGATAGCCACGCCCCCAGATTACTACTGA |
| | | TATTGTTTACTT (SEQ ID NO: 362) |
| DTS.250 | NR1I2.v2/CEBPA.v6/ | CAGAGGTCACAGAGTTCAAGCAGATTACTAC |
| | CEBPA.v4/FOXA1.v2/ | TGATATGGTATGATTTTGTAATGGGGTAGGA |
| | SREBFI.v2 | AGATTACTACTGATAATTACAAAATAGATTA |
| | | CTACTGATATTGTTTACTTAGATTACTACTGA |
| | | TAATCACGTGAT (SEQ ID NO: 363) |
| DTS.251 | NR3C1.v4/CREB3L3.v5/ | AGAACATTTTGTACGAGATTACTACTGATAC |
| | PPARA.v6/CEBPA.v3/ | AAACGTGGTTTAGATTACTACTGATACAAAA |
| | HNF4A.v2 | CTAGGTCAAAGGTCAAGATTACTACTGATAA |
| | | TTGCACAATAGATTACTACTGATATCGAGCG |
| | | CTGGGCAAAGGTCACCTGC (SEQ ID NO: 364) |
| DTS.252 | HNF1A.v2/NR3C1.v6/ | GTTAATAATCTACAGATTACTACTGATAAGA |
| | HNF4A.v10/CEBPA.v5/ | ACATCCCTGTACAAGATTACTACTGATAAAC |
| | HNF1A.v5 | ACGGGAGGTCAAAGATTGCGCCCAGATTACT |
| | | ACTGATATGTTTGTTAAGGCAGATTACTACT |
| | | GATAAATTATTTATTACCA (SEQ ID NO: 365) |
| DTS.253 | NR3Cl.v5/HNFIA.v4/ | AAGAACATTTTGTACGTAGATTACTACTGAT |
| | ETS1.v2/CREB1.v1/ | AGGTTAATAATTAACAGATTACTACTGATAA |
| | CEBPA.v5 | CCGGAAGTACTTCCGGTAGATTACTACTGAT |
| | | ATGACGTCAAGATTACTACTGATATGTTTGTT |
| | | AAGGC (SEQ ID NO: 366) |
| DTS.254 | CEBPA.v3/CREB3L3.v3/ | ATTGCACAATAGATTACTACTGATACCACGC |
| | NR1I3.v5/PPARA.v6/ | TGAGATTACTACTGATAGCAATAAAATCTGG |
| | ONECUT1.v4 | GTCACAGGAGTTGGAAGATTACTACTGATAC |
| | | AAAACTAGGTCAAAGGTCAAGATTACTACTG |
| | | ATAGAAAAAAAAATCAATATCGGGCCT (SEQ |
| | | ID NO: 367) |
| DTS.255 | NR113.v3/CREB1.v1/ | GCATTACAGACTGGGTGACAGAGTGAGACA |
| | PPARA.v2/ONECUT1.v1/ | GATTACTACTGATATGACGTCAAGATTACTA |
| | CREB3L3.v4 | CTGATAAGGTCAAAGGTCAAGATTACTACTG |
| | | ATATATTGATTAGATTACTACTGATACCATG |
| | | AACTTTG (SEQ ID NO: 368) |
| DTS.256 | HNF4A.v7/PPARA.v7/ | AGGTCAAAGTCCAAGATTACTACTGATAAAC |
| | CEBPA.v6/HNF1A.v7/ | TAGGTCAAAGGTCAAAGAGATTACTACTGAT |
| | NR3C1.v4 | ATGGTATGATTTTGTAATGGGGTAGGAAGAT |
| | | TACTACTGATATGGTTAATATTCACCAGCAG |
| | | ATTACTACTGATAAGAACATTTTGTACG (SEQ |
| | | ID NO: 369) |
| DTS.257 | HNFIA.v8/CREB1.v3/ | GTTTATCAGTGACTAGTCATTGATAGATTACT |
| | POU2F1.v4/FOXA1.v1/ | ACTGATAGCACGTCAAGATTACTACTGATAA |
| | NR113.v1 | TATGATTATGCAAATTTATAGAAGATTACTA |
| | | CTGATATGTTTACTTTAGATTACTACTGATAC |
| | | AGAGTTCATGAGAGTTCAAGC (SEQ ID |
| | | NO: 370) |
| DTS.258 | CREB3L3.v6/HNF4A.v9/ | TCCACGTGGTATTAGATTACTACTGATACGC |
| | CEBPA.v2/CEBPA.v2/ | CCCAGCACACATGATCAGAAGATTACTACTG |
| | NR1I2.v2 | ATAATTGTGCAATAGATTACTACTGATAATT |
| | | GTGCAATAGATTACTACTGATACAGAGGTCA |
| | | CAGAGTTCAAGC (SEQ ID NO: 371) |
| DTS.259 | MLXIPL.v1/CEBPA.v5/ | ATCACGTGATTATCACGTGATAGATTACTAC |
| | ETS1.v1/HNF4A.v10/ | TGATATGTTTGTTAAGGCAGATTACTACTGA |
| | HNF4A.v6 | TAACAGGAAGTAGATTACTACTGATAAACAC |
| | | GGGAGGTCAAAGATTGCGCCCAGATTACTAC |
| | | TGATAAGGTTAAAGGTCT (SEQ ID NO: 372) |
| DTS.260 | NR113.v5/NR3C1.v2/ | GCAATAAAATCTGGGTCACAGGAGTTGGAA |
| | CREB3L3.v5/NR112.v2/ | GATTACTACTGATAAGAACAAAATGTTCTAG |
| | CEBPA.v2 | ATTACTACTGATACAAACGTGGTTTAGATTA |
| | | CTACTGATACAGAGGTCACAGAGTTCAAGCA |
| | | GATTACTACTGATAATTGTGCAAT (SEQ ID |
| | | NO: 373) |
| DTS.261 | ELK1.v1/HNF4A.v6/ | CACTTCCGCCGGAAGTGAGATTACTACTGAT |
| | POU2F1.v2/NR1I3.v4/ | AAGGTTAAAGGTCTAGATTACTACTGATAAA |
| | CREB3L3.v8 | TATGCAAATTAGAGATTACTACTGATAAATT |
| | | ATGGTTCTGGGTGATTCAAGTAACAAGATTA |
| | | CTACTGATACACACGTGATC (SEQ ID NO: 374) |
| DTS.262 | HNF4A.v3/HNF1A.v6/ | TCGAAGGGCAGGGGTCAAGGGTTCAGTAGA |
| | NR1H3.v2/HNF4A.v7/ | TTACTACTGATAAGTATGGTTAATGATCTAC |
| | PPARA.v3 | AGAGATTACTACTGATAAAACTAGGTCACGA |
| | | AAGGTCAAAGTCAGATTACTACTGATAAGGT |
| | | CAAAGTCCAAGATTACTACTGATAAGGTCAA |
| | | TGACCT (SEQ ID NO: 375) |
| DTS.263 | ONECUT1.v5/ELF5.v2/ | GTCTGCTAAGTCAATAATCAGAATAGATTAC |
| | CREB3L3.v6/PPARA.v7/ | TACTGATAAACCCGGAAGTGAGATTACTACT |
| | HNF4A.v10 | GATATCCACGTGGTATTAGATTACTACTGAT |
| | | AAACTAGGTCAAAGGTCAAAGAGATTACTAC |
| | | TGATAAACACGGGAGGTCAAAGATTGCGCCC |
| | | (SEQ ID NO: 376) |
| DTS.264 | CREB3L3.v3/FOXA1.v1/ | CCACGCTGAGATTACTACTGATATGTTTACTT |
| | SREBF1.v1/HNFIA.v5/ | TAGATTACTACTGATAATCACCCCACAGATT |
| | SPI.v2 | ACTACTGATAAATTATTTATTACCAAGATTA |
| | | CTACTGATAGCCACGCCCCC(SEQ ID NO: 377) |
| DTS.265 | NR1H3.v1/FOXA1.v2/ | AATAGAGGTCACTAAAGGTCAAGCAGATTAC |
| | HNF1A.v10/HNF1A.v8/ | TACTGATATTGTTTACTTAGATTACTACTGAT |
| | NR3C1.v5 | AATATTTTAGAGAAGAATTAACCTTTAGATT |
| | | ACTACTGATAGTTTATCAGTGACTAGTCATT |
| | | GATAGATTACTACTGATAAAGAACATTTTGT |
| | | ACGT (SEQ ID NO: 378) |
| DTS.266 | HNF4A.v5/PPARA.v4/ | TCGAGCGCAGGTCAAAAGGTCACCTGCAGAT |
| | ONECUT1.v4/POU2F1.v2/ | TACTACTGATAGTGTCAAAGGTCAAGATTAC |
| | CREBI.v3 | TACTGATAGAAAAAAAAATCAATATCGGGCC |
| | | TAGATTACTACTGATAAATATGCAAATTAGA |
| | | GATTACTACTGATAGCACGTCA (SEQ ID |
| | | NO: 379) |
| DTS.267 | ELF5.v2/HNF1A.v3/ | AACCCGGAAGTGAGATTACTACTGATAGTTA |
| | HNF4A.v9/HNF1A.v3/ | CTTATTCTCAGATTACTACTGATACGCCCCAG |
| | ELF5.v2 | CACACATGATCAGAAGATTACTACTGATAGT |
| | | TACTTATTCTCAGATTACTACTGATAAACCCG |
| | | GAAGTG (SEQ ID NO: 380) |
| DTS.268 | PPARA.v7/HNFIA.v7/ | AACTAGGTCAAAGGTCAAAGAGATTACTACT |
| | HNFIA.v2/HNF4A.v3/ | GATATGGTTAATATTCACCAGCAGATTACTA |
| | ELK1.v1 | CTGATAGTTAATAATCTACAGATTACTACTG |
| | | ATATCGAAGGGCAGGGGTCAAGGGTTCAGT |
| | | AGATTACTACTGATACACTTCCGCCGGAAGT |
| | | G (SEQ ID NO: 381) |
| DTS.269 | FOXAl.v2/CREB3L3.v1/ | TTGTTTACTTAGATTACTACTGATACCACGTT |
| | CEBPA.v3/ETS1.v2/ | GAGATTACTACTGATAATTGCACAATAGATT |
| | PPARA.v5 | ACTACTGATAACCGGAAGTACTTCCGGTAGA |
| | | TTACTACTGATAAACTAGGTCAAAGGTCA |
| | | (SEQ ID NO: 382) |
| DTS.270 | HNFIA.v3/CEBPA.v2/ | GTTACTTATTCTCAGATTACTACTGATAATTG |
| | EGR3.v2/NR3C1.v4/ | TGCAATAGATTACTACTGATATACGCCCACG |
| | NR1H3.v1 | CATTAGATTACTACTGATAAGAACATTTTGT |
| | | ACGAGATTACTACTGATAAATAGAGGTCACT |
| | | AAAGGTCAAGC (SEQ ID NO: 383) |
| DTS.271 | ETS1.v2/HNF1A.v2/ | ACCGGAAGTACTTCCGGTAGATTACTACTGA |
| | CREB1.v3/HNF4A.v4/ | TAGTTAATAATCTACAGATTACTACTGATAG |
| | CREB3L3.v1 | CACGTCAAGATTACTACTGATATCGAGCGCA |
| | | GGTCAAAGGTCACCTGCAGATTACTACTGAT |
| | | ACCACGTTG (SEQ ID NO: 384) |
| DTS.272 | CREB3L3.v8/ELK1.v1/ | CACACGTGATCAGATTACTACTGATACACTT |
| | NR3Cl.v3/HNF1A.v9/ | CCGCCGGAAGTGAGATTACTACTGATAAAGA |
| | CREB3L3.v6 | ACAAAATGTTCTTAGATTACTACTGATATGA |
| | | TAGCCAACTGCAGCTAATAATAAACCAAGAT |
| | | TACTACTGATATCCACGTGGTATT (SEQ ID |
| | | NO: 385) |
| DTS.273 | CEBPA.v4/NR.1H3.v1/ | ATTACAAAATAGATTACTACTGATAAATAGA |
| | SP1.v2/ELKI.v1/NR113.v5 | GGTCACTAAAGGTCAAGCAGATTACTACTGA |
| | | TAGCCACGCCCCCAGATTACTACTGATACAC |
| | | TTCCGCCGGAAGTGAGATTACTACTGATAGC |
| | | AATAAAATCTGGGTCACAGGAGTTGGA (SEQ |
| | | ID NO: 386) |
| DTS.274 | NR1I2.v1/HNFIA.v10/ | AGGCAGAGGGCAGAAAGGTCAAGGGAGATT |
| | NRIH3.v1/HNFIA.v4/ | ACTACTGATAATATTTTAGAGAAGAATTAAC |
| | HNFIA.v7 | CTTTAGATTACTACTGATAAATAGAGGTCAC |
| | | TAAAGGTCAAGCAGATTACTACTGATAGGTT |
| | | AATAATTAACAGATTACTACTGATATGGTTA |
| | | ATATTCACCAGC (SEQ ID NO: 387) |
| DTS.275 | NR1I3.v1/CEBPA.v4/ | CAGAGTTCATGAGAGTTCAAGCAGATTACTA |
| | HNF4A.v6/CREB3L3.v8/ | CTGATAATTACAAAATAGATTACTACTGATA |
| | NR1I3.v3 | AGGTTAAAGGTCTAGATTACTACTGATACAC |
| | | ACGTGATCAGATTACTACTGATAGCATTACA |
| | | GACTGGGTGACAGAGTGAGAC (SEQ ID |
| | | NO: 388) |
| DTS.276 | FOXA1.v1/HNF4A.v4/ | TGTTTACTTTAGATTACTACTGATATCGAGCG |
| | HNFIA.v4/HNF4A.v2/ | CAGGTCAAAGGTCACCTGCAGATTACTACTG |
| | HNF1A.v6 | ATAGGTTAATAATTAACAGATTACTACTGAT |
| | | ATCGAGCGCTGGGCAAAGGTCACCTGCAGAT |
| | | TACTACTGATAAGTATGGTTAATGATCTACA |
| | | G (SEQ ID NO: 389) |
| DTS.277 | HNF4A.v8/SREBF1.v1/ | TGGGTCCAGAGGGCAAAAAGATTACTACTGA |
| | FOXAl.v2/CREB3L3.v7/ | TAATCACCCCACAGATTACTACTGATATTGTT |
| | ONECUT1.v1 | TACTTAGATTACTACTGATATACACGTAATC |
| | | AGATTACTACTGATATATTGATT (SEQ ID |
| | | NO: 390) |
| DTS.278 | TBP.v2/TBP.v2/TBP.v2/ | TTATATAAAATAAGATTACTACTGATATTAT |
| | TBP.v2/TBP.v2 | ATAAAATAAGATTACTACTGATATTATATAA |
| | | AATAAGATTACTACTGATATTATATAAAATA |
| | | AGATTACTACTGATATTATATAAAATA (SEQ |
| | | ID NO: 391) |
| DTS.279 | TBP.v2/TBP.v2/ | TTATATAAAATAAGATTACTACTGATATTAT |
| | PPARA.v7/TBP.v2/ | ATAAAATAAGATTACTACTGATAAACTAGGT |
| | TBP.v2 | CAAAGGTCAAAGAGATTACTACTGATATTAT |
| | | ATAAAATAAGATTACTACTGATATTATATAA |
| | | AATA (SEQ ID NO: 392) |
| DTS.280 | PPARA.v7/TBP.v2/ | AACTAGGTCAAAGGTCAAAGAGATTACTACT |
| | TBP.v2/TBP.v2/ | GATATTATATAAAATAAGATTACTACTGATA |
| | PPARA.v7 | TTATATAAAATAAGATTACTACTGATATTAT |
| | | ATAAAATAAGATTACTACTGATAAACTAGGT |
| | | CAAAGGTCAAAG (SEQ ID NO: 393) |
| DTS.281 | CEBPA.v6/CEBPA.v6/ | TGGTATGATTTTGTAATGGGGTAGGAAGATT |
| | CEBPA.v6/CEBPA.v6/ | ACTACTGATATGGTATGATTTTGTAATGGGG |
| | CEBPA.v6 | TAGGAAGATTACTACTGATATGGTATGATTT |
| | | TGTAATGGGGTAGGAAGATTACTACTGATAT |
| | | GGTATGATTTTGTAATGGGGTAGGAAGATTA |
| | | CTACTGATATGGTATGATTTTGTAATGGGGT |
| | | AGGA (SEQ ID NO: 394) |
| DTS.282 | CEBPA.v6/CEBPA.v6/ | TGGTATGATTTTGTAATGGGGTAGGAAGATT |
| | PPARA.v7/CEBPA.v6/ | ACTACTGATATGGTATGATTTTGTAATGGGG |
| | CEBPA.v6 | TAGGAAGATTACTACTGATAAACTAGGTCAA |
| | | AGGTCAAAGAGATTACTACTGATATGGTATG |
| | | ATTTTGTAATGGGGTAGGAAGATTACTACTG |
| | | ATATGGTATGATTTTGTAATGGGGTAGGA |
| | | (SEQ ID NO: 395) |
| DTS.283 | PPARA.v7/CEBPA.v6/ | AACTAGGTCAAAGGTCAAAGAGATTACTACT |
| | CEBPA.v6/CEBPA.v6/ | GATATGGTATGATTTTGTAATGGGGTAGGAA |
| | PPARA.v7 | GATTACTACTGATATGGTATGATTTTGTAATG |
| | | GGGTAGGAAGATTACTACTGATATGGTATGA |
| | | TTTTGTAATGGGGTAGGAAGATTACTACTGA |
| | | TAAACTAGGTCAAAGGTCAAAG (SEQ ID |
| | | NO: 396) |
| DTS.284 | CEBPA.v6/CEBPA.v6/ | TGGTATGATTTTGTAATGGGGTAGGAAGATT |
| | NR3Cl.v3/CEBPA.v6/ | ACTACTGATATGGTATGATTTTGTAATGGGG |
| | CEBPA.v6 | TAGGAAGATTACTACTGATAAAGAACAAAAT |
| | | GTTCTTAGATTACTACTGATATGGTATGATTT |
| | | TGTAATGGGGTAGGAAGATTACTACTGATAT |
| | | GGTATGATTTTGTAATGGGGTAGGA (SEQ ID |
| | | NO: 397) |
| DTS.285 | NR3C1.v3/CEBPA.v6/ | AAGAACAAAATGTTCTTAGATTACTACTGAT |
| | CEBPA.v6/CEBPA.v6/ | ATGGTATGATTTTGTAATGGGGTAGGAAGAT |
| | NR3C1.v3 | TACTACTGATATGGTATGATTTTGTAATGGG |
| | | GTAGGAAGATTACTACTGATATGGTATGATT |
| | | TTGTAATGGGGTAGGAAGATTACTACTGATA |
| | | AAGAACAAAATGTTCTT (SEQ ID NO: 398) |
| DTS.286 | NR3C1.v3/CEBPA.v6/ | AAGAACAAAATGTTCTTAGATTACTACTGAT |
| | CEBPA.v6/CEBPA.v6/ | ATGGTATGATTTTGTAATGGGGTAGGAAGAT |
| | PPARA.v7 | TACTACTGATATGGTATGATTTTGTAATGGG |
| | | GTAGGAAGATTACTACTGATATGGTATGATT |
| | | TTGTAATGGGGTAGGAAGATTACTACTGATA |
| | | AACTAGGTCAAAGGTCAAAG (SEQ ID |
| | | NO: 399) |
| DTS.287 | NR3C1.v3/CEBPA.v6/ | AAGAACAAAATGTTCTTAGATTACTACTGAT |
| | HNFIA.v6/CEBPA.v6/ | ATGGTATGATTTTGTAATGGGGTAGGAAGAT |
| | PPARA.v7 | TACTACTGATAAGTATGGTTAATGATCTACA |
| | | GAGATTACTACTGATATGGTATGATTTTGTA |
| | | ATGGGGTAGGAAGATTACTACTGATAAACTA |
| | | GGTCAAAGGTCAAAG (SEQ ID NO: 400 ) |
| DTS.288 | NR3C1.v3/CEBPA.v6/ | AAGAACAAAATGTTCTTAGATTACTACTGAT |
| | HNF4A.v4/CEBPA.v6/ | ATGGTATGATTTTGTAATGGGGTAGGAAGAT |
| | PPARA.v7 | TACTACTGATATCGAGCGCAGGTCAAAGGTC |
| | | ACCTGCAGATTACTACTGATATGGTATGATT |
| | | TTGTAATGGGGTAGGAAGATTACTACTGATA |
| | | AACTAGGTCAAAGGTCAAAG (SEQ ID |
| | | NO: 401) |
| DTS.289 | NR3Cl.v3/CEBPA.v6/ | AAGAACAAAATGTTCTTAGATTACTACTGAT |
| | CREB3L3.v6/CEBPA.v6/ | ATGGTATGATTTTGTAATGGGGTAGGAAGAT |
| | PPARA.v7 | TACTACTGATATCCACGTGGTATTAGATTACT |
| | | ACTGATATGGTATGATTTTGTAATGGGGTAG |
| | | GAAGATTACTACTGATAAACTAGGTCAAAGG |
| | | TCAAAG (SEQ ID NO: 402) |
| DTS.290 | NR3C1.v3/TBP.v2/ | AAGAACAAAATGTTCTTAGATTACTACTGAT |
| | CREB3L3.v6/CEBPA.v6/ | ATTATATAAAATAAGATTACTACTGATATCC |
| | PPARA.v7 | ACGTGGTATTAGATTACTACTGATATGGTAT |
| | | GATTTTGTAATGGGGTAGGAAGATTACTACT |
| | | GATAAACTAGGTCAAAGGTCAAAG (SEQ ID |
| | | NO: 403) |
| DTS.291 | HNF1A.v6/TBP.v2/ | AGTATGGTTAATGATCTACAGAGATTACTAC |
| | CREB3L3.v6/CEBPA.v6/ | TGATATTATATAAAATAAGATTACTACTGAT |
| | PPARA.v7 | ATCCACGTGGTATTAGATTACTACTGATATG |
| | | GTATGATTTTGTAATGGGGTAGGAAGATTAC |
| | | TACTGATAAACTAGGTCAAAGGTCAAAG |
| | | (SEQ ID NO: 404) |
| DTS.292 | HNF4A.v4/TBP.v2/ | TCGAGCGCAGGTCAAAGGTCACCTGCAGATT |
| | CREB3L3.v6/CEBPA.v6/ | ACTACTGATATTATATAAAATAAGATTACTA |
| | PPARA.v7 | CTGATATCCACGTGGTATTAGATTACTACTG |
| | | ATATGGTATGATTTTGTAATGGGGTAGGAAG |
| | | ATTACTACTGATAAACTAGGTCAAAGGTCAA |
| | | AG (SEQ ID NO: 405) |
| DTS.293 | MLXIPL.v1/TBP.v2/ | ATCACGTGATTATCACGTGATAGATTACTAC |
| | CREB3L3.v6/CEBPA.v6/ | TGATATTATATAAAATAAGATTACTACTGAT |
| | PPARA.v7 | ATCCACGTGGTATTAGATTACTACTGATATG |
| | | GTATGATTTTGTAATGGGGTAGGAAGATTAC |
| | | TACTGATAAACTAGGTCAAAGGTCAAAG |
| | | (SEQ ID NO: 406) |
| DTS.294 | MLXIPL.v1/SREBF1.v1/ | ATCACGTGATTATCACGTGATAGATTACTAC |
| | CREB3L3.v6/CEBPA.v6/ | TGATAATCACCCCACAGATTACTACTGATAT |
| | PPARA.v7 | CCACGTGGTATTAGATTACTACTGATATGGT |
| | | ATGATTTTGTAATGGGGTAGGAAGATTACTA |
| | | CTGATAAACTAGGTCAAAGGTCAAAG (SEQ |
| | | ID NO: 407) |
| DTS.295 | NR1I3.v2/CEBPA.v6/ | GCCCCCAGGGCTGAGTGACAGAAAAACAGA |
| | MLXIPL.v1/CREB3L3.v6/ | GATTACTACTGATATGGTATGATTTTGTAATG |
| | SREBF1.v1 | GGGTAGGAAGATTACTACTGATAATCACGTG |
| | | ATTATCACGTGATAGATTACTACTGATATCC |
| | | ACGTGGTATTAGATTACTACTGATAATCACC |
| | | CCAC (SEQ ID NO: 408) |
| DTS.296 | NR1I3.v2/CEBPA.v6/ | GCCCCCAGGGCTGAGTGACAGAAAAACAGA |
| | MLXIPL.v1/CREB3L3.v6/ | GATTACTACTGATATGGTATGATTTTGTAATG |
| | HNFIA.v6 | GGGTAGGAAGATTACTACTGATAATCACGTG |
| | | ATTATCACGTGATAGATTACTACTGATATCC |
| | | ACGTGGTATTAGATTACTACTGATAAGTATG |
| | | GTTAATGATCTACAG (SEQ ID NO: 409) |
| DTS.297 | NR113.v2/CEBPA.v6/ | GCCCCCAGGGCTGAGTGACAGAAAAACAGA |
| | HNF1A.v6/CREB3L3.v6/ | GATTACTACTGATATGGTATGATTTTGTAATG |
| | HNF4A.v4 | GGGTAGGAAGATTACTACTGATAAGTATGGT |
| | | TAATGATCTACAGAGATTACTACTGATATCC |
| | | ACGTGGTATTAGATTACTACTGATATCGAGC |
| | | GCAGGTCAAAGGTCACCTGC (SEQ ID NO: 410) |
| DTS.298 | NFKB1.v2/NFKB1.v2/ | GGGACTTTCCAGATTACTACTGATAGGGACT |
| | NFKB1.v2/NFKB1.v2/ | TTCCAGATTACTACTGATAGGGACTTTCCAG |
| | NFKB1.v2 | ATTACTACTGATAGGGACTTTCCAGATTACT |
| | | ACTGATAGGGACTTTCC (SEQ ID NO: 411) |
| DTS.299 | ElA.v1/hBov1.v1/ | ACACAGGAAGTGACAATTTTCGCGCGGTTTT |
| | HS.CRM8.v1 | AGGCGGATGTTGTAGTAAATTTGGGCGTAAC |
| | | CGAGTAAGATTTGGCCATTTTCGCGGGAAAA |
| | | CTGAATAAGAGGAAGTGAAATCTGAATAATT |
| | | TTAGATTACTACTGATAGTGGTTGTACAGAC |
| | | GCCATCTTGGAATCCAATATGTCTGCCGGCT |
| | | CAGTCATGCCTGCGCTGCGCGCAGCGCGCTG |
| | | CGCGCGCGCATGATCTAATCGCCGGCAGACA |
| | | TATTGGATTCCAAGATGGCGTCTGTACAACC |
| | | ACGTCACATATAAGATTACTACTGATAGGGG |
| | | AGGCTGCTGGTGAATATTAACCAAGGTCACC |
| | | CCCAGTTATCGGAGGAGCAAACAGGGACTA |
| | | AGTCCAC (SEQ ID NO: 412) |
| DTS.300 | spacer | AGATTACTACTGATAAGATTACTACTGATAA |
| | | GATTACTACTGATAAGATTACTACTGATA |
| | | (SEQ ID NO: 413) |

### Example 7: In Vitro Co-Transfection of NTF-Expressing mRNA Improves DNA Nuclear Translocation and Gene Expression

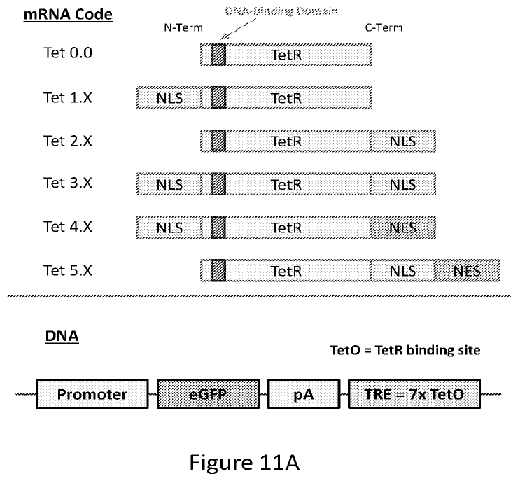

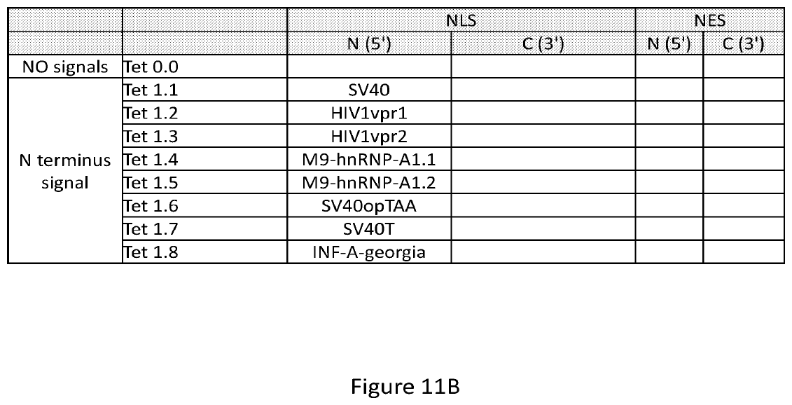

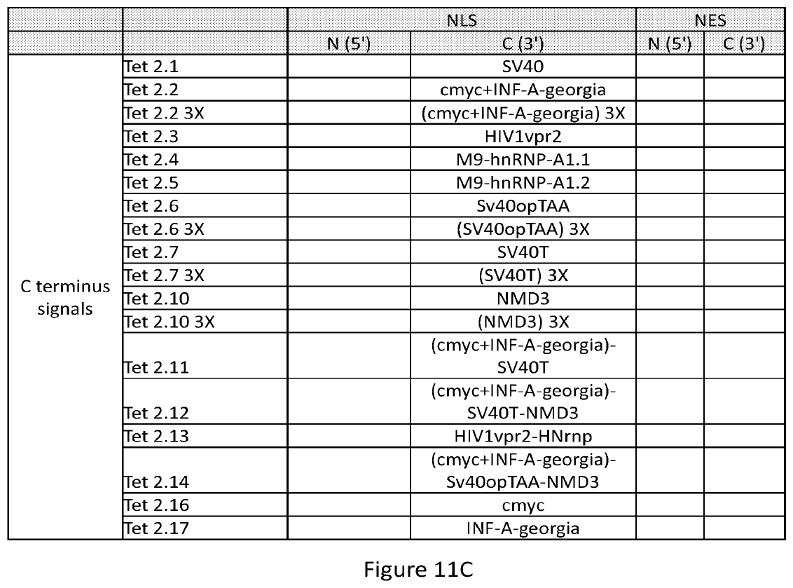

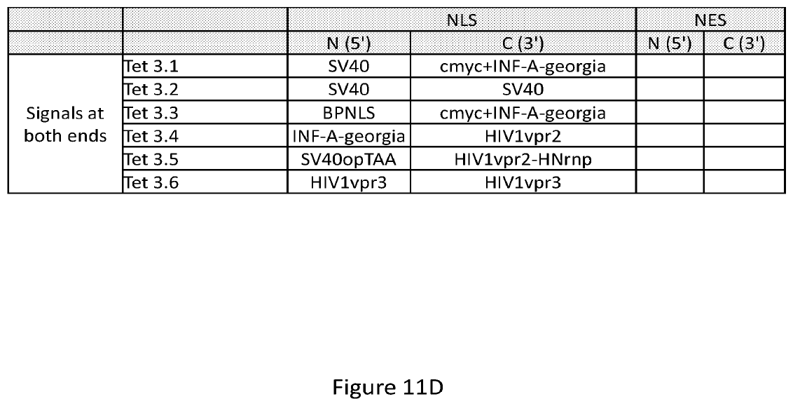

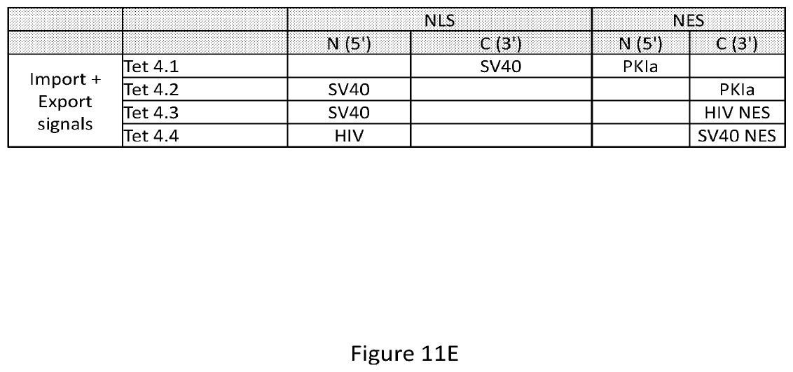

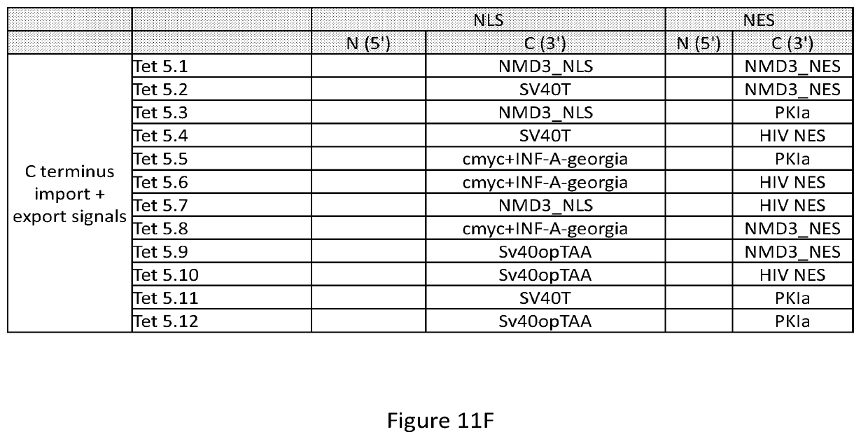

mRNAs were designed to express TetR fusion proteins with nuclear localization signals (NLSs) and in some instances nuclear export signals (NESs) placed at the N-terminus, C-terminus, or both (FIG. 11A-C) as outlined in Table 13.

TABLE 13
TetR-NLS fusion proteins

| Designation | Description | Sequence |
|---|---|---|
| 2.2 | c-myc NLS - Influenza A | PAAKRVKLDASQGTKRSYEQMETDGERQ |
| | NLS fusion | (SEQ ID NO: 414) |
| 2.6 | Optimized SV40 long | PSSDDEATADSQHAAPPKKKRKVEDPKDFPS |
| | NLS | ELLS (SEQ ID NO: 415) |
| 2.7 | SV40 long NLS | KKKSSSDDEATADSQHSTPPKKKRKVEDPKD |
| | | FPSELLS (SEQ ID NO: 416) |
| 2.11 | c-myc NLS - Influenza A | PAAKRVKLDASQGTKRSYEQMETDGERQKK |
| | NLS - SV40 long NLS | KSSSDDEATADSQHSTPPKKKRKVEDPKDFP |
| | fusion | SELLS (SEQ ID NO: 417) |
| 2.18 | SV40 short NLS - | PKKKRKVASQGTKRSYEQMETDGERQ (SEQ |
| | Influenza A NLS fusion | ID NO: 418) |
| 2.19 | Influenza A NLS - SV40 | MASQGTKRSYEQMETDGERQPKKKRKV |
| | short NLS fusion | (SEQ ID NO: 419) |
| 2.20 | c-myc NLS - Influenza A | PAAKRVKLDASQGTKRSYEQMETDGERQPA |
| | NLS - c-myc NLS fusion | AKRVKLD (SEQ ID NO: 420) |
| 2.21 | Influenza A NLS - c-myc | MASQGTKRSYEQMETDGERQPAAKRVKLD |
| | NLS fusion | (SEQ ID NO: 421) |
| 2.22 | c-myc NLS - 4XGS linker | MPAAKRVKLDGSGSGSGSASQGTKRSYEQM |
| | - Influenza A NLS fusion | ETDGERQ (SEQ ID NO: 422) |
| 2.27c | c-myc NLS - variant | PAAKRVKLDSQGTKRSYEQM (SEQ ID |
| | Influenza A NLS fusion | NO: 423) |
| | containing only the | |
| | importin binding site | |
| 2.28c | c-myc NLS - variant | PAAKRVKLDSQGTKRSYEQMTKRSYEQMSQ |
| | Influenza A NLS fusion | GTKRSYEQMTKRSYEQM (SEQ ID NO: 424) |
| | containing tandem | |
| | repetition of importin | |
| | alpha and beta binding | |
| | sites | |
| 2.40 | Linker - Influenza A NLS | GGGGSGGGGSGGGGSGGGGSPAAKRVKLDA |
| | fusion | SQGTKRSYEQMETDGERQ (SEQ ID NO: 425) |
| 2.52 | Influenza A NLS2-linker - | RKTRGNEGRWFNRDNIGKRQSGSGSTATGSG |
| | c-myc NLS fusion | PAAKRVKLD (SEQ ID NO: 426) |
| 2.64 | 4XGGGGS linker - | MGGGGSGGGGSGGGGSGGGGSAARLHRFK |
| | Importin Beta binding site | NKGKDSTEMRRRRIEVNVELRKAKKDDQML |
| | fusion | KRRN (SEQ ID NO: 427) |
| 2.65 | point mutant c-myc NLS | PAAKRAKLDASQGTKRSYEQMETDGERQ |
| | fused to Influenza A NLS | (SEQ ID NO: 428) |
| 2.67 | variant c-myc NLS - | PAAKRAKLDASQGTKRSYEQMETDPPPRG |
| | Influenza A NLS fusion | (SEQ ID NO: 429) |
| 2.75 | Covid NLSI, Covid | SGTNGTKRFDLGVYYHKNNKLALHRSYLTP |
| | NLS2, Covid NLS3, | TNSPRRARSVASQGTKRSYEQMETDGERQ |
| | Covid NLS4, Influenza A | (SEQ ID NO: 430) |
| | NLS fusion | |
| 2.85 | c-myc NLS - PRE-S2/UC | PAAKRVKLDPLSSIFSRIGDPSQGTKRSYEQM |
| | NLS fusion | ETDGERQ (SEQ ID NO: 431) |
| 2.86 | c-myc NLS - PRE- | PAAKRVKLDPLSSIFSRIGDPSTPPKKKRKVT |
| | S2/SV40 NLS fusion | DGERQ (SEQ ID NO: 432) |
| 2.87 | c-myc NLS - APNP/UC | PAAKRVKLDRQIKIWFQNRRMKWKKSQGTK |
| | NLS fusion | RSYEQMETDGERQ (SEQ ID NO: 433) |
| 2.88 | c-myc NLS - variant | PAAKRVKLDATQGTKRSYEQMETDGERQ |
| | Influenza A NLS fusion | (SEQ ID NO: 434) |
| 2.90 | SV40 short NLS - | PKKKRKVEDPASQGTKRSYEQMETDGERQ |
| | Influenza A NLS2 fusion | (SEQ ID NO: 435) |
| 2.91 | c-myc NLS - variant | PAAKRVKLDASPKKKRKYEQMETDGERQ |
| | Influenza A NLS fusion | (SEQ ID NO: 436) |
| 2.92 | c-myc NLS - SV40 short | PAAKRVKLDPKKKRKVEDPASQGTKRSYEQ |
| | NLS - Influenza A NLS | METDGERQ (SEQ ID NO: 437) |
| | fusion | |
| 2.94 | c-myc NLS - variant | PKKKRKVEDPSQGTKRSYEQMETDGERQ |
| | Influenza A NLS fusion | (SEQ ID NO: 438) |
| 2.97 | c-myc NLS - human | PAAKRVKLDFGNYNNQSSNFGPMKGGNFGG |
| | heterogeneous nuclear | RSSGPY (SEQ ID NO: 439) |
| | ribonucleoprotein Al | |
| | fusion | |
| 1.10 | Influenza A NLS - c-myc | MASQGTKRSYEQMETDGERQPAAKRVKLD |
| | NLS fusion | (SEQ ID NO: 440) |
| 4.1 | Phosphokinase alpha NES | PKKKRKVEDP (SEQ ID NO: 441) |
| | at N-terminus, SV40 short | |
| | NLS C-terminus | |
| 5.4 | SV40 long NLS - HIV | KKKSSSDDEATADSQHSTPPKKKRKVEDPKD |
| | NES fusion | FPSELLSLPPLERLTL (SEQ ID NO: 442) |
| 5.6 | c-myc NLS - Influenza A | PAAKRVKLDASQGTKRSYEQMETDGERQLP |
| | NLS - HIV NES fusion | PLERLTL (SEQ ID NO: 443) |

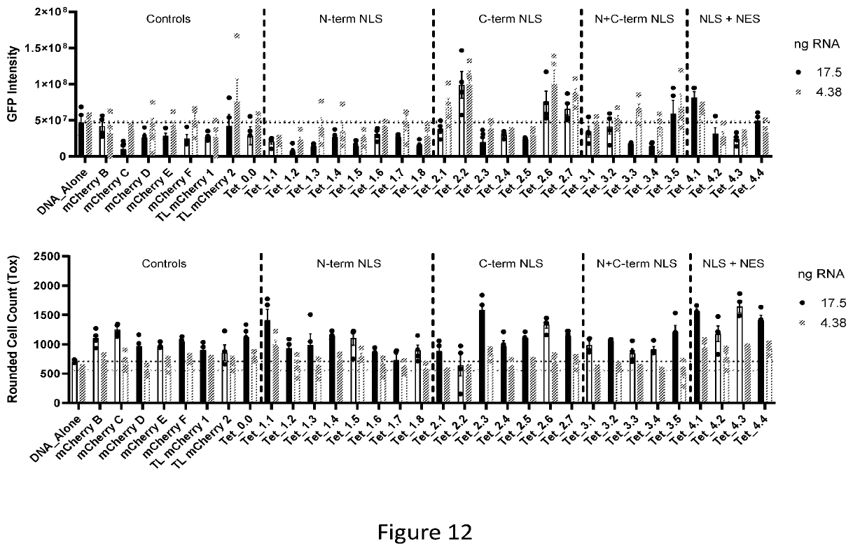

Growth-arrested HepG2 cells were cotransfected with these different TetR fusion proteins and a plasmid NTDNA comprising the tetracycline response element (TRE) (containing 7 copies of the TetO sequence TCGAGTTTACTCCCTATCAGTGATAGAGAACG (SEQ ID NO:444) with a 4 nucleotide spacer TATG between each) as a DTS and a GFP expression cassette. Forty-eight hours after transfection, GFP expression was assessed by measuring GFP fluorescence intensity; toxicity was assessed by counting the rounded (dead) cells. The C-terminal NLS conjugates 2.2, 2.6, and 2.7 produced the highest increase in GFP intensity relative to DNA alone and DNA+mCherry mRNA negative controls (FIG. 12).

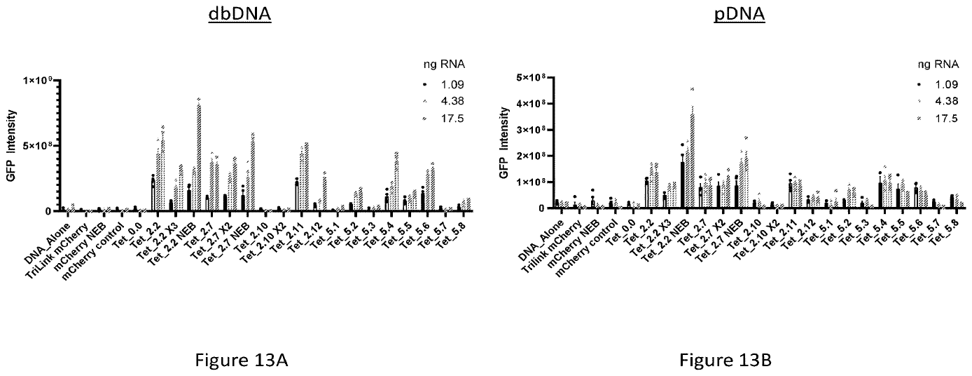

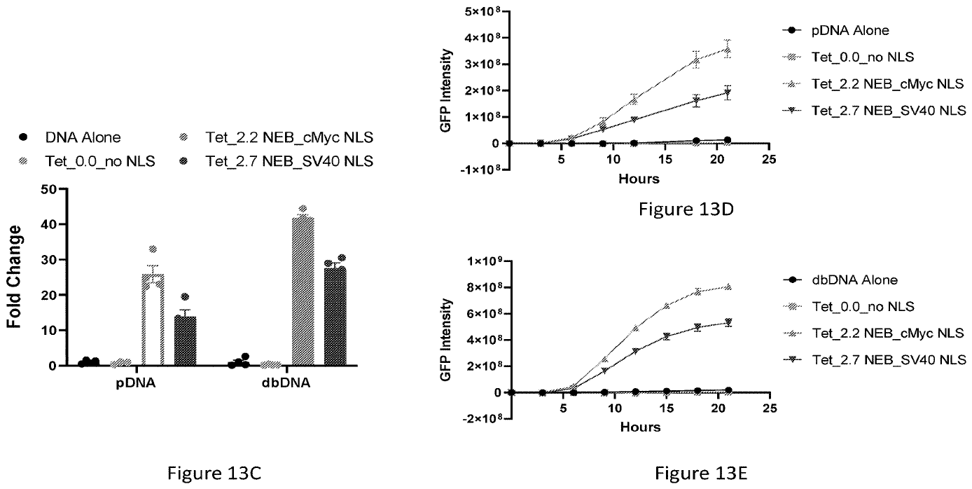

In a follow-up experiment, growth arrested HepG2 cells were transfected with mRNAs expressing TetR fusion proteins in family 2.X (C-terminal NLS) and family 5.X (C-terminal NLS and NES). Plasmid NTDNAs comprising DTSs with TetR binding sites, e.g. the tetracycline response element (TRE) that contains 7 copies of the TetO sequence, were delivered along with mRNA expressing TetR-NLS fusion proteins to growth-arrested HepG2 cells. GFP expression was assessed by measuring GFP fluorescence intensity for 21 h post-transfection. TetO DNA co-transfected with TetR mRNA significantly increases GFP expression compared to the DNA alone negative control for both a dbDNA containing TetO sequences (FIG. 13A) and a pDNA containing TetO sequences (FIG. 13BC). Several of the TetR mRNAs produced significant increases in gene expression (including 2.2, 2.7, 2.11, 5.4, and 5.6), with the magnitude of gene expression increasing with increasing amounts of mRNA (FIGS. 13A and 13B). The mRNA produced with the IVT kit from NEB (2.2 and 2.7 labeled ‘NEB’) outperformed the mRNA produced with the IVT kit from ThermoFisher (all others). Compared to TetO DNA alone, co-transfection with TetR mRNA 2.2 and 2.7 increased gene expression approximately 10-fold to 40-fold at 21 h (FIG. 13C). Over the full 21 h time course, TetO DNA co-transfected with TetR mRNA drives faster and higher GFP expression than DNA alone (FIGS. 13D and 13E).

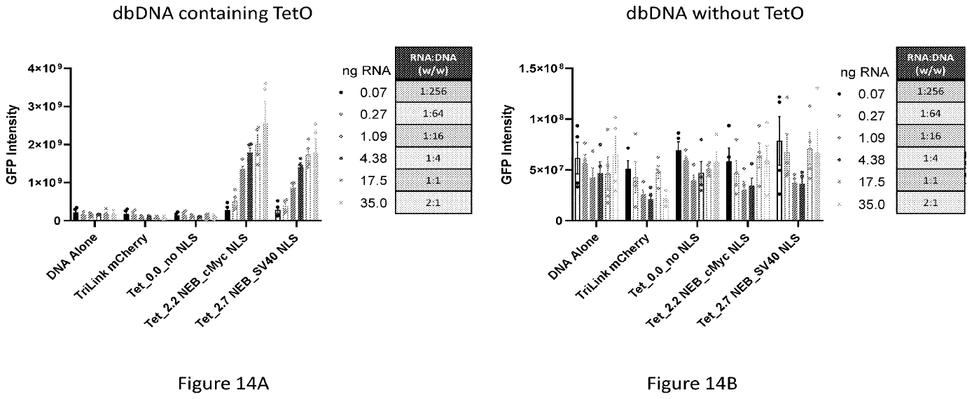

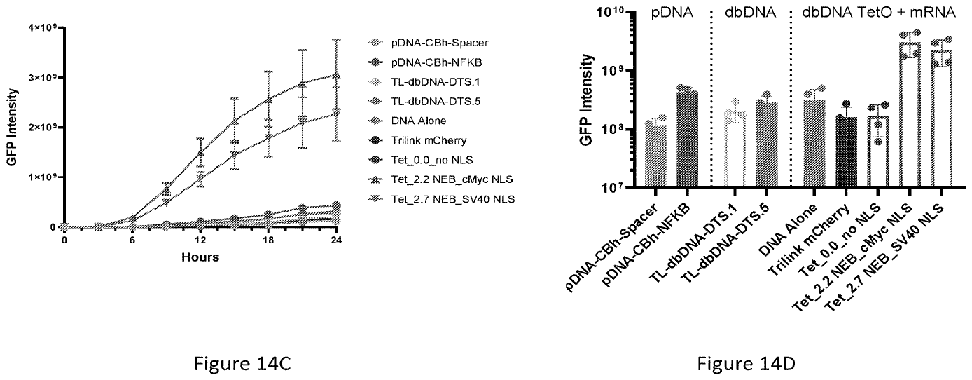

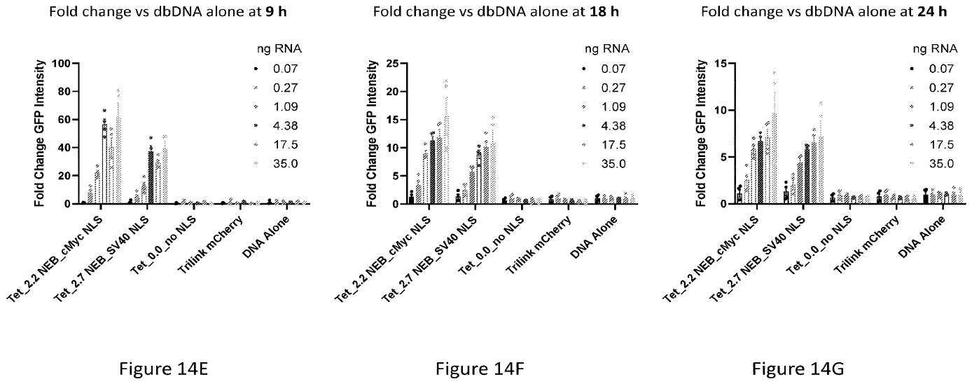

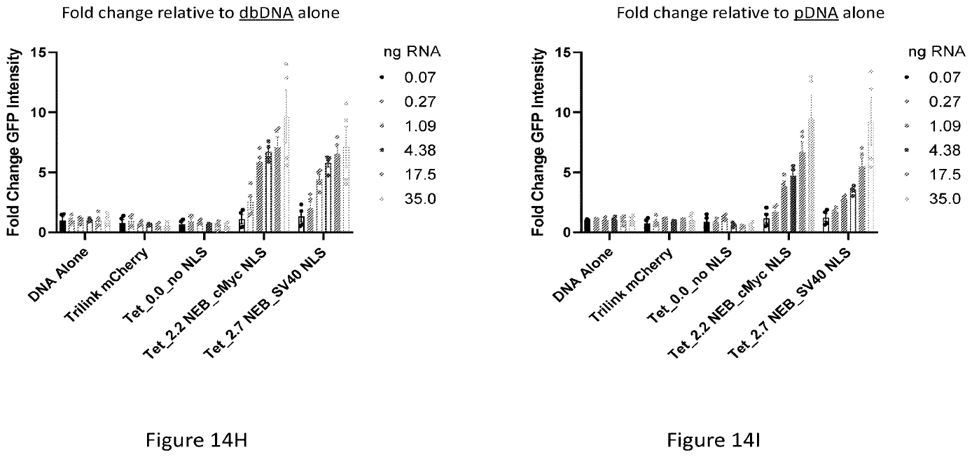

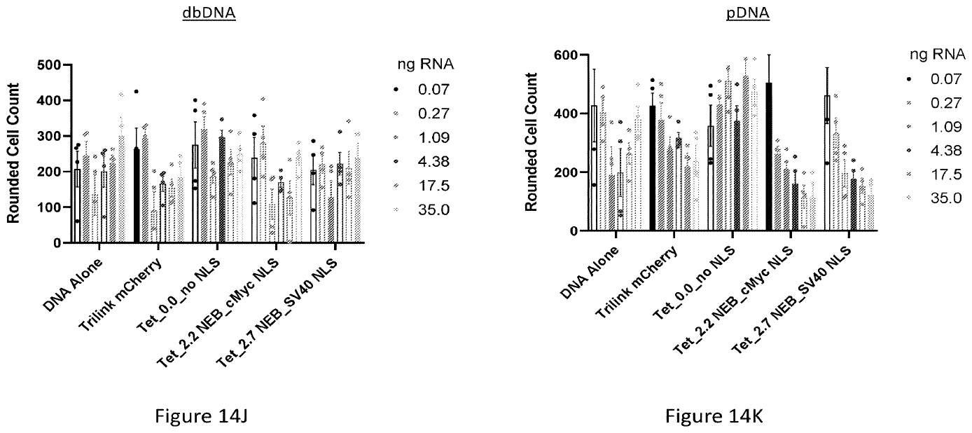

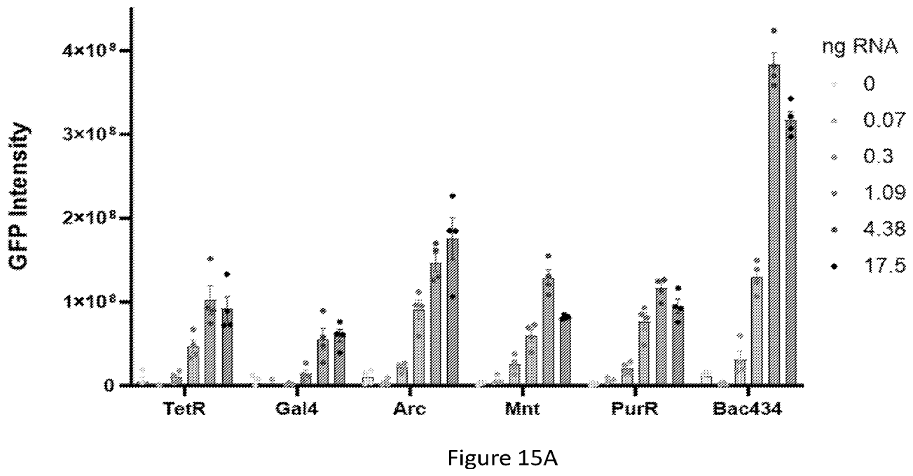

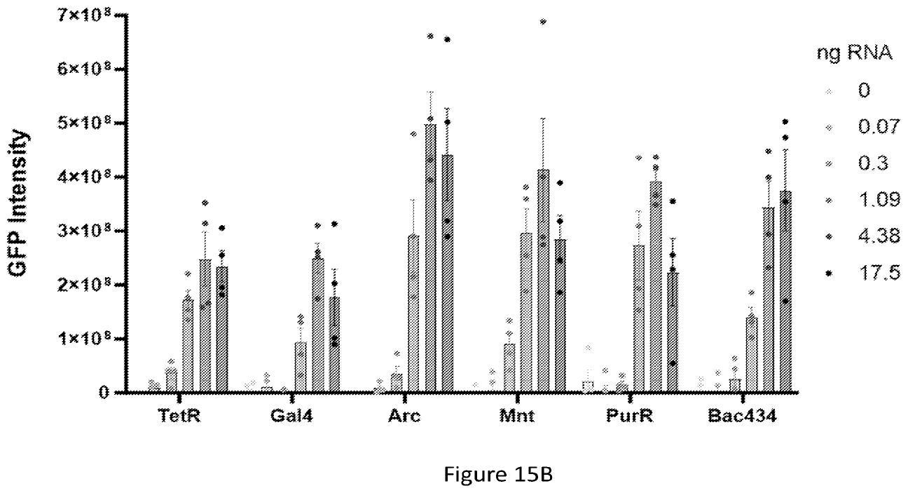

The dose-response and time course of gene expression from the two best TetR-NLS fusion proteins (2.2 and 2.7) were evaluated further. Plasmid NTDNAs comprising DTSs with TetR binding sites, e.g. the tetracycline response element (TRE) that contains 7 copies of the TetO sequence, were delivered along with mRNA expressing TetR-NLS fusion proteins to growth-arrested HepG2 cells. GFP expression was assessed by measuring GFP fluorescence intensity for 24 h post-transfection. dbDNA containing TetO sequences co-transfected with TetR mRNA significantly increases GFP expression compared to the DNA alone negative control (FIG. 14A). The increase in gene expression is dependent on the TetO sequences, because dbDNA lacking the TetO sequences did not show increased gene expression when co-transfected with TetR mRNA (FIG. 14B). Furthermore, the increase in gene expression is also dependent on the NLS fused to TetR, because the Tet 0.0 mRNA that has no NLS did not improve gene expression (FIG. 14A). The magnitude of gene expression increased with increasing amounts of mRNA over a wide range of RNA amounts (FIG. 14A). Over the full 24 h time course, TetO dbDNA co-transfected with TetR mRNA drives faster and higher GFP expression than DNA alone (FIG. 14C). Furthermore, the level of gene expression achieved with TetO dbDNA+TetR mRNA was significantly better than all negative controls by at least 10-fold, including a plasmid DNA containing an NFKB DTS (FIG. 14D). The fold change of increasing GFP intensity with TetO dbDNA+TetR mRNA vs dbDNA alone varies with time; for the high RNA input of Tet_2.2 the fold change decreases from 60-fold at 9 h (FIG. 14E) to 15-fold at 18 h (FIG. 13F) to 10-fold at 24 h (FIG. 14G) as the signal from DNA alone increases (most likely due to low levels of cell division allowing DNA to access the nucleus in a small fraction of cells). The backbone architecture of the DNA was not found to be impactful, with both a dbDNA and pDNA containing TetO sequences showing similar increases in GFP expression when combined with TetR mRNA (FIG. 14H and 14I). Finally, the DNA+mRNA co-transfections were very well tolerated at all DNA: mRNA ratios, with very low rounded cell counts (FIGS. 14J and 14K).

Co-delivery of NTF-expressing mRNA and NTDNA results in nuclear translocation and gene expression in vivo. mRNAs encoding TetR proteins as described above are formulated in LNPs to create mRNA/LNP formulations. DNA (Plasmid, doggybone, or nanoplasmid) comprising the tetracycline response element (TRE) as a DTS and a reporter expression cassette encoding GFP or Factor IX are formulated in LNPs to create DNA/LNP formulations. The mRNA/LNP and DNA/LNP formulations are then mixed to create an mRNA/DNA/LNP preparation. Three cohorts of adult mice (Cohort 1: mRNA/LNP; Cohort 2: DNA/LNP; Cohort 3: mRNA/DNA/LNP admixture) are injected at each of 3 doses (0.1 mg/kg, 0.3 mg/kg, and 1 mg/kg). Serum levels of Factor IX are measured 4 hours, 3 days, and 7 days. GFP is detected by histology post-mortem. No Factor IX or GFP reporter is detected in samples from cohort 1. Very low levels of Factor IX reporter are detected in serum cohort 2, in very few cells by GFP. A 2-5-fold increase in Factor IX reporter and GFP+ cells is observed over cohort 2 in samples from cohort 3, with a dose-response observed.

Coadministration of NTF-expressing mRNA and NTDNA as a single LNP formulation improves DNA nuclear translocation and gene expression. mRNAs encoding TetR proteins are coformulated with DNA (Plasmid, doggybone, or nanoplasmid) comprising the tetracycline response element (TRE) as a DTS and a reporter expression cassette encoding GFP or Factor IX to create mRNA/DNA/LNP formulations. Three cohorts of adult mice (Cohort 1: mRNA/LNP; Cohort 2: DNA/LNP; Cohort 3: mRNA/DNA/LNP formulation) are injected at each of 3 doses (0.1 mg/kg, 0.3 mg/kg, and 1 mg/kg). Serum levels of Factor IX are measured 4 hours, 3 days, and 7 days. GFP is detected by histology post-mortem. No Factor IX or GFP reporter is detected in samples from cohort 1. Very low levels of Factor IX reporter are detected in serum cohort 2, in very few cells by GFP. A 20-fold increase in Factor IX reporter and GFP+ cells over cohort 2 is observed in samples from cohort 3, which is a 5-10-fold increase in efficacy over the admixed preparations.

### Example 8: Assessment of Different NTFs for their Ability to Facilitate DNA Nuclear Translocation and Gene Expression In Vitro. Co-Transfection of DNA with mRNA Expressing Several Different NTFs Improves DNA Nuclear Translocation and Gene Expression

In this study, the ability of several NTFs were assessed for their ability to promote DNA nuclear translocation in vitro. To this end, DNAs comprising a GFP expression cassette were engineered to comprise a DTS that included 7 repeats of the DNA binding domains for TetR, Gal4, Arc, Mnt, PurR, or Bac434. mRNAs comprising a sequence encoding the TetR, Gal4, Arc, Mnt, PurR and Bac434 protein (including the DNA binding domain capable of recognizing and binding the aforementioned DNA binding domains) fused to a nuclear localization signal (NLS) were synthesized.

Growth-arrested HepG2 cells and primary human hepatocytes (PHH) were co-transfected with mRNA encoding the NTF protein along with DNA comprising a cognate DTS and a GFP expression cassette. Each NTF mRNA was paired with its cognate DNA in the co-transfection, e.g., the TetR mRNA was paired with DNA comprising a DTS with 7 repeats of a TetR TFBS, the Gal4 mRNA was paired with DNA comprising a DTS with 7 repeats of a Gal4 TFBS, the Arc mRNA was paired with DNA comprising a DTS with 7 repeats of an Arc TFBS, the Mnt mRNA was paired with DNA comprising a DTS with 7 repeats of a Mnt TFBS, the PurR mRNA was paired with DNA comprising a DTS with 7 repeats of a PurR TFBS, the Bac434 mRNA was paired with DNA comprising a DTS with 7 repeats of a Bac434 TFBS. Eighteen hours after transfection, GFP expression was assessed by measuring GFP fluorescence intensity. All of the NTF proteins increased nuclear translocation and gene expression when paired with their cognate DNA in growth-arrested HepG2 cells (FIG. 15A) and PHH (FIG. 15B). The magnitude of the improvement in gene expression increased with increasing amounts of mRNA in the co-transfection (FIGS. 15A and 15B). Gene expression increases of approximately 10-fold to 40-fold, depending on the NTF and DNA used, were observed with the highest amount of mRNA co-transfected (e.g., 17.5 ng RNA) relative to DNA alone (e.g., 0 ng RNA) in both cell types (FIGS. 15A and 15B).

### Example 9: Investigation into the Impact of Including Multiple TFBS Repeats in the DTS on the Efficacy of DNA Translocation

In this study, DNAs were engineered to comprise DTSs having different numbers of TFBS repeats for TetR or Gal4, and the impact of multiple sequences assessed in vitro by co-transfection of mRNA encoding TetR or Gal4 NTF. Growth-arrested HepG2 cells were co-transfected with mRNA encoding the NTF protein along with DNA comprising a cognate DTS and a GFP expression cassette. Each NTF mRNA was paired with a cognate DNA in the co-transfection, where the cognate DNA comprised a DTS with varying numbers of cognate TFBS repeats. For example, the TetR mRNA was paired with DNAs comprising DTSs with 2, 3, 4, 5, 6, or 7 repeats of a TetR TFBS (e.g., TetO). Twenty-four hours after transfection, GFP expression was assessed by measuring GFP fluorescence intensity.

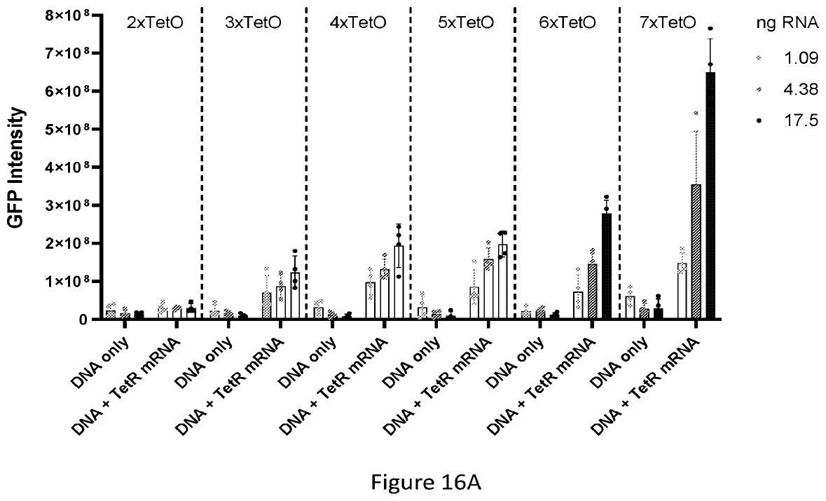

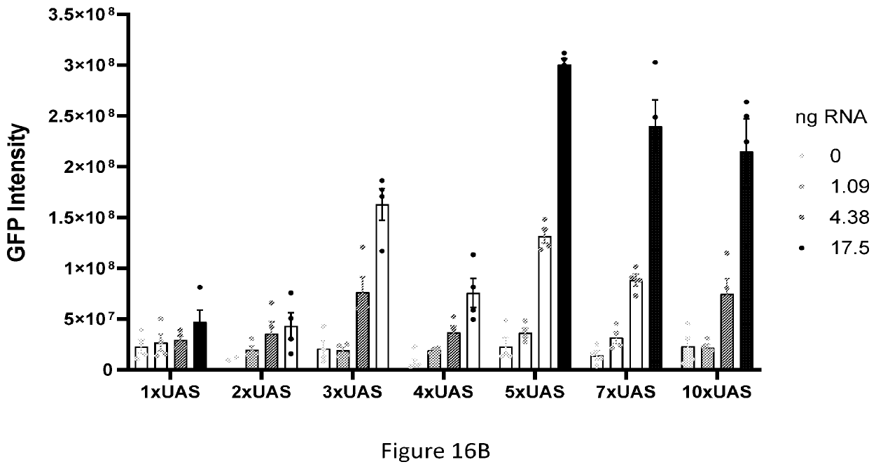

Co-transfection of TetR mRNA with DNA comprising a DTS with 2 repeats of TetO did not substantially increase gene expression over DNA alone, but co-transfection of TetR mRNA with DNA comprising a DTS with 3, 4, 5, 6, or 7 repeats of TetO did substantially increase gene expression compared to DNA alone (FIG. 16A). The increase in gene expression appeared to increase with the number of TetO repeats in the DTS, with 7 repeats producing an increase in GFP expression greater than 10-fold at the highest amount of RNA (e.g., 17.5 ng RNA) compared to DNA alone (FIG. 16A).

The Gal4 mRNA was paired with DNAs comprising DTSs with 1, 2, 3, 4, 5, 7, or 10 repeats of a Gal4 TFBS (e.g., UAS). Twelve hours after transfection, GFP expression was assessed by measuring GFP fluorescence intensity. Co-transfection of Gal4 mRNA with DNA comprising a DTS with 1 or 2 repeats of UAS did not substantially increase gene expression over DNA alone, but co-transfection of Gal4 mRNA with DNA comprising a DTS with 3, 4, 5, 7, or 10 repeats of UAS did substantially increase gene expression compared to DNA alone (FIG. 16B). The increase in gene expression appeared to increase with the number of UAS repeats in the DTS up to 5 repeats, while the increase in gene expression appeared saturable as 5, 7, and 10 repeats performed similarly (FIG. 16B). Co-transfection of Gal4 mRNA and DNA comprising a DTS of 5, 7, or 10 repeats produced an increase in GFP expression greater than 10-fold at the highest amount of RNA (e.g., 17.5 ng RNA) compared to DNA alone (e.g., 0 ng RNA) in HepG2 cells (FIG. 16B).

This study indicates that increasing the number of repeats improves nuclear translocation efficiency, although the effect may be saturable.

### Example 10: Assessment of Different Strategies for the In Vivo Administration of DNA and mRNA

Several different strategies for administering the DNA encoding the EPO transgene and the mRNA encoding the TetR translocation factor were investigated. First, a control LNP was formulated with EPO-TetO DNA alone, where the DNA comprises a DTS with 7xTetO repeats. The same EPO-TetO DNA was also co-formulated into an LNP along with mRNA expressing TetR protein. The same EPO-TetO DNA and mRNA expressing TetR protein were also independently formulated into separate LNPs and then admixed together into the same dosing solution for co-dosing. And finally, in a separate group, the LNP with TetR mRNA was pre-dosed 1 h prior to the LNP with EPO-TetO DNA. Adult BALB/c mice were dosed with LNPs by tail vein injection at 5 mL/kg dosing volume. The serum levels of human EPO protein were measured 3 days post-dose. In all groups, the total amount of DNA administered was 0.5 mg/kg (mpk) and the total amount of mRNA administered was 0.5 mg/kg (mpk), except for the DNA alone group where there was no mRNA dosed.

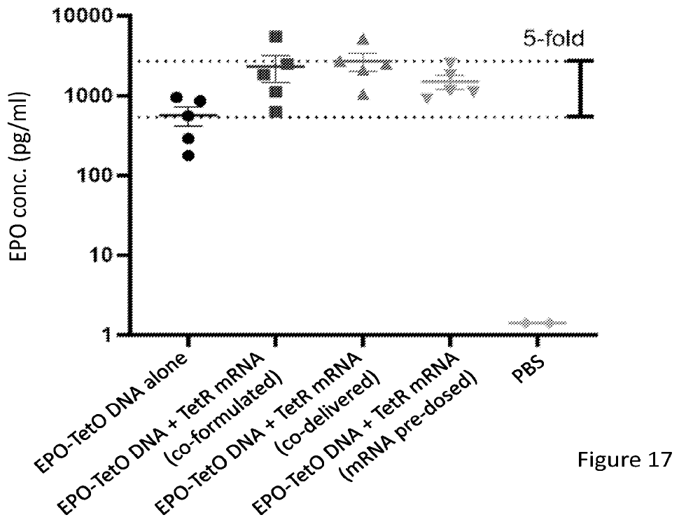

When TetR mRNA was co-formulated into an LNP with EPO-TetO DNA or separately formulated into LNPs, admixed, and co-dosed, the expression of the TetR protein from the mRNA increased the expression of EPO by approximately 5-fold compared to the LNP with DNA alone (FIG. 17A). When the TetR mRNA-containing LNP was pre-dosed 1 h prior to the EPO-TetO DNA-containing LNP, the expression of the TetR protein from the mRNA increased the expression of EPO compared to DNA alone, but not quite to the same degree as was observed with co-formulation or co-dosing (FIG. 17A). All three strategies for incorporating the TetR mRNA successfully increased the expression of the TetO DNA.

### Example 11: Assessment of Different NTFs for their Ability to Facilitate DNA Nuclear Translocation and Gene Expression

LNPs were co-formulated with DNA and mRNA expressing several different NTFs to assess the ability of those other NTFs to improve DNA nuclear translocation and gene expression. Adult BALB/c mice were dosed with LNPs, either formulated with DNA alone or co-formulated with DNA and mRNA, by tail vein injection at 5 mL/kg dosing volume. The LNPs with DNA alone were administered at 0.5 mg/kg DNA, and the co-formulated LNPs were administered at 0.5 mg/kg DNA and 1.5 mg/kg mRNA (3:1 RNA:DNA weight ratio). In this study, each DNA payload comprises a human Factor IX (FIX) expression cassette. Accordingly, the plasma levels of human FIX protein were measured up to 28 days post-dose.

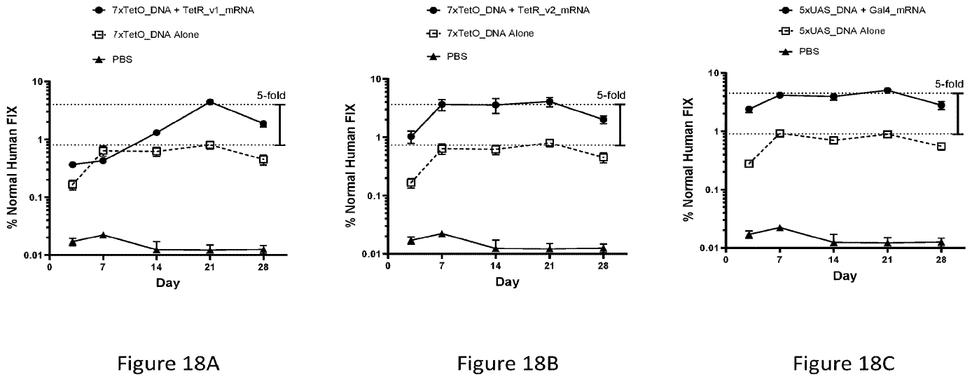

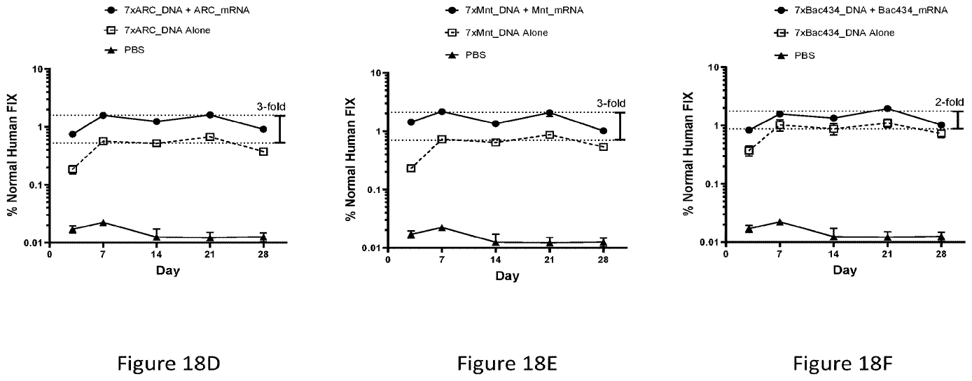

Dosing co-formulated LNPs with TetR mRNA and DNA comprising a TetO DTS increased FIX levels by approximately 5-fold compared to the TetO DNA alone LNP. Interestingly, the “v2” TetR mRNA provided a faster and more sustained increase in gene expression compared to the “v1” TetR mRNA (FIGS. 18A and 18B). Dosing co-formulated LNPs with Gal4 mRNA and DNA having a DTS comprising a UAS sequence increased FIX levels by approximately 5-fold compared to the UAS DNA alone LNP (FIG. 18C). Dosing co-formulated LNPs with Arc mRNA and a DNA having a DTS comprising an Arc binding sequence increased FIX levels by approximately 3-fold compared to the Arc DNA alone LNP (FIG. 18D). Dosing co-formulated LNPs with Mnt mRNA and a DNA having a DTS comprising a Mnt binding sequence increased FIX levels by approximately 3-fold compared to the Mnt DNA alone LNP (FIG. 18E). Dosing co-formulated LNPs with Bac434 mRNA and a DNA having a DTS comprising a Bac434 binding sequence increased FIX levels by approximately 2-fold compared to the Bac434 DNA alone LNP (FIG. 18F). This data demonstrates that multiple families of NTFs are able to improve nuclear translocation and gene expression in mice, similar to the observations in vitro.

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. Moreover, nothing disclosed herein is intended to be dedicated to the public regardless of whether such disclosure is explicitly recited in the claims.

The scope of the present invention, therefore, is not intended to be limited to the exemplary embodiments shown and described herein. Rather, the scope and spirit of present invention is embodied by the appended claims. In the claims, 35 U.S.C. § 112 (f) or 35 U.S.C. § 112 (6) is expressly defined as being invoked for a limitation in the claim only when the exact phrase “means for” or the exact phrase “step for” is recited at the beginning of such limitation in the claim; if such exact phrase is not used in a limitation in the claim, then 35 U.S.C. § 112 (f) or 35 U.S.C. § 112 (6) is not invoked.

1. A method of delivering a cargo nucleic acid sequence into the nucleus of a cell, the method comprising:
contacting the cell with a nuclear targeted deoxyribonucleic acid (NTDNA) comprising
a) a DNA nuclear targeting sequence (DTS) comprising a targeting factor binding sequence (TFBS) for a nuclear targeting factor (NTF), and
b) a cargo nucleic acid heterologous to the DTS;
to deliver the cargo nucleic acid sequence to the nucleus of the cell.

2. The method according to claim 1, wherein the cargo nucleic acid sequence comprises an expression cassette.

3. The method according to claim 1, wherein the cargo nucleic acid comprises a promoter that is heterologous to the DTS.

4. The method according to claim 1, wherein the cargo nucleic acid comprises a coding sequence that is heterologous to the DTS.

5. The method according to claim 1, wherein the cargo nucleic acid sequence is flanked by sequences that are homologous to genomic sequences of the cell.

6. The method according to claim 1, wherein the DTS comprises 2 or more different targeting factor binding sequences (TFBS).

7. The method according to claim 1, wherein the DTS comprises 2 or more copies of the same TFBS.

8. The method according to claim 6, wherein the DTS comprises 5-100 nucleotides between each TFBS.

9. The method according to claim 1, wherein the DTS comprises one or more TFBSs selected from Table 1 or Table 2.

10. The method according to claim 1, wherein the DTS comprises a TFBS for a NTF selected from the group consisting of HNF1A, PPARA, HNF4A, CEBPA, NR3C1, ONECUT1, TBP, NFkB, a TetR protein, a ZF-CCR5 protein, a TALE protein, a GAL4 protein, an Arc protein, a Mnt protein, a PurR protein, a Bac434 protein, a GCN4 protein, a LacR protein, a I-SceI D44A protein, and a Cas9 protein.

11. The method according to claim 10, wherein:
(a) when the TFBS is for HNF1A, the sequence is selected from GGTTAATAATTAAC and AGTATGGTTAATGATCTACAG;
(b) when the TFBS is for PPARA, the sequence is selected from AACTAGGTCAAAGGTCA, CAAAACTAGGTCAAAGGTCA, and AACTAGGTCAAAGGTCAAAG;
(c) when the TFBS is for HNF4A, the sequence is selected from TCGAGCGCTGGGCAAAGGTCACCTGC, TCGAGCGCAGGTCAAAGGTCACCTGC, and AGGTCAAAGTCCA;
(d) when the TFBS is for CEBPA, the sequence is TGGTATGATTTTGTAATGGGGTAGGA;
(e) when the TFBS is for NR3C1, the sequence is selected from AGAACAAAATGTTCT, AAGAACAAAATGTTCTT, AGAACATTTTGTACG, and AAGAACATTTTGTACGT;
(f) when the TFBS is for ONECUT1, the sequence is GTCTGCTAAGTCAATAATCAGAAT;
(g) when the TFBS is for TBP, the sequence is TATAAAA;
(h) when the TFBS is for NFKB, the sequence is GGGACTTTCC;
(i) when the TFBS is for a TetR protein, the sequence comprises TCCCTATCAGTGATAGAGA;
(j) when the TFBS is for a ZF-CCR5 protein, the sequence comprises AAACTGCAAAAG;
(k) when the TFBS is for a TALE protein, the sequence comprises TTCATTACACCTGCAGCT, ATAAACCCCCTCCAA, or TCGAGTTTACTCCCTATCAGTGATAGAGAACG;
(l) when the TFBS is for a GAL4 protein, the sequence comprises CGG-N11-CCG;
(m) when the TFBS is for an Arc protein, the sequence comprises RYRVTAGANNNNNTCTABYRY;
(n) when the TFBS is for a Mnt protein, the sequence comprises GGNCCACNGTGGNCC;
(o) when the TFBS is for a PurR protein, the sequence comprises ACGCAAACGTTTTCGT;
(p) when the TFBS is for Bac434 protein, the sequence comprises ACAAGAAAGTTTGT, ACAAGATACATTGT, or ACAAGAAAAACTGT;
(q) when the TFBS is for a GCN4 protein, the sequence comprises TGACTC;
(r) when the TFBS is for a LacR protein, the sequence comprises TTGTTATCCGCTCACAA; and(s)
when the TFBS is for an I-SceI protein the sequence comprises TAGGGATAACAGGGTAAT.

12. (canceled)

13. (canceled)

14. The method according to claim 1, wherein the method further comprises contacting the cell with the NTF.

15. The method according to claim 14, wherein the NTF is provided to the cell as an mRNA.

16. The method according to claim 14, wherein the contacting with NTDNA occurs before the contacting with NTF.

17. The method according to claim 14, wherein the contacting with NTDNA occurs after the contacting with NTF.

18. The method according to claim 14, wherein the contacting with NTDNA occurs concurrently with the contacting with NTF.

19. The method according to claim 18, wherein the contacting comprises contacting with a composition comprising the NTDNA and the NTF.

20. The method according to claim 19, wherein the composition comprises a lipid nanoparticle (LNP) co-formulated with the NTDNA and NTF.

21. The method according to claim 19, wherein the pharmaceutic composition comprises a first lipid nanoparticle (LNP) formulated with the NTDNA and a second LNP formulated with the NTF.

22-26. (canceled)

27. The method according to claim 1, wherein the method further comprises contacting the cell with an inducing agent that activates the NTF to which the DTS binds to mediate nuclear entry of the NTDNA.

28-70. (canceled)


SEQUENCE LISTING (455 sequences)

SEQ ID NO: 1 - skipped

SEQ ID NO: 2 - DNA, 10 bp, synthetic construct
  source 1..10: mol_type = other DNA
attgcgcaat

SEQ ID NO: 3 - DNA, 10 bp, synthetic construct
  source 1..10: mol_type = other DNA
attgtgcaat

SEQ ID NO: 4 - DNA, 10 bp, synthetic construct
  source 1..10: mol_type = other DNA
attgcacaat

SEQ ID NO: 5 - DNA, 10 bp, synthetic construct
  source 1..10: mol_type = other DNA
attacaaaat

SEQ ID NO: 6 - DNA, 13 bp, synthetic construct
  source 1..13: mol_type = other DNA
tgtttgttaaggc

SEQ ID NO: 7 - DNA, 26 bp, synthetic construct
  source 1..26: mol_type = other DNA
tggtatgattttgtaatggggtagga

SEQ ID NO: 8 - skipped

SEQ ID NO: 9 - DNA, 10 bp, synthetic construct
  source 1..10: mol_type = other DNA
ctgacgtcag

SEQ ID NO: 10-13 - skipped (4 entries)

SEQ ID NO: 14 - DNA, 12 bp, synthetic construct
  source 1..12: mol_type = other DNA
ccatgaactttg

SEQ ID NO: 15 - DNA, 12 bp, synthetic construct
  source 1..12: mol_type = other DNA
caaacgtggttt

SEQ ID NO: 16 - DNA, 13 bp, synthetic construct
  source 1..13: mol_type = other DNA
tccacgtggtatt

SEQ ID NO: 17 - DNA, 11 bp, synthetic construct
  source 1..11: mol_type = other DNA
tacacgtaatc

SEQ ID NO: 18 - DNA, 11 bp, synthetic construct
  source 1..11: mol_type = other DNA
cacacgtgatc

SEQ ID NO: 19 - skipped

SEQ ID NO: 20 - DNA, 18 bp, synthetic construct
  source 1..18: mol_type = other DNA
aaaaatggcgccaaaatg

SEQ ID NO: 21 - DNA, 10 bp, synthetic construct
  source 1..10: mol_type = other DNA
tccctaggga

SEQ ID NO: 22 - DNA, 10 bp, synthetic construct
  source 1..10: mol_type = other DNA
cgcccacgca

SEQ ID NO: 23 - DNA, 14 bp, synthetic construct
  source 1..14: mol_type = other DNA
tacgcccacgcatt

SEQ ID NO: 24 - skipped

SEQ ID NO: 25 - DNA, 12 bp, synthetic construct
  source 1..12: mol_type = other DNA
aacccggaagtg

SEQ ID NO: 26 - skipped

SEQ ID NO: 27 - DNA, 17 bp, synthetic construct
  source 1..17: mol_type = other DNA
cacttccgccggaagtg

SEQ ID NO: 28 - skipped

SEQ ID NO: 29 - DNA, 18 bp, synthetic construct
  source 1..18: mol_type = other DNA
accggaagtacttccggt

SEQ ID NO: 30 - skipped

SEQ ID NO: 31 - DNA, 10 bp, synthetic construct
  source 1..10: mol_type = other DNA
tgtttacttt

SEQ ID NO: 32 - DNA, 10 bp, synthetic construct
  source 1..10: mol_type = other DNA
ttgtttactt

SEQ ID NO: 33-36 - skipped (4 entries)

SEQ ID NO: 37 - DNA, 13 bp, synthetic construct
  source 1..13: mol_type = other DNA
gttaatgattaac

SEQ ID NO: 38 - DNA, 13 bp, synthetic construct
  source 1..13: mol_type = other DNA
gttaataatctac

SEQ ID NO: 39 - DNA, 13 bp, synthetic construct
  source 1..13: mol_type = other DNA
gttacttattctc

SEQ ID NO: 40 - DNA, 14 bp, synthetic construct
  source 1..14: mol_type = other DNA
ggttaataattaac

SEQ ID NO: 41 - DNA, 15 bp, synthetic construct
  source 1..15: mol_type = other DNA
aattatttattacca

SEQ ID NO: 42 - DNA, 21 bp, synthetic construct
  source 1..21: mol_type = other DNA
agtatggttaatgatctacag

SEQ ID NO: 43 - DNA, 18 bp, synthetic construct
  source 1..18: mol_type = other DNA
tggttaatattcaccagc

SEQ ID NO: 44 - DNA, 24 bp, synthetic construct
  source 1..24: mol_type = other DNA
gtttatcagtgactagtcattgat

SEQ ID NO: 45 - DNA, 30 bp, synthetic construct
  source 1..30: mol_type = other DNA
tgatagccaactgcagctaataataaacca

SEQ ID NO: 46 - DNA, 25 bp, synthetic construct
  source 1..25: mol_type = other DNA
atattttagagaagaattaaccttt

SEQ ID NO: 47 - DNA, 13 bp, synthetic construct
  source 1..13: mol_type = other DNA
agtccaaagttca

SEQ ID NO: 48 - DNA, 26 bp, synthetic construct
  source 1..26: mol_type = other DNA
tcgagcgctgggcaaaggtcacctgc

SEQ ID NO: 49 - DNA, 27 bp, synthetic construct
  source 1..27: mol_type = other DNA
tcgaagggcaggggtcaagggttcagt

SEQ ID NO: 50 - DNA, 26 bp, synthetic construct
  source 1..26: mol_type = other DNA
tcgagcgcaggtcaaaggtcacctgc

SEQ ID NO: 51 - DNA, 27 bp, synthetic construct
  source 1..27: mol_type = other DNA
tcgagcgcaggtcaaaaggtcacctgc

SEQ ID NO: 52 - DNA, 13 bp, synthetic construct
  source 1..13: mol_type = other DNA
aggttaaaggtct

SEQ ID NO: 53 - DNA, 13 bp, synthetic construct
  source 1..13: mol_type = other DNA
aggtcaaagtcca

SEQ ID NO: 54 - DNA, 18 bp, synthetic construct
  source 1..18: mol_type = other DNA
tgggtccagagggcaaaa

SEQ ID NO: 55 - DNA, 22 bp, synthetic construct
  source 1..22: mol_type = other DNA
cgccccagcacacatgatcaga

SEQ ID NO: 56 - DNA, 26 bp, synthetic construct
  source 1..26: mol_type = other DNA
aacacgggaggtcaaagattgcgccc

SEQ ID NO: 57 - skipped

SEQ ID NO: 58 - DNA, 12 bp, synthetic construct
  source 1..12: mol_type = other DNA
gaaagtgaaagt

SEQ ID NO: 59-60 - skipped (2 entries)

SEQ ID NO: 61 - DNA, 11 bp, synthetic construct
  source 1..11: mol_type = other DNA
agcccccggcg

SEQ ID NO: 62-63 - skipped (2 entries)

SEQ ID NO: 64 - DNA, 12 bp, synthetic construct
  source 1..12: mol_type = other DNA
tttccatggaaa

SEQ ID NO: 65 - DNA, 15 bp, synthetic construct
  source 1..15: mol_type = other DNA
ttggctatatgccaa

SEQ ID NO: 66-67 - skipped (2 entries)

SEQ ID NO: 68 - DNA, 13 bp, synthetic construct
  source 1..13: mol_type = other DNA
tccattgatttag

SEQ ID NO: 69 - DNA, 14 bp, synthetic construct
  source 1..14: mol_type = other DNA
aaaaaatcaataat

SEQ ID NO: 70 - DNA, 24 bp, synthetic construct
  source 1..24: mol_type = other DNA
gaaaaaaaaatcaatatcgggcct

SEQ ID NO: 71 - DNA, 24 bp, synthetic construct
  source 1..24: mol_type = other DNA
gtctgctaagtcaataatcagaat

SEQ ID NO: 72 - DNA, 12 bp, synthetic construct
  source 1..12: mol_type = other DNA
gtcatgcgtgac

SEQ ID NO: 73 - DNA, 14 bp, synthetic construct
  source 1..14: mol_type = other DNA
cccccttgggcccc

SEQ ID NO: 74 - skipped

SEQ ID NO: 75 - DNA, 14 bp, synthetic construct
  source 1..14: mol_type = other DNA
aatatgcaaattag

SEQ ID NO: 76 - DNA, 16 bp, synthetic construct
  source 1..16: mol_type = other DNA
ttatgcatatgcataa

SEQ ID NO: 77 - DNA, 23 bp, synthetic construct
  source 1..23: mol_type = other DNA
atatgattatgcaaatttataga

SEQ ID NO: 78 - DNA, 14 bp, synthetic construct
  source 1..14: mol_type = other DNA
aggtcatcaggtca

SEQ ID NO: 79 - DNA, 13 bp, synthetic construct
  source 1..13: mol_type = other DNA
aggtcaaaggtca

SEQ ID NO: 80 - DNA, 13 bp, synthetic construct
  source 1..13: mol_type = other DNA
aggtcaatgacct

SEQ ID NO: 81 - DNA, 13 bp, synthetic construct
  source 1..13: mol_type = other DNA
gtgtcaaaggtca

SEQ ID NO: 82 - DNA, 17 bp, synthetic construct
  source 1..17: mol_type = other DNA
aactaggtcaaaggtca

SEQ ID NO: 83 - DNA, 20 bp, synthetic construct
  source 1..20: mol_type = other DNA
caaaactaggtcaaaggtca

SEQ ID NO: 84 - DNA, 20 bp, synthetic construct
  source 1..20: mol_type = other DNA
aactaggtcaaaggtcaaag

SEQ ID NO: 85-86 - skipped (2 entries)

SEQ ID NO: 87 - DNA, 11 bp, synthetic construct
  source 1..11: mol_type = other DNA
gccacgccccc

SEQ ID NO: 88 - DNA, 13 bp, synthetic construct
  source 1..13: mol_type = other DNA
aaaagaggaagtg

SEQ ID NO: 89 - DNA, 17 bp, synthetic construct
  source 1..17: mol_type = other DNA
aaacaataacattgttt

SEQ ID NO: 90-91 - skipped (2 entries)

SEQ ID NO: 92 - DNA, 10 bp, synthetic construct
  source 1..10: mol_type = other DNA
acttcaaagg

SEQ ID NO: 93 - skipped

SEQ ID NO: 94 - DNA, 20 bp, synthetic construct
  source 1..20: mol_type = other DNA
ggacatgcccgggcatgtcc

SEQ ID NO: 95 - DNA, 10 bp, synthetic construct
  source 1..10: mol_type = other DNA
gtcacgtgac

SEQ ID NO: 96 - skipped

SEQ ID NO: 97 - DNA, 12 bp, synthetic construct
  source 1..12: mol_type = other DNA
gatgacgtcatc

SEQ ID NO: 98 - DNA, 11 bp, synthetic construct
  source 1..11: mol_type = other DNA
gccgccatttt

SEQ ID NO: 99 - DNA, 14 bp, synthetic construct
  source 1..14: mol_type = other DNA
gcacccaagggtgc

SEQ ID NO: 100 - DNA, 13 bp, synthetic construct
  source 1..13: mol_type = other DNA
agtccaaagttca

SEQ ID NO: 101 - DNA, 13 bp, synthetic construct
  source 1..13: mol_type = other DNA
tgacctttgacct

SEQ ID NO: 102 - DNA, 15 bp, synthetic construct
  source 1..15: mol_type = other DNA
aggtcacagtgacct

SEQ ID NO: 103 - skipped

SEQ ID NO: 104 - DNA, 15 bp, synthetic construct
  source 1..15: mol_type = other DNA
ggaacattatgtacc

SEQ ID NO: 105 - DNA, 15 bp, synthetic construct
  source 1..15: mol_type = other DNA
agaacaaaatgttct

SEQ ID NO: 106 - DNA, 17 bp, synthetic construct
  source 1..17: mol_type = other DNA
aagaacaaaatgttctt

SEQ ID NO: 107 - DNA, 15 bp, synthetic construct
  source 1..15: mol_type = other DNA
agaacattttgtacg

SEQ ID NO: 108 - DNA, 17 bp, synthetic construct
  source 1..17: mol_type = other DNA
aagaacattttgtacgt

SEQ ID NO: 109 - DNA, 16 bp, synthetic construct
  source 1..16: mol_type = other DNA
agaacatccctgtaca

SEQ ID NO: 110 - DNA, 15 bp, synthetic construct
  source 1..15: mol_type = other DNA
agaacatgatgttct

SEQ ID NO: 111 - DNA, 24 bp, synthetic construct
  source 1..24: mol_type = other DNA
aatagaggtcactaaaggtcaagc

SEQ ID NO: 112 - DNA, 26 bp, synthetic construct
  source 1..26: mol_type = other DNA
aaactaggtcacgaaaggtcaaagtc

SEQ ID NO: 113 - DNA, 16 bp, synthetic construct
  source 1..16: mol_type = other DNA
tgacctctactgacct

SEQ ID NO: 114 - DNA, 21 bp, synthetic construct
  source 1..21: mol_type = other DNA
atcacgtgattatcacgtgat

SEQ ID NO: 115 - DNA, 10 bp, synthetic construct
  source 1..10: mol_type = other DNA
gggaatttcc

SEQ ID NO: 116 - DNA, 10 bp, synthetic construct
  source 1..10: mol_type = other DNA
gggactttcc

SEQ ID NO: 117 - DNA, 25 bp, synthetic construct
  source 1..25: mol_type = other DNA
aggcagagggcagaaaggtcaaggg

SEQ ID NO: 118 - DNA, 21 bp, synthetic construct
  source 1..21: mol_type = other DNA
cagaggtcacagagttcaagc

SEQ ID NO: 119 - DNA, 22 bp, synthetic construct
  source 1..22: mol_type = other DNA
cagagttcatgagagttcaagc

SEQ ID NO: 120 - DNA, 29 bp, synthetic construct
  source 1..29: mol_type = other DNA
gcccccagggctgagtgacagaaaaacag

SEQ ID NO: 121 - DNA, 29 bp, synthetic construct
  source 1..29: mol_type = other DNA
gcattacagactgggtgacagagtgagac

SEQ ID NO: 122 - DNA, 29 bp, synthetic construct
  source 1..29: mol_type = other DNA
aattatggttctgggtgattcaagtaaca

SEQ ID NO: 123 - DNA, 29 bp, synthetic construct
  source 1..29: mol_type = other DNA
gcaataaaatctgggtcacaggagttgga

SEQ ID NO: 124 - DNA, 29 bp, synthetic construct
  source 1..29: mol_type = other DNA
cctcttctctgtgggtgaccagcgtccta

SEQ ID NO: 125 - DNA, 14 bp, synthetic construct
  source 1..14: mol_type = other DNA
aggtcatcaggtca

SEQ ID NO: 126 - DNA, 13 bp, synthetic construct
  source 1..13: mol_type = other DNA
aggtcaaaggtca

SEQ ID NO: 127 - DNA, 13 bp, synthetic construct
  source 1..13: mol_type = other DNA
aggtcaatgacct

SEQ ID NO: 128 - DNA, 13 bp, synthetic construct
  source 1..13: mol_type = other DNA
gtgtcaaaggtca

SEQ ID NO: 129 - DNA, 17 bp, synthetic construct
  source 1..17: mol_type = other DNA
aactaggtcaaaggtca

SEQ ID NO: 130 - DNA, 20 bp, synthetic construct
  source 1..20: mol_type = other DNA
caaaactaggtcaaaggtca

SEQ ID NO: 131 - DNA, 20 bp, synthetic construct
  source 1..20: mol_type = other DNA
aactaggtcaaaggtcaaag

SEQ ID NO: 132 - DNA, 16 bp, synthetic construct
  source 1..16: mol_type = other DNA
tgaccttttgaccttt

SEQ ID NO: 133 - DNA, 14 bp, synthetic construct
  source 1..14: mol_type = other DNA
tgacctttgacccc

SEQ ID NO: 134 - DNA, 10 bp, synthetic construct
  source 1..10: mol_type = other DNA
atcaccccac

SEQ ID NO: 135 - DNA, 10 bp, synthetic construct
  source 1..10: mol_type = other DNA
atcacgtgat

SEQ ID NO: 136 - DNA, 16 bp, synthetic construct
  source 1..16: mol_type = other DNA
gagttcattgagttca

SEQ ID NO: 137 - DNA, 21 bp, synthetic construct
  source 1..21: mol_type = other DNA
caggggtcatcgggttcaaac

SEQ ID NO: 138 - DNA, 161 bp, synthetic construct
  source 1..161: mol_type = other DNA
ggttaataattaacagattactactgatacctcttctctgtgggtgaccagcgtcctaagattactactgataaactaggtcaaaggtcaagattactactgataagtatggttaatgatctacagagattactactgatacaaaactaggtcaaaggtca

SEQ ID NO: 139 - DNA, 157 bp, synthetic construct
  source 1..157: mol_type = other DNA
tgtttactttagattactactgatatcgagcgcaggtcaaaggtcacctgcagattactactgataggttaataattaacagattactactgatatcgagcgctgggcaaaggtcacctgcagattactactgataagtatggttaatgatctacag

SEQ ID NO: 140 - DNA, 110 bp, synthetic construct
  source 1..110: mol_type = other DNA
gggactttccagattactactgatagggactttccagattactactgatagggactttccagattactactgatagggactttccagattactactgatagggactttcc

SEQ ID NO: 141 - DNA, 149 bp, synthetic construct
  source 1..149: mol_type = other DNA
ctgacgtcagagattactactgatacaaaactaggtcaaaggtcaagattactactgatagtctgctaagtcaataatcagaatagattactactgatacgccccagcacacatgatcagaagattactactgataaggtcaaaggtca

SEQ ID NO: 142 - DNA, 15 bp, synthetic construct
  source 1..15: mol_type = other DNA
yctatcantgataga

SEQ ID NO: 143 - DNA, 19 bp, synthetic construct
  source 1..19: mol_type = other DNA
tccctatcagtgatagaga

SEQ ID NO: 144 - DNA, 32 bp, synthetic construct
  source 1..32: mol_type = other DNA
tcgagtttactccctatcagtgatagagaacg

SEQ ID NO: 145 - DNA, 15 bp, synthetic construct
  source 1..15: mol_type = other DNA
yctatcantgataga

SEQ ID NO: 146 - DNA, 19 bp, synthetic construct
  source 1..19: mol_type = other DNA
tccctatcagtgatagaga

SEQ ID NO: 147 - DNA, 82 bp, synthetic construct
  source 1..82: mol_type = other DNA
ctcgagtttactccctatcagtgatagagaacgtatgtcgagtttactccctatcagtgatagagaacgatgtcgagtttac

SEQ ID NO: 148 - DNA, 118 bp, synthetic construct
  source 1..118: mol_type = other DNA
ctcgagtttactccctatcagtgatagagaacgtatgtcgagtttactccctatcagtgatagagaacgatgtcgagtttactccctatcagtgatagagaacgtatgtcgagtttac

SEQ ID NO: 149 - DNA, 154 bp, synthetic construct
  source 1..154: mol_type = other DNA
ctcgagtttactccctatcagtgatagagaacgtatgtcgagtttactccctatcagtgatagagaacgatgtcgagtttactccctatcagtgatagagaacgtatgtcgagtttactccctatcagtgatagagaacgtatgtcgagtttac

SEQ ID NO: 150 - DNA, 189 bp, synthetic construct
  source 1..189: mol_type = other DNA
ctcgagtttactccctatcagtgatagagaacgtatgtcgagtttactccctatcagtgatagagaacgatgtcgagtttactccctatcagtgatagagaacgtatgtcgagtttactccctatcagtgatagagaacgtatgtcgagtttactccctatcagtgatagagaacgtatgtcgagttta

SEQ ID NO: 151 - DNA, 225 bp, synthetic construct
  source 1..225: mol_type = other DNA
ctcgagtttactccctatcagtgatagagaacgtatgtcgagtttactccctatcagtgatagagaacgatgtcgagtttactccctatcagtgatagagaacgtatgtcgagtttactccctatcagtgatagagaacgtatgtcgagtttactccctatcagtgatagagaacgtatgtcgagtttatccctatcagtgatagagaacgtatgtcgagtttac

SEQ ID NO: 152 - DNA, 329 bp, synthetic construct
  source 1..329: mol_type = other DNA
ctcgagtttactccctatcagtgatagagaacgtatgtcgagtttactccctatcagtgatagagaacgatgtcgagtttactccctatcagtgatagagaacgtatgtcgagtttactccctatcagtgatagagaacgtatgtcgagtttactccctatcagtgatagagaacgtatgtcgagtttatccctatcagtgatagagaacgtatgtcgagtttactccctatcagtgatagagaacgtatgtcgaggtaggcgtgtacggtgggaggcctatataagcagagctcgtttagtgaaccgtcagatcgcctggagaattca

SEQ ID NO: 153 - DNA, 259 bp, synthetic construct
  source 1..259: mol_type = other DNA
ctcgagtttactccctatcagtgatagagaacgtatgtcgagtttactccctatcagtgatagagaacgatgtcgagtttactccctatcagtgatagagaacgtatgtcgagtttactccctatcagtgatagagaacgtatgtcgagtttactccctatcagtgatagagaacgtatgtcgagtttatccctatcagtgatagagaacgtatgtcgagtttactccctatcagtgatagagaacgtatgtcgaggta

SEQ ID NO: 154 - DNA, 32 bp, synthetic construct; identical to SEQ ID NO: 144
  source 1..32: mol_type = other DNA

SEQ ID NO: 155 - DNA, 17 bp, synthetic construct
  source 1..17: mol_type = other DNA
cggaggactgtcctccg

SEQ ID NO: 156 - skipped

SEQ ID NO: 157 - DNA, 17 bp, synthetic construct
  source 1..17: mol_type = other DNA
cggtggcttctaatccg

SEQ ID NO: 158 - DNA, 17 bp, synthetic construct
  source 1..17: mol_type = other DNA
cgggtgacagccctccg

SEQ ID NO: 159 - DNA, 17 bp, synthetic construct
  source 1..17: mol_type = other DNA
cggaggagagtcttccg

SEQ ID NO: 160 - DNA, 17 bp, synthetic construct
  source 1..17: mol_type = other DNA
cggagtactgtcctccg

SEQ ID NO: 161 - DNA, 17 bp, synthetic construct
  source 1..17: mol_type = other DNA
cggaggactgtcctccg

SEQ ID NO: 162 - DNA, 37 bp, synthetic construct
  source 1..37: mol_type = other DNA
acactagagtcggaggactgtcctccgtgagtcctag

SEQ ID NO: 163 - DNA, 44 bp, synthetic construct
  source 1..44: mol_type = other DNA
cggaggactgtcctccgtgagtcctagcggaggactgtcctccg

SEQ ID NO: 164 - DNA, 71 bp, synthetic construct
  source 1..71: mol_type = other DNA
cggaggactgtcctccgtgagtcctagcggaggactgtcctccgtcttcacaggcggaggactgtcctccg

SEQ ID NO: 165 - DNA, 98 bp, synthetic construct
  source 1..98: mol_type = other DNA
cggaggactgtcctccgtgagtcctagcggaggactgtcctccgtcttcacaggcggaggactgtcctccgtagggttcctcggaggactgtcctccg

SEQ ID NO: 166 - DNA, 125 bp, synthetic construct
  source 1..125: mol_type = other DNA
cggaggactgtcctccgtgagtcctagcggaggactgtcctccgtcttcacaggcggaggactgtcctccgtagggttcctcggaggactgtcctccgacactagagtcggaggactgtcctccg

SEQ ID NO: 167 - DNA, 125 bp, synthetic construct
  source 1..125: mol_type = other DNA
cggtggcttctaatccgtgagtcctagcgggtgacagccctccgtcttcacaggcggaggagagtcttccgtagggttcctcggagtactgtcctccgacactagagtcggaggactgtcctccg

SEQ ID NO: 168 - DNA, 152 bp, synthetic construct
  source 1..152: mol_type = other DNA
cggaggactgtcctccgtgagtcctagcggaggactgtcctccgtcttcacaggcggaggactgtcctccgtagggttcctcggaggactgtcctccgacactagagtcggaggactgtcctccggatagtatcacggaggactgtcctccg

SEQ ID NO: 169 - DNA, 179 bp, synthetic construct
  source 1..179: mol_type = other DNA
cggaggactgtcctccgtgagtcctagcggaggactgtcctccgtcttcacaggcggaggactgtcctccgtagggttcctcggaggactgtcctccgacactagagtcggaggactgtcctccggatagtatcacggaggactgtcctccgagcaaacgaacggaggactgtcctccg

SEQ ID NO: 170 - DNA, 260 bp, synthetic construct
  source 1..260: mol_type = other DNA
cggaggactgtcctccgtgagtcctagcggaggactgtcctccgtcttcacaggcggaggactgtcctccgtagggttcctcggaggactgtcctccgacactagagtcggaggactgtcctccggatagtatcacggaggactgtcctccgaatggactaacggaggactgtcctccgagcaaacgaacggaggactgtcctccgtacaccgatccggaggactgtcctccggcgctcggcacggaggactgtcctccg

SEQ ID NO: 171 - DNA, 530 bp, synthetic construct
  source 1..530: mol_type = other DNA
cggaggactgtcctccgtgagtcctagcggaggactgtcctccgtcttcacaggcggaggactgtcctccgtagggttcctcggaggactgtcctccgacactagagtcggaggactgtcctccggatagtatcacggaggactgtcctccgaatggactaacggaggactgtcctccgactttagcgccggaggactgtcctccgtacaccgatccggaggactgtcctccggcgctcggcacggaggactgtcctccgcgaaaacgggcggaggactgtcctccgtgcgtggattcggaggactgtcctccgagccaggagccggaggactgtcctccgaagaccacggcggaggactgtcctccgaccagtaccccggaggactgtcctccgatgtcttgtacggaggactgtcctccgctgactatcgcggaggactgtcctccgcctacgtcatcggaggactgtcctccgatagaaacctcggaggactgtcctccgcatgtctaaccggaggactgtcctccg

SEQ ID NO: 172 - protein, 21 aa, synthetic construct
RYRVTAGANNNNNTCTABYRY

SEQ ID NO: 173 - DNA, 21 bp, synthetic construct
  source 1..21: mol_type = other DNA
atgatagaagcactctactat

SEQ ID NO: 174 - DNA, 207 bp, synthetic construct
  source 1..207: mol_type = other DNA
atgatagaagcactctactattgagtcctagatgatagaagcactctactattcttcacaggatgatagaagcactctactattagggttcctatgatagaagcactctactatacactagagtatgatagaagcactctactatgatagtatcaatgatagaagcactctactatagcaaacgaaatgatagaagcactctactat

SEQ ID NO: 175 - DNA, 21 bp, synthetic construct
  source 1..21: mol_type = other DNA
ataggtccacggtggaccata

SEQ ID NO: 176 - DNA, 207 bp, synthetic construct
  source 1..207: mol_type = other DNA
ataggtccacggtggaccatatgagtcctagataggtccacggtggaccatatcttcacaggataggtccacggtggaccatatagggttcctataggtccacggtggaccataacactagagtataggtccacggtggaccatagatagtatcaataggtccacggtggaccataagcaaacgaaataggtccacggtggaccata

SEQ ID NO: 177 - DNA, 172 bp, synthetic construct
  source 1..172: mol_type = other DNA
acgcaaacgttttcgttgagtcctagacgcaaacgttttcgttcttcacaggacgcaaacgttttcgttagggttcctacgcaaacgttttcgtacactagagtacgcaaacgttttcgtgatagtatcaacgcaaacgttttcgtagcaaacgaaacgcaaacgttttcgt

SEQ ID NO: 178 - DNA, 14 bp, synthetic construct
  source 1..14: mol_type = other DNA
acaagaaagtttgt

SEQ ID NO: 179 - DNA, 14 bp, synthetic construct
  source 1..14: mol_type = other DNA
acaagatacattgt

SEQ ID NO: 180 - DNA, 14 bp, synthetic construct
  source 1..14: mol_type = other DNA
acaagaaaaactgt

SEQ ID NO: 181 - DNA, 14 bp, synthetic construct
  source 1..14: mol_type = other DNA
acaagaaagtttgt

SEQ ID NO: 182 - DNA, 14 bp, synthetic construct
  source 1..14: mol_type = other DNA
acaagatacattgt

SEQ ID NO: 183 - DNA, 14 bp, synthetic construct
  source 1..14: mol_type = other DNA
acaagaaaaactgt

SEQ ID NO: 184 - DNA, 14 bp, synthetic construct
  source 1..14: mol_type = other DNA
acaagaaagtttgt

SEQ ID NO: 185 - DNA, 14 bp, synthetic construct
  source 1..14: mol_type = other DNA
acaagatacattgt

SEQ ID NO: 186 - DNA, 14 bp, synthetic construct
  source 1..14: mol_type = other DNA
acaagaaaaactgt

SEQ ID NO: 187 - DNA, 14 bp, synthetic construct
  source 1..14: mol_type = other DNA
acaagaaagtttgt

SEQ ID NO: 188 - DNA, 14 bp, synthetic construct
  source 1..14: mol_type = other DNA
acaagatacattgt

SEQ ID NO: 189 - DNA, 14 bp, synthetic construct
  source 1..14: mol_type = other DNA
acaagaaaaactgt

SEQ ID NO: 190 - DNA, 59 bp, synthetic construct
  source 1..59: mol_type = other DNA
acaagaaaaactgtatttgacaaacaagatacattgtatgaaaatacaagaaagtttgt

SEQ ID NO: 191 - DNA, 158 bp, synthetic construct
  source 1..158: mol_type = other DNA
acaagaaagtttgttgagtcctagacaagaaagtttgttcttcacaggacaagaaagtttgttagggttcctacaagaaagtttgtacactagagtacaagaaagtttgtgatagtatcaacaagaaagtttgtagcaaacgaaacaagaaagtttgt

SEQ ID NO: 192 - DNA, 158 bp, synthetic construct
  source 1..158: mol_type = other DNA
acaagaaaaactgttgagtcctagacaagaaaaactgttcttcacaggacaagaaaaactgttagggttcctacaagaaaaactgtacactagagtacaagaaaaactgtgatagtatcaacaagaaaaactgtagcaaacgaaacaagaaaaactgt

SEQ ID NO: 193 - DNA, 217 bp, synthetic construct
  source 1..217: mol_type = other DNA
cccaatcttcacaagaaaaactgtatttgacaaacaagatacattgtatgaaaatacaagaaagtttgttgatggaggcacaagaaaaactgtatttgacaaacaagatacattgtatgaaaatacaagaaagtttgttgatggaggcacaagaaaaactgtatttgacaaacaagatacattgtatgaaaatacaagaaagtttgttgatggaggc

SEQ ID NO: 194 - DNA, 137 bp, synthetic construct
  source 1..137: mol_type = other DNA
agtgactcatttgagtcctagagtgactcatttcttcacaggagtgactcatttagggttcctagtgactcattacactagagtagtgactcattgatagtatcaagtgactcattagcaaacgaaagtgactcatt

SEQ ID NO: 195 - DNA, 17 bp, synthetic construct
  source 1..17: mol_type = other DNA
ttgttatccgctcacaa

SEQ ID NO: 196 - DNA, 17 bp, synthetic construct
  source 1..17: mol_type = other DNA
ttgttatccgctcacaa

SEQ ID NO: 197 - DNA, 233 bp, synthetic construct
  source 1..233: mol_type = other DNA
ttgttatccgctcacaattccacatgtggccacaaattgttatccgctcacaattccacatgtggccacaaattgttatccgctcacaattccacatgtggccacaaattgttatccgctcacaattccacatgtggccacaaattgttatccgctcacaattccacatgtggccacaaattgttatccgctcacaattccacatgtggccacaaattgttatccgctcacaa

SEQ ID NO: 198 - DNA, 18 bp, synthetic construct
  source 1..18: mol_type = other DNA
tagggataacagggtaat

SEQ ID NO: 199 - protein, 9 aa, synthetic construct
PKKKRKVED

SEQ ID NO: 200 - protein, 38 aa, synthetic construct
KKKSSSDDEATADSQHSTPPKKKRKVEDPKDFPSELLS

SEQ ID NO: 201 - protein, 35 aa, synthetic construct
PSSDDEATADSQHAAPPKKKRKVEDPKDFPSELLS

SEQ ID NO: 202 - protein, 41 aa, synthetic construct
GNQSSNFGPMKGGNFGGRSSGPYGGGGQYFAKPRNQGGYGG

SEQ ID NO: 203 - protein, 45 aa, synthetic construct
FGNYNNQSSNFGPMKGGNFGGRSSGPYGGGGQYFAKPRNQGGYGG

SEQ ID NO: 204 - protein, 58 aa, synthetic construct
FGNYNNQSSNFGPMKGGNFGGRSSGPYGGGGQYFAKPRNQGGYGGSSSSSSYGSGRRF

SEQ ID NO: 205 - protein, 42 aa, synthetic construct
DTWTGVEALIRILQQLLFIHFRIGCRHSRIGIIQQRRTRNGA

SEQ ID NO: 206 - protein, 46 aa, synthetic construct
GDTWAGVEAIIRILQQLLFIHFRIGCRHSRIGVTRQRRARNGASRS

SEQ ID NO: 207 - protein, 95 aa, synthetic construct
EQAPEDQGPQREPHNEWTLELLEELKNEAVRHFPRIWLHGLGQHIYETYGDTWAGVEAIIRILQQLLFIHFRIGCRHSRIGVTRQRRARNGASRS

SEQ ID NO: 208 - protein, 19 aa, synthetic construct
ASQGTKRSYEQMETDGERQ

SEQ ID NO: 209 - protein, 26 aa, synthetic construct
PKKKRKVASQGTKRSYEQMETDGERQ

SEQ ID NO: 210 - protein, 27 aa, synthetic construct
MASQGTKRSYEQMETDGERQPKKKRKV

SEQ ID NO: 211 - protein, 9 aa, synthetic construct
PAAKRVKLD

SEQ ID NO: 212 - protein, 28 aa, synthetic construct
PAAKRVKLDASQGTKRSYEQMETDGERQ

SEQ ID NO: 213 - protein, 66 aa, synthetic construct
PAAKRVKLDASQGTKRSYEQMETDGERQKKKSSSDDEATADSQHSTPPKKKRKVEDPKDFPSELLS

SEQ ID NO: 214 - protein, 37 aa, synthetic construct
PAAKRVKLDASQGTKRSYEQMETDGERQPAAKRVKLD

SEQ ID NO: 215 - protein, 29 aa, synthetic construct
MASQGTKRSYEQMETDGERQPAAKRVKLD

SEQ ID NO: 216 - protein, 18 aa, synthetic construct
KRTADGSEFESPKKKRKV

SEQ ID NO: 217 - protein, 15 aa, synthetic construct
NELALKLAGLDINKT

SEQ ID NO: 218 - protein, 38 aa, synthetic construct
EHVNKMNSDRVPDVVLIKKSYDRTKRQRRRNWKLKELA

SEQ ID NO: 219 - protein, 8 aa, synthetic construct
WSHPQFEK

SEQ ID NO: 220 - protein, 16 aa, synthetic construct
WSHPQFEKWSHPQFEK

SEQ ID NO: 221 - protein, 24 aa, synthetic construct
WSHPQFEKWSHPQFEKWSHPQFEK

SEQ ID NO: 222 - skipped

SEQ ID NO: 223 - protein, 6 aa, synthetic construct
GGSGGS

SEQ ID NO: 224 - protein, 9 aa, synthetic construct
GGSGGSGGS

SEQ ID NO: 225 - protein, 5 aa, synthetic construct
GGGGS

SEQ ID NO: 226 - protein, 10 aa, synthetic construct
GGGGSGGGGS

SEQ ID NO: 227 - protein, 15 aa, synthetic construct
GGGGSGGGGSGGGGS

SEQ ID NO: 228 - protein, 20 aa, synthetic construct
GGGGSGGGGSGGGGSGGGGS

SEQ ID NO: 229 - protein, 12 aa, synthetic construct
QSGSGSTATGSG

SEQ ID NO: 230 - protein, 11 aa, synthetic construct
GTPTPTPTPTG

SEQ ID NO: 231 - protein, 11 aa, synthetic construct
SGGGSGGSGSS

SEQ ID NO: 232 - protein, 12 aa, synthetic construct
GSAGSAAGSGEF

SEQ ID NO: 233 - RNA, 19 nt, synthetic construct
  source 1..19: mol_type = other RNA
taatacgactcactatagg

SEQ ID NO: 234 - RNA, 19 nt, synthetic construct
  source 1..19: mol_type = other RNA
taatacgactcactataag

SEQ ID NO: 235 - RNA, 22 nt, synthetic construct
  source 1..22: mol_type = other RNA
taatacgactcactataagtca

SEQ ID NO: 236 - skipped

SEQ ID NO: 237 - RNA, 10 nt, synthetic construct
  source 1..10: mol_type = other RNA
gggataatag

SEQ ID NO: 238 - skipped

SEQ ID NO: 239 - RNA, 12 nt, synthetic construct
  source 1..12: mol_type = other RNA
gccgccaccatg

SEQ ID NO: 240-241 - skipped (2 entries)

SEQ ID NO: 242 - RNA, 10 nt, synthetic construct
  source 1..10: mol_type = other RNA
agaccagaca

SEQ ID NO: 243 - RNA, 15 nt, synthetic construct
  source 1..15: mol_type = other RNA
ggactggactggact

SEQ ID NO: 244 - RNA, 22 nt, synthetic construct
  source 1..22: mol_type = other RNA
taatgtgagttagctcactcat

SEQ ID NO: 245 - RNA, 10 nt, synthetic construct
  source 1..10: mol_type = other RNA
agatcgagga

SEQ ID NO: 246 - RNA, 21 nt, synthetic construct
  source 1..21: mol_type = other RNA
cggggaactctccaattcccc

SEQ ID NO: 247 - RNA, 31 nt, synthetic construct
  source 1..31: mol_type = other RNA
actcttctggtccccacagactcagagagaa

SEQ ID NO: 248 - RNA, 62 nt, synthetic construct
  source 1..62: mol_type = other RNA
aagagagactcagacacccctggtcttctcaactcttctggtccccacagactcagagagaa

SEQ ID NO: 249 - skipped

SEQ ID NO: 250 - RNA, 50 nt, synthetic construct
  source 1..50: mol_type = other RNA
ccgtgacgcagggataaagcaatcaaggttgtgtcagtgacgtgctataa

SEQ ID NO: 251 - RNA, 92 nt, synthetic construct
  source 1..92: mol_type = other RNA
gagccagataaaaagccagctccagcaggcgctgctcactcctccccatcctctccctctgtccctctgtccctctgaccctgcactgtccc

SEQ ID NO: 252 - RNA, 37 nt, synthetic construct
  source 1..37: mol_type = other RNA
acaggattgtctcccgggctggcagcagggccccagc

SEQ ID NO: 253 - RNA, 122 nt, synthetic construct
  source 1..122: mol_type = other RNA
aacttgtttattgcagcttataatggttacaaataaagcaatagcatcacaaatttcacaaataaagcatttttttcactgcattctagttgtggtttgtccaaactcatcaatgtatctta

SEQ ID NO: 254 - RNA, 134 nt, synthetic construct
  source 1..134: mol_type = other RNA
ctggtactgcatgcacgcaatgctagctgcccctttcccgtcctggtaccccgagtctcccccgacctcgggtcccaggtatgctcccacctccacctgcccactcaccacctctgctagttccagacacctcc

SEQ ID NO: 255 - RNA, 139 nt, synthetic construct
  source 1..139: mol_type = other RNA
caagcacgcagcaatgcagctcaaaaccttagcctagcacacccccacgggaaacagcagtgattaacctttagcaataaacgaaagtttaactaagctatactaaccccagggttggtcaatttcgtccagccacacc

SEQ ID NO: 256 - RNA, 111 nt, synthetic construct
  source 1..111: mol_type = other RNA
gctggagcctcggtggccatgcttcttgccccttgggcctccccccagcccctcctccccttcctgcacccgtacccccgtggtctttgaataaagtctgagtgggcggca

SEQ ID NO: 257 - RNA, 50 nt, synthetic construct
  source 1..50: mol_type = other RNA
ccgtaaatcggatagggcggaaacgatggacacgaagtctatagtcgggt

SEQ ID NO: 258 - RNA, 50 nt, synthetic construct
  source 1..50: mol_type = other RNA
ctcacgggatgacccacattgcgatgatgtagttccgggccgccaaatag

SEQ ID NO: 259 - RNA, 50 nt, synthetic construct
  source 1..50: mol_type = other RNA
aagtactgcaggcccgaatcactcggccaaatggcctgcataatgctgat

SEQ ID NO: 260 - RNA, 50 nt, synthetic construct
  source 1..50: mol_type = other RNA
taaacttccaagtcgcctcgcaggctcccaccaatgccgccgcctaaggg

SEQ ID NO: 261 - RNA, 50 nt, synthetic construct
  source 1..50: mol_type = other RNA
ctcaggagggtggtaagccaccgcccacgcgtggggcactcgtgtattct

SEQ ID NO: 262 - RNA, 50 nt, synthetic construct
  source 1..50: mol_type = other RNA
agcggacatcagccattgaatacatgaaacaaatcgcaaaagcaatagct

SEQ ID NO: 263 - RNA, 50 nt, synthetic construct
  source 1..50: mol_type = other RNA
cgtcgctccggggagctgtgtgtacgagtaaaaaatttagatcaccaaaa

SEQ ID NO: 264 - RNA, 50 nt, synthetic construct
  source 1..50: mol_type = other RNA
acaatcgccagggacagataggaagttatagacagcaggccgacacaagt

SEQ ID NO: 265 - RNA, 50 nt, synthetic construct
  source 1..50: mol_type = other RNA
gaaaaatctatagcagaagtcagcggtagacgcacggcatagcatccaac

SEQ ID NO: 266 - RNA, 50 nt, synthetic construct
  source 1..50: mol_type = other RNA
gggcgctcgagcaggttcagaaggagatcaaaaacccccaaggatcaaac

SEQ ID NO: 267 - DNA, 19 bp, synthetic construct
  source 1..19: mol_type = other DNA
tccctatcagtgatagaga

SEQ ID NO: 268 - protein, 207 aa, Escherichia coli
MSRLDKSKVINSALELLNEVGIEGLTTRKLAQKLGVEQPTLYWHVKNKRALLDALAIEMLDRHHTHFCPLEGESWQDFLRNNAKSFRCALLSHRDGAKVHLGTRPTEKQYETLENQLAFLCQQGFSLENALYALSAVGHFTLGCVLEDQEHQVAKEERETPTTDSMPPLLRQAIELFDHQGAEPAFLFGLELIICGLEKQLKCESGS

SEQ ID NO: 269 - RNA, 621 nt, synthetic construct
  source 1..621: mol_type = other RNA
atgagcagactggacaagagcaaagtgatcaacagcgccctggaactgctgaacgaagtgggcatcgagggcctgaccacaagaaagctggcccagaagctgggcgtcgagcagcctacactgtactggcacgtgaagaacaagcgggccctgctggatgccctggccattgagatgctggaccggcaccacacacacttttgccctctggaaggcgagagctggcaggacttcctgagaaacaacgccaagagcttcagatgcgccctgctgagccatagagatggcgccaaagtgcacctgggcaccagacctacagagaagcagtacgagacactggaaaaccagctggccttcctgtgccagcagggattcagcctggaaaacgccctgtatgccctgtctgccgtgggccactttacactgggatgcgtgctggaagatcaagagcaccaggtggccaaagaggaaagagagacacccaccaccgacagcatgcctccactgctgagacaggccatcgagctgttcgatcaccaaggcgccgaacctgcctttctgttcggactggaactgatcatctgcggcctcgaaaagcagctgaagtgcgagagcggctcc

SEQ ID NO: 270 - RNA, 621 nt, synthetic construct
  source 1..621: mol_type = other RNA
atgagcaggctggacaagagcaaggtgatcaacagcgccctggagctgctgaacgaggtgggcatcgagggcctgaccaccaggaagctggcccagaagctgggcgtggagcagcccaccctgtactggcacgtgaagaacaagagggccctgctggacgccctggccatcgagatgctggacaggcaccacacccacttctgccccctggagggcgagagctggcaggacttcctgaggaacaacgccaagagcttcaggtgcgccctgctgagccacagggacggcgccaaggtgcacctgggcaccaggcccaccgagaagcagtacgagacgctggagaaccagctggccttcctgtgccagcagggcttcagcctggagaacgccctgtacgccctgagcgccgtgggccacttcaccctgggctgcgtgctggaggaccaggagcaccaggtggccaaggaggagagggagacgcccaccaccgacagcatgccccccctgctgaggcaggccatcgagctgttcgaccaccagggcgccgagcccgccttcctgttcggcctggagctgatcatctgcggcctggagaagcagctgaagtgcgagagcggcagc

SEQ ID NO: 271 - RNA, 621 nt, synthetic construct
  source 1..621: mol_type = other RNA
atgagcaggctggataagagcaaggtgattaactccgccctggagctgctgaacgaagtgggaatcgagggcctgactacaaggaagttggcccagaagctgggcgtggagcagcctaccctgtactggcacgtgaagaacaagagagccctgctggacgccctggccatcgagatgctggacaggcatcacactcacttttgccctctcgagggcgagagctggcaggacttcctgaggaacaacgccaagtcatttcggtgtgccctgctgagccatcgagatggggccaaggtgcacctgggaaccagacccacagagaagcagtacgagacactggaaaaccagctggctttcctgtgccagcagggcttctccctggagaacgccctgtacgccctgagcgccgtgggccatttcaccctgggttgtgtgctggaggatcaggagcaccaggtggccaaggaggaaagggaaacccctaccaccgactcaatgccacctctgctgcgccaggccatcgagctgtttgaccaccagggagccgagcccgccttcctgttcgggctggagctgattatttgcggcctggagaagcagctgaagtgtgagagtggcagc

SEQ ID NO: 272 - RNA, 621 nt, synthetic construct
  source 1..621: mol_type = other RNA
atgtcccgactggacaaatccaaggttatcaatagcgccctggagctgctcaatgaagtgggtatcgagggcctgacgacacgcaagcttgcacagaagttgggcgtcgagcagccaactctgtattggcatgtgaagaacaagagggccctgctcgacgccctcgcgattgaaatgctggacagacaccacacacatttttgtcctttggagggcgagtcctggcaggactttcttagaaataacgccaagtccttccgctgcgcactgctcagccaccgagatggggctaaagtgcatcttggaacacgccccaccgagaaacagtacgaaactctcgagaatcagcttgcatttctgtgtcagcaagggttcagtctggaaaacgccctgtacgctctgtccgccgtggggcattttacactgggatgtgtgctggaagaccaagagcatcaggtcgccaaggaagaaagagagactccgacaaccgactccatgcctcccttgctccggcaggccattgagctgttcgaccaccagggcgccgaacctgccttcttgttcggtcttgagctgatcatctgcggactggaaaagcaactgaaatgtgagagcgggtcc

SEQ ID NO: 273 - RNA, 621 nt, synthetic construct
  source 1..621: mol_type = other RNA
atgagtcgacttgataagagcaaggttataaattccgctttggaactgttgaatgaggtgggcatcgagggtcttacgacccgcaaattggcacaaaagttgggagttgaacagccaacgctttattggcacgttaaaaataagcgggctctgctggacgctctggctatagaaatgcttgaccggcatcatacccatttttgtcctctggaaggcgaatcttggcaggactttctcaggaataacgccaaatcttttagatgcgctcttttgtcacaccgcgatggagccaaggtccatcttggcacgcggccaacggagaaacaatatgagaccctcgaaaaccagctcgccttcctctgtcaacagggattcagtcttgagaatgccctgtacgctctttctgcggtcgggcattttacactcggatgtgttttggaggaccaagaacatcaagtagcgaaagaggaacgggaaactcccacgaccgattcaatgccgccactcctccgacaagctattgaattgttcgatcatcaaggggctgaacctgccttcctcttcggtctggaacttataatctgcgggctcgagaaacagttgaaatgcgagtcaggatca

SEQ ID NO: 274 - RNA, 621 nt, synthetic construct
  source 1..621: mol_type = other RNA
atgagcagactggacaagagcaaggtgatcaacagcgccctggagctgctgaacgaggtgggcatcgagggcctgaccacaagaaagctggctcagaagctgggcgtggagcagcccaccctgtactggcacgtgaagaacaagagagccctgctggacgccctggccatcgagatgctggacagacaccacacccacttctgccccctggagggcgagagctggcaagacttcctgagaaacaacgccaagagcttcagatgcgccctgctgagccacagagacggcgccaaggtgcacctgggcacaagacccaccgagaagcagtacgaaaccctggagaatcagctggccttcctgtgtcagcaaggcttcagcctggagaacgccctgtacgccctgagcgccgtgggccacttcaccctgggctgcgtgctggaggaccaagagcaccaagtggccaaggaggagagagaaacccccaccaccgacagcatgccccccctgctgagacaagccatcgagctgttcgaccaccaaggcgccgagcccgccttcctgttcggcctggagctgatcatctgcggcctggagaagcagctgaagtgcgaaagcggcagc

SEQ ID NO: 275 - RNA, 621 nt, synthetic construct
  source 1..621: mol_type = other RNA
atgagcagactggataagagcaaggtcatcaacagcgccctcgaactgctgaacgaggtgggcatcgaaggccttacaacaagaaagctggctcagaagctgggcgtggaacaacctaccctgtattggcatgtgaagaacaagcgggctctgctggacgccctggccatcgagatgctggacagacaccacacccacttttgcccactggagggcgagagctggcaggacttcctgagaaataacgccaagtccttccggtgcgccctcctgagccaccgggacggcgccaaggtgcacctgggaaccagacctaccgagaagcagtacgagacactggaaaaccagctggccttcctgtgccagcagggcttcagcctggagaatgccctgtacgccctgtctgccgtgggccacttcaccctgggttgtgtgctggaagatcaggagcaccaagtggccaaagaggaaagagagacacctacaaccgacagcatgccccccctgctgcggcaggccattgagctgttcgaccaccagggagccgagcctgcttttctgttcggcctggagctgatcatctgcggcctggaaaaacagctgaagtgtgaatctggctcc

SEQ ID NO: 276 - RNA, 621 nt, synthetic construct
  source 1..621: mol_type = other RNA
atgagcagactggacaagagcaaagtgatcaacagcgccctggaactgctgaacgaagtgggcatcgagggcctgaccacaagaaagctggcccagaagctgggcgtcgagcagcctacactgtactggcacgtgaagaacaagcgggccctgctggatgccctggccattgagatgctggaccggcaccacacacacttttgccctctggaaggcgagagctggcaggacttcctgagaaacaacgccaagagcttcagatgcgccctgctgagccatagagatggcgccaaagtgcacctgggcaccagacctacagagaagcagtacgagacactggaaaaccagctggccttcctgtgccagcagggattcagcctggaaaacgccctgtatgccctgtctgccgtgggccactttacactgggatgcgtgctggaagatcaagagcaccaggtggccaaagaggaaagagagacacccaccaccgacagcatgcctccactgctgagacaggccatcgagctgttcgatcaccaaggcgccgaacctgcctttctgttcggactggaactgatcatctgcggcctcgagaagcagctgaagtgcgagtctggatct

SEQ ID NO: 277 - RNA, 621 nt, synthetic construct
  source 1..621: mol_type = other RNA
atgagcaggctggacaagagcaaggtgatcaacagcgccctggagctgctgaacgaggtgggcatcgagggcctgaccaccaggaagctggcccagaagctgggcgtggagcagcccaccctgtactggcacgtgaagaacaagagggccctgctggacgccctggccatcgagatgctggacaggcaccacacccacttctgccccctggagggcgagagctggcaggacttcctgaggaacaacgccaagagcttcaggtgcgccctgctgagccacagggacggcgccaaggtgcacctgggcaccaggcccaccgagaagcagtacgagaccctggagaaccagctggccttcctgtgccagcagggcttcagcctggagaacgccctgtacgccctgagcgccgtgggccacttcaccctgggctgcgtgctggaggaccaggagcaccaggtggccaaggaggagagggagacccccaccaccgacagcatgccccccctgctgaggcaggccatcgagctgttcgaccaccagggcgccgagcccgccttcctgttcggcctggagctgatcatctgcggcctggagaagcagctgaagtgcgagagcggcagc

SEQ ID NO: 278 - RNA, 621 nt, synthetic construct
  source 1..621: mol_type = other RNA
atgtcccgcctggacaagtctaaggtgatcaattccgccctggaactgctgaatgaggtgggaatcgagggcctgaccacaaggaagctggcccagaagctgggcgtggagcagcctacactgtactggcatgtgaagaacaagcgggccctcctggacgccctggccatcgaaatgctggaccgccaccacacccacttctgtcccctggagggcgagagctggcaggatttcctgcggaataatgccaagagcttcagatgtgctctgctgagccacagagatggcgcaaaggtgcacctgggcaccagacccaccgagaagcagtatgagactctggagaaccagctggcattcctgtgccaacaggggttttccctggagaacgccctgtacgccctgagcgccgtgggacacttcaccctgggctgtgtgctggaggaccaggagcaccaggtggccaaggaggaaagagagacaccaactaccgactccatgcctcctctcctccggcaggccattgaactgtttgatcatcagggcgccgagccagcctttctgtttggcctggaactgatcatctgtggcctggagaagcagctgaagtgcgaatctggcagc

SEQ ID NO: 279 - RNA, 621 nt, synthetic construct
  source 1..621: mol_type = other RNA
atgtctaggttggataaatcaaaggtcatcaattctgctctggagctcctcaatgaagtgggaatcgaaggtctcactactcggaagctggcacaaaaactcggggtggaacaacctaccctttattggcacgtgaaaaacaaaagggccttgctggacgctctggccatagagatgttggaccgacatcacacgcatttctgccctctggagggggaatcctggcaagatttcctgcgcaataacgcaaagtctttccgatgcgctctgctctctcatagggacggcgctaaggtccacctgggaaccagacctacggagaagcagtatgaaaccctggagaaccagctggcattcctctgccagcagggcttcagtctcgagaacgcactgtacgcactgtctgctgtgggtcacttcaccctgggctgtgtgttggaggaccaggagcaccaggtggccaaggaagagcgcgaaacccctacaacggattctatgccccccttgcttaggcaggccatcgagctcttcgatcaccagggggccgaacctgcctttttgtttggcctggaactcattatatgtgggctggagaagcagctgaaatgtgagagtggaagc

SEQ ID NO: 280 - RNA, 621 nt, synthetic construct
  source 1..621: mol_type = other RNA
atgtcacgattggacaaaagtaaggttataaactctgctctcgaacttctgaacgaagttgggatcgaaggtttgaccacccgcaagttggctcaaaaacttggtgtggagcagcctaccctgtattggcacgtaaagaacaagagagccttgctggacgcccttgcaatcgagatgctggacagacaccacacccatttttgtcccttggaaggggagagttggcaagattttctgcggaacaatgctaagagcttccgctgtgccctgctttcccatcgagatggcgcaaaagtgcacttgggaactcggcccactgagaagcaatacgagactcttgagaaccaactcgcatttctttgtcaacaaggattttccctggagaatgctctgtatgctttgagtgcagtaggccatttcaccctcggctgcgtactggaggaccaagagcatcaagttgccaaagaagaacgggaaaccccaactacagatagcatgcctcctttgctccgacaggccatagaactctttgatcatcaaggtgcagagccagcattcctcttcggccttgagctcattatttgcggacttgagaagcaactgaagtgtgaaagcggaagt

SEQ ID NO: 281 - RNA, 621 nt, synthetic construct
  source 1..621: mol_type = other RNA
atgtctaggctggacaagagcaaggtgatcaacagcgccctggagctgctgaacgaggtgggcatcgagggcctgaccacaaggaagctggctcagaagctgggcgtggagcagcctaccctgtactggcacgtgaagaacaagagggccctgctggacgccctggccatcgagatgctggataggcaccacacccacttctgccctctggagggcgagagctggcaagacttcctgaggaacaacgccaagagctttaggtgcgccctgctgagccatagggacggcgccaaggtgcacctgggcacaaggcctaccgagaagcagtacgaaaccctggagaatcagctggccttcctgtgtcagcaaggcttcagcctggagaacgccctgtacgccctgagcgccgtgggccacttcaccctgggctgcgtgctggaggaccaagagcaccaagtggccaaggaggagagggaaacccctaccaccgacagcatgcctcctctgctgaggcaagccatcgagctgttcgaccaccaaggcgccgagcctgccttcctgttcggcctggagctgatcatctgcggcctggagaagcagctgaagtgcgaaagcggcagc

SEQ ID NO: 282 - RNA, 621 nt, synthetic construct
  source 1..621: mol_type = other RNA
atgtctcgtctcgacaagtcaaaagtcatcaatagtgctctggagctgcttaatgaagtagggatagaaggtctgaccaccaggaagctggcccagaagctgggagttgaacaacctaccctctactggcacgtgaaaaacaagcgcgcacttttagacgccctggcgattgagatgctggatcggcaccatacacatttctgccccctagaaggagaatcctggcaggactttctccgaaacaacgccaaatccttccgctgtgcactgctgagccatcgagatggagcgaaagtgcacctggggacgcggcctactgagaaacagtacgaaactctagagaaccagttggccttcctctgccagcagggattcagtttagagaatgcactctatgctctctctgcagtgggccacttcacattgggctgcgttttggaagaccaggagcatcaggtggccaaggaggaaagagagacacctactacagatagcatgcctccactgctgagacaagccattgaactgtttgaccaccagggtgctgagccagcctttttgtttggtttagaacttatcatctgtggcctagagaagcagctgaaatgtgagtctggcagc

SEQ ID NO: 283 - RNA, 621 nt, synthetic construct
  source 1..621: mol_type = other RNA
atgagcagactggacaagagcaaagtgatcaacagcgccctggaactgctgaacgaagtgggcatcgagggcctgaccacaagaaagctggctcagaagctgggcgtcgagcagcctacactgtactggcacgtgaagaacaagagagccctgctggacgccctggccatcgagatgctggatagacaccacacacacttttgccctctggaaggcgagagctggcaggacttcctgagaaacaacgccaagagcttcagatgcgccctgctgagccatagagatggcgccaaagtgcacctgggcaccagacctacagagaagcagtacgagacactggaaaaccagctggccttcctgtgccagcagggattctctctggaaaacgccctgtacgccctgagcgctgtgggccactttacactgggatgcgtgctggaagatcaagagcaccaggtggccaaagaggaaagagagacacccaccaccgacagcatgcctccactgctgagacaggctatcgagctgttcgaccaccaaggcgccgagcctgcttttctgttcggactggaactgatcatctgcggcctcgagaagcagctgaagtgcgagtctggatct

SEQ ID NO: 284 - protein, 112 aa, synthetic construct
MRPFQCRICMRNFSRSDNLSVHIRTHTGEKPFACDICGRKFAQKINLQVHTKIHTGEKPFQCRICMRNFSRSDVLSEHIRTHTGEKPFACDICGRKFAQRNHRTTHTKIHLR

SEQ ID NO: 285 - protein, 131 aa, synthetic construct
MAPKKKRKVGIHGVPAAMAERPFQCRICMRNFSRSDNLSVHIRTHTGEKPFACDICGRKFAQKINLQVHTKIHTGEKPFQCRICMRNFSRSDVLSEHIRTHTGEKPFACDICGRKFAQRNHRTTHTKIHLR

SEQ ID NO: 286 - DNA, 18 bp, synthetic construct
  source 1..18: mol_type = other DNA
ttcattacacctgcagct

SEQ ID NO: 287 - DNA, 15 bp, synthetic construct
  source 1..15: mol_type = other DNA
ataaaccccctccaa

SEQ ID NO: 288 - DNA, 32 bp, synthetic construct; identical to SEQ ID NO: 144
  source 1..32: mol_type = other DNA

SEQ ID NO: 289 - protein, 34 aa, synthetic construct
TMVLAQNRKKSLHCFEGLFTAVVTSNSDHLVRSQ

SEQ ID NO: 290 - protein, 511 aa, synthetic construct
MLTPDQVVAIASNIGGKQALETVQRLLPVLCQDHGLTPDQVVAIASNGGGKQALETVQRLLPVLCQDHGLTPDQVVAIASNIGGKQALETVQRLLPVLCQDHGLTPDQVVAIASNIGGKQALETVQRLLPVLCQDHGLTPDQVVAIASNIGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNIGGKQALETVQRLLPVLCQDHGLTPDQVVAIASNIGGKQALETVQRLLPVLCQDHG

SEQ ID NO: 291 - protein, 65 aa, Saccharomyces cerevisiae
MKLLSSIEQACDICRLKKLKCSKEKPKCAKCLKNNWECRYSPKTKRSPLTRAHLTEVESRLERLE

SEQ ID NO: 292 - protein, 147 aa, synthetic construct
MKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVS

SEQ ID NO: 293 - RNA, 441 nt, synthetic construct
  source 1..441: mol_type = other RNA
atgaaactgctgtcatccatcgagcaagcttgtgacatatgccggctgaagaaattgaaatgtagcaaggagaagccaaaatgtgcgaaatgcctcaagaataattgggaatgcagatattcccctaagactaagcgtagccccttgacccgggcccaccttactgaggtcgagagcagattagagcgcctcgagcaattgttcctgctcatcttccccagagaggatctggatatgatcttaaagatggacagccttcaagacattaaggctctgctcactgggctgttcgtgcaggacaacgtcaacaaggacgctgtgacggaccgcctggcttccgtagaaacggacatgcccctgactctgaggcaacaccgcatttctgctacgagttcttccgaggaatcctcaaataagggacagcgtcaactgaccgtctct

SEQ ID NO: 294 - RNA, 441 nt, synthetic construct
  source 1..441: mol_type = other RNA
atgaagctgctgagcagcatcgagcaggcctgcgacatctgcaggctgaagaagctgaagtgcagcaaggagaagcccaagtgcgccaagtgcctgaagaacaactgggagtgcaggtacagccccaagaccaagaggagccccctgaccagggcccacctgaccgaggtggagagcaggctggagaggctggagcagctgttcctgctgatcttccccagggaggacctggacatgatcctgaagatggacagcctgcaggacatcaaggccctgctgaccggcctgttcgtgcaggacaacgtgaacaaggacgccgtgaccgacaggctggccagcgtggagacggacatgcccctgaccctgaggcagcacaggatcagcgccaccagcagcagcgaggagagcagcaacaagggccagaggcagctgaccgtgagc

SEQ ID NO: 295 - RNA, 441 nt, synthetic construct
  source 1..441: mol_type = other RNA
atgaagctgctgtctagcatcgagcaggcgtgtgacatctgcagactgaagaaactgaaatgcagtaaagagaagccaaagtgtgctaagtgtctgaaaaacaattgggagtgtagatactcccccaagacaaagagaagcccactgacaagggcccacctgaccgaggtggagagtagactggagagactggagcagctgtttctgctgatcttcccaagggaggacctggacatgattctgaagatggactccctgcaggatatcaaagccctgctgaccggactgttcgtgcaggacaacgtgaacaaggacgccgtgaccgacaggctggccagcgtggagacggacatgcccctgacactgagacagcaccggatcagcgccaccagctcttccgaggagagttccaacaagggacagcggcagttgacagtgtcc

SEQ ID NO: 296 - RNA, 441 nt, synthetic construct
  source 1..441: mol_type = other RNA
atgaagctgctgagcagcatcgagcaagcctgcgacatctgtaggctgaagaagctgaagtgcagcaaggagaagcctaagtgcgccaagtgcctgaagaacaactgggagtgtaggtacagccctaagacgaagaggagccctctgacaagggcccacctgaccgaggtggagtctaggctggagaggctggagcagctgttcctgctgatcttccctagggaggacctggacatgatcctgaagatggacagcctgcaagacatcaaggccctgctgaccggcctgttcgtgcaagacaacgtgaacaaggacgccgtgaccgataggctggctagcgtggaaaccgacatgcctctgaccctgaggcagcataggatcagcgccacaagcagcagcgaggagagcagcaacaagggacagaggcagctgaccgtgagc

SEQ ID NO: 297 - RNA, 441 nt, synthetic construct
  source 1..441: mol_type = other RNA
atgaagttactatcttcgattgagcaagcctgtgacatctgcagacttaagaaactgaaatgcagcaaagagaagcccaaatgtgctaagtgcctgaagaacaactgggagtgtcgttacagcccaaagaccaagcgctcacctcttactcgagctcacctgacagaggtggaaagtcgcctggagcggttagagcagctgttcctgctcatcttccccagagaagacctggacatgatcctcaagatggacagcctgcaggatattaaagccctgctcacaggcctgttcgtgcaggacaatgtcaataaagatgctgtgactgatagacttgcatctgtagaaacggacatgcctctgactctgcggcagcatcggatatctgccacctcctcctcagaagaaagtagcaacaagggccagaggcagttgaccgtttct

SEQ ID NO: 298 - protein, 21 aa, synthetic construct
RYRVTAGANNNNNTCTABYRY

SEQ ID NO: 299 - protein, 53 aa, unidentified
  source 1..53: note = Salmonella phage
MKGMSKMPQFNLRWPREVLDLVRKVAEENGRSVNSEIYQRVMESFKKEGRIGA

SEQ ID NO: 300 - protein, 58 aa, synthetic construct
MKGMSKMPQFNLRWPREVLDLVRKVAEENGRSVNSEIYQRVMASFAKEGRIAAKNQHE

SEQ ID NO: 301 - RNA, 159 nt, Unidentified
  source 1..159: mol_type = mRNA; note = Salmonella phage
atgaagggcatgagcaagatgccccagttcaacctgcgctggccccgcgaggtgctggacctggtgcgcaaggtggccgaggagaacggccgcagcgtgaacagcgagatctaccagcgcgtgatggagagcttcaagaaggagggccgcatcggcgcc

SEQ ID NO: 302 - RNA, 174 nt, synthetic construct
  source 1..174: mol_type = other RNA
atgaagggcatgagcaagatgccccagttcaacctgcgctggccccgcgaggtgctggacctggtgcgcaaggtggccgaggagaacggccgcagcgtgaacagcgagatctaccagcgcgtgatggccagcttcgccaaggagggccgcatcgccgccaagaaccagcacgag

SEQ ID NO: 303 - DNA, 15 bp, synthetic construct
  source 1..15: mol_type = other DNA
ggnccacngtggncc

SEQ ID NO: 304 - DNA, 21 bp, synthetic construct
  source 1..21: mol_type = other DNA
ataggtccacggtggaccata

SEQ ID NO: 305 - protein, 83 aa, Salmonella sp.
MARDDPHFNFRMPMEVREKLKFRAEANGRSMNSELLQIVQDALSKPSPVTGYRNDAERLADEQSELVKKMVFDTLKDLYKKTT

SEQ ID NO: 306 - RNA, 249 nt, synthetic construct
  source 1..249: mol_type = other RNA
atggcccgcgacgacccccacttcaacttccgcatgcccatggaggtgcgcgagaagctgaagttccgcgccgaggccaacggccgcagcatgaacagcgagctgctgcagatcgtgcaggacgccctgagcaagcccagccccgtgaccggctaccgcaacgacgccgagcgcctggccgacgagcagagcgagctggtgaagaagatggtgttcgacaccctgaaggacctgtacaagaagaccacc

SEQ ID NO: 307 - DNA, 16 bp, synthetic construct
  source 1..16: mol_type = other DNA
acgcaaacgttttcgt

SEQ ID NO: 308 - protein, 341 aa, Escherichia coli
MATIKDVAKRANVSTTTVSHVINKTRFVAEETRNAVWAAIKELHYSPSAVARSLKVNHTKSIGLLATSSEAAYFAEIIEAVEKNCFQKGYTLILGNAWNNLEKQRAYLSMMAQKRVDGLLVMCSEYPEPLLAMLEEYRHIPMVVMDWGEAKADFTDAVIDNAFEGGYMAGRYLIERGHREIGVIPGPLERNTGAGRLAGFMKAMEEAMIKVPESWIVQGDFEPESGYRAMQQILSQPHRPTAVFCGGDIMAMGALCAADEMGLRVPQDVSLIGYDNVRNARYFTPALTTIHQPKDSLGETAFNMLLDRIVNKREEPQSIEVHPRLIERRSVADGPFRDYRR

SEQ ID NO: 309 - RNA, 1023 nt, synthetic construct
  source 1..1023: mol_type = other RNA
atggccaccatcaaggacgtggccaagcgcgccaacgtgagcaccaccaccgtgagccacgtgatcaacaagacccgcttcgtggccgaggagacgcgcaacgccgtgtgggccgccatcaaggagctgcactacagccccagcgccgtggcccgcagcctgaaggtgaaccacaccaagagcatcggcctgctggccaccagcagcgaggccgcctacttcgccgagatcatcgaggccgtggagaagaactgcttccagaagggctacaccctgatcctgggcaacgcctggaacaacctggagaagcagcgcgcctacctgagcatgatggcccagaagcgcgtggacggcctgctggtgatgtgcagcgagtaccccgagcccctgctggccatgctggaggagtaccgccacatccccatggtggtgatggactggggcgaggccaaggccgacttcaccgacgccgtgatcgacaacgccttcgagggcggctacatggccggccgctacctgatcgagcgcggccaccgcgagatcggcgtgatccccggccccctggagcgcaacaccggcgccggccgcctggccggcttcatgaaggccatggaggaggccatgatcaaggtgcccgagagctggatcgtgcagggcgacttcgagcccgagagcggctaccgcgccatgcagcagatcctgagccagccccaccgccccaccgccgtgttctgcggcggcgacatcatggccatgggcgccctgtgcgccgccgacgagatgggcctgcgcgtgccccaggacgtgagcctgatcggctacgacaacgtgcgcaacgcccgctacttcacccccgccctgaccaccatccaccagcccaaggacagcctgggcgagacggccttcaacatgctgctggaccgcatcgtgaacaagcgcgaggagccccagagcatcgaggtgcacccccgcctgatcgagcgccgcagcgtggccgacggccccttccgcgactaccgccgc

SEQ ID NO: 310 - RNA, 1080 nt, synthetic construct
  source 1..1080: mol_type = other RNA
atgaaaccagtcacactctatgatgtagcggaatatgctggggtatcctatcaaacagttagtagagttgttaatcaagcttcacatgtttccgccaaaacgagggaaaaggttgaagcagctatggctgaacttaattatataccaaatcgagtagcgcaacaactcgcagggaaacaatctttactcattggagtcgcaacatcctcactcgctctccatgctccttctcaaatagtcgctgctattaaaagtagagctgatcaattaggggcatctgtcgtagtctctatggtcgaaagatctggtgtagaagcttgtaaagctgcagtccataatctcctcgctcaacgggtcagcgggcttataattaattatcctcttgatgatcaagatgcaatagctgtcgaagcagcttgtactaatgtccctgcgttgtttcttgatgtctcagatcaaacaccaataaattcaattatatttagtcatgaagatggtacaaggctcggggttgaacatctcgtagcactcggtcatcaacaaatagcactccttgctgggccactctcttccgtttctgcacggctcaggctcgcaggatggcataaatatttgacacgtaatcaaattcaacctatagctgaaagggaaggtgattggtccgctatgagtgggtttcaacaaacaatgcaaatgttgaatgaagggatagttccaactgcaatgctcgttgctaatgatcaaatggcgcttggtgcgatgcgagctattacagaatccggacttcgggttggtgctgatatatccgtagtcggatatgatgatacagaagattcctcatgttatattccacctcttacaactattaaacaagattttaggcttctcgggcaaacatccgtcgatagacttttacaactctcccaaggacaagcagtaaaaggaaatcaattattgccggttagtcttgtcaaacgtaaaacaacgcttgcgccaaatacacaaacggcttctcctagagcacttgctgatagtcttatgcaactcgcacgacaagtctctcgattggaatctggacaa

SEQ ID NO: 311 - DNA, 18 bp, synthetic construct
  source 1..18: mol_type = other DNA
tagggataacagggtaat

SEQ ID NO: 312 - protein, 235 aa, synthetic construct
MKNIKKNQVMNLGPNSKLLKEYKSQLIELNIEQFEAGIGLILGAAYIRSRDEGKTYCMQFEWKNKAYMDHVCLLYDQWVLSPPHKKERVNHLGNLVITWGAQTFKHQAFNKLANLFIVNNKKTIPNNLVENYLTPMSLAYWFMDDGGKWDYNKNSTNKSIVLNTQSFTFEEVEYLVKGLRNKFQLNCYVKINKNKPIIYIDSMSYLIFYNLIKPYLIPQMMYKLPNTISSETFLK

SEQ ID NO: 313 - protein, 273 aa, synthetic construct
MPKKKRKVPKKHAAPPKKKRKVEDPRFMYPYDVPDYAGMKNIKKNQVMNLGPNSKLLKEYKSQLIELNIEQFEAGIGLILGAAYIRSRDEGKTYCMQFEWKNKAYMDHVCLLYDQWVLSPPHKKERVNHLGNLVITWGAQTFKHQAFNKLANLFIVNNKKTIPNNLVENYLTPMSLAYWFMDDGGKWDYNKNSTNKSIVLNTQSFTFEEVEYLVKGLRNKFQLNCYVKINKNKPIIYIDSMSYLIFYNLIKPYLIPQMMYKLPNTISSETFLK

SEQ ID NO: 314 - DNA, 157 bp, synthetic construct
  source 1..157: mol_type = other DNA
atcacgtgatagattactactgataatcacgtgattatcacgtgatagattactactgatatgatagccaactgcagctaataataaaccaagattactactgataccatgaactttgagattactactgatagtctgctaagtcaataatcagaat

SEQ ID NO: 315 - DNA, 147 bp, synthetic construct
  source 1..147: mol_type = other DNA
tccattgatttagagattactactgatagcaataaaatctgggtcacaggagttggaagattactactgatactgacgtcagagattactactgatagtctgctaagtcaataatcagaatagattactactgatatacacgtaatc

SEQ ID NO: 316 - DNA, 149 bp, synthetic construct; identical to SEQ ID NO: 141
  source 1..149: mol_type = other DNA

SEQ ID NO: 317 - DNA, 133 bp, synthetic construct
  source 1..133: mol_type = other DNA
tacgcccacgcattagattactactgatatattgattagattactactgataagtatggttaatgatctacagagattactactgatatacgcccacgcattagattactactgataagaacatccctgtaca

SEQ ID NO: 318 - DNA, 144 bp, synthetic construct
  source 1..144: mol_type = other DNA
caaacgtggtttagattactactgataaacacgggaggtcaaagattgcgcccagattactactgatatggttaatattcaccagcagattactactgataatcacgtgatagattactactgataaccggaagtacttccggt

SEQ ID NO: 319 - DNA, 151 bp, synthetic construct
  source 1..151: mol_type = other DNA
acaggaagtagattactactgataagaacattttgtacgagattactactgataaacccggaagtgagattactactgatatggtatgattttgtaatggggtaggaagattactactgataaattatggttctgggtgattcaagtaaca

SEQ ID NO: 320 - DNA, 149 bp, synthetic construct
  source 1..149: mol_type = other DNA
attgtgcaatagattactactgatacagaggtcacagagttcaagcagattactactgatatattgattagattactactgatatcgagcgcaggtcaaaaggtcacctgcagattactactgataatatgattatgcaaatttataga

SEQ ID NO: 321 - DNA, 113 bp, synthetic construct
  source 1..113: mol_type = other DNA
tgacgtcaagattactactgataccatgaactttgagattactactgatatgtttactttagattactactgatatccacgtggtattagattactactgataattacaaaat

SEQ ID NO: 322 - DNA, 160 bp, synthetic construct
  source 1..160: mol_type = other DNA
aaactaggtcacgaaaggtcaaagtcagattactactgatattatgcatatgcataaagattactactgataatcacgtgattatcacgtgatagattactactgataaggttaaaggtctagattactactgatagtttatcagtgactagtcattgat

SEQ ID NO: 323 - DNA, 151 bp, synthetic construct
  source 1..151: mol_type = other DNA
tcgagcgcaggtcaaaggtcacctgcagattactactgatatcgagcgctgggcaaaggtcacctgcagattactactgatacacttccgccggaagtgagattactactgataaaaaaatcaataatagattactactgataccacgctg

SEQ ID NO: 324 - DNA, 125 bp, synthetic construct
  source 1..125: mol_type = other DNA
atatgattatgcaaatttatagaagattactactgatacacacgtgatcagattactactgataccacgtagagattactactgataccacgttgagattactactgataagaacaaaatgttct

SEQ ID NO: 325 - DNA, 157 bp, synthetic construct
  source 1..157: mol_type = other DNA
caaaactaggtcaaaggtcaagattactactgataaactaggtcaaaggtcaagattactactgatatcgaagggcaggggtcaagggttcagtagattactactgataatattttagagaagaattaacctttagattactactgataccacgtag

SEQ ID NO: 326 - DNA, 140 bp, synthetic construct
  source 1..140: mol_type = other DNA
cgccccagcacacatgatcagaagattactactgatagtttatcagtgactagtcattgatagattactactgataaggtcaaagtccaagattactactgataccacgctgagattactactgatagttacttattctc

SEQ ID NO: 327 - DNA, 135 bp, synthetic construct
  source 1..135: mol_type = other DNA
ccacgtagagattactactgataatcacgtgatagattactactgataaaaaatggcgccaaaatgagattactactgatagtgtcaaaggtcaagattactactgataaaactaggtcacgaaaggtcaaagtc

SEQ ID NO: 328 - DNA, 184 bp, synthetic construct
  source 1..184: mol_type = other DNA
gcccccagggctgagtgacagaaaaacagagattactactgatagtctgctaagtcaataatcagaatagattactactgatattatgcatatgcataaagattactactgataaaactaggtcacgaaaggtcaaagtcagattactactgatacctcttctctgtgggtgaccagcgtccta

SEQ ID NO: 329 - DNA, 145 bp, synthetic construct
  source 1..145: mol_type = other DNA
ttatgcatatgcataaagattactactgatagccacgcccccagattactactgataaggtcaatgacctagattactactgatacctcttctctgtgggtgaccagcgtcctaagattactactgatattatgcatatgcataa

SEQ ID NO: 330 - DNA, 148 bp, synthetic construct
  source 1..148: mol_type = other DNA
aattatttattaccaagattactactgataaagaacattttgtacgtagattactactgatagtttatcagtgactagtcattgatagattactactgatacagagttcatgagagttcaagcagattactactgatatgtttacttt

SEQ ID NO: 331 - DNA, 146 bp, synthetic construct
  source 1..146: mol_type = other DNA
aattatggttctgggtgattcaagtaacaagattactactgataaaaaaatcaataatagattactactgataagaacatccctgtacaagattactactgataaggtcatcaggtcaagattactactgatagtgtcaaaggtca

SEQ ID NO: 332 - DNA, 147 bp, synthetic construct
  source 1..147: mol_type = other DNA
tggttaatattcaccagcagattactactgataacaggaagtagattactactgataaattatggttctgggtgattcaagtaacaagattactactgatacaggggtcatcgggttcaaacagattactactgataattgcacaat

SEQ ID NO: 333 - DNA, 161 bp, synthetic construct
  source 1..161: mol_type = other DNA
tgtttgttaaggcagattactactgataaaactaggtcacgaaaggtcaaagtcagattactactgataaggcagagggcagaaaggtcaagggagattactactgatactgacgtcagagattactactgatatcgaagggcaggggtcaagggttcagt

SEQ ID NO: 334 - DNA, 135 bp, synthetic construct
  source 1..135: mol_type = other DNA
gccacgcccccagattactactgataaggtcaatgacctagattactactgatagttacttattctcagattactactgataaactaggtcaaaggtcaagattactactgatacaggggtcatcgggttcaaac

SEQ ID NO: 335 - DNA, 138 bp, synthetic construct
  source 1..138: mol_type = other DNA
tattgattagattactactgataaattatggttctgggtgattcaagtaacaagattactactgatatccattgatttagagattactactgataatcaccccacagattactactgataaaaaatggcgccaaaatg

SEQ ID NO: 336 - DNA, 128 bp, synthetic construct
  source 1..128: mol_type = other DNA
aggtcaaaggtcaagattactactgatatccacgtggtattagattactactgataaaaaaatcaataatagattactactgataaaaaatggcgccaaaatgagattactactgatactgacgtcag

SEQ ID NO: 337 - DNA, 160 bp, synthetic construct
  source 1..160: mol_type = other DNA
tcgagcgctgggcaaaggtcacctgcagattactactgatagcccccagggctgagtgacagaaaaacagagattactactgataagaacaaaatgttctagattactactgataaagaacattttgtacgtagattactactgataaggtcaaagtcca

SEQ ID NO: 338 - DNA, 144 bp, synthetic construct
  source 1..144: mol_type = other DNA
aaaaatggcgccaaaatgagattactactgataaatatgcaaattagagattactactgataaggtcatcaggtcaagattactactgataaatagaggtcactaaaggtcaagcagattactactgatatacgcccacgcatt

SEQ ID NO: 339 - DNA, 131 bp, synthetic construct
  source 1..131: mol_type = other DNA
aggttaaaggtctagattactactgatatacgcccacgcattagattactactgataccacgttgagattactactgatagaaaaaaaaatcaatatcgggcctagattactactgatacaaacgtggttt

SEQ ID NO: 340 - DNA, 132 bp, synthetic construct
  source 1..132: mol_type = other DNA
ccacgttgagattactactgataaggcagagggcagaaaggtcaagggagattactactgataccatgaactttgagattactactgatagttaataatctacagattactactgataggttaataattaac

SEQ ID NO: 341 - DNA, 171 bp, synthetic construct
  source 1..171: mol_type = other DNA
aatatgcaaattagagattactactgatagaaaaaaaaatcaatatcgggcctagattactactgataaagaacattttgtacgtagattactactgatagcattacagactgggtgacagagtgagacagattactactgatatcgagcgcaggtcaaaaggtcacctgc

SEQ ID NO: 342 - DNA, 151 bp, synthetic construct
  source 1..151: mol_type = other DNA
ccatgaactttgagattactactgatacagagttcatgagagttcaagcagattactactgataaactaggtcaaaggtcaaagagattactactgatagcacgtcaagattactactgatagcccccagggctgagtgacagaaaaacag

SEQ ID NO: 343 - DNA, 147 bp, synthetic construct
  source 1..147: mol_type = other DNA
atcaccccacagattactactgatatcgaagggcaggggtcaagggttcagtagattactactgatacacacgtgatcagattactactgataaggtcaatgacctagattactactgatatcgagcgcaggtcaaaggtcacctgc

SEQ ID NO: 344 - DNA, 148 bp, synthetic construct
  source 1..148: mol_type = other DNA
gtgtcaaaggtcaagattactactgatatgggtccagagggcaaaaagattactactgatatgggtccagagggcaaaaagattactactgatagcaataaaatctgggtcacaggagttggaagattactactgataatcaccccac

SEQ ID NO: 345 - DNA, 143 bp, synthetic construct
  source 1..143: mol_type = other DNA
aaaaaatcaataatagattactactgataaggtcatcaggtcaagattactactgatatgtttgttaaggcagattactactgataaagaacaaaatgttcttagattactactgataaggcagagggcagaaaggtcaaggg

SEQ ID NO: 346 - DNA, 161 bp, synthetic construct; identical to SEQ ID NO: 138
  source 1..161: mol_type = other DNA

SEQ ID NO: 347 - DNA, 164 bp, synthetic construct
  source 1..164: mol_type = other DNA
tgatagccaactgcagctaataataaaccaagattactactgataaattatttattaccaagattactactgatagcattacagactgggtgacagagtgagacagattactactgatattatgcatatgcataaagattactactgataaaaaaatcaataat

SEQ ID NO: 348 - DNA, 156 bp, synthetic construct
  source 1..156: mol_type = other DNA
aacacgggaggtcaaagattgcgcccagattactactgatatacacgtaatcagattactactgatacaggggtcatcgggttcaaacagattactactgataaggcagagggcagaaaggtcaagggagattactactgatagttaataatctac

SEQ ID NO: 349 - DNA, 172 bp, synthetic construct
  source 1..172: mol_type = other DNA
gaaaaaaaaatcaatatcgggcctagattactactgatagcattacagactgggtgacagagtgagacagattactactgataaattatttattaccaagattactactgatatgggtccagagggcaaaaagattactactgatatggtatgattttgtaatggggtagga

SEQ ID NO: 350 - DNA, 140 bp, synthetic construct
  source 1..140: mol_type = other DNA
gcacgtcaagattactactgataaaaaatggcgccaaaatgagattactactgatatcgagcgcaggtcaaaaggtcacctgcagattactactgataacaggaagtagattactactgatatgggtccagagggcaaaa

SEQ ID NO: 351 - DNA, 152 bp, synthetic construct
  source 1..152: mol_type = other DNA
agtatggttaatgatctacagagattactactgataatatgattatgcaaatttatagaagattactactgataccacgctgagattactactgataattacaaaatagattactactgatatgatagccaactgcagctaataataaacca

SEQ ID NO: 352 - DNA, 137 bp, synthetic construct
  source 1..137: mol_type = other DNA
aactaggtcaaaggtcaagattactactgataaggtcaaaggtcaagattactactgatatacacgtaatcagattactactgataagaacaaaatgttctagattactactgataatcacgtgattatcacgtgat

SEQ ID NO: 353 - DNA, 148 bp, synthetic construct
  source 1..148: mol_type = other DNA
cctcttctctgtgggtgaccagcgtcctaagattactactgataaggtcaaagtccaagattactactgatatgacgtcaagattactactgatagcccccagggctgagtgacagaaaaacagagattactactgataacaggaagt

SEQ ID NO: 354 - DNA, 177 bp, synthetic construct
  source 1..177: mol_type = other DNA
agaacaaaatgttctagattactactgatatgatagccaactgcagctaataataaaccaagattactactgatagcccccagggctgagtgacagaaaaacagagattactactgataatcacgtgattatcacgtgatagattactactgatacgccccagcacacatgatcaga

SEQ ID NO: 355 - DNA, 147 bp, synthetic construct
  source 1..147: mol_type = other DNA
tggtatgattttgtaatggggtaggaagattactactgataattgcacaatagattactactgatacctcttctctgtgggtgaccagcgtcctaagattactactgataccacgtagagattactactgataaggtcatcaggtca

SEQ ID NO: 356 - DNA, 156 bp, synthetic construct
  source 1..156: mol_type = other DNA
aggtcatcaggtcaagattactactgatatccattgatttagagattactactgatacagaggtcacagagttcaagcagattactactgataatatgattatgcaaatttatagaagattactactgataatattttagagaagaattaaccttt

SEQ ID NO: 357 - DNA, 141 bp, synthetic construct
  source 1..141: mol_type = other DNA
aggtcaatgacctagattactactgataaccggaagtacttccggtagattactactgatatcgagcgcaggtcaaaggtcacctgcagattactactgataagaacatccctgtacaagattactactgatatgacgtca

SEQ ID NO: 358 - DNA, 127 bp, synthetic construct
  source 1..127: mol_type = other DNA
aagaacaaaatgttcttagattactactgataccacgtagagattactactgataagaacattttgtacgagattactactgataaggtcaaaggtcaagattactactgataaatatgcaaattag

SEQ ID NO: 359 - DNA, 126 bp, synthetic construct
  source 1..126: mol_type = other DNA
tacacgtaatcagattactactgatactgacgtcagagattactactgatagtgtcaaaggtcaagattactactgataaacccggaagtgagattactactgataaactaggtcaaaggtcaaag

SEQ ID NO: 360 - DNA, 163 bp, synthetic construct
  source 1..163: mol_type = other DNA
caggggtcatcgggttcaaacagattactactgatatcgagcgcaggtcaaaaggtcacctgcagattactactgatatcgagcgctgggcaaaggtcacctgcagattactactgatacaaacgtggtttagattactactgataaagaacaaaatgttctt

SEQ ID NO: 361 - DNA, 142 bp, synthetic construct
  source 1..142: mol_type = other DNA
atattttagagaagaattaacctttagattactactgatacaggggtcatcgggttcaaacagattactactgataatcacgtgatagattactactgatatccattgatttagagattactactgatatccattgatttag

SEQ ID NO: 362 - DNA, 136 bp, synthetic construct
  source 1..136: mol_type = other DNA
agaacatccctgtacaagattactactgataaagaacaaaatgttcttagattactactgatacagagttcatgagagttcaagcagattactactgatagccacgcccccagattactactgatattgtttactt

SEQ ID NO: 363 - DNA, 137 bp, synthetic construct
  source 1..137: mol_type = other DNA
cagaggtcacagagttcaagcagattactactgatatggtatgattttgtaatggggtaggaagattactactgataattacaaaatagattactactgatattgtttacttagattactactgataatcacgtgat

SEQ ID NO: 364 - DNA, 143 bp, synthetic construct
  source 1..143: mol_type = other DNA
agaacattttgtacgagattactactgatacaaacgtggtttagattactactgatacaaaactaggtcaaaggtcaagattactactgataattgcacaatagattactactgatatcgagcgctgggcaaaggtcacctgc

SEQ ID NO: 365 - DNA, 143 bp, synthetic construct
  source 1..143: mol_type = other DNA
gttaataatctacagattactactgataagaacatccctgtacaagattactactgataaacacgggaggtcaaagattgcgcccagattactactgatatgtttgttaaggcagattactactgataaattatttattacca

SEQ ID NO: 366 - DNA, 130 bp, synthetic construct
  source 1..130: mol_type = other DNA
aagaacattttgtacgtagattactactgataggttaataattaacagattactactgataaccggaagtacttccggtagattactactgatatgacgtcaagattactactgatatgtttgttaaggc

SEQ ID NO: 367 - DNA, 151 bp, synthetic construct
  source 1..151: mol_type = other DNA
attgcacaatagattactactgataccacgctgagattactactgatagcaataaaatctgggtcacaggagttggaagattactactgatacaaaactaggtcaaaggtcaagattactactgatagaaaaaaaaatcaatatcgggcct

SEQ ID NO: 368 - DNA, 130 bp, synthetic construct
  source 1..130: mol_type = other DNA
gcattacagactgggtgacagagtgagacagattactactgatatgacgtcaagattactactgataaggtcaaaggtcaagattactactgatatattgattagattactactgataccatgaactttg

SEQ ID NO: 369 - DNA, 152 bp, synthetic construct
  source 1..152: mol_type = other DNA
aggtcaaagtccaagattactactgataaactaggtcaaaggtcaaagagattactactgatatggtatgattttgtaatggggtaggaagattactactgatatggttaatattcaccagcagattactactgataagaacattttgtacg

SEQ ID NO: 370 - DNA, 147 bp, synthetic construct
  source 1..147: mol_type = other DNA
gtttatcagtgactagtcattgatagattactactgatagcacgtcaagattactactgataatatgattatgcaaatttatagaagattactactgatatgtttactttagattactactgatacagagttcatgagagttcaagc

SEQ ID NO: 371 - DNA, 136 bp, synthetic construct
  source 1..136: mol_type = other DNA
tccacgtggtattagattactactgatacgccccagcacacatgatcagaagattactactgataattgtgcaatagattactactgataattgtgcaatagattactactgatacagaggtcacagagttcaagc

SEQ ID NO: 372 - DNA, 142 bp, synthetic construct
  source 1..142: mol_type = other DNA
atcacgtgattatcacgtgatagattactactgatatgtttgttaaggcagattactactgataacaggaagtagattactactgataaacacgggaggtcaaagattgcgcccagattactactgataaggttaaaggtct

SEQ ID NO: 373 - DNA, 147 bp, synthetic construct
  source 1..147: mol_type = other DNA
gcaataaaatctgggtcacaggagttggaagattactactgataagaacaaaatgttctagattactactgatacaaacgtggtttagattactactgatacagaggtcacagagttcaagcagattactactgataattgtgcaat

SEQ ID NO: 374 - DNA, 144 bp, synthetic construct
  source 1..144: mol_type = other DNA
cacttccgccggaagtgagattactactgataaggttaaaggtctagattactactgataaatatgcaaattagagattactactgataaattatggttctgggtgattcaagtaacaagattactactgatacacacgtgatc

SEQ ID NO: 375 - DNA, 160 bp, synthetic construct
  source 1..160: mol_type = other DNA
tcgaagggcaggggtcaagggttcagtagattactactgataagtatggttaatgatctacagagattactactgataaaactaggtcacgaaaggtcaaagtcagattactactgataaggtcaaagtccaagattactactgataaggtcaatgacct

SEQ ID NO: 376 - DNA, 155 bp, synthetic construct
  source 1..155: mol_type = other DNA
gtctgctaagtcaataatcagaatagattactactgataaacccggaagtgagattactactgatatccacgtggtattagattactactgataaactaggtcaaaggtcaaagagattactactgataaacacgggaggtcaaagattgcgccc

SEQ ID NO: 377 - DNA, 114 bp, synthetic construct
  source 1..114: mol_type = other DNA
ccacgctgagattactactgatatgtttactttagattactactgataatcaccccacagattactactgataaattatttattaccaagattactactgatagccacgccccc

SEQ ID NO: 378 - DNA, 160 bp, synthetic construct
  source 1..160: mol_type = other DNA
aatagaggtcactaaaggtcaagcagattactactgatattgtttacttagattactactgataatattttagagaagaattaacctttagattactactgatagtttatcagtgactagtcattgatagattactactgataaagaacattttgtacgt

SEQ ID NO: 379 - DNA, 146 bp, synthetic construct
  source 1..146: mol_type = other DNA
tcgagcgcaggtcaaaaggtcacctgcagattactactgatagtgtcaaaggtcaagattactactgatagaaaaaaaaatcaatatcgggcctagattactactgataaatatgcaaattagagattactactgatagcacgtca

SEQ ID NO: 380 - DNA, 132 bp, synthetic construct
  source 1..132: mol_type = other DNA
aacccggaagtgagattactactgatagttacttattctcagattactactgatacgccccagcacacatgatcagaagattactactgatagttacttattctcagattactactgataaacccggaagtg

SEQ ID NO: 381 - DNA, 155 bp, synthetic construct
  source 1..155: mol_type = other DNA
aactaggtcaaaggtcaaagagattactactgatatggttaatattcaccagcagattactactgatagttaataatctacagattactactgatatcgaagggcaggggtcaagggttcagtagattactactgatacacttccgccggaagtg

SEQ ID NO: 382 - DNA, 123 bp, synthetic construct
  source 1..123: mol_type = other DNA
ttgtttacttagattactactgataccacgttgagattactactgataattgcacaatagattactactgataaccggaagtacttccggtagattactactgataaactaggtcaaaggtca

SEQ ID NO: 383 - DNA, 136 bp, synthetic construct
  source 1..136: mol_type = other DNA
gttacttattctcagattactactgataattgtgcaatagattactactgatatacgcccacgcattagattactactgataagaacattttgtacgagattactactgataaatagaggtcactaaaggtcaagc

SEQ ID NO: 384 - DNA, 133 bp, synthetic construct
  source 1..133: mol_type = other DNA
accggaagtacttccggtagattactactgatagttaataatctacagattactactgatagcacgtcaagattactactgatatcgagcgcaggtcaaaggtcacctgcagattactactgataccacgttg

SEQ ID NO: 385 - DNA, 148 bp, synthetic construct
  source 1..148: mol_type = other DNA
cacacgtgatcagattactactgatacacttccgccggaagtgagattactactgataaagaacaaaatgttcttagattactactgatatgatagccaactgcagctaataataaaccaagattactactgatatccacgtggtatt

SEQ ID NO: 386 - DNA, 151 bp, synthetic construct
  source 1..151: mol_type = other DNA
attacaaaatagattactactgataaatagaggtcactaaaggtcaagcagattactactgatagccacgcccccagattactactgatacacttccgccggaagtgagattactactgatagcaataaaatctgggtcacaggagttgga

SEQ ID NO: 387 - DNA, 166 bp, synthetic construct
  source 1..166: mol_type = other DNA
aggcagagggcagaaaggtcaagggagattactactgataatattttagagaagaattaacctttagattactactgataaatagaggtcactaaaggtcaagcagattactactgataggttaataattaacagattactactgatatggttaatattcaccagc

SEQ ID NO: 388 - DNA, 145 bp, synthetic construct
  source 1..145: mol_type = other DNA
cagagttcatgagagttcaagcagattactactgataattacaaaatagattactactgataaggttaaaggtctagattactactgatacacacgtgatcagattactactgatagcattacagactgggtgacagagtgagac

SEQ ID NO: 389 - DNA, 157 bp, synthetic construct; identical to SEQ ID NO: 139
  source 1..157: mol_type = other DNA

SEQ ID NO: 390 - DNA, 117 bp, synthetic construct
  source 1..117: mol_type = other DNA
tgggtccagagggcaaaaagattactactgataatcaccccacagattactactgatattgtttacttagattactactgatatacacgtaatcagattactactgatatattgatt

SEQ ID NO: 391 - DNA, 120 bp, synthetic construct
  source 1..120: mol_type = other DNA
ttatataaaataagattactactgatattatataaaataagattactactgatattatataaaataagattactactgatattatataaaataagattactactgatattatataaaata

SEQ ID NO: 392 - DNA, 128 bp, synthetic construct
  source 1..128: mol_type = other DNA
ttatataaaataagattactactgatattatataaaataagattactactgataaactaggtcaaaggtcaaagagattactactgatattatataaaataagattactactgatattatataaaata

SEQ ID NO: 393 - DNA, 136 bp, synthetic construct
  source 1..136: mol_type = other DNA
aactaggtcaaaggtcaaagagattactactgatattatataaaataagattactactgatattatataaaataagattactactgatattatataaaataagattactactgataaactaggtcaaaggtcaaag

SEQ ID NO: 394 - DNA, 190 bp, synthetic construct
  source 1..190: mol_type = other DNA
tggtatgattttgtaatggggtaggaagattactactgatatggtatgattttgtaatggggtaggaagattactactgatatggtatgattttgtaatggggtaggaagattactactgatatggtatgattttgtaatggggtaggaagattactactgatatggtatgattttgtaatggggtagga

SEQ ID NO: 395 - DNA, 184 bp, synthetic construct
  source 1..184: mol_type = other DNA
tggtatgattttgtaatggggtaggaagattactactgatatggtatgattttgtaatggggtaggaagattactactgataaactaggtcaaaggtcaaagagattactactgatatggtatgattttgtaatggggtaggaagattactactgatatggtatgattttgtaatggggtagga

SEQ ID NO: 396 - DNA, 178 bp, synthetic construct
  source 1..178: mol_type = other DNA
aactaggtcaaaggtcaaagagattactactgatatggtatgattttgtaatggggtaggaagattactactgatatggtatgattttgtaatggggtaggaagattactactgatatggtatgattttgtaatggggtaggaagattactactgataaactaggtcaaaggtcaaag

SEQ ID NO: 397 - DNA, 181 bp, synthetic construct
  source 1..181: mol_type = other DNA
tggtatgattttgtaatggggtaggaagattactactgatatggtatgattttgtaatggggtaggaagattactactgataaagaacaaaatgttcttagattactactgatatggtatgattttgtaatggggtaggaagattactactgatatggtatgattttgtaatggggtagga

SEQ ID NO: 398 - DNA, 172 bp, synthetic construct
  source 1..172: mol_type = other DNA
aagaacaaaatgttcttagattactactgatatggtatgattttgtaatggggtaggaagattactactgatatggtatgattttgtaatggggtaggaagattactactgatatggtatgattttgtaatggggtaggaagattactactgataaagaacaaaatgttctt

SEQ ID NO: 399 - DNA, 175 bp, synthetic construct
  source 1..175: mol_type = other DNA
aagaacaaaatgttcttagattactactgatatggtatgattttgtaatggggtaggaagattactactgatatggtatgattttgtaatggggtaggaagattactactgatatggtatgattttgtaatggggtaggaagattactactgataaactaggtcaaaggtcaaag

SEQ ID NO: 400 - DNA, 170 bp, synthetic construct
  source 1..170: mol_type = other DNA
aagaacaaaatgttcttagattactactgatatggtatgattttgtaatggggtaggaagattactactgataagtatggttaatgatctacagagattactactgatatggtatgattttgtaatggggtaggaagattactactgataaactaggtcaaaggtcaaag

SEQ ID NO: 401 - DNA, 175 bp, synthetic construct
  source 1..175: mol_type = other DNA
aagaacaaaatgttcttagattactactgatatggtatgattttgtaatggggtaggaagattactactgatatcgagcgcaggtcaaaggtcacctgcagattactactgatatggtatgattttgtaatggggtaggaagattactactgataaactaggtcaaaggtcaaag

SEQ ID NO: 402 - DNA, 162 bp, synthetic construct
  source 1..162: mol_type = other DNA
aagaacaaaatgttcttagattactactgatatggtatgattttgtaatggggtaggaagattactactgatatccacgtggtattagattactactgatatggtatgattttgtaatggggtaggaagattactactgataaactaggtcaaaggtcaaag

SEQ ID NO: 403 - DNA, 148 bp, synthetic construct
  source 1..148: mol_type = other DNA
aagaacaaaatgttcttagattactactgatattatataaaataagattactactgatatccacgtggtattagattactactgatatggtatgattttgtaatggggtaggaagattactactgataaactaggtcaaaggtcaaag

SEQ ID NO: 404 - DNA, 152 bp, synthetic construct
  source 1..152: mol_type = other DNA
agtatggttaatgatctacagagattactactgatattatataaaataagattactactgatatccacgtggtattagattactactgatatggtatgattttgtaatggggtaggaagattactactgataaactaggtcaaaggtcaaag

SEQ ID NO: 405 - DNA, 157 bp, synthetic construct
  source 1..157: mol_type = other DNA
tcgagcgcaggtcaaaggtcacctgcagattactactgatattatataaaataagattactactgatatccacgtggtattagattactactgatatggtatgattttgtaatggggtaggaagattactactgataaactaggtcaaaggtcaaag

SEQ ID NO: 406 - DNA, 152 bp, synthetic construct
  source 1..152: mol_type = other DNA
atcacgtgattatcacgtgatagattactactgatattatataaaataagattactactgatatccacgtggtattagattactactgatatggtatgattttgtaatggggtaggaagattactactgataaactaggtcaaaggtcaaag

SEQ ID NO: 407 - DNA, 150 bp, synthetic construct
  source 1..150: mol_type = other DNA
atcacgtgattatcacgtgatagattactactgataatcaccccacagattactactgatatccacgtggtattagattactactgatatggtatgattttgtaatggggtaggaagattactactgataaactaggtcaaaggtcaaag

SEQ ID NO: 408 - DNA, 159 bp, synthetic construct
  source 1..159: mol_type = other DNA
gcccccagggctgagtgacagaaaaacagagattactactgatatggtatgattttgtaatggggtaggaagattactactgataatcacgtgattatcacgtgatagattactactgatatccacgtggtattagattactactgataatcaccccac

SEQ ID NO: 409 - DNA, 170 bp, synthetic construct
  source 1..170: mol_type = other DNA
gcccccagggctgagtgacagaaaaacagagattactactgatatggtatgattttgtaatggggtaggaagattactactgataatcacgtgattatcacgtgatagattactactgatatccacgtggtattagattactactgataagtatggttaatgatctacag

SEQ ID NO: 410 - DNA, 175 bp, synthetic construct
  source 1..175: mol_type = other DNA
gcccccagggctgagtgacagaaaaacagagattactactgatatggtatgattttgtaatggggtaggaagattactactgataagtatggttaatgatctacagagattactactgatatccacgtggtattagattactactgatatcgagcgcaggtcaaaggtcacctgc

SEQ ID NO: 411 - DNA, 110 bp, synthetic construct; identical to SEQ ID NO: 140
  source 1..110: mol_type = other DNA

SEQ ID NO: 412 - DNA, 378 bp, synthetic construct
  source 1..378: mol_type = other DNA
acacaggaagtgacaattttcgcgcggttttaggcggatgttgtagtaaatttgggcgtaaccgagtaagatttggccattttcgcgggaaaactgaataagaggaagtgaaatctgaataattttagattactactgatagtggttgtacagacgccatcttggaatccaatatgtctgccggctcagtcatgcctgcgctgcgcgcagcgcgctgcgcgcgcgcatgatctaatcgccggcagacatattggattccaagatggcgtctgtacaaccacgtcacatataagattactactgataggggaggctgctggtgaatattaaccaaggtcacccccagttatcggaggagcaaacagggactaagtccac

SEQ ID NO: 413 - DNA, 60 bp, synthetic construct
  source 1..60: mol_type = other DNA
agattactactgataagattactactgataagattactactgataagattactactgata

SEQ ID NO: 414 - protein, 28 aa, synthetic construct
PAAKRVKLDASQGTKRSYEQMETDGERQ

SEQ ID NO: 415 - protein, 35 aa, synthetic construct; identical to SEQ ID NO: 201

SEQ ID NO: 416 - protein, 38 aa, synthetic construct; identical to SEQ ID NO: 200

SEQ ID NO: 417 - protein, 66 aa, synthetic construct; identical to SEQ ID NO: 213

SEQ ID NO: 418 - protein, 26 aa, synthetic construct
PKKKRKVASQGTKRSYEQMETDGERQ

SEQ ID NO: 419 - protein, 27 aa, synthetic construct
MASQGTKRSYEQMETDGERQPKKKRKV

SEQ ID NO: 420 - protein, 37 aa, synthetic construct; identical to SEQ ID NO: 214

SEQ ID NO: 421 - protein, 29 aa, synthetic construct
MASQGTKRSYEQMETDGERQPAAKRVKLD

SEQ ID NO: 422 - protein, 37 aa, synthetic construct
MPAAKRVKLDGSGSGSGSASQGTKRSYEQMETDGERQ

SEQ ID NO: 423 - protein, 20 aa, synthetic construct
PAAKRVKLDSQGTKRSYEQM

SEQ ID NO: 424 - protein, 47 aa, synthetic construct
PAAKRVKLDSQGTKRSYEQMTKRSYEQMSQGTKRSYEQMTKRSYEQM

SEQ ID NO: 425 - protein, 48 aa, synthetic construct
GGGGSGGGGSGGGGSGGGGSPAAKRVKLDASQGTKRSYEQMETDGERQ

SEQ ID NO: 426 - protein, 40 aa, synthetic construct
RKTRGNEGRWFNRDNIGKRQSGSGSTATGSGPAAKRVKLD

SEQ ID NO: 427 - protein, 63 aa, synthetic construct
MGGGGSGGGGSGGGGSGGGGSAARLHRFKNKGKDSTEMRRRRIEVNVELRKAKKDDQMLKRRN

SEQ ID NO: 428 - protein, 28 aa, synthetic construct
PAAKRAKLDASQGTKRSYEQMETDGERQ

SEQ ID NO: 429 - protein, 29 aa, synthetic construct
PAAKRAKLDASQGTKRSYEQMETDPPPRG

SEQ ID NO: 430 - protein, 59 aa, synthetic construct
SGTNGTKRFDLGVYYHKNNKLALHRSYLTPTNSPRRARSVASQGTKRSYEQMETDGERQ

SEQ ID NO: 431 - protein, 39 aa, synthetic construct
PAAKRVKLDPLSSIFSRIGDPSQGTKRSYEQMETDGERQ

SEQ ID NO: 432 - protein, 37 aa, synthetic construct
PAAKRVKLDPLSSIFSRIGDPSTPPKKKRKVTDGERQ

SEQ ID NO: 433 - protein, 43 aa, synthetic construct
PAAKRVKLDRQIKIWFQNRRMKWKKSQGTKRSYEQMETDGERQ

SEQ ID NO: 434 - protein, 28 aa, synthetic construct
PAAKRVKLDATQGTKRSYEQMETDGERQ

SEQ ID NO: 435 - protein, 29 aa, synthetic construct
PKKKRKVEDPASQGTKRSYEQMETDGERQ

SEQ ID NO: 436 - protein, 28 aa, synthetic construct
PAAKRVKLDASPKKKRKYEQMETDGERQ

SEQ ID NO: 437 - protein, 38 aa, synthetic construct
PAAKRVKLDPKKKRKVEDPASQGTKRSYEQMETDGERQ

SEQ ID NO: 438 - protein, 28 aa, synthetic construct
PKKKRKVEDPSQGTKRSYEQMETDGERQ

SEQ ID NO: 439 - protein, 36 aa, synthetic construct
PAAKRVKLDFGNYNNQSSNFGPMKGGNFGGRSSGPY

SEQ ID NO: 440 - protein, 29 aa, synthetic construct
MASQGTKRSYEQMETDGERQPAAKRVKLD

SEQ ID NO: 441 - protein, 10 aa, synthetic construct
PKKKRKVEDP

SEQ ID NO: 442 - protein, 47 aa, synthetic construct
KKKSSSDDEATADSQHSTPPKKKRKVEDPKDFPSELLSLPPLERLTL

SEQ ID NO: 443 - protein, 37 aa, synthetic construct
PAAKRVKLDASQGTKRSYEQMETDGERQLPPLERLTL

SEQ ID NO: 444 - DNA, 32 bp, synthetic construct; identical to SEQ ID NO: 144
  source 1..32: mol_type = other DNA

SEQ ID NO: 445 - DNA, 14 bp, synthetic construct
  source 1..14: mol_type = other DNA
acaagaaagtttgt

SEQ ID NO: 446 - DNA, 14 bp, synthetic construct
  source 1..14: mol_type = other DNA
acaagatacattgt

SEQ ID NO: 447 - DNA, 14 bp, synthetic construct
  source 1..14: mol_type = other DNA
acaagaaaaactgt

SEQ ID NO: 448 - protein, 95 aa, Escherichia coli Phage 434
MSISSRVKSKRIQLGLNQAELAQKVGTTQQSIEQLENGKTKRPRFLPELASALGVSVDWLLNGTSDSNVRFVGHVEPKGKYPLISMVRAGSWCEA

SEQ ID NO: 449 - RNA, 285 nt, synthetic construct
  source 1..285: mol_type = other RNA
atgagcatcagcagccgcgtgaagagcaagcgcatccagctgggcctgaaccaggccgagctggcccagaaggtgggcaccacccagcagagcatcgagcagctggagaacggcaagaccaagcgcccccgcttcctgcccgagctggccagcgccctgggcgtgagcgtggactggctgctgaacggcaccagcgacagcaacgtgcgcttcgtgggccacgtggagcccaagggcaagtaccccctgatcagcatggtgcgcgccggcagctggtgcgaggcc

SEQ ID NO: 450 - DNA, 11 bp, synthetic construct
  source 1..11: mol_type = other DNA
agtgactcatt

SEQ ID NO: 451 - protein, 281 aa, Saccharomyces cerevisiae [sequence omitted]

SEQ ID NO: 452 - RNA, 843 nt, synthetic construct
  source 1..843: mol_type = other RNA
atgtccgaatatcaaccatctttatttgcattgaatccaatggggttttccccacttgatggatccaaatccacaaatgaaaatgtttctgcatcaacaagtactgcaaagccaatggtcgggcaattgatctttgataaatttattaagacagaagaagatccgattataaaacaagatactcccagtaatttggattttgattttgcgttgcctcaaacagctacagctccagatgctaaaacagtcttaccaattccagaactcgatgatgctgtcgtcgaaagtttcttttcaagttccacggatagtacaccaatgtttgagtatgaaaatttggaagataattcaaaagaatggacttctctctttgacaatgacattccagtcactactgatgatgtttcccttgcagataaagctattgaatctacagaagaagtatctttagtcccatccaatcttgaagtttccactacgagttttctccctacgcctgtacttgaagatgcaaaattgacgcaaacgcggaaagtcaagaaaccaaattctgttgtcaagaaatctcatcatgttggaaaagatgatgaatctaggctcgatcatcttggagttgtcgcttataataggaaacaaagatctataccactctcccctatagtcccagaaagtagtgatcctgctgctttaaaacgggctaggaatacagaagcggctcgaaggtccagggcccggaaattacaacggatgaaacaactcgaagataaagtagaagaattgctttctaagaattatcatcttgagaatgaagttgcacggttgaagaaactcgttggggaacgg

SEQ ID NO: 453 - DNA, 17 bp, synthetic construct
  source 1..17: mol_type = other DNA
ttgttatccgctcacaa

SEQ ID NO: 454 - protein, 360 aa, Escherichia coli
MKPVTLYDVAEYAGVSYQTVSRVVNQASHVSAKTREKVEAAMAELNYIPNRVAQQLAGKQSLLIGVATSSLALHAPSQIVAAIKSRADQLGASVVVSMVERSGVEACKAAVHNLLAQRVSGLIINYPLDDQDAIAVEAACTNVPALFLDVSDQTPINSIIFSHEDGTRLGVEHLVALGHQQIALLAGPLSSVSARLRLAGWHKYLTRNQIQPIAEREGDWSAMSGFQQTMQMLNEGIVPTAMLVANDQMALGAMRAITESGLRVGADISVVGYDDTEDSSCYIPPLTTIKQDFRLLGQTSVDRLLQLSQGQAVKGNQLLPVSLVKRKTTLAPNTQTASPRALADSLMQLARQVSRLESGQ

SEQ ID NO: 455 - DNA, 12 bp, synthetic construct
  source 1..12: mol_type = other DNA
aaactgcaaaag